SEQUENCE LISTING (1195 sequences)

SEQ ID NO: 1 - protein, 12 aa, Artificial Sequence
  synthetic peptide
VRLIVAVRIWRR

SEQ ID NO: 2 - protein, 12 aa, Artificial Sequence
  synthetic peptide
RARIGIWKKWWA

SEQ ID NO: 3 - protein, 12 aa, Artificial Sequence
  synthetic peptide
  misc_feature: D-amino acid
RRWIRVAVILRV

SEQ ID NO: 4 - protein, 12 aa, Artificial Sequence
  synthetic peptide
  misc_feature: D-amino acid
VQWRAIRVRVIR

SEQ ID NO: 5 - protein, 12 aa, Artificial Sequence
  synthetic peptide
  misc_feature: D-amino acid
KRIRWVILWRQV

SEQ ID NO: 6 - protein, 12 aa, Artificial Sequence
  synthetic peptide
  misc_feature (12)..(12): Xaa is L-Ornithine
VRLIVAVRIWRX

SEQ ID NO: 7 - protein, 12 aa, Artificial Sequence
  synthetic peptide
  misc_feature (1)..(1): Xaa is L-2,3-diaminopropionic acid
XRLIVAVRIWRR

SEQ ID NO: 8 - protein, 12 aa, Artificial Sequence
  synthetic peptide
  misc_feature (12)..(12): Xaa is L-Homoarginine
VRLIVAVRIWRX

SEQ ID NO: 9 - protein, 12 aa, Artificial Sequence
  synthetic peptide
  misc_feature (6)..(6): Xaa is L-Ornithine
VRLIVXVRIWRR

SEQ ID NO: 10 - protein, 12 aa, Artificial Sequence
  synthetic peptide
  misc_feature (12)..(12): Xaa is L-2,3-diaminopropionic acid
VRLIVAVRIWRX

SEQ ID NO: 11 - protein, 12 aa, Artificial Sequence
  synthetic peptide
  misc_feature (11)..(11): Xaa is L-Homoarginine
VRLIVAVRIWXR

SEQ ID NO: 12 - protein, 12 aa, Artificial Sequence
  synthetic peptide
  misc_feature (11)..(11): Xaa is L-Ornithine
VRLIVAVRIWXR

SEQ ID NO: 13 - protein, 12 aa, Artificial Sequence
  synthetic peptide
  misc_feature (11)..(11): Xaa is L-2,3-diaminopropionic acid
VRLIVAVRIWXR

SEQ ID NO: 14 - protein, 12 aa, Artificial Sequence
  synthetic peptide
  misc_feature (1)..(1): Xaa is L-Ornithine
XRLIVAVRIWRR

SEQ ID NO: 15 - protein, 12 aa, Artifical Sequence
  synthetic peptide
  misc_feature (8)..(8): Xaa is L-Homoarginine
VRLIVAVXIWRR

SEQ ID NO: 16 - protein, 12 aa, Artificial Sequence
  synthetic peptide
  misc_feature (8)..(8): Xaa is L-Ornithine
VRLIVAVXIWRR

SEQ ID NO: 17 - protein, 12 aa, Artificial Sequence
  synthetic peptide
  misc_feature (1)..(1): Xaa is L-2-Amino-4-guanidinobutyric acid
XRLIVAVRIWRR

SEQ ID NO: 18 - protein, 12 aa, Artificial Sequence
  synthetic peptide
  misc_feature (10)..(10): Xaa is L-2,3-diaminopropionic acid
VRLIVAVRIXRR

SEQ ID NO: 19 - protein, 12 aa, Artificial Sequence
  synthetic peptide
  misc_feature (1)..(1): Xaa is L-2-amino-3-guanidinopropionic acid
XRLIVAVRIWRR

SEQ ID NO: 20 - protein, 12 aa, Artificial Sequence
  synthetic peptide
  misc_feature (6)..(6): Xaa is L-Homoarginine
VRLIVXVRIWRR

SEQ ID NO: 21 - protein, 12 aa, Artificial Sequence
  synthetic peptide
  misc_feature (1)..(1): Xaa is L-2,4-diaminobutyric acid
XRLIVAVRIWRR

SEQ ID NO: 22 - protein, 12 aa, Artificial Sequence
  synthetic peptide
  misc_feature (2)..(2): Xaa is L-Ornithine
VXLIVAVRIWRR

SEQ ID NO: 23 - protein, 12 aa, Artificial Sequence
  synthetic peptide
  misc_feature (1)..(1): Xaa is L-Homoarginine
XRLIVAVRIWRR

SEQ ID NO: 24 - protein, 12 aa, Artificial Sequence
  synthetic peptide
VIKWLLKILRAI

SEQ ID NO: 25 - protein, 12 aa, Artificial Sequence
  synthetic peptide
ILVRWIRWRIQW

SEQ ID NO: 26 - protein, 12 aa, Artificial Sequence
  synthetic peptide
WKKVQWLKRLLL

SEQ ID NO: 27 - protein, 12 aa, Artificial Sequence
  synthetic peptide
IQRWWKVWLKVI

SEQ ID NO: 28 - protein, 12 aa, Artificial Sequence
  synthetic peptide
RRQWRGWVRIWL

SEQ ID NO: 29 - protein, 12 aa, Artificial Sequence
  synthetic peptide
IWLRLKVVLKRK

SEQ ID NO: 30 - protein, 12 aa, Artificial Sequence
  synthetic peptide
VLKIKVKIWVVK

SEQ ID NO: 31 - protein, 12 aa, Artificial Sequence
  synthetic peptide
KKWQLLIKWKLR

SEQ ID NO: 32 - protein, 12 aa, Artificial Sequence
  synthetic peptide
AVAKWALKLWKQ

SEQ ID NO: 33 - protein, 12 aa, Artificial Sequence
  synthetic peptide
QLARLARVVWGL

SEQ ID NO: 34 - protein, 12 aa, Artificial Sequence
  synthetic peptide
VLQIKKVLRLLL

SEQ ID NO: 35 - protein, 12 aa, Artificial Sequence
  synthetic peptide
RVKAIKWRKIVV

SEQ ID NO: 36 - protein, 12 aa, Artificial Sequence
  synthetic peptide
LWQLWLKLKLKG

SEQ ID NO: 37 - protein, 12 aa, Artificial Sequence
  synthetic peptide
KIQRRAWKQWRK

SEQ ID NO: 38 - protein, 12 aa, Artificial Sequence
  synthetic peptide
KIVIRIILQVIK

SEQ ID NO: 39 - protein, 12 aa, Artificial Sequence
  synthetic peptide
AVKWLGWILAKK

SEQ ID NO: 40 - protein, 12 aa, Artificial Sequence
  synthetic peptide
LAGLIVKWAGVR

SEQ ID NO: 41 - protein, 12 aa, Artificial Sequence
  synthetic peptide
WVGVIIKWGLKL

SEQ ID NO: 42 - protein, 12 aa, Artificial Sequence
  synthetic peptide
WQGWAKIWVVRI

SEQ ID NO: 43 - protein, 12 aa, Artificial Sequence
  synthetic peptide
LIVIQLLKKWWK

SEQ ID NO: 44 - protein, 12 aa, Artificial Sequence
  synthetic peptide
RRIIKILLWKLR

SEQ ID NO: 45 - protein, 12 aa, Artificial Sequence
  synthetic peptide
IAWQLLWGWRVR

SEQ ID NO: 46 - protein, 12 aa, Artificial Sequence
  synthetic peptide
VQRIIWLRVKIV

SEQ ID NO: 47 - protein, 12 aa, Artificial Sequence
  synthetic peptide
IKIIWKALGQVI

SEQ ID NO: 48 - protein, 12 aa, Artificial Sequence
  synthetic peptide
IQLKLIWVKRKW

SEQ ID NO: 49 - protein, 12 aa, Artificial Sequence
  synthetic peptide
VIKVLIKRWLKL

SEQ ID NO: 50 - protein, 12 aa, Artificial Sequence
  synthetic peptide
VQWIQIVVWRKR

SEQ ID NO: 51 - protein, 12 aa, Artificial Sequence
  Synthetic peptide
GLIIKIIKKRLW

SEQ ID NO: 52 - protein, 12 aa, Artificial Sequence
  synthetic peptide
VKGAIKRGIWVK

SEQ ID NO: 53 - protein, 12 aa, Artificial Sequence
  synthetic peptide
KVQIIKQLIAKK

SEQ ID NO: 54 - protein, 12 aa, Artificial Sequence
  synthetic peptide
KRLQWVKVKKIR

SEQ ID NO: 55 - protein, 12 aa, Artificial Sequence
  synthetic peptide
IVKWIAQWKLVG

SEQ ID NO: 56 - protein, 12 aa, Artificial Sequence
  synthetic peptide
KKQKKIWRRILV

SEQ ID NO: 57 - protein, 12 aa, Artificial Sequence
  synthetic peptide
GRVLKIVWRKGR

SEQ ID NO: 58 - protein, 12 aa, Artificial Sequence
  synthetic peptide
RQVRVKRWRARW

SEQ ID NO: 59 - protein, 12 aa, Artificial Sequence
  synthetic peptide
KVVWWKVIIKVL

SEQ ID NO: 60 - protein, 12 aa, Artificial Sequence
  synthetic peptide
ALAIKVWIKILQ

SEQ ID NO: 61 - protein, 12 aa, Artificial Sequence
  synthetic peptide
IRILVLRKAIIV

SEQ ID NO: 62 - protein, 12 aa, Artificial Sequence
  synthetic peptide
IVKKVKLIWGVK

SEQ ID NO: 63 - protein, 12 aa, Artificial Sequence
  synthetic peptide
VVGLRVRWVRLW

SEQ ID NO: 64 - protein, 12 aa, Artificial Sequence
  synthetic peptide
WAVRALKVKWAL

SEQ ID NO: 65 - protein, 12 aa, Artificial Sequence
  synthetic peptide
WWIKIVVIRVRR

SEQ ID NO: 66 - protein, 12 aa, Artificial Sequence
  synthetic peptide
QIIKVVWRAVII

SEQ ID NO: 67 - protein, 12 aa, Artificial Sequence
  synthetic peptide
QQVKWWLIRWLA

SEQ ID NO: 68 - protein, 12 aa, Artificial Sequence
  synthetic peptide
IKWVLRKIVQII

SEQ ID NO: 69 - protein, 12 aa, Artificial Sequence
  synthetic peptide
VARWKIIIAKLW

SEQ ID NO: 70 - protein, 12 aa, Artificial Sequence
  synthetic peptide
KIWGLLKLGIAL

SEQ ID NO: 71 - protein, 12 aa, Artificial Sequence
  synthetic peptide
RARQIRWLRKRV

SEQ ID NO: 72 - protein, 12 aa, Artificial Sequence
  synthetic peptide
RVLIKWKKVIVV

SEQ ID NO: 73 - protein, 12 aa, Artificial Sequence
  synthetic peptide
LKLKAILKIIRV

SEQ ID NO: 74 - protein, 12 aa, Artificial Sequence
  synthetic peptide
VKLIVAVKIWKK

SEQ ID NO: 75 - protein, 12 aa, Artificial Sequence
  synthetic peptide
  misc_feature (2)..(2): Xaa is L-2,3-diaminopropionic acid
  misc_feature (8)..(8): Xaa is L-2,3-diaminopropionic acid
  misc_feature (11)..(12): Xaa is L-2,3-diaminopropionic acid
VXLIVAVXIWXX

SEQ ID NO: 76 - protein, 12 aa, Artificial Sequence
  synthetic peptide
  misc_feature (2)..(2): Xaa is L-Ornithine
  misc_feature (8)..(8): Xaa is L-Ornithine
  misc_feature (11)..(12): Xaa is L-Ornithine
VXLIVAVXIWXX

SEQ ID NO: 77 - protein, 12 aa, Artificial Sequence
  synthetic peptide
VKLIVKVKIWKK

SEQ ID NO: 78 - protein, 12 aa, Artificial Sequence
  synthetic peptide
  misc_feature (2)..(2): Xaa is L-2,3-diaminopropionic acid
  misc_feature (6)..(6): Xaa is L-2,3-diaminopropionic acid
  misc_feature (8)..(8): Xaa is L-2,3-diaminopropionic acid
  misc_feature (11)..(12): Xaa is L-2,3-diaminopropionic acid
VXLIVXVXIWXX

SEQ ID NO: 79 - protein, 12 aa, Artificial Sequence
  synthetic peptide
  misc_feature: Xaa is L-Ornithine
  misc_feature (2)..(2): Xaa is L-Ornithine
  misc_feature (6)..(6): Xaa is L-Ornithine
  misc_feature (8)..(8): Xaa is L-Ornithine
  misc_feature (11)..(12): Xaa is L-Ornithine
VXLIVXVXIWXX

SEQ ID NO: 80 - protein, 12 aa, Artificial Sequence
  synthetic peptide
  misc_feature: D-amino acid
RRLRRVAVILRV

SEQ ID NO: 81 - protein, 12 aa, Artificial Sequence
  synthetic peptide
  misc_feature: D-amino acid
RRWRRWGVILRV

SEQ ID NO: 82 - protein, 12 aa, Artificial Sequence
  synthetic peptide
  misc_feature: D-amino acid
RRWRRWAVILRV

SEQ ID NO: 83 - protein, 12 aa, Artificial Sequence
  synthetic peptide
  misc_feature: D-amino acid
RRLARVAVILRV

SEQ ID NO: 84 - protein, 12 aa, Artificial Sequence
  synthetic peptide
  misc_feature: D-amino acid
RRWARVGVILRV

SEQ ID NO: 85 - protein, 12 aa, Artificial Sequence
  synthetic peptide
  misc_feature: D-amino acid
RRWARWAVILRV

SEQ ID NO: 86 - protein, 12 aa, Artificial Sequence
  synthetic peptide
  misc_feature: D-amino acid
RRWARWRVILRV

SEQ ID NO: 87 - protein, 12 aa, Artificial Sequence
  synthetic peptide
  misc_feature: D-amino acid
RRWRRVRVILRV

SEQ ID NO: 88 - protein, 12 aa, Artificial Sequence
  synthetic peptide
  misc_feature: D-amino acid
RRWARVRVILRV

SEQ ID NO: 89 - protein, 12 aa, Artificial Sequence
  synthetic peptide
  misc_feature: D-amino acid
RRLIRVRVILRV

SEQ ID NO: 90 - protein, 12 aa, Artificial Sequence
  synthetic peptide
  misc_feature: D-amino acid
RRWIRWRVILRV

SEQ ID NO: 91 - protein, 12 aa, Artificial Sequence
  synthetic peptide
  misc_feature: D-amino acid
RRWRRVKVILRV

SEQ ID NO: 92 - protein, 12 aa, Artificial Sequence
  synthetic peptide
  misc_feature: D-amino acid
RRWARVKVILRV

SEQ ID NO: 93 - protein, 12 aa, Artificial Sequence
  synthetic peptide
  misc_feature: D-amino acid
RRLIRVKVILRV

SEQ ID NO: 94 - protein, 12 aa, Artificial Sequence
  synthetic peptide
  misc_feature: D-amino acid
RRWIRWKVILRV

SEQ ID NO: 95 - protein, 12 aa, Artificial Sequence
  synthetic peptide
  misc_feature: D-amino acid
RRWRRVGVILRV

SEQ ID NO: 96 - protein, 12 aa, Artificial Sequence
  synthetic peptide
  misc_feature: D-amino acid
RRLRRVRVILRV

SEQ ID NO: 97 - protein, 12 aa, Artificial Sequence
  synthetic peptide
  misc_feature: D-amino acid
RRLRRVKVILRV

SEQ ID NO: 98 - protein, 12 aa, Artificial Sequence
  synthetic peptide
  misc_feature: D-amino acid
RRWRRWRVILRV

SEQ ID NO: 99 - protein, 12 aa, Artificial Sequence
  synthetic peptide
  misc_feature: D-amino acid
RRWRRWKVILRV

SEQ ID NO: 100 - protein, 12 aa, Artificial Sequence
  synthetic peptide
  misc_feature: D-amino acid
VQWRRIIVRVIR

SEQ ID NO: 101 - protein, 12 aa, Artificial Sequence
  synthetic peptide
  misc_feature: D-amino acid
VQWRRILVRVIR

SEQ ID NO: 102 - protein, 12 aa, Artificial Sequence
  synthetic peptide
  misc_feature: D-amino acid
VQWRRIRVIVIR

SEQ ID NO: 103 - protein, 12 aa, Artificial Sequence
  synthetic peptide
  MISC_FEATURE: D-amino acid
VQWRRIIVIVIR

SEQ ID NO: 104 - protein, 12 aa, Artificial Sequence
  synthetic peptide
  misc_feature: D-amino acid
VQWRRILVIVIR

SEQ ID NO: 105 - protein, 12 aa, Artificial Sequence
  synthetic peptide
  misc_feature: D-amino acid
VQWRVIIVRVIR

SEQ ID NO: 106 - protein, 12 aa, Artificial Sequence
  synthetic peptide
  misc_feature: D-amino acid
VQWRLIIVRVIR

SEQ ID NO: 107 - protein, 12 aa, Artificial Sequence
  synthetic peptide
  misc_feature: D-amino acid
VQWRVIRVIVIR

SEQ ID NO: 108 - protein, 12 aa, Artificial Sequence
  synthetic peptide
  misc_feature: D-amino acid
VQWRLIRVIVIR

SEQ ID NO: 109 - protein, 12 aa, Artificial Sequence
  synthetic peptide
  misc_feature: D-amino acid
KRRRRVILWRQV

SEQ ID NO: 110 - protein, 12 aa, Artificial Sequence
  synthetic peptide
  misc_feature: D-amino acid
KRRRWVILVRQV

SEQ ID NO: 111 - protein, 12 aa, Artificial Sequence
  synthetic peptide
  misc_feature: D-amino acid
KRRRWVILIRQV

SEQ ID NO: 112 - protein, 12 aa, Artificial Sequence
  synthetic peptide
  misc_feature: D-amino acid
KRRRWVILLRQV

SEQ ID NO: 113 - protein, 12 aa, Artificial Sequence
  synthetic peptide
  misc_feature: D-amino acid
KRQRRVILKRQV

SEQ ID NO: 114 - protein, 12 aa, Artificial Sequence
  synthetic peptide
  misc_feature: D-amino acid
KRIRRVILWRKV

SEQ ID NO: 115 - protein, 12 aa, Artificial Sequence
  synthetic peptide
  misc_feature: D-amino acid
KRRRWVILWRKV

SEQ ID NO: 116 - protein, 12 aa, Artificial Sequence
  synthetic peptide
  misc_feature: D-amino acid
KRGRWVILWRKV

SEQ ID NO: 117 - protein, 12 aa, Artificial Sequence
  synthetic peptide
  misc_feature: D-amino acid
KRQRWVILWRKV

SEQ ID NO: 118 - protein, 12 aa, Artificial Sequence
  synthetic peptide
  misc_feature: D-amino acid
KRARWVILWRKV

SEQ ID NO: 119 - protein, 12 aa, Artificial Sequence
  synthetic peptide
  misc_feature: D-amino acid
KRIRWVILVRKV

SEQ ID NO: 120 - protein, 12 aa, Artificial Sequence
  synthetic peptide
  misc_feature: D-amino acid
KRIRWVILIRKV

SEQ ID NO: 121 - protein, 12 aa, Artificial Sequence
  synthetic peptide
  misc_feature: D-amino acid
KRIRWVILLRKV

SEQ ID NO: 122 - protein, 12 aa, Artificial Sequence
  synthetic peptide
  misc_feature: D-amino acid
KRQRRVILWRQV

SEQ ID NO: 123 - protein, 12 aa, Artificial Sequence
  synthetic peptide
  misc_feature: D-amino acid
KRGRRVILWRQV

SEQ ID NO: 124 - protein, 12 aa, Artificial Sequence
  synthetic peptide
  misc_feature: D-amino acid
KRARRVILWRQV

SEQ ID NO: 125 - protein, 12 aa, Artificial Sequence
  synthetic peptide
  misc_feature: D-amino acid
KRIRRVILVRQV

SEQ ID NO: 126 - protein, 12 aa, Artificial Sequence
  synthetic peptide
  misc_feature: D-amino acid
KRIRRVILIRQV

SEQ ID NO: 127 - protein, 12 aa, Artificial Sequence
  synthetic peptide
  misc_feature: D-amino acid
KRIRRVILLRQV

SEQ ID NO: 128 - protein, 12 aa, Artificial Sequence
  synthetic peptide
  misc_feature: D-amino acid
KRQRWVILVRQV

SEQ ID NO: 129 - protein, 12 aa, Artificial Sequence
  synthetic peptide
  misc_feature: D-amino acid
KRGRWVILVRQV

SEQ ID NO: 130 - protein, 12 aa, Artificial Sequence
  synthetic peptide
  misc_feature: D-amino acid
KRARWVILVRQV

SEQ ID NO: 131 - protein, 12 aa, Artificial Sequence
  synthetic peptide
  misc_feature: D-amino acid
KRQRWVILIRQV

SEQ ID NO: 132 - protein, 12 aa, Artificial Sequence
  synthetic peptide
  misc_feature: D-amino acid
KRGRWVILIRQV

SEQ ID NO: 133 - protein, 12 aa, Artificial Sequence
  synthetic peptide
  misc_feature: D-amino acid
KRARWVILIRQV

SEQ ID NO: 134 - protein, 12 aa, Artificial Sequence
  synthetic peptide
  misc_feature: D-amino acid
KRQRWVILLRQV

SEQ ID NO: 135 - protein, 12 aa, Artificial Sequence
  synthetic peptide
  misc_feature: D-amino acid
KRGRWVILLRQV

SEQ ID NO: 136 - protein, 12 aa, Artificial Sequence
  synthetic peptide
  misc_feature: D-amino acid
KRARWVILLRQV

SEQ ID NO: 137 - protein, 12 aa, Artificial Sequence
  synthetic peptide
  misc_feature: D-amino acid
KRQRRVILVRQV

SEQ ID NO: 138 - protein, 12 aa, Artificial Sequence
  synthetic peptide
  misc_feature: D-amino acid
KRGRRVILVRQV

SEQ ID NO: 139 - protein, 12 aa, Artificial Sequence
  synthetic peptide
  misc_feature: D-amino acid
KRARRVILVRQV

SEQ ID NO: 140 - protein, 12 aa, Artificial Sequence
  synthetic peptide
  misc_feature: D-amino acid
KRQRRVILIRQV

SEQ ID NO: 141 - protein, 12 aa, Artificial Sequence
  synthetic peptide
  misc_feature: D-amino acid
KRGRRVILIRQV

SEQ ID NO: 142 - protein, 12 aa, Artificial Sequence
  synthetic peptide
  misc_feature: D-amino acid
KRARRVILIRQV

SEQ ID NO: 143 - protein, 12 aa, Artificial Sequence
  synthetic peptide
  misc_feature: D-amino acid
KRQRRVILLRQV

SEQ ID NO: 144 - protein, 12 aa, Artificial Sequence
  synthetic peptide
  misc_feature: D-amino acid
KRGRRVILLRQV

SEQ ID NO: 145 - protein, 12 aa, Artificial Sequence
  synthetic peptide
  misc_feature: D-amino acid
KRARRVILLRQV

SEQ ID NO: 146 - protein, 12 aa, Artificial Sequence
  synthetic peptide
  misc_feature: D-amino acid
VIKWLLKILRAI

SEQ ID NO: 147 - protein, 12 aa, Artificial Sequence
  synthetic peptide
  misc_feature: D-amino acid
ILVRWIRWRIQW

SEQ ID NO: 148 - protein, 12 aa, Artificial Sequence
  synthetic peptide
  misc_feature: D-amino acid
WKKVQWLKRLLL

SEQ ID NO: 149 - protein, 12 aa, Artificial Sequence
  synthetic peptide
  misc_feature: D-amino acid
IQRWWKVWLKVI

SEQ ID NO: 150 - protein, 12 aa, Artificial Sequence
  synthetic peptide
  misc_feature: D-amino acid
RRQWRGWVRIWL

SEQ ID NO: 151 - protein, 12 aa, Artificial Sequence
  synthetic peptide
  misc_feature: D-amino acid
IWLRLKVVLKRK

SEQ ID NO: 152 - protein, 12 aa, Artificial Sequence
  synthetic peptide
  misc_feature: D-amino acid
VLKIKVKIWVVK

SEQ ID NO: 153 - protein, 12 aa, Artificial Sequence
  synthetic peptide
  misc_feature: D-amino acid
KKWQLLIKWKLR

SEQ ID NO: 154 - protein, 12 aa, Artificial Sequence
  synthetic peptide
  misc_feature: D-amino acid
AVAKWALKLWKQ

SEQ ID NO: 155 - protein, 12 aa, Artificial Sequence
  synthetic peptide
  misc_feature: D-amino acid
QLARLARVVWGL

SEQ ID NO: 156 - protein, 12 aa, Artificial Sequence
  synthetic peptide
  misc_feature: D-amino acid
VLQIKKVLRLLL

SEQ ID NO: 157 - protein, 12 aa, Artificial Sequence
  synthetic peptide
  misc_feature: D-amino acid
RVKAIKWRKIVV

SEQ ID NO: 158 - protein, 12 aa, Artificial Sequence
  synthetic peptide
  misc_feature: D-amino acid
LWQLWLKLKLKG

SEQ ID NO: 159 - protein, 12 aa, Artificial Sequence
  synthetic peptide
  misc_feature: D-amino acid
KIQRRAWKQWRK

SEQ ID NO: 160 - protein, 12 aa, Artificial Sequence
  synthetic peptide
  misc_feature: D-amino acid
KIVIRIILQVIK

SEQ ID NO: 161 - protein, 12 aa, Artificial Sequence
  synthetic peptide
  misc_feature: D-amino acid
AVKWLGWILAKK

SEQ ID NO: 162 - protein, 12 aa, Artificial Sequence
  synthetic peptide
  misc_feature: D-amino acid
LAGLIVKWAGVR

SEQ ID NO: 163 - protein, 12 aa, Artificial Sequence
  synthetic peptide
  misc_feature: D-amino acid
WVGVIIKWGLKL

SEQ ID NO: 164 - protein, 12 aa, Artificial Sequence
  synthetic peptide
  misc_feature: D-amino acid
WQGWAKIWVVRI

SEQ ID NO: 165 - protein, 12 aa, Artificial Sequence
  synthetic peptide
  misc_feature: D-amino acid
LIVIQLLKKWWK

SEQ ID NO: 166 - protein, 12 aa, Artificial Sequence
  synthetic peptide
  misc_feature: D-amino acid
RRIIKILLWKLR

SEQ ID NO: 167 - protein, 12 aa, Artificial Sequence
  synthetic peptide
  misc_feature: D-amino acid
IAWQLLWGWRVR

SEQ ID NO: 168 - protein, 12 aa, Artificial Sequence
  synthetic peptide
  misc_feature: D-amino acid
VQRIIWLRVKIV

SEQ ID NO: 169 - protein, 12 aa, Artificial Sequence
  synthetic peptide
  misc_feature: D-amino acid
IKIIWKALGQVI

SEQ ID NO: 170 - protein, 12 aa, Artificial Sequence
  synthetic peptide
  misc_feature: D-amino acid
IQLKLIWVKRKW

SEQ ID NO: 171 - protein, 12 aa, Artificial Sequence
  synthetic peptide
  misc_feature: D-amino acid
VIKVLIKRWLKL

SEQ ID NO: 172 - protein, 12 aa, Artificial Sequence
  synthetic peptide
  misc_feature: D-amino acid
VQWIQIVVWRKR

SEQ ID NO: 173 - protein, 12 aa, Artificial Sequence
  synthetic peptide
  misc_feature: D-amino acid
GLIIKIIKKRLW

SEQ ID NO: 174 - protein, 12 aa, Artificial Sequence
  synthetic peptide
  misc_feature: D-amino acid
VKGAIKRGIWVK

SEQ ID NO: 175 - protein, 12 aa, Artificial Sequence
  synthetic peptide
  misc_feature: D-amino acid
KVQIIKQLIAKK

SEQ ID NO: 176 - protein, 12 aa, Artificial Sequence
  synthetic peptide
  misc_feature: D-amino acid
KRLQWVKVKKIR

SEQ ID NO: 177 - protein, 12 aa, Artificial Sequence
  synthetic peptide
  misc_feature: D-amino acid
IVKWIAQWKLVG

SEQ ID NO: 178 - protein, 12 aa, Artificial Sequence
  synthetic peptide
  misc_feature: D-amino acid
KKQKKIWRRILV

SEQ ID NO: 179 - protein, 12 aa, Artificial Sequence
  synthetic peptide
  misc_feature: D-amino acid
GRVLKIVWRKGR

SEQ ID NO: 180 - protein, 12 aa, Artificial Sequence
  synthetic peptide
  misc_feature: D-amino acid
RQVRVKRWRARW

SEQ ID NO: 181 - protein, 12 aa, Artificial Sequence
  synthetic peptide
  misc_feature: D-amino acid
KVVWWKVIIKVL

SEQ ID NO: 182 - protein, 12 aa, Artificial Sequence
  synthetic peptide
  misc_feature: D-amino acid
ALAIKVWIKILQ

SEQ ID NO: 183 - protein, 12 aa, Artificial Sequence
  synthetic peptide
  misc_feature: D-amino acid
IRILVLRKAIIV

SEQ ID NO: 184 - protein, 12 aa, Artificial Sequence
  synthetic peptide
  misc_feature: D-amino acid
IVKKVKLIWGVK

SEQ ID NO: 185 - protein, 12 aa, Artificial Sequence
  synthetic peptide
  misc_feature: D-amino acid
VVGLRVRWVRLW

SEQ ID NO: 186 - protein, 12 aa, Artificial Sequence
  synthetic peptide
  misc_feature: D-amino acid
WAVRALKVKWAL

SEQ ID NO: 187 - protein, 12 aa, Artificial Sequence
  synthetic peptide
  misc_feature: D-amino acid
WWIKIVVIRVRR

SEQ ID NO: 188 - protein, 12 aa, Artificial Sequence
  synthetic peptide
  misc_feature: D-amino acid
QIIKVVWRAVII

SEQ ID NO: 189 - protein, 12 aa, Artificial Sequence
  synthetic peptide
  misc_feature: D-amino acid
QQVKWWLIRWLA

SEQ ID NO: 190 - protein, 12 aa, Artificial Sequence
  synthetic peptide
  misc_feature: D-amino acid
IKWVLRKIVQII

SEQ ID NO: 191 - protein, 12 aa, Artificial Sequence
  synthetic peptide
  misc_feature: D-amino acid
VARWKIIIAKLW

SEQ ID NO: 192 - protein, 12 aa, Artificial Sequence
  synthetic peptide
  misc_feature: D-amino acid
KIWGLLKLGIAL

SEQ ID NO: 193 - protein, 12 aa, Artificial Sequence
  synthetic peptide
  misc_feature: D-amino acid
RARQIRWLRKRV

SEQ ID NO: 194 - protein, 12 aa, Artificial Sequence
  synthetic peptide
  misc_feature: D-amino acid
RVLIKWKKVIVV

SEQ ID NO: 195 - protein, 12 aa, Artificial Sequence
  synthetic peptide
  misc_feature: D-amino acid
LKLKAILKIIRV

SEQ ID NO: 196 - protein, 12 aa, Artificial Sequence
  synthetic peptide
  misc_feature: D-amino acid
IARLIKLLWKIV

SEQ ID NO: 197 - protein, 12 aa, Artificial Sequence
  synthetic peptide
  misc_feature: D-amino acid
WQIRWRIWRVLI

SEQ ID NO: 198 - protein, 12 aa, Artificial Sequence
  synthetic peptide
  misc_feature: D-amino acid
LLLRKLWQVKKW

SEQ ID NO: 199 - protein, 12 aa, Artificial Sequence
  synthetic peptide
  misc_feature: D-amino acid
IVKLWVKWWRQI

SEQ ID NO: 200 - protein, 12 aa, Artificial Sequence
  synthetic peptide
  misc_feature: D-amino acid
LWIRVWGRWQRR

SEQ ID NO: 201 - protein, 12 aa, Artificial Sequence
  synthetic peptide
  misc_feature: D-amino acid
KRKLVVKLRLWI

SEQ ID NO: 202 - protein, 12 aa, Artificial Sequence
  synthetic peptide
  misc_feature: D-amino acid
KVVWIKVKIKLV

SEQ ID NO: 203 - protein, 12 aa, Artificial Sequence
  Synthetic peptide
  misc_feature: D-amino acid
RLKWKILLQWKK

SEQ ID NO: 204 - protein, 12 aa, Artificial Sequence
  synthetic peptide
  misc_feature: D-amino acid
QKWLKLAWKAVA

SEQ ID NO: 205 - protein, 12 aa, Artificial Sequence
  synthetic peptide
  misc_feature: D-amino acid
LGWVVRALRALQ

SEQ ID NO: 206 - protein, 12 aa, Artificial Sequence
  synthetic peptide
  misc_feature: D-amino acid
LLLRLVKKIQLV

SEQ ID NO: 207 - protein, 12 aa, Artificial Sequence
  synthetic peptide
  misc_feature: D-amino acid
VVIKRWKIAKVR

SEQ ID NO: 208 - protein, 12 aa, Artificial Sequence
  synthetic peptide
  misc_feature: D-amino acid
GKLKLKLWLQWL

SEQ ID NO: 209 - protein, 12 aa, Artificial Sequence
  synthetic peptide
  misc_feature: D-amino acid
KRWQKWARRQIK

SEQ ID NO: 210 - protein, 12 aa, Artificial Sequence
  synthetic peptide
  misc_feature: D-amino acid
KIVQLIIRIVIK

SEQ ID NO: 211 - protein, 12 aa, Artificial Sequence
  synthetic peptide
  misc_feature: D-amino acid
KKALIWGLWKVA

SEQ ID NO: 212 - protein, 12 aa, Artificial Sequence
  synthetic peptide
  misc_feature: D-amino acid
RVGAWKVILGAL

SEQ ID NO: 213 - protein, 12 aa, Artificial Sequence
  synthetic peptide
  misc_feature: D-amino acid
LKLGWKIIVGVW

SEQ ID NO: 214 - protein, 12 aa, Artificial Sequence
  synthetic peptide
  misc_feature: D-amino acid
IRVVWIKAWGQW

SEQ ID NO: 215 - protein, 12 aa, Artificial Sequence
  synthetic peptide
  misc_feature: D-amino acid
KWWKKLLQIVIL

SEQ ID NO: 216 - protein, 12 aa, Artificial Sequence
  synthetic peptide
  misc_feature: D-amino acid
RLKWLLIKIIRR

SEQ ID NO: 217 - protein, 12 aa, Artificial Sequence
  synthetic peptide
  misc_feature: D-amino acid
RVRWGWLLQWAI

SEQ ID NO: 218 - protein, 12 aa, Artificial Sequence
  synthetic peptide
  misc_feature: D-amino acid
VIKVRLWIIRQV

SEQ ID NO: 219 - protein, 12 aa, Artificial Sequence
  synthetic peptide
  misc_feature: D-amino acid
IVQGLAKWIIKI

SEQ ID NO: 220 - protein, 12 aa, Artificial Sequence
  synthetic peptide
  misc_feature: D-amino acid
WKRKVWILKLQI

SEQ ID NO: 221 - protein, 12 aa, Artificial Sequence
  synthetic peptide
  misc_feature: D-amino acid
LKLWRKILVKIV

SEQ ID NO: 222 - protein, 12 aa, Artificial Sequence
  synthetic peptide
  misc_feature: D-amino acid
RKRWVVIQIWQV

SEQ ID NO: 223 - protein, 12 aa, Artificial Sequence
  synthetic peptide
  misc_feature: D-amino acid
WLRKKIIKIILG

SEQ ID NO: 224 - protein, 12 aa, Artificial Sequence
  synthetic peptide
  misc_feature: D-amino acid
KVWIGRKIAGKV

SEQ ID NO: 225 - protein, 12 aa, Artificial Sequence
  synthetic peptide
  misc_feature: D-amino acid
KKAILQKIIQVK

SEQ ID NO: 226 - protein, 12 aa, Artificial Sequence
  synthetic peptide
  misc_feature: D-amino acid
RIKKVKVWQLRK

SEQ ID NO: 227 - protein, 12 aa, Artificial Sequence
  synthetic peptide
  misc_feature: D-amino acid
GVLKWQAIWKVI

SEQ ID NO: 228 - protein, 12 aa, Artificial Sequence
  synthetic peptide
  misc_feature: D-amino acid
VLIRRWIKKQKK

SEQ ID NO: 229 - protein, 12 aa, Artificial Sequence
  synthetic peptide
  misc_feature: D-amino acid
RGKRWVIKLVRG

SEQ ID NO: 230 - protein, 12 aa, Artificial Sequence
  synthetic peptide
  misc_feature: D-amino acid
WRARWRKVRVQR

SEQ ID NO: 231 - protein, 12 aa, Artificial Sequence
  synthetic peptide
  misc_feature: D-amino acid
LVKIIVKWWVVK

SEQ ID NO: 232 - protein, 12 aa, Artificial Sequence
  synthetic peptide
  misc_feature: D-amino acid
QLIKIWVKIALA

SEQ ID NO: 233 - protein, 12 aa, Artificial Sequence
  synthetic peptide
  misc_feature: D-amino acid
VIIAKRLVLIRI

SEQ ID NO: 234 - protein, 12 aa, Artificial Sequence
  synthetic peptide
  misc_feature: D-amino acid
KVGWILKVKKVI

SEQ ID NO: 235 - protein, 12 aa, Artificial Sequence
  synthetic peptide
  misc_feature: D-amino acid
WLRVWRVRLGVV

SEQ ID NO: 236 - protein, 12 aa, Artificial Sequence
  synthetic peptide
  misc_feature: D-amino acid
LAWKVKLARVAW

SEQ ID NO: 237 - protein, 12 aa, Artificial Sequence
  synthetic peptide
  misc_feature: D-amino acid
RRVRIVVIKIWW

SEQ ID NO: 238 - protein, 12 aa, Artificial Sequence
  synthetic peptide
  misc_feature: D-amino acid
IIVARWVVKIIQ

SEQ ID NO: 239 - protein, 12 aa, Artificial Sequence
  synthetic peptide
  misc_feature: D-amino acid
ALWRILWWKVQQ

SEQ ID NO: 240 - protein, 12 aa, Artificial Sequence
  synthetic peptide
  misc_feature: D-amino acid
IIQVIKRLVWKI

SEQ ID NO: 241 - protein, 12 aa, Artificial Sequence
  synthetic peptide
  misc_feature: D-amino acid
WLKAIIIKWRAV

SEQ ID NO: 242 - protein, 12 aa, Artificial Sequence
  synthetic peptide
  misc_feature: D-amino acid
LAIGLKLLGWIK

SEQ ID NO: 243 - protein, 12 aa, Artificial Sequence
  synthetic peptide
  misc_feature: D-amino acid
VRKRLWRIQRAR

SEQ ID NO: 244 - protein, 12 aa, Artificial Sequence
  synthetic peptide
  misc_feature: D-amino acid
VVIVKKWKILVR

SEQ ID NO: 245 - protein, 12 aa, Artificial Sequence
  synthetic peptide
  misc_feature: D-amino acid
VRIIKLIAKLKL

SEQ ID NO: 246 - protein, 12 aa, Artificial Sequence
  synthetic peptide
GLVRWIRWRIQW

SEQ ID NO: 247 - protein, 12 aa, Artificial Sequence
  synthetic peptide
IGVRWIRWRIQW

SEQ ID NO: 248 - protein, 12 aa, Artificial Sequence
  synthetic peptide
ILGRWIRWRIQW

SEQ ID NO: 249 - protein, 12 aa, Artificial Sequence
  synthetic peptide
ILVGWIRWRIQW

SEQ ID NO: 250 - protein, 12 aa, Artificial Sequence
  synthetic peptide
ILVRGIRWRIQW

SEQ ID NO: 251 - protein, 12 aa, Artificial Sequence
  synthetic peptide
ILVRWGRWRIQW

SEQ ID NO: 252 - protein, 12 aa, Artificial Sequence
  synthetic peptide
ILVRWIGWRIQW

SEQ ID NO: 253 - protein, 12 aa, Artificial Sequence
  synthetic peptide
ILVRWIRGRIQW

SEQ ID NO: 254 - protein, 12 aa, Artificial Sequence
  synthetic peptide
ILVRWIRWGIQW

SEQ ID NO: 255 - protein, 12 aa, Artificial Sequence
  synthetic peptide
ILVRWIRWRGQW

SEQ ID NO: 256 - protein, 12 aa, Artificial Sequence
  synthetic peptide
ILVRWIRWRIGW

SEQ ID NO: 257 - protein, 12 aa, Artificial Sequence
  synthetic peptide
ILVRWIRWRIQG

SEQ ID NO: 258 - protein, 12 aa, Artificial Sequence
  synthetic peptide
ALVRWIRWRIQW

SEQ ID NO: 259 - protein, 12 aa, Artificial Sequence
  synthetic peptide
IAVRWIRWRIQW

SEQ ID NO: 260 - protein, 12 aa, Artificial Sequence
  synthetic peptide
ILARWIRWRIQW

SEQ ID NO: 261 - protein, 12 aa, Artificial Sequence
  synthetic peptide
ILVAWIRWRIQW

SEQ ID NO: 262 - protein, 12 aa, Artificial Sequence
  synthetic peptide
ILVRAIRWRIQW

SEQ ID NO: 263 - protein, 12 aa, Artificial Sequence
  synthetic peptide
ILVRWARWRIQW

SEQ ID NO: 264 - protein, 12 aa, Artificial Sequence
  synthetic peptide
ILVRWIAWRIQW

SEQ ID NO: 265 - protein, 12 aa, Artificial Sequence
  synthetic peptide
ILVRWIRARIQW

SEQ ID NO: 266 - protein, 12 aa, Artificial Sequence
  synthetic peptide
ILVRWIRWAIQW

SEQ ID NO: 267 - protein, 12 aa, Artificial Sequence
  synthetic peptide
ILVRWIRWRAQW

SEQ ID NO: 268 - protein, 12 aa, Artificial Sequence
  synthetic peptide
ILVRWIRWRIAW

SEQ ID NO: 269 - protein, 12 aa, Artificial Sequence
  synthetic peptide
ILVRWIRWRIQA

SEQ ID NO: 270 - protein, 12 aa, Artificial Sequence
  synthetic peptide
RLVRWIRWRIQW

SEQ ID NO: 271 - protein, 12 aa, Artificial Sequence
  synthetic peptide
IRVRWIRWRIQW

SEQ ID NO: 272 - protein, 12 aa, Artificial Sequence
  synthetic peptide
ILRRWIRWRIQW

SEQ ID NO: 273 - protein, 12 aa, Artificial Sequence
  synthetic peptide
ILVRRIRWRIQW

SEQ ID NO: 274 - protein, 12 aa, Artificial Sequence
  synthetic peptide
ILVRWRRWRIQW

SEQ ID NO: 275 - protein, 12 aa, Artificial Sequence
  synthetic peptide
ILVRWIRRRIQW

SEQ ID NO: 276 - protein, 12 aa, Artificial Sequence
  synthetic peptide
ILVRWIRWRRQW

SEQ ID NO: 277 - protein, 12 aa, Artificial Sequence
  synthetic peptide
ILVRWIRWRIRW

SEQ ID NO: 278 - protein, 12 aa, Artificial Sequence
  synthetic peptide
ILVRWIRWRIQR

SEQ ID NO: 279 - protein, 12 aa, Artificial Sequence
  synthetic peptide
KLVRWIRWRIQW

SEQ ID NO: 280 - protein, 12 aa, Artificial Sequence
  synthetic peptide
IKVRWIRWRIQW

SEQ ID NO: 281 - protein, 12 aa, Artificial Sequence
  synthetic peptide
ILKRWIRWRIQW

SEQ ID NO: 282 - protein, 12 aa, Artificial Sequence
  synthetic peptide
ILVKWIRWRIQW

SEQ ID NO: 283 - protein, 12 aa, Artificial Sequence
  synthetic peptide
ILVRKIRWRIQW

SEQ ID NO: 284 - protein, 12 aa, Artificial Sequence
  synthetic peptide
ILVRWKRWRIQW

SEQ ID NO: 285 - protein, 12 aa, Artificial Sequence
  synthetic peptide
ILVRWIKWRIQW

SEQ ID NO: 286 - protein, 12 aa, Artificial Sequence
  synthetic peptide
ILVRWIRKRIQW

SEQ ID NO: 287 - protein, 12 aa, Artificial Sequence
  synthetic peptide
ILVRWIRWKIQW

SEQ ID NO: 288 - protein, 12 aa, Artificial Sequence
  synthetic peptide
ILVRWIRWRKQW

SEQ ID NO: 289 - protein, 12 aa, Artificial Sequence
  synthetic peptide
ILVRWIRWRIKW

SEQ ID NO: 290 - protein, 12 aa, Artificial Sequence
  synthetic peptide
ILVRWIRWRIQK

SEQ ID NO: 291 - protein, 12 aa, Artificial Sequence
  synthetic peptide
VLVRWIRWRIQW

SEQ ID NO: 292 - protein, 12 aa, Artificial Sequence
  synthetic peptide
IVVRWIRWRIQW

SEQ ID NO: 293 - protein, 12 aa, Artificial Sequence
  synthetic peptide
ILVVWIRWRIQW

SEQ ID NO: 294 - protein, 12 aa, Artificial Sequence
  synthetic peptide
ILVRVIRWRIQW

SEQ ID NO: 295 - protein, 12 aa, Artificial Sequence
  synthetic peptide
ILVRWVRWRIQW

SEQ ID NO: 296 - protein, 12 aa, Artificial Sequence
  synthetic peptide
ILVRWIVWRIQW

SEQ ID NO: 297 - protein, 12 aa, Artificial Sequence
  synthetic peptide
ILVRWIRVRIQW

SEQ ID NO: 298 - protein, 12 aa, Artificial Sequence
  synthetic peptide
ILVRWIRWVIQW

SEQ ID NO: 299 - protein, 12 aa, Artificial Sequence
  synthetic peptide
ILVRWIRWRVQW

SEQ ID NO: 300 - protein, 12 aa, Artificial Sequence
  synthetic peptide
ILVRWIRWRIVW

SEQ ID NO: 301 - protein, 12 aa, Artificial Sequence
  synthetic peptide
ILVRWIRWRIQV

SEQ ID NO: 302 - protein, 12 aa, Artificial Sequence
  synthetic peptide
IIVRWIRWRIQW

SEQ ID NO: 303 - protein, 12 aa, Artificial Sequence
  synthetic peptide
ILIRWIRWRIQW

SEQ ID NO: 304 - protein, 12 aa, Artificial Sequence
  synthetic peptide
ILVIWIRWRIQW

SEQ ID NO: 305 - protein, 12 aa, Artificial Sequence
  synthetic peptide
ILVRIIRWRIQW

SEQ ID NO: 306 - protein, 12 aa, Artificial Sequence
  synthetic peptide
ILVRWIIWRIQW

SEQ ID NO: 307 - protein, 12 aa, Artificial Sequence
  synthetic peptide
ILVRWIRIRIQW

SEQ ID NO: 308 - protein, 12 aa, Artificial Sequence
  synthetic peptide
ILVRWIRWIIQW

SEQ ID NO: 309 - protein, 12 aa, Artificial Sequence
  synthetic peptide
ILVRWIRWRIIW

SEQ ID NO: 310 - protein, 12 aa, Artificial Sequence
  synthetic peptide
ILVRWIRWRIQI

SEQ ID NO: 311 - protein, 12 aa, Artificial Sequence
  synthetic peptide
LLVRWIRWRIQW

SEQ ID NO: 312 - protein, 12 aa, Artificial Sequence
  synthetic peptide
ILLRWIRWRIQW

SEQ ID NO: 313 - protein, 12 aa, Artificial Sequence
  synthetic peptide
ILVLWIRWRIQW

SEQ ID NO: 314 - protein, 12 aa, Artificial Sequence
  synthetic peptide
ILVRLIRWRIQW

SEQ ID NO: 315 - protein, 12 aa, Artificial Sequence
  synthetic peptide
ILVRWLRWRIQW

SEQ ID NO: 316 - protein, 12 aa, Artificial Sequence
  synthetic peptide
ILVRWILWRIQW

SEQ ID NO: 317 - protein, 12 aa, Artificial Sequence
  synthetic peptide
ILVRWIRLRIQW

SEQ ID NO: 318 - protein, 12 aa, Artificial Sequence
  synthetic peptide
ILVRWIRWLIQW

SEQ ID NO: 319 - protein, 12 aa, Artificial Sequence
  synthetic peptide
ILVRWIRWRLQW

SEQ ID NO: 320 - protein, 12 aa, Artificial Sequence
  synthetic peptide
ILVRWIRWRILW

SEQ ID NO: 321 - protein, 12 aa, Artificial Sequence
  synthetic peptide
ILVRWIRWRIQL

SEQ ID NO: 322 - protein, 12 aa, Artificial Sequence
  synthetic peptide
WLVRWIRWRIQW

SEQ ID NO: 323 - protein, 12 aa, Artificial Sequence
  synthetic peptide
IWVRWIRWRIQW

SEQ ID NO: 324 - protein, 12 aa, Artificial Sequence
  synthetic peptide
ILWRWIRWRIQW

SEQ ID NO: 325 - protein, 12 aa, Artificial Sequence
  synthetic peptide
ILVWWIRWRIQW

SEQ ID NO: 326 - protein, 12 aa, Artificial Sequence
  synthetic peptide
ILVRWWRWRIQW

SEQ ID NO: 327 - protein, 12 aa, Artificial Sequence
  synthetic peptide
ILVRWIWWRIQW

SEQ ID NO: 328 - protein, 12 aa, Artificial Sequence
  synthetic peptide
ILVRWIRWWIQW

SEQ ID NO: 329 - protein, 12 aa, Artificial Sequence
  synthetic peptide
ILVRWIRWRWQW

SEQ ID NO: 330 - protein, 12 aa, Artificial Sequence
  synthetic peptide
ILVRWIRWRIWW

SEQ ID NO: 331 - protein, 12 aa, Artificial Sequence
  synthetic peptide
QLVRWIRWRIQW

SEQ ID NO: 332 - protein, 12 aa, Artificial Sequence
  synthetic peptide
IQVRWIRWRIQW

SEQ ID NO: 333 - protein, 12 aa, Artificial Sequence
  synthetic peptide
ILQRWIRWRIQW

SEQ ID NO: 334 - protein, 12 aa, Artificial Sequence
  synthetic peptide
ILVQWIRWRIQW

SEQ ID NO: 335 - protein, 12 aa, Artificial Sequence
  synthetic peptide
ILVRQIRWRIQW

SEQ ID NO: 336 - protein, 12 aa, Artificial Sequence
  synthetic peptide
ILVRWQRWRIQW

SEQ ID NO: 337 - protein, 12 aa, Artificial Sequence
  synthetic peptide
ILVRWIQWRIQW

SEQ ID NO: 338 - protein, 12 aa, Artificial Sequence
  synthetic peptide
ILVRWIRQRIQW

SEQ ID NO: 339 - protein, 12 aa, Artificial Sequence
  synthetic peptide
ILVRWIRWQIQW

SEQ ID NO: 340 - protein, 12 aa, Artificial Sequence
  synthetic peptide
ILVRWIRWRQQW

SEQ ID NO: 341 - protein, 12 aa, Artificial Sequence
  synthetic peptide
ILVRWIRWRIQQ

SEQ ID NO: 342 - protein, 12 aa, Artificial Sequence
  synthetic peptide
GLKIKVKIWVVK

SEQ ID NO: 343 - protein, 12 aa, Artificial Sequence
  synthetic peptide
VGKIKVKIWVVK

SEQ ID NO: 344 - protein, 12 aa, Artificial Sequence
  synthetic peptide
VLGIKVKIWVVK

SEQ ID NO: 345 - protein, 12 aa, Artificial Sequence
  synthetic peptide
VLKGKVKIWVVK

SEQ ID NO: 346 - protein, 12 aa, Artificial Sequence
  synthetic peptide
VLKIGVKIWVVK

SEQ ID NO: 347 - protein, 12 aa, Artificial Sequence
  synthetic peptide
VLKIKGKIWVVK

SEQ ID NO: 348 - protein, 12 aa, Artificial Sequence
  synthetic peptide
VLKIKVGIWVVK

SEQ ID NO: 349 - protein, 12 aa, Artificial Sequence
  synthetic peptide
VLKIKVKGWVVK

SEQ ID NO: 350 - protein, 12 aa, Artificial Sequence
  synthetic peptide
VLKIKVKIGVVK

SEQ ID NO: 351 - protein, 12 aa, Artificial Sequence
  synthetic peptide
VLKIKVKIWGVK

SEQ ID NO: 352 - protein, 12 aa, Artificial Sequence
  synthetic peptide
VLKIKVKIWVGK

SEQ ID NO: 353 - protein, 12 aa, Artificial Sequence
  synthetic peptide
VLKIKVKIWVVG

SEQ ID NO: 354 - protein, 12 aa, Artificial Sequence
  synthetic peptide
ALKIKVKIWVVK

SEQ ID NO: 355 - protein, 12 aa, Artificial Sequence
  synthetic peptide
VAKIKVKIWVVK

SEQ ID NO: 356 - protein, 12 aa, Artificial Sequence
  synthetic peptide
VLAIKVKIWVVK

SEQ ID NO: 357 - protein, 12 aa, Artificial Sequence
  synthetic peptide
VLKAKVKIWVVK

SEQ ID NO: 358 - protein, 12 aa, Artificial Sequence
  synthetic peptide
VLKIAVKIWVVK

SEQ ID NO: 359 - protein, 12 aa, Artificial Sequence
  synthetic peptide
VLKIKAKIWVVK

SEQ ID NO: 360 - protein, 12 aa, Artificial Sequence
  synthetic peptide
VLKIKVAIWVVK

SEQ ID NO: 361 - protein, 12 aa, Artificial Sequence
  synthetic peptide
VLKIKVKAWVVK

SEQ ID NO: 362 - protein, 12 aa, Artificial Sequence
  synthetic peptide
VLKIKVKIAVVK

SEQ ID NO: 363 - protein, 12 aa, Artificial Sequence
  synthetic peptide
VLKIKVKIWAVK

SEQ ID NO: 364 - protein, 12 aa, Artificial Sequence
  synthetic peptide
VLKIKVKIWVAK

SEQ ID NO: 365 - protein, 12 aa, Artificial Sequence
  synthetic peptide
VLKIKVKIWVVA

SEQ ID NO: 366 - protein, 12 aa, Artificial Sequence
  synthetic peptide
RLKIKVKIWVVK

SEQ ID NO: 367 - protein, 12 aa, Artificial Sequence
  synthetic peptide
VRKIKVKIWVVK

SEQ ID NO: 368 - protein, 12 aa, Artificial Sequence
  synthetic peptide
VLRIKVKIWVVK

SEQ ID NO: 369 - protein, 12 aa, Artificial Sequence
  synthetic peptide
VLKRKVKIWVVK

SEQ ID NO: 370 - protein, 12 aa, Artificial Sequence
  synthetic peptide
VLKIRVKIWVVK

SEQ ID NO: 371 - protein, 12 aa, Artificial Sequence
  synthetic peptide
VLKIKRKIWVVK

SEQ ID NO: 372 - protein, 12 aa, Artificial Sequence
  synthetic peptide
VLKIKVRIWVVK

SEQ ID NO: 373 - protein, 12 aa, Artificial Sequence
  synthetic peptide
VLKIKVKRWVVK

SEQ ID NO: 374 - protein, 12 aa, Artificial Sequence
  synthetic peptide
VLKIKVKIRVVK

SEQ ID NO: 375 - protein, 12 aa, Artificial Sequence
  synthetic peptide
VLKIKVKIWRVK

SEQ ID NO: 376 - protein, 12 aa, Artificial Sequence
  synthetic peptide
VLKIKVKIWVRK

SEQ ID NO: 377 - protein, 12 aa, Artificial Sequence
  synthetic peptide
VLKIKVKIWVVR

SEQ ID NO: 378 - protein, 12 aa, Artificial Sequence
  synthetic peptide
KLKIKVKIWVVK

SEQ ID NO: 379 - protein, 12 aa, Artificial Sequence
  synthetic peptide
VKKIKVKIWVVK

SEQ ID NO: 380 - protein, 12 aa, Artificial Sequence
  synthetic peptide
VLKKKVKIWVVK

SEQ ID NO: 381 - protein, 12 aa, Artificial Sequence
  synthetic peptide
VLKIKKKIWVVK

SEQ ID NO: 382 - protein, 12 aa, Artificial Sequence
  synthetic peptide
VLKIKVKKWVVK

SEQ ID NO: 383 - protein, 12 aa, Artificial Sequence
  synthetic peptide
VLKIKVKIKVVK

SEQ ID NO: 384 - protein, 12 aa, Artificial Sequence
  synthetic peptide
VLKIKVKIWKVK

SEQ ID NO: 385 - protein, 12 aa, Artificial Sequence
  synthetic peptide
VLKIKVKIWVKK

SEQ ID NO: 386 - protein, 12 aa, Artificial Sequence
  synthetic peptide
VVKIKVKIWVVK

SEQ ID NO: 387 - protein, 12 aa, Artificial Sequence
  synthetic peptide
VLVIKVKIWVVK

SEQ ID NO: 388 - protein, 12 aa, Artificial Sequence
  synthetic peptide
VLKVKVKIWVVK

SEQ ID NO: 389 - protein, 12 aa, Artificial Sequence
  Synthetic peptide
VLKIVVKIWVVK

SEQ ID NO: 390 - protein, 12 aa, Artificial Sequence
  synthetic peptide
VLKIKVVIWVVK

SEQ ID NO: 391 - protein, 12 aa, Artificial Sequence
  synthetic peptide
VLKIKVKVWVVK

SEQ ID NO: 392 - protein, 12 aa, Artificial Sequence
  synthetic peptide
VLKIKVKIVVVK

SEQ ID NO: 393 - protein, 12 aa, Artificial Sequence
  synthetic peptide
VLKIKVKIWVVV

SEQ ID NO: 394 - protein, 12 aa, Artificial Sequence
  synthetic peptide
ILKIKVKIWVVK

SEQ ID NO: 395 - protein, 12 aa, Artificial Sequence
  synthetic peptide
VIKIKVKIWVVK

SEQ ID NO: 396 - protein, 12 aa, Artificial Sequence
  synthetic peptide
VLIIKVKIWVVK

SEQ ID NO: 397 - protein, 12 aa, Artificial Sequence
  synthetic peptide
VLKIIVKIWVVK

SEQ ID NO: 398 - protein, 12 aa, Artificial Sequence
  synthetic peptide
VLKIKIKIWVVK

SEQ ID NO: 399 - protein, 12 aa, Artificial Sequence
  synthetic peptide
VLKIKVIIWVVK

SEQ ID NO: 400 - protein, 12 aa, Artificial Sequence
  synthetic peptide
VLKIKVKIIVVK

SEQ ID NO: 401 - protein, 12 aa, Artificial Sequence
  synthetic peptide
VLKIKVKIWIVK

SEQ ID NO: 402 - protein, 12 aa, Artificial Sequence
  synthetic peptide
VLKIKVKIWVIK

SEQ ID NO: 403 - protein, 12 aa, Artificial Sequence
  synthetic peptide
VLKIKVKIWVVI

SEQ ID NO: 404 - protein, 12 aa, Artificial Sequence
  synthetic peptide
LLKIKVKIWVVK

SEQ ID NO: 405 - protein, 12 aa, Artificial Sequence
  synthetic peptide
VLLIKVKIWVVK

SEQ ID NO: 406 - protein, 12 aa, Artificial Sequence
  synthetic peptide
VLKLKVKIWVVK

SEQ ID NO: 407 - protein, 12 aa, Artificial Sequence
  synthetic peptide
VLKILVKIWVVK

SEQ ID NO: 408 - protein, 12 aa, Artificial Sequence
  synthetic peptide
VLKIKLKIWVVK

SEQ ID NO: 409 - protein, 12 aa, Artificial Sequence
  synthetic peptide
VLKIKVLIWVVK

SEQ ID NO: 410 - protein, 12 aa, Artificial Sequence
  synthetic peptide
VLKIKVKLWVVK

SEQ ID NO: 411 - protein, 12 aa, Artificial Sequence
  synthetic peptide
VLKIKVKILVVK

SEQ ID NO: 412 - protein, 12 aa, Artificial Sequence
  synthetic peptide
VLKIKVKIWLVK

SEQ ID NO: 413 - protein, 12 aa, Artificial Sequence
  synthetic peptide
VLKIKVKIWVLK

SEQ ID NO: 414 - protein, 12 aa, Artificial Sequence
  synthetic peptide
VLKIKVKIWVVL

SEQ ID NO: 415 - protein, 12 aa, Artificial Sequence
  synthetic peptide
WLKIKVKIWVVK

SEQ ID NO: 416 - protein, 12 aa, Artificial Sequence
  synthetic peptide
VWKIKVKIWVVK

SEQ ID NO: 417 - protein, 12 aa, Artificial Sequence
  synthetic peptide
VLWIKVKIWVVK

SEQ ID NO: 418 - protein, 12 aa, Artificial Sequence
  synthetic peptide
VLKWKVKIWVVK

SEQ ID NO: 419 - protein, 12 aa, Artificial Sequence
  synthetic peptide
VLKIWVKIWVVK

SEQ ID NO: 420 - protein, 12 aa, Artificial Sequence
  synthetic peptide
VLKIKWKIWVVK

SEQ ID NO: 421 - protein, 12 aa, Artificial Sequence
  synthetic peptide
VLKIKVWIWVVK

SEQ ID NO: 422 - protein, 12 aa, Artificial Sequence
  synthetic peptide
VLKIKVKWWVVK

SEQ ID NO: 423 - protein, 12 aa, Artificial Sequence
  synthetic peptide
VLKIKVKIWWVK

SEQ ID NO: 424 - protein, 12 aa, Artificial Sequence
  synthetic peptide
VLKIKVKIWVWK

SEQ ID NO: 425 - protein, 12 aa, Artificial Sequence
  synthetic peptide
VLKIKVKIWVVW

SEQ ID NO: 426 - protein, 12 aa, Artificial Sequence
  synthetic peptide
QLKIKVKIWVVK

SEQ ID NO: 427 - protein, 12 aa, Artificial Sequence
  synthetic peptide
VQKIKVKIWVVK

SEQ ID NO: 428 - protein, 12 aa, Artificial Sequence
  synthetic peptide
VLQIKVKIWVVK

SEQ ID NO: 429 - protein, 12 aa, Artificial Sequence
  synthetic peptide
VLKQKVKIWVVK

SEQ ID NO: 430 - protein, 12 aa, Artificial Sequence
  synthetic peptide
VLKIQVKIWVVK

SEQ ID NO: 431 - protein, 12 aa, Artificial Sequence
  synthetic peptide
VLKIKQKIWVVK

SEQ ID NO: 432 - protein, 12 aa, Artificial Sequence
  synthetic peptide
VLKIKVQIWVVK

SEQ ID NO: 433 - protein, 12 aa, Artificial Sequence
  synthetic peptide
VLKIKVKQWVVK

SEQ ID NO: 434 - protein, 24 aa, Artificial Sequence
  synthetic peptide
VLKIKVKIQVVKVLKIKVKIWQVK

SEQ ID NO: 435 - protein, 12 aa, Artificial Sequence
  synthetic peptide
VLKIKVKIWQVK

SEQ ID NO: 436 - protein, 12 aa, Artificial Sequence
  synthetic peptide
VLKIKVKIWVQK

SEQ ID NO: 437 - protein, 12 aa, Artificial Sequence
  synthetic peptide
VLKIKVKIWVVQ

SEQ ID NO: 438 - protein, 12 aa, Artificial Sequence
  synthetic peptide
VRLIVAVRIWRR

SEQ ID NO: 439 - protein, 12 aa, Artificial Sequence
  synthetic peptide
GRLIVAVRIWRR

SEQ ID NO: 440 - protein, 12 aa, Artificial Sequence
  synthetic peptide
VGLIVAVRIWRR

SEQ ID NO: 441 - protein, 12 aa, Artificial Sequence
  synthetic peptide
VRGIVAVRIWRR

SEQ ID NO: 442 - protein, 12 aa, Artificial Sequence
  synthetic peptide
VRLGVAVRIWRR

SEQ ID NO: 443 - protein, 12 aa, Artificial Sequence
  synthetic peptide
VRLIGAVRIWRR

SEQ ID NO: 444 - protein, 12 aa, Artificial Sequence
  synthetic peptide
VRLIVGVRIWRR

SEQ ID NO: 445 - protein, 12 aa, Artificial Sequence
  synthetic peptide
VRLIVAGRIWRR

SEQ ID NO: 446 - protein, 12 aa, Artificial Sequence
  synthetic peptide
VRLIVAVGIWRR

SEQ ID NO: 447 - protein, 12 aa, Artificial Sequence
  synthetic peptide
VRLIVAVRGWRR

SEQ ID NO: 448 - protein, 12 aa, Artificial Sequence
  synthetic peptide
VRLIVAVRIGRR

SEQ ID NO: 449 - protein, 12 aa, Artificial Sequence
  synthetic peptide
VRLIVAVRIWGR

SEQ ID NO: 450 - protein, 12 aa, Artificial Sequence
  synthetic peptide
VRLIVAVRIWRG

SEQ ID NO: 451 - protein, 12 aa, Artificial Sequence
  synthetic peptide
ARLIVAVRIWRR

SEQ ID NO: 452 - protein, 12 aa, Artificial Sequence
  synthetic peptide
VALIVAVRIWRR

SEQ ID NO: 453 - protein, 12 aa, Artificial Sequence
  synthetic peptide
VRAIVAVRIWRR

SEQ ID NO: 454 - protein, 12 aa, Artificial Sequence
  synthetic peptide
VRLAVAVRIWRR

SEQ ID NO: 455 - protein, 13 aa, Artificial Sequence
  synthetic peptide
ZVRLIAAVRIWRR

SEQ ID NO: 456 - protein, 12 aa, Artificial Sequence
  synthetic peptide
VRLIVAARIWRR

SEQ ID NO: 457 - protein, 12 aa, Artificial Sequence
  synthetic peptide
VRLIVAVAIWRR

SEQ ID NO: 458 - protein, 12 aa, Artificial Sequence
  synthetic peptide
VRLIVAVRAWRR

SEQ ID NO: 459 - protein, 12 aa, Artificial Sequence
  synthetic peptide
VRLIVAVRIARR

SEQ ID NO: 460 - protein, 12 aa, Artificial Sequence
  synthetic peptide
VRLIVAVRIWAR

SEQ ID NO: 461 - protein, 12 aa, Artificial Sequence
  synthetic peptide
VRLIVAVRIWRA

SEQ ID NO: 462 - protein, 12 aa, Artificial Sequence
  synthetic peptide
RRLIVAVRIWRR

SEQ ID NO: 463 - protein, 12 aa, Artificial Sequence
  synthetic peptide
VRRIVAVRIWRR

SEQ ID NO: 464 - protein, 12 aa, Artificial Sequence
  synthetic peptide
VRLRVAVRIWRR

SEQ ID NO: 465 - protein, 12 aa, Artificial Sequence
  synthetic peptide
VRLIRAVRIWRR

SEQ ID NO: 466 - protein, 12 aa, Artificial Sequence
  synthetic peptide
VRLIVRVRIWRR

SEQ ID NO: 467 - protein, 12 aa, Artificial Sequence
  synthetic peptide
VRLIVARRIWRR

SEQ ID NO: 468 - protein, 12 aa, Artificial Sequence
  synthetic peptide
VRLIVAVRRWRR

SEQ ID NO: 469 - protein, 12 aa, Artificial Sequence
  synthetic peptide
VRLIVAVRIRRR

SEQ ID NO: 470 - protein, 12 aa, Artificial Sequence
  synthetic peptide
KRLIVAVRIWRR

SEQ ID NO: 471 - protein, 12 aa, Artificial Sequence
  synthetic peptide
VKLIVAVRIWRR

SEQ ID NO: 472 - protein, 12 aa, Artificial Sequence
  synthetic peptide
VRKIVAVRIWRR

SEQ ID NO: 473 - protein, 12 aa, Artificial Sequence
  synthetic peptide
VRLKVAVRIWRR

SEQ ID NO: 474 - protein, 12 aa, Artificial Sequence
  synthetic peptide
VRLIKAVRIWRR

SEQ ID NO: 475 - protein, 12 aa, Artificial Sequence
  synthetic peptide
VRLIVKVRIWRR

SEQ ID NO: 476 - protein, 12 aa, Artificial Sequence
  synthetic peptide
VRLIVAKRIWRR

SEQ ID NO: 477 - protein, 12 aa, Artificial Sequence
  synthetic peptide
VRLIVAVKIWRR

SEQ ID NO: 478 - protein, 12 aa, Artificial Sequence
  synthetic peptide
VRLIVAVRKWRR

SEQ ID NO: 479 - protein, 12 aa, Artificial Sequence
  synthetic peptide
VRLIVAVRIKRR

SEQ ID NO: 480 - protein, 12 aa, Artificial Sequence
  synthetic peptide
VRLIVAVRIWKR

SEQ ID NO: 481 - protein, 12 aa, Artificial Sequence
  synthetic peptide
VRLIVAVRIWRK

SEQ ID NO: 482 - protein, 12 aa, Artificial Sequence
  synthetic peptide
LRLIVAVRIWRR

SEQ ID NO: 483 - protein, 12 aa, Artificial Sequence
  synthetic peptide
VLLIVAVRIWRR

SEQ ID NO: 484 - protein, 12 aa, Artificial Sequence
  synthetic peptide
VRLLVAVRIWRR

SEQ ID NO: 485 - protein, 12 aa, Artificial Sequence
  synthetic peptide
VRLILAVRIWRR

SEQ ID NO: 486 - protein, 12 aa, Artificial Sequence
  synthetic peptide
VRLIVLVRIWRR

SEQ ID NO: 487 - protein, 12 aa, Artificial Sequence
  synthetic peptide
VRLIVALRIWRR

SEQ ID NO: 488 - protein, 12 aa, Artificial Sequence
  synthetic peptide
VRLIVAVLIWRR

SEQ ID NO: 489 - protein, 12 aa, Artificial Sequence
  synthetic peptide
VRLIVAVRLWRR

SEQ ID NO: 490 - protein, 12 aa, Artificial Sequence
  synthetic peptide
VRLIVAVRILRR

SEQ ID NO: 491 - protein, 12 aa, Artificial Sequence
  synthetic peptide
VRLIVAVRIWLR

SEQ ID NO: 492 - protein, 12 aa, Artificial Sequence
  synthetic peptide
VRLIVAVRIWRL

SEQ ID NO: 493 - protein, 12 aa, Artificial Sequence
  synthetic peptide
IRLIVAVRIWRR

SEQ ID NO: 494 - protein, 12 aa, Artificial Sequence
  synthetic peptide
VILIVAVRIWRR

SEQ ID NO: 495 - protein, 12 aa, Artificial Sequence
  synthetic peptide
VRIIVAVRIWRR

SEQ ID NO: 496 - protein, 12 aa, Artificial Sequence
  synthetic peptide
VRLIIAVRIWRR

SEQ ID NO: 497 - protein, 12 aa, Artificial Sequence
  synthetic peptide
VRLIVIVRIWRR

SEQ ID NO: 498 - protein, 12 aa, Artificial Sequence
  synthetic peptide
VRLIVAIRIWRR

SEQ ID NO: 499 - protein, 12 aa, Artificial Sequence
  synthetic peptide
VRLIVAVIIWRR

SEQ ID NO: 500 - protein, 12 aa, Artificial Sequence
  synthetic peptide
VRLIVAVRIIRR

SEQ ID NO: 501 - protein, 12 aa, Artificial Sequence
  synthetic peptide
VRLIVAVRIWIR

SEQ ID NO: 502 - protein, 12 aa, Artificial Sequence
  synthetic peptide
VRLIVAVRIWRI

SEQ ID NO: 503 - protein, 12 aa, Artificial Sequence
  synthetic peptide
VVLIVAVRIWRR

SEQ ID NO: 504 - protein, 12 aa, Artificial Sequence
  synthetic peptide
VRVIVAVRIWRR

SEQ ID NO: 505 - protein, 12 aa, Artificial Sequence
  synthetic peptide
VRLVVAVRIWRR

SEQ ID NO: 506 - protein, 12 aa, Artificial Sequence
  synthetic peptide
VRLIVVVRIWRR

SEQ ID NO: 507 - protein, 12 aa, Artificial Sequence
  synthetic peptide
VRLIVAVVIWRR

SEQ ID NO: 508 - protein, 12 aa, Artificial Sequence
  synthetic peptide
VRLIVAVRVWRR

SEQ ID NO: 509 - protein, 12 aa, Artificial Sequence
  synthetic peptide
VRLIVAVRIVRR

SEQ ID NO: 510 - protein, 12 aa, Artificial Sequence
  synthetic peptide
VRLIVAVRIWVR

SEQ ID NO: 511 - protein, 12 aa, Artificial Sequence
  synthetic peptide
VRLIVAVRIWRV

SEQ ID NO: 512 - protein, 13 aa, Artificial Sequence
  synthetic peptide
ZWRLIVAVRIWRR

SEQ ID NO: 513 - protein, 12 aa, Artificial Sequence
  synthetic peptide
VWLIVAVRIWRR

SEQ ID NO: 514 - protein, 12 aa, Artificial Sequence
  synthetic peptide
VRWIVAVRIWRR

SEQ ID NO: 515 - protein, 12 aa, Artificial Sequence
  synthetic peptide
VRLWVAVRIWRR

SEQ ID NO: 516 - protein, 12 aa, Artificial Sequence
  synthetic peptide
VRLIWAVRIWRR

SEQ ID NO: 517 - protein, 12 aa, Artificial Sequence
  synthetic peptide
VRLIVWVRIWRR

SEQ ID NO: 518 - protein, 12 aa, Artificial Sequence
  synthetic peptide
VRLIVAWRIWRR

SEQ ID NO: 519 - protein, 12 aa, Artificial Sequence
  synthetic peptide
VRLIVAVWIWRR

SEQ ID NO: 520 - protein, 12 aa, Artificial Sequence
  synthetic peptide
VRLIVAVRWWRR

SEQ ID NO: 521 - protein, 12 aa, Artificial Sequence
  synthetic peptide
VRLIVAVRIWWR

SEQ ID NO: 522 - protein, 12 aa, Artificial Sequence
  synthetic peptide
VRLIVAVRIWRW

SEQ ID NO: 523 - protein, 12 aa, Artificial Sequence
  synthetic peptide
QRLIVAVRIWRR

SEQ ID NO: 524 - protein, 12 aa, Artificial Sequence
  synthetic peptide
VQLIVAVRIWRR

SEQ ID NO: 525 - protein, 12 aa, Artificial Sequence
  synthetic peptide
VRQIVAVRIWRR

SEQ ID NO: 526 - protein, 12 aa, Artificial Sequence
  synthetic peptide
VRLQVAVRIWRR

SEQ ID NO: 527 - protein, 12 aa, Artificial Sequence
  synthetic peptide
VRLIQAVRIWRR

SEQ ID NO: 528 - protein, 12 aa, Artificial Sequence
  synthetic peptide
VRLIVQVRIWRR

SEQ ID NO: 529 - protein, 12 aa, Artificial Sequence
  synthetic peptide
VRLIVAQRIWRR

SEQ ID NO: 530 - protein, 12 aa, Artificial Sequence
  synthetic peptide
VRLIVAVQIWRR

SEQ ID NO: 531 - protein, 12 aa, Artificial Sequence
  synthetic peptide
VRLIVAVRQWRR

SEQ ID NO: 532 - protein, 12 aa, Artificial Sequence
  synthetic peptide
VRLIVAVRIQRR

SEQ ID NO: 533 - protein, 12 aa, Artificial Sequence
  synthetic peptide
VRLIVAVRIWQR

SEQ ID NO: 534 - protein, 12 aa, Artificial Sequence
  synthetic peptide
VRLIVAVRIWRQ

SEQ ID NO: 535 - protein, 12 aa, Artificial Sequence
  synthetic peptide
LKIWKIVRWRLR

SEQ ID NO: 536 - protein, 12 aa, Artificial Sequence
  synthetic peptide
LLLLRIRLLKWR

SEQ ID NO: 537 - protein, 12 aa, Artificial Sequence
  synthetic peptide
VILLRAKLIKVR

SEQ ID NO: 538 - protein, 12 aa, Artificial Sequence
  synthetic peptide
IWWKIGKWLVRK

SEQ ID NO: 539 - protein, 12 aa, Artificial Sequence
  synthetic peptide
VIAKVIVRKVKK

SEQ ID NO: 540 - protein, 12 aa, Artificial Sequence
  synthetic peptide
VRWQKLRRWVIR

SEQ ID NO: 541 - protein, 12 aa, Artificial Sequence
  synthetic peptide
QIWIKIKILKLK

SEQ ID NO: 542 - protein, 12 aa, Artificial Sequence
  synthetic peptide
VLKVIIRRWRLI

SEQ ID NO: 543 - protein, 12 aa, Artificial Sequence
  synthetic peptide
RRWLKLRIVLLK

SEQ ID NO: 544 - protein, 12 aa, Artificial Sequence
  synthetic peptide
IVKVVVQKIKGK

SEQ ID NO: 545 - protein, 12 aa, Artificial Sequence
  synthetic peptide
KQLLRLLIKIVK

SEQ ID NO: 546 - protein, 12 aa, Artificial Sequence
  synthetic peptide
VIIRWLGIRLKW

SEQ ID NO: 547 - protein, 12 aa, Artificial Sequence
  synthetic peptide
IVQKVWIWRVRK

SEQ ID NO: 548 - protein, 12 aa, Artificial Sequence
  synthetic peptide
AWRIRLKWLKLQ

SEQ ID NO: 549 - protein, 12 aa, Artificial Sequence
  synthetic peptide
RQIWKWILQKIK

SEQ ID NO: 550 - protein, 12 aa, Artificial Sequence
  synthetic peptide
LWRWVVRKIRRW

SEQ ID NO: 551 - protein, 12 aa, Artificial Sequence
  synthetic peptide
KILKALIQKVQR

SEQ ID NO: 552 - protein, 12 aa, Artificial Sequence
  synthetic peptide
IIWRILVQVLQK

SEQ ID NO: 553 - protein, 12 aa, Artificial Sequence
  synthetic peptide
IWIIKVLWLKWK

SEQ ID NO: 554 - protein, 12 aa, Artificial Sequence
  synthetic peptide
WWKLILWQVKQA

SEQ ID NO: 555 - protein, 12 aa, Artificial Sequence
  synthetic peptide
RWKVIIWRIQIW

SEQ ID NO: 556 - protein, 12 aa, Artificial Sequence
  synthetic peptide
KWWVKLLVKRIQ

SEQ ID NO: 557 - protein, 12 aa, Artificial Sequence
  synthetic peptide
RIVRVRGVIIKW

SEQ ID NO: 558 - protein, 12 aa, Artificial Sequence
  synthetic peptide
WIRWLALRIRWL

SEQ ID NO: 559 - protein, 12 aa, Artificial Sequence
  synthetic peptide
LVAKIVVVKWKQ

SEQ ID NO: 560 - protein, 12 aa, Artificial Sequence
  synthetic peptide
LQRVKLKWWKWG

SEQ ID NO: 561 - protein, 12 aa, Artificial Sequence
  synthetic peptide
VVKIWIQRIKLV

SEQ ID NO: 562 - protein, 12 aa, Artificial Sequence
  synthetic peptide
AVQWRIRWWKLK

SEQ ID NO: 563 - protein, 12 aa, Artificial Sequence
  synthetic peptide
QILKLKIKRLRI

SEQ ID NO: 564 - protein, 12 aa, Artificial Sequence
  synthetic peptide
KQVVKWRLKKVK

SEQ ID NO: 565 - protein, 12 aa, Artificial Sequence
  synthetic peptide
AWRAILWKLKWR

SEQ ID NO: 566 - protein, 12 aa, Artificial Sequence
  synthetic peptide
WIKLLVKIWKVK

SEQ ID NO: 567 - protein, 12 aa, Artificial Sequence
  Synthetic peptide
ILRVKLRLIQRK

SEQ ID NO: 568 - protein, 12 aa, Artificial Sequence
  synthetic peptide
VQVIKAWAKILK

SEQ ID NO: 569 - protein, 12 aa, Artificial Sequence
  synthetic peptide
LIAIKVKIRWIK

SEQ ID NO: 570 - protein, 12 aa, Artificial Sequence
  synthetic peptide
IIKLKVVLIKIQ

SEQ ID NO: 571 - protein, 12 aa, Artificial Sequence
  synthetic peptide
IIIKVIGRRVQW

SEQ ID NO: 572 - protein, 12 aa, Artificial Sequence
  synthetic peptide
AVVWKLWWIKKK

SEQ ID NO: 573 - protein, 12 aa, Artificial Sequence
  synthetic peptide
QWLIKRVLWKVR

SEQ ID NO: 574 - protein, 12 aa, Artificial Sequence
  synthetic peptide
GWIWRLLWRVIK

SEQ ID NO: 575 - protein, 12 aa, Artificial Sequence
  synthetic peptide
AWKIIGLIIRQV

SEQ ID NO: 576 - protein, 12 aa, Artificial Sequence
  synthetic peptide
KVQRWIVRLIKV

SEQ ID NO: 577 - protein, 12 aa, Artificial Sequence
  synthetic peptide
VVIQQLALWKAK

SEQ ID NO: 578 - protein, 12 aa, Artificial Sequence
  synthetic peptide
RIWRLKWRRWQK

SEQ ID NO: 579 - protein, 12 aa, Artificial Sequence
  synthetic peptide
IWKLRVVIVQKI

SEQ ID NO: 580 - protein, 12 aa, Artificial Sequence
  synthetic peptide
KIRIKLWRVRWK

SEQ ID NO: 581 - protein, 12 aa, Artificial Sequence
  synthetic peptide
ALQKVRVLVAKI

SEQ ID NO: 582 - protein, 12 aa, Artificial Sequence
  synthetic peptide
KKGVKIKLLLVK

SEQ ID NO: 583 - protein, 12 aa, Artificial Sequence
  synthetic peptide
IKWVRWWLKRLQ

SEQ ID NO: 584 - protein, 12 aa, Artificial Sequence
  synthetic peptide
WWQRVRILWLRK

SEQ ID NO: 585 - protein, 12 aa, Artificial Sequence
  synthetic peptide
VKGWRWRKWRIQ

SEQ ID NO: 586 - protein, 12 aa, Artificial Sequence
  synthetic peptide
KWWRIRGKWARL

SEQ ID NO: 587 - protein, 12 aa, Artificial Sequence
  synthetic peptide
VVRIAWKRVQIK

SEQ ID NO: 588 - protein, 12 aa, Artificial Sequence
  synthetic peptide
KILRWKRWRWRI

SEQ ID NO: 589 - protein, 12 aa, Artificial Sequence
  synthetic peptide
AIIGLKVWLIRI

SEQ ID NO: 590 - protein, 12 aa, Artificial Sequence
  synthetic peptide
LIWRVIIGIWQK

SEQ ID NO: 591 - protein, 12 aa, Artificial Sequence
  synthetic peptide
WALKVKVWLIGW

SEQ ID NO: 592 - protein, 12 aa, Artificial Sequence
  synthetic peptide
IIRIGKWGAKVI

SEQ ID NO: 593 - protein, 12 aa, Artificial Sequence
  synthetic peptide
LILKILLKVWKG

SEQ ID NO: 594 - protein, 12 aa, Artificial Sequence
  synthetic peptide
VALVGWWRLQLK

SEQ ID NO: 595 - protein, 12 aa, Artificial Sequence
  synthetic peptide
LLAVKLKVGVAR

SEQ ID NO: 596 - protein, 12 aa, Artificial Sequence
  synthetic peptide
LVKWKLAWAKGL

SEQ ID NO: 597 - protein, 12 aa, Artificial Sequence
  synthetic peptide
AIWKVKAWVVGW

SEQ ID NO: 598 - protein, 12 aa, Artificial Sequence
  synthetic peptide
VAKIAVKWLQLI

SEQ ID NO: 599 - protein, 12 aa, Artificial Sequence
  synthetic peptide
VWGVWLWRIKKL

SEQ ID NO: 600 - protein, 12 aa, Artificial Sequence
  synthetic peptide
GRVIKVKGWLVV

SEQ ID NO: 601 - protein, 12 aa, Artificial Sequence
  synthetic peptide
ILRGIVGLAKIK

SEQ ID NO: 602 - protein, 12 aa, Artificial Sequence
  Synthetic peptide
WLGKAALKILQL

SEQ ID NO: 603 - protein, 12 aa, Artificial Sequence
  synthetic peptide
VQAWKAWIARVK

SEQ ID NO: 604 - protein, 12 aa, Artificial Sequence
  synthetic peptide
KAAKVIWWGIAA

SEQ ID NO: 605 - protein, 12 aa, Artificial Sequence
  synthetic peptide
IAKVVWWKVWGL

SEQ ID NO: 606 - protein, 12 aa, Artificial Sequence
  synthetic peptide
IVAIKAKVQALR

SEQ ID NO: 607 - protein, 12 aa, Artificial Sequence
  synthetic peptide
GVLKAVVVKVKL

SEQ ID NO: 608 - protein, 12 aa, Artificial Sequence
  synthetic peptide
WWKIGAVLIKRA

SEQ ID NO: 609 - protein, 12 aa, Artificial Sequence
  synthetic peptide
WLKAWGGKIRVL

SEQ ID NO: 610 - protein, 12 aa, Artificial Sequence
  synthetic peptide
ALRARVAVAKIK

SEQ ID NO: 611 - protein, 12 aa, Artificial Sequence
  synthetic peptide
VKIGRWVQWAWK

SEQ ID NO: 612 - protein, 12 aa, Artificial Sequence
  synthetic peptide
VVWIKIALGLLK

SEQ ID NO: 613 - protein, 12 aa, Artificial Sequence
  synthetic peptide
LIILKIWWWGAK

SEQ ID NO: 614 - protein, 12 aa, Artificial Sequence
  synthetic peptide
AWVLKVWIWKGQ

SEQ ID NO: 615 - protein, 12 aa, Artificial Sequence
  synthetic peptide
KWLIIGIWWVKG

SEQ ID NO: 616 - protein, 12 aa, Artificial Sequence
  synthetic peptide
IAKIWILGLKVK

SEQ ID NO: 617 - protein, 12 aa, Artificial Sequence
  synthetic peptide
IARWGLLAAKGI

SEQ ID NO: 618 - protein, 12 aa, Artificial Sequence
  synthetic peptide
LVRVGGIVVKKW

SEQ ID NO: 619 - protein, 12 aa, Artificial Sequence
  synthetic peptide
LWGIKGWKLKLW

SEQ ID NO: 620 - protein, 12 aa, Artificial Sequence
  synthetic peptide
VVIKILIGVLRA

SEQ ID NO: 621 - protein, 12 aa, Artificial Sequence
  synthetic peptide
LKIWKIAAKVGQ

SEQ ID NO: 622 - protein, 12 aa, Artificial Sequence
  synthetic peptide
KIAKIAALKIRA

SEQ ID NO: 623 - protein, 28 aa, Artificial Sequence
  Synthetic peptide
SYNTHETICPEPTIDELALKGVLKWLKG

SEQ ID NO: 624 - protein, 12 aa, Artificial Sequence
  synthetic peptide
AVLLKVGVWLVR

SEQ ID NO: 625 - protein, 12 aa, Artificial Sequence
  synthetic peptide
VKLWKILGVAVK

SEQ ID NO: 626 - protein, 12 aa, Artificial Sequence
  synthetic peptide
AWGWKVKVIGAK

SEQ ID NO: 627 - protein, 12 aa, Artificial Sequence
  synthetic peptide
WAWKVVGLILKI

SEQ ID NO: 628 - protein, 12 aa, Artificial Sequence
  synthetic peptide
GIRKLIWWGRAV

SEQ ID NO: 629 - protein, 12 aa, Artificial Sequence
  synthetic peptide
LWVGRVLGIILK

SEQ ID NO: 630 - protein, 12 aa, Artificial Sequence
  synthetic peptide
GVWVRLALWKLV

SEQ ID NO: 631 - protein, 12 aa, Artificial Sequence
  synthetic peptide
IQVARAGAAIIR

SEQ ID NO: 632 - protein, 12 aa, Artificial Sequence
  synthetic peptide
IKVQLLGVWVIR

SEQ ID NO: 633 - protein, 12 aa, Artificial Sequence
  synthetic peptide
GIIKWVAKWVRI

SEQ ID NO: 634 - protein, 12 aa, Artificial Sequence
  synthetic peptide
ILVIKAGGLLIK

SEQ ID NO: 635 - protein, 12 aa, Artificial Sequence
  synthetic peptide
LIVKAIAVRGKI

SEQ ID NO: 636 - protein, 12 aa, Artificial Sequence
  synthetic peptide
VAGIKWAVWKLR

SEQ ID NO: 637 - protein, 12 aa, Artificial Sequence
  synthetic peptide
WGVVAKWWKIQI

SEQ ID NO: 638 - protein, 12 aa, Artificial Sequence
  synthetic peptide
KIWKLAIVGWKI

SEQ ID NO: 639 - protein, 12 aa, Artificial Sequence
  synthetic peptide
KIIIKLLGWGVK

SEQ ID NO: 640 - protein, 12 aa, Artificial Sequence
  synthetic peptide
WWIKGIIIKLKK

SEQ ID NO: 641 - protein, 12 aa, Artificial Sequence
  synthetic peptide
LLRAWIVRIQGL

SEQ ID NO: 642 - protein, 12 aa, Artificial Sequence
  synthetic peptide
IIKIQIWWIRII

SEQ ID NO: 643 - protein, 12 aa, Artificial Sequence
  synthetic peptide
WLVKIVVQWAQK

SEQ ID NO: 644 - protein, 12 aa, Artificial Sequence
  synthetic peptide
GAIIKWKLIAVR

SEQ ID NO: 645 - protein, 12 aa, Artificial Sequence
  synthetic peptide
AWKVGVWLVRAG

SEQ ID NO: 646 - protein, 12 aa, Artificial Sequence
  synthetic peptide
WWVVKAKIALAR

SEQ ID NO: 647 - protein, 12 aa, Artificial Sequence
  synthetic peptide
RWGLKWVAWKAI

SEQ ID NO: 648 - protein, 12 aa, Artificial Sequence
  synthetic peptide
LIAKIRVIVGRA

SEQ ID NO: 649 - protein, 12 aa, Artificial Sequence
  synthetic peptide
VIQVIRIWGARL

SEQ ID NO: 650 - protein, 12 aa, Artificial Sequence
  synthetic peptide
GILKLKVVWGKI

SEQ ID NO: 651 - protein, 12 aa, Artificial Sequence
  synthetic peptide
WIRLGILAVKAW

SEQ ID NO: 652 - protein, 12 aa, Artificial Sequence
  synthetic peptide
AIVKVKIAWGRI

SEQ ID NO: 653 - protein, 12 aa, Artificial Sequence
  synthetic peptide
AALAKLKIWGLG

SEQ ID NO: 654 - protein, 12 aa, Artificial Sequence
  synthetic peptide
ILKAIIKIIQWG

SEQ ID NO: 655 - protein, 12 aa, Artificial Sequence
  synthetic peptide
ALKLAVLGIRLL

SEQ ID NO: 656 - protein, 12 aa, Artificial Sequence
  synthetic peptide
WGWRAIARIWQI

SEQ ID NO: 657 - protein, 12 aa, Artificial Sequence
  synthetic peptide
IVIKGGIWKIAR

SEQ ID NO: 658 - protein, 12 aa, Artificial Sequence
  synthetic peptide
KILKAVLGGIRW

SEQ ID NO: 659 - protein, 12 aa, Artificial Sequence
  synthetic peptide
AWAVKWRVARVK

SEQ ID NO: 660 - protein, 12 aa, Artificial Sequence
  synthetic peptide
IWVKAKLARIKA

SEQ ID NO: 661 - protein, 12 aa, Artificial Sequence
  synthetic peptide
IQVVKLWKWQLA

SEQ ID NO: 662 - protein, 12 aa, Artificial Sequence
  synthetic peptide
LLIKGVVIKIQV

SEQ ID NO: 663 - protein, 12 aa, Artificial Sequence
  synthetic peptide
AVKWVVAGARLV

SEQ ID NO: 664 - protein, 12 aa, Artificial Sequence
  synthetic peptide
AIIKILWRLWKI

SEQ ID NO: 665 - protein, 12 aa, Artificial Sequence
  synthetic peptide
AGWKWQVWRAKK

SEQ ID NO: 666 - protein, 12 aa, Artificial Sequence
  synthetic peptide
LIKWAIWKVGKI

SEQ ID NO: 667 - protein, 12 aa, Artificial Sequence
  synthetic peptide
GLQKWVWRIWKI

SEQ ID NO: 668 - protein, 12 aa, Artificial Sequence
  synthetic peptide
AVRIKVWKWGRR

SEQ ID NO: 669 - protein, 12 aa, Artificial Sequence
  synthetic peptide
VQAARGLAKKAR

SEQ ID NO: 670 - protein, 12 aa, Artificial Sequence
  Synthetic peptide
VLRGKLLWVQVI

SEQ ID NO: 671 - protein, 12 aa, Artificial Sequence
  synthetic peptide
AWAKAKGKGVRV

SEQ ID NO: 672 - protein, 12 aa, Artificial Sequence
  synthetic peptide
AVKWKIWAGQIL

SEQ ID NO: 673 - protein, 12 aa, Artificial Sequence
  synthetic peptide
ILRILIKVVRKK

SEQ ID NO: 674 - protein, 12 aa, Artificial Sequence
  synthetic peptide
KALKVIWRGVKG

SEQ ID NO: 675 - protein, 12 aa, Artificial Sequence
  synthetic peptide
KIIIIKVWLGRA

SEQ ID NO: 676 - protein, 12 aa, Artificial Sequence
  synthetic peptide
GVKRVAVWRAKK

SEQ ID NO: 677 - protein, 12 aa, Artificial Sequence
  synthetic peptide
ILVKIWIRWGRA

SEQ ID NO: 678 - protein, 12 aa, Artificial Sequence
  synthetic peptide
LIRWRAVVLKVA

SEQ ID NO: 679 - protein, 12 aa, Artificial Sequence
  synthetic peptide
AAVKRIRIWLGK

SEQ ID NO: 680 - protein, 12 aa, Artificial Sequence
  synthetic peptide
RKGVKIAVRAGR

SEQ ID NO: 681 - protein, 12 aa, Artificial Sequence
  synthetic peptide
RVGVKIIWQKAK

SEQ ID NO: 682 - protein, 12 aa, Artificial Sequence
  synthetic peptide
AGLKKKIWVGRK

SEQ ID NO: 683 - protein, 12 aa, Artificial Sequence
  synthetic peptide
KKVRIGGWIARI

SEQ ID NO: 684 - protein, 12 aa, Artificial Sequence
  synthetic peptide
GKLGKLVWWKRR

SEQ ID NO: 685 - protein, 12 aa, Artificial Sequence
  synthetic peptide
WRLIKIWLWRKQ

SEQ ID NO: 686 - protein, 12 aa, Artificial Sequence
  synthetic peptide
KIALARLWAWRR

SEQ ID NO: 687 - protein, 12 aa, Artificial Sequence
  synthetic peptide
VVIKLKIRRGRR

SEQ ID NO: 688 - protein, 12 aa, Artificial Sequence
  synthetic peptide
IRIARWAIKIWQ

SEQ ID NO: 689 - protein, 12 aa, Artificial Sequence
  synthetic peptide
ALQAIKVWAWKL

SEQ ID NO: 690 - protein, 12 aa, Artificial Sequence
  synthetic peptide
VLQLGIWAAKRK

SEQ ID NO: 691 - protein, 12 aa, Artificial Sequence
  synthetic peptide
VIAKIGIWIGRV

SEQ ID NO: 692 - protein, 12 aa, Artificial Sequence
  synthetic peptide
QIWKKKAWAGRI

SEQ ID NO: 693 - protein, 12 aa, Artificial Sequence
  synthetic peptide
ALVGKIGIWRIL

SEQ ID NO: 694 - protein, 12 aa, Artificial Sequence
  synthetic peptide
LRAIKIKWWRWV

SEQ ID NO: 695 - protein, 12 aa, Artificial Sequence
  synthetic peptide
GAVLKLVWRLVR

SEQ ID NO: 696 - protein, 12 aa, Artificial Sequence
  synthetic peptide
AKIQKIIWGRIR

SEQ ID NO: 697 - protein, 12 aa, Artificial Sequence
  synthetic peptide
ALQKLWAKRARW

SEQ ID NO: 698 - protein, 12 aa, Artificial Sequence
  synthetic peptide
RRGRLKLWIVRI

SEQ ID NO: 699 - protein, 12 aa, Artificial Sequence
  synthetic peptide
RGARWGIKKAKV

SEQ ID NO: 700 - protein, 12 aa, Artificial Sequence
  synthetic peptide
KKLWKAKAKVVR

SEQ ID NO: 701 - protein, 12 aa, Artificial Sequence
  synthetic peptide
LWKKVVWIGKKK

SEQ ID NO: 702 - protein, 12 aa, Artificial Sequence
  synthetic peptide
QLARRKAWKVKG

SEQ ID NO: 703 - protein, 12 aa, Artificial Sequence
  synthetic peptide
KVIQKAAIQVWK

SEQ ID NO: 704 - protein, 12 aa, Artificial Sequence
  synthetic peptide
RGKIALWGWKRI

SEQ ID NO: 705 - protein, 12 aa, Artificial Sequence
  synthetic peptide
AAILKIGIWLGK

SEQ ID NO: 706 - protein, 12 aa, Artificial Sequence
  synthetic peptide
LLWIKIAWIRGK

SEQ ID NO: 707 - protein, 12 aa, Artificial Sequence
  synthetic peptide
KVIGRVVLWGIK

SEQ ID NO: 708 - protein, 12 aa, Artificial Sequence
  synthetic peptide
IAKLKIVGGKKK

SEQ ID NO: 709 - protein, 12 aa, Artificial Sequence
  synthetic peptide
KVVRWAKWVAKK

SEQ ID NO: 710 - protein, 12 aa, Artificial Sequence
  synthetic peptide
WIAKIKVWKILK

SEQ ID NO: 711 - protein, 12 aa, Artificial Sequence
  synthetic peptide
AIRGVVVRWKQA

SEQ ID NO: 712 - protein, 12 aa, Artificial Sequence
  synthetic peptide
GALAKARVKGAK

SEQ ID NO: 713 - protein, 12 aa, Artificial Sequence
  synthetic peptide
AQIWRLKIWWVR

SEQ ID NO: 714 - protein, 12 aa, Artificial Sequence
  synthetic peptide
LQRALVGWVKWK

SEQ ID NO: 715 - protein, 12 aa, Artificial Sequence
  synthetic peptide
ALAKIAGGKVRI

SEQ ID NO: 716 - protein, 12 aa, Artificial Sequence
  synthetic peptide
KQRAKIWRLRKV

SEQ ID NO: 717 - protein, 12 aa, Artificial Sequence
  synthetic peptide
VQRVAGKKARRI

SEQ ID NO: 718 - protein, 12 aa, Artificial Sequence
  synthetic peptide
GIRVIWQVIKKR

SEQ ID NO: 719 - protein, 12 aa, Artificial Sequence
  synthetic peptide
LARLAWWRQKAR

SEQ ID NO: 720 - protein, 12 aa, Artificial Sequence
  synthetic peptide
RIVRAAWKGARI

SEQ ID NO: 721 - protein, 12 aa, Artificial Sequence
  synthetic peptide
VGRARIAIIKWL

SEQ ID NO: 722 - protein, 12 aa, Artificial Sequence
  synthetic peptide
VWGKVVLWGKKR

SEQ ID NO: 723 - protein, 12 aa, Artificial Sequence
  synthetic peptide
IAKVVWVRAQRG

SEQ ID NO: 724 - protein, 12 aa, Artificial Sequence
  synthetic peptide
KRLARAAGIIAR

SEQ ID NO: 725 - protein, 12 aa, Artificial Sequence
  synthetic peptide
KAVKIAIKVWKR

SEQ ID NO: 726 - protein, 12 aa, Artificial Sequence
  synthetic peptide
KWIGAKIWIAKG

SEQ ID NO: 727 - protein, 12 aa, Artificial Sequence
  synthetic peptide
LALGKIWKGRAI

SEQ ID NO: 728 - protein, 12 aa, Artificial Sequence
  synthetic peptide
VARQRIIWWKWR

SEQ ID NO: 729 - protein, 12 aa, Artificial Sequence
  synthetic peptide
LWKWKLWRAVRL

SEQ ID NO: 730 - protein, 12 aa, Artificial Sequence
  synthetic peptide
IAKAWWKRLQAI

SEQ ID NO: 731 - protein, 12 aa, Artificial Sequence
  synthetic peptide
AQKIKWRVWKGL

SEQ ID NO: 732 - protein, 12 aa, Artificial Sequence
  synthetic peptide
LKVVKVAAKLGR

SEQ ID NO: 733 - protein, 12 aa, Artificial Sequence
  synthetic peptide
AARIRAWAGWGK

SEQ ID NO: 734 - protein, 12 aa, Artificial Sequence
  synthetic peptide
LVARVKIKGIRI

SEQ ID NO: 735 - protein, 12 aa, Artificial Sequence
  synthetic peptide
LIQKAKVKWVRQ

SEQ ID NO: 736 - protein, 12 aa, Artificial Sequence
  synthetic peptide
ARQVKIGIWILR

SEQ ID NO: 737 - protein, 12 aa, Artificial Sequence
  synthetic peptide
IWVWKIIQWRLR

SEQ ID NO: 738 - protein, 12 aa, Artificial Sequence
  synthetic peptide
WILQKWLWIRLQ

SEQ ID NO: 739 - protein, 12 aa, Artificial Sequence
  synthetic peptide
LQKLLKWLVQKW

SEQ ID NO: 740 - protein, 12 aa, Artificial Sequence
  synthetic peptide
LVKVIVIKIQKW

SEQ ID NO: 741 - protein, 12 aa, Artificial Sequence
  synthetic peptide
VIIKWRVIIAKR

SEQ ID NO: 742 - protein, 12 aa, Artificial Sequence
  synthetic peptide
ILRLLWWKVVIR

SEQ ID NO: 743 - protein, 12 aa, Artificial Sequence
  synthetic peptide
ILLQRLKLWIQR

SEQ ID NO: 744 - protein, 12 aa, Artificial Sequence
  synthetic peptide
ILIKIWAGVVQK

SEQ ID NO: 745 - protein, 12 aa, Artificial Sequence
  synthetic peptide
IWAQKAVVVKIK

SEQ ID NO: 746 - protein, 12 aa, Artificial Sequence
  synthetic peptide
WIRIIIRVIKIA

SEQ ID NO: 747 - protein, 12 aa, Artificial Sequence
  synthetic peptide
WQRLLKWLGKRK

SEQ ID NO: 748 - protein, 12 aa, Artificial Sequence
  synthetic peptide
VKKGLGWLVKIK

SEQ ID NO: 749 - protein, 12 aa, Artificial Sequence
  synthetic peptide
VVLKWIIRKIKI

SEQ ID NO: 750 - protein, 12 aa, Artificial Sequence
  synthetic peptide
LLKLLGQLAKVV

SEQ ID NO: 751 - protein, 12 aa, Artificial Sequence
  synthetic peptide
IIIKIVGVVWKW

SEQ ID NO: 752 - protein, 12 aa, Artificial Sequence
  synthetic peptide
RALAKALLARIK

SEQ ID NO: 753 - protein, 12 aa, Artificial Sequence
  synthetic peptide
KILKILARVLRW

SEQ ID NO: 754 - protein, 12 aa, Artificial Sequence
  synthetic peptide
VAKLRLQLIKLV

SEQ ID NO: 755 - protein, 12 aa, Artificial Sequence
  synthetic peptide
VVKAVIGKIKIQ

SEQ ID NO: 756 - protein, 12 aa, Artificial Sequence
  synthetic peptide
LKWLKVGIRKVR

SEQ ID NO: 757 - protein, 12 aa, Artificial Sequence
  synthetic peptide
WLIKARVQLLRK

SEQ ID NO: 758 - protein, 12 aa, Artificial Sequence
  synthetic peptide
ILLLKVLLVKWR

SEQ ID NO: 759 - protein, 12 aa, Artificial Sequence
  synthetic peptide
QWVKVLIIKWKK

SEQ ID NO: 760 - protein, 12 aa, Artificial Sequence
  synthetic peptide
QGWRIRLKWIKI

SEQ ID NO: 761 - protein, 12 aa, Artificial Sequence
  synthetic peptide
IIKLKLRVAQKL

SEQ ID NO: 762 - protein, 12 aa, Artificial Sequence
  synthetic peptide
VLGWKIKGAKVK

SEQ ID NO: 763 - protein, 12 aa, Artificial Sequence
  synthetic peptide
IWLKWKIQIAQA

SEQ ID NO: 764 - protein, 12 aa, Artificial Sequence
  synthetic peptide
LWIRILVRVVRL

SEQ ID NO: 765 - protein, 12 aa, Artificial Sequence
  synthetic peptide
LIIGVKGWKLKV

SEQ ID NO: 766 - protein, 12 aa, Artificial Sequence
  synthetic peptide
WIVQKIKRWLVK

SEQ ID NO: 767 - protein, 12 aa, Artificial Sequence
  synthetic peptide
LLWLRARWKIVK

SEQ ID NO: 768 - protein, 12 aa, Artificial Sequence
  synthetic peptide
LWKIVLIGGKWW

SEQ ID NO: 769 - protein, 12 aa, Artificial Sequence
  synthetic peptide
RWALRIVWRRWQ

SEQ ID NO: 770 - protein, 12 aa, Artificial Sequence
  synthetic peptide
RVIRIIIAKLKL

SEQ ID NO: 771 - protein, 12 aa, Artificial Sequence
  synthetic peptide
LRIVWVKLWILK

SEQ ID NO: 772 - protein, 12 aa, Artificial Sequence
  synthetic peptide
ALKIVIWQIKQL

SEQ ID NO: 773 - protein, 12 aa, Artificial Sequence
  synthetic peptide
VKVQRWRVLVIK

SEQ ID NO: 774 - protein, 12 aa, Artificial Sequence
  synthetic peptide
IIILKWIWRAIK

SEQ ID NO: 775 - protein, 12 aa, Artificial Sequence
  synthetic peptide
ALVVKVVIGKIR

SEQ ID NO: 776 - protein, 12 aa, Artificial Sequence
  synthetic peptide
KIVKWVLVKVKI

SEQ ID NO: 777 - protein, 12 aa, Artificial Sequence
  synthetic peptide
IRLIRLKIIIAK

SEQ ID NO: 778 - protein, 12 aa, Artificial Sequence
  synthetic peptide
IKIVKVLGLALK

SEQ ID NO: 779 - protein, 12 aa, Artificial Sequence
  synthetic peptide
WLQKVKLWKVIK

SEQ ID NO: 780 - protein, 12 aa, Artificial Sequence
  synthetic peptide
ALRLVVKVWKKR

SEQ ID NO: 781 - protein, 12 aa, Artificial Sequence
  synthetic peptide
LKVLKVKILGAK

SEQ ID NO: 782 - protein, 12 aa, Artificial Sequence
  synthetic peptide
LLKLQWWAVKKA

SEQ ID NO: 783 - protein, 12 aa, Artificial Sequence
  synthetic peptide
IVRIKIWVKKVL

SEQ ID NO: 784 - protein, 12 aa, Artificial Sequence
  synthetic peptide
VIILKVLAQVVK

SEQ ID NO: 785 - protein, 12 aa, Artificial Sequence
  synthetic peptide
KQKIRVWIAKRI

SEQ ID NO: 786 - protein, 12 aa, Artificial Sequence
  synthetic peptide
IAKIKVILVQLQ

SEQ ID NO: 787 - protein, 12 aa, Artificial Sequence
  synthetic peptide
VLKIKWGRVRWW

SEQ ID NO: 788 - protein, 12 aa, Artificial Sequence
  synthetic peptide
IGKLKLQLIKLR

SEQ ID NO: 789 - protein, 12 aa, Artificial Sequence
  synthetic peptide
VIRKIKIWKVQK

SEQ ID NO: 790 - protein, 12 aa, Artificial Sequence
  synthetic peptide
VLVKVKLRAVRI

SEQ ID NO: 791 - protein, 12 aa, Artificial Sequence
  synthetic peptide
GARIWLQKIKLA

SEQ ID NO: 792 - protein, 12 aa, Artificial Sequence
  synthetic peptide
VWKVIWRAGQLK

SEQ ID NO: 793 - protein, 12 aa, Artificial Sequence
  synthetic peptide
RIIQRWVKWILQ

SEQ ID NO: 794 - protein, 12 aa, Artificial Sequence
  synthetic peptide
IVIAKVWIVRKA

SEQ ID NO: 795 - protein, 12 aa, Artificial Sequence
  synthetic peptide
LQKWKIKKVRIR

SEQ ID NO: 796 - protein, 12 aa, Artificial Sequence
  synthetic peptide
RLVKIRLWRLWK

SEQ ID NO: 797 - protein, 12 aa, Artificial Sequence
  synthetic peptide
ALWIKILKWVWK

SEQ ID NO: 798 - protein, 12 aa, Artificial Sequence
  synthetic peptide
VWVWRVWVRIAR

SEQ ID NO: 799 - protein, 12 aa, Artificial Sequence
  synthetic peptide
KIIKGVARRIRR

SEQ ID NO: 800 - protein, 12 aa, Artificial Sequence
  synthetic peptide
LIIRAVWKIVRK

SEQ ID NO: 801 - protein, 12 aa, Artificial Sequence
  synthetic peptide
GVRLKWLLWRRR

SEQ ID NO: 802 - protein, 12 aa, Artificial Sequence
  synthetic peptide
RLIIKAKIRKVK

SEQ ID NO: 803 - protein, 12 aa, Artificial Sequence
  synthetic peptide
LLWGKGKWRAWK

SEQ ID NO: 804 - protein, 12 aa, Artificial Sequence
  synthetic peptide
RQIRIWVWRIQK

SEQ ID NO: 805 - protein, 12 aa, Artificial Sequence
  synthetic peptide
VIKVKWWGVRVL

SEQ ID NO: 806 - protein, 12 aa, Artificial Sequence
  synthetic peptide
VIKWRIWRRKIR

SEQ ID NO: 807 - protein, 12 aa, Artificial Sequence
  synthetic peptide
LLLKLIIRLARR

SEQ ID NO: 808 - protein, 12 aa, Artificial Sequence
  synthetic peptide
VAKLQIWLVKQK

SEQ ID NO: 809 - protein, 12 aa, Artificial Sequence
  synthetic peptide
RGVKWKWKIVKK

SEQ ID NO: 810 - protein, 12 aa, Artificial Sequence
  synthetic peptide
IQKWVVIRWRLR

SEQ ID NO: 811 - protein, 12 aa, Artificial Sequence
  synthetic peptide
LAKWLRWIWRRQ

SEQ ID NO: 812 - protein, 12 aa, Artificial Sequence
  synthetic peptide
VQIWKLKLLKAK

SEQ ID NO: 813 - protein, 12 aa, Artificial Sequence
  synthetic peptide
IGRWKVQKAKWK

SEQ ID NO: 814 - protein, 12 aa, Artificial Sequence
  synthetic peptide
VIIGKVKWRLIK

SEQ ID NO: 815 - protein, 12 aa, Artificial Sequence
  synthetic peptide
VWWRLWVQRAQI

SEQ ID NO: 816 - protein, 12 aa, Artificial Sequence
  synthetic peptide
AQVLKVVKWKIR

SEQ ID NO: 817 - protein, 12 aa, Artificial Sequence
  synthetic peptide
AAVRLKGIIIKL

SEQ ID NO: 818 - protein, 12 aa, Artificial Sequence
  synthetic peptide
KGAKWIVKKVKR

SEQ ID NO: 819 - protein, 12 aa, Artificial Sequence
  synthetic peptide
AWIRLKAWRVRR

SEQ ID NO: 820 - protein, 12 aa, Artificial Sequence
  synthetic peptide
KLWWKVLLKILK

SEQ ID NO: 821 - protein, 12 aa, Artificial Sequence
  synthetic peptide
KWVKVRVVWLKW

SEQ ID NO: 822 - protein, 12 aa, Artificial Sequence
  synthetic peptide
GIKLAWKKGRKL

SEQ ID NO: 823 - protein, 12 aa, Artificial Sequence
  synthetic peptide
VVWVGVGIWRAK

SEQ ID NO: 824 - protein, 12 aa, Artificial Sequence
  synthetic peptide
LGVVWKLWIIRV

SEQ ID NO: 825 - protein, 12 aa, Artificial Sequence
  synthetic peptide
VGKAVARIGRRV

SEQ ID NO: 826 - protein, 12 aa, Artificial Sequence
  synthetic peptide
KAIRLGAVKGKK

SEQ ID NO: 827 - protein, 12 aa, Artificial Sequence
  synthetic peptide
WVKAVWRRAQWL

SEQ ID NO: 828 - protein, 12 aa, Artificial Sequence
  synthetic peptide
RVWVIGIWGVRK

SEQ ID NO: 829 - protein, 12 aa, Artificial Sequence
  synthetic peptide
LVRLAAKRARGI

SEQ ID NO: 830 - protein, 12 aa, Artificial Sequence
  synthetic peptide
AALKVAGLAIKQ

SEQ ID NO: 831 - protein, 12 aa, Artificial Sequence
  synthetic peptide
VVRGLGLIAKLV

SEQ ID NO: 832 - protein, 12 aa, Artificial Sequence
  synthetic peptide
VVALKLWKVRRG

SEQ ID NO: 833 - protein, 12 aa, Artificial Sequence
  synthetic peptide
KAAQLGLWVWKK

SEQ ID NO: 834 - protein, 12 aa, Artificial Sequence
  synthetic peptide
RVLKLVGIAAKR

SEQ ID NO: 835 - protein, 12 aa, Artificial Sequence
  synthetic peptide
IGKAVIWRGKRL

SEQ ID NO: 836 - protein, 12 aa, Artificial Sequence
  synthetic peptide
AAKGAWKRIKGA

SEQ ID NO: 837 - protein, 12 aa, Artificial Sequence
  synthetic peptide
IKALKVAVRAVQ

SEQ ID NO: 838 - protein, 12 aa, Artificial Sequence
  synthetic peptide
LLQKAAIKWAKK

SEQ ID NO: 839 - protein, 12 aa, Artificial Sequence
  synthetic peptide
RVIKALIGKGRK

SEQ ID NO: 840 - protein, 12 aa, Artificial Sequence
  synthetic peptide
LLRAAWGVWRKV

SEQ ID NO: 841 - protein, 12 aa, Artificial Sequence
  synthetic peptide
KLVRVWARLGQK

SEQ ID NO: 842 - protein, 12 aa, Artificial Sequence
  synthetic peptide
RKIAKAGIWVGI

SEQ ID NO: 843 - protein, 12 aa, Artificial Sequence
  synthetic peptide
GAIIKVWAGRKL

SEQ ID NO: 844 - protein, 12 aa, Artificial Sequence
  synthetic peptide
ILWKAAWKAGRV

SEQ ID NO: 845 - protein, 12 aa, Artificial Sequence
  synthetic peptide
WLAKLAAVRIQR

SEQ ID NO: 846 - protein, 12 aa, Artificial Sequence
  synthetic peptide
GIQRIRVIWAKA

SEQ ID NO: 847 - protein, 12 aa, Artificial Sequence
  synthetic peptide
VKVQKWVAKVAR

SEQ ID NO: 848 - protein, 12 aa, Artificial Sequence
  synthetic peptide
KVGVRGAIRKIR

SEQ ID NO: 849 - protein, 12 aa, Artificial Sequence
  synthetic peptide
IARAKIKWAKIL

SEQ ID NO: 850 - protein, 12 aa, Artificial Sequence
  synthetic peptide
VLGKAVVKGVKV

SEQ ID NO: 851 - protein, 12 aa, Artificial Sequence
  synthetic peptide
WKKAKIWIVQVK

SEQ ID NO: 852 - protein, 12 aa, Artificial Sequence
  synthetic peptide
AAVRAKLKKVRI

SEQ ID NO: 853 - protein, 12 aa, Artificial Sequence
  synthetic peptide
ILWLKVGWWVQK

SEQ ID NO: 854 - protein, 12 aa, Artificial Sequence
  synthetic peptide
RWRRIGWWLIKQ

SEQ ID NO: 855 - protein, 12 aa, Artificial Sequence
  synthetic peptide
AAKAKARAVKVL

SEQ ID NO: 856 - protein, 12 aa, Artificial Sequence
  synthetic peptide
KVAAKAAIGVWK

SEQ ID NO: 857 - protein, 12 aa, Artificial Sequence
  synthetic peptide
ALAGALKIWLGR

SEQ ID NO: 858 - skipped

SEQ ID NO: 859 - protein, 12 aa, Artificial Sequence
  synthetic peptide
GLQRIIVWRLVV

SEQ ID NO: 860 - protein, 12 aa, Artificial Sequence
  synthetic peptide
KIGKAAKWILKI

SEQ ID NO: 861 - protein, 12 aa, Artificial Sequence
  synthetic peptide
WWRIAAVKGRRV

SEQ ID NO: 862 - protein, 12 aa, Artificial Sequence
  synthetic peptide
AVKALIKILKAQ

SEQ ID NO: 863 - protein, 12 aa, Artificial Sequence
  synthetic peptide
IGLGRVAWAKAR

SEQ ID NO: 864 - protein, 12 aa, Artificial Sequence
  synthetic peptide
LVRWAWLGARQV

SEQ ID NO: 865 - protein, 12 aa, Artificial Sequence
  synthetic peptide
GKAIKLALKLLQ

SEQ ID NO: 866 - protein, 12 aa, Artificial Sequence
  synthetic peptide
AAAKVKALGLKA

SEQ ID NO: 867 - protein, 12 aa, Artificial Sequence
  synthetic peptide
VALAKVWIGKVG

SEQ ID NO: 868 - protein, 12 aa, Artificial Sequence
  synthetic peptide
AARAAGIWIRVW

SEQ ID NO: 869 - protein, 12 aa, Artificial Sequence
  synthetic peptide
AVIGKWGVWRAR

SEQ ID NO: 870 - protein, 12 aa, Artificial Sequence
  synthetic peptide
QKAKAVVWRGKK

SEQ ID NO: 871 - protein, 12 aa, Artificial Sequence
  synthetic peptide
VRAQRVKILIVQ

SEQ ID NO: 872 - protein, 12 aa, Artificial Sequence
  synthetic peptide
VVRAGGRLIRAV

SEQ ID NO: 873 - protein, 12 aa, Artificial Sequence
  synthetic peptide
IALIKKGGWLLK

SEQ ID NO: 874 - protein, 12 aa, Artificial Sequence
  synthetic peptide
LKGVLARVWVIQ

SEQ ID NO: 875 - protein, 12 aa, Artificial Sequence
  synthetic peptide
KVIKGIIVGLRL

SEQ ID NO: 876 - protein, 12 aa, Artificial Sequence
  synthetic peptide
VAILKGWVLKIL

SEQ ID NO: 877 - protein, 12 aa, Artificial Sequence
  synthetic peptide
VIRVVWLKVQLG

SEQ ID NO: 878 - protein, 12 aa, Artificial Sequence
  synthetic peptide
IVRLIIQIWRLV

SEQ ID NO: 879 - protein, 12 aa, Artificial Sequence
  synthetic peptide
KIQLVKIWLVKI

SEQ ID NO: 880 - protein, 12 aa, Artificial Sequence
  synthetic peptide
AIIRALWKVVKR

SEQ ID NO: 881 - protein, 12 aa, Artificial Sequence
  synthetic peptide
QIVKRKIWLAKK

SEQ ID NO: 882 - protein, 12 aa, Artificial Sequence
  synthetic peptide
IQVLKIWGIKAK

SEQ ID NO: 883 - protein, 12 aa, Artificial Sequence
  synthetic peptide
LRWWKWLAKIVR

SEQ ID NO: 884 - protein, 12 aa, Artificial Sequence
  synthetic peptide
LVKWKIRVGQVI

SEQ ID NO: 885 - protein, 12 aa, Artificial Sequence
  synthetic peptide
IVKVIWLKVKKQ

SEQ ID NO: 886 - protein, 12 aa, Artificial Sequence
  synthetic peptide
IGIWKVWIQIWK

SEQ ID NO: 887 - protein, 12 aa, Artificial Sequence
  synthetic peptide
RRLGKILWWKVI

SEQ ID NO: 888 - protein, 12 aa, Artificial Sequence
  synthetic peptide
KIWIKAVWQILK

SEQ ID NO: 889 - protein, 12 aa, Artificial Sequence
  synthetic peptide
IQVWRIKWQKLR

SEQ ID NO: 890 - protein, 12 aa, Artificial Sequence
  synthetic peptide
AVRLLIKIWRVK

SEQ ID NO: 891 - protein, 12 aa, Artificial Sequence
  synthetic peptide
GVKGIALKIKVL

SEQ ID NO: 892 - protein, 12 aa, Artificial Sequence
  synthetic peptide
WLIVKWKVWKAR

SEQ ID NO: 893 - protein, 12 aa, Artificial Sequence
  synthetic peptide
KWLAWKVWAIKQ

SEQ ID NO: 894 - protein, 12 aa, Artificial Sequence
  synthetic peptide
VIRIKLKIWQWQ

SEQ ID NO: 895 - protein, 12 aa, Artificial Sequence
  synthetic peptide
VILKVVWAGAKI

SEQ ID NO: 896 - protein, 12 aa, Artificial Sequence
  synthetic peptide
LKVWRVILWGRR

SEQ ID NO: 897 - protein, 12 aa, Artificial Sequence
  synthetic peptide
RRVRAKVWVLRW

SEQ ID NO: 898 - protein, 12 aa, Artificial Sequence
  synthetic peptide
KWQKIWIGRVKV

SEQ ID NO: 899 - protein, 12 aa, Artificial Sequence
  synthetic peptide
LKLQKIAALKIR

SEQ ID NO: 900 - protein, 12 aa, Artificial Sequence
  synthetic peptide
WWIQVIIWRIRL

SEQ ID NO: 901 - protein, 12 aa, Artificial Sequence
  synthetic peptide
VWKIWVKRVRVI

SEQ ID NO: 902 - protein, 12 aa, Artificial Sequence
  synthetic peptide
ILVQRVKILIWK

SEQ ID NO: 903 - protein, 12 aa, Artificial Sequence
  synthetic peptide
ILKVKWIVGRKQ

SEQ ID NO: 904 - protein, 12 aa, Artificial Sequence
  synthetic peptide
VIAKLWRIKWKW

SEQ ID NO: 905 - protein, 12 aa, Artificial Sequence
  synthetic peptide
VIIRWLGIRLKW

SEQ ID NO: 906 - protein, 12 aa, Artificial Sequence
  synthetic peptide
VIVRAVKQRLKL

SEQ ID NO: 907 - protein, 12 aa, Artificial Sequence
  synthetic peptide
IIWKVGRIGAKL

SEQ ID NO: 908 - protein, 12 aa, Artificial Sequence
  synthetic peptide
AIVWGARLRLKW

SEQ ID NO: 909 - protein, 12 aa, Artificial Sequence
  synthetic peptide
IAWKWARVWIKA

SEQ ID NO: 910 - protein, 12 aa, Artificial Sequence
  synthetic peptide
GVLKAVVVKVKL

SEQ ID NO: 911 - protein, 12 aa, Artificial Sequence
  synthetic peptide
LLIRAIKVGLQV

SEQ ID NO: 912 - protein, 12 aa, Artificial Sequence
  synthetic peptide
LIVKAIAVRGKI

SEQ ID NO: 913 - protein, 12 aa, Artificial Sequence
  synthetic peptide
GIVRLAKLKGLV

SEQ ID NO: 914 - protein, 12 aa, Artificial Sequence
  synthetic peptide
LLIKGVVIKIQV

SEQ ID NO: 915 - protein, 12 aa, Artificial Sequence
  synthetic peptide
ILVRAVRGKALV

SEQ ID NO: 916 - protein, 12 aa, Artificial Sequence
  synthetic peptide
ALAKIAGGKVRI

SEQ ID NO: 917 - protein, 12 aa, Artificial Sequence
  synthetic peptide
IAARWIKAKWRG

SEQ ID NO: 918 - protein, 12 aa, Artificial Sequence
  synthetic peptide
GGVKVLRVKVRV

SEQ ID NO: 919 - protein, 12 aa, Artificial Sequence
  synthetic peptide
GILRLAKVKIKG

SEQ ID NO: 920 - protein, 12 aa, Artificial Sequence
  synthetic peptide
IQVRIVRVKWKI

SEQ ID NO: 921 - protein, 12 aa, Artificial Sequence
  synthetic peptide
GALRKIRVKWRV

SEQ ID NO: 922 - protein, 12 aa, Artificial Sequence
  synthetic peptide
AVIKLGKIAWRG

SEQ ID NO: 923 - protein, 12 aa, Artificial Sequence
  synthetic peptide
LVLLGIRAKVRA

SEQ ID NO: 924 - protein, 12 aa, Artificial Sequence
  synthetic peptide
VWAKIWRLKAAI

SEQ ID NO: 925 - protein, 12 aa, Artificial Sequence
  synthetic peptide
IAGKLVRVIWQI

SEQ ID NO: 926 - protein, 12 aa, Artificial Sequence
  synthetic peptide
KAGKLLKLKVQV

SEQ ID NO: 927 - protein, 12 aa, Artificial Sequence
  synthetic peptide
QIKIKVKILLIK

SEQ ID NO: 928 - protein, 12 aa, Artificial Sequence
  synthetic peptide
IIKIKQKVLLVK

SEQ ID NO: 929 - protein, 12 aa, Artificial Sequence
  synthetic peptide
LWKIRLKLVVKK

SEQ ID NO: 930 - protein, 12 aa, Artificial Sequence
  synthetic peptide
IIKIWWKIGWLK

SEQ ID NO: 931 - protein, 12 aa, Artificial Sequence
  synthetic peptide
WGKIKWRLLVGW

SEQ ID NO: 932 - protein, 12 aa, Artificial Sequence
  synthetic peptide
LLAVKLKVGVAR

SEQ ID NO: 933 - protein, 12 aa, Artificial Sequence
  synthetic peptide
LVRIKAGGLLVK

SEQ ID NO: 934 - protein, 12 aa, Artificial Sequence
  synthetic peptide
AWGWKVKVIGAK

SEQ ID NO: 935 - protein, 12 aa, Artificial Sequence
  synthetic peptide
VWKAKGLAAIGR

SEQ ID NO: 936 - protein, 12 aa, Artificial Sequence
  synthetic peptide
VIKAALKALWVK

SEQ ID NO: 937 - protein, 12 aa, Artificial Sequence
  synthetic peptide
GAIIKWKLIAVR

SEQ ID NO: 938 - protein, 12 aa, Artificial Sequence
  synthetic peptide
WWVVKAKIALAR

SEQ ID NO: 939 - protein, 12 aa, Artificial Sequence
  synthetic peptide
AAKAKIKGLGIW

SEQ ID NO: 940 - protein, 12 aa, Artificial Sequence
  synthetic peptide
AARIRAWAGWGK

SEQ ID NO: 941 - protein, 12 aa, Artificial Sequence
  synthetic peptide
ALKGLLKIVVIR

SEQ ID NO: 942 - protein, 12 aa, Artificial Sequence
  synthetic peptide
LLKIILKIVLLR

SEQ ID NO: 943 - protein, 12 aa, Artificial Sequence
  synthetic peptide
IIKGKLKVGLLL

SEQ ID NO: 944 - protein, 12 aa, Artificial Sequence
  synthetic peptide
LLRIRVKAVIVK

SEQ ID NO: 945 - protein, 12 aa, Artificial Sequence
  synthetic peptide
AIKVRIKIQLLK

SEQ ID NO: 946 - protein, 12 aa, Artificial Sequence
  synthetic peptide
GIKWKWGVLVIR

SEQ ID NO: 947 - protein, 12 aa, Artificial Sequence
  synthetic peptide
VVKWKLKKIIAR

SEQ ID NO: 948 - protein, 12 aa, Artificial Sequence
  synthetic peptide
IVKQKVKVAGVK

SEQ ID NO: 949 - protein, 12 aa, Artificial Sequence
  synthetic peptide
VIRGKQKVIVLR

SEQ ID NO: 950 - protein, 12 aa, Artificial Sequence
  synthetic peptide
RGIVKRWIRLLK

SEQ ID NO: 951 - protein, 12 aa, Artificial Sequence
  synthetic peptide
RIIVKIIKKGIK

SEQ ID NO: 952 - protein, 12 aa, Artificial Sequence
  synthetic peptide
GWIWRLLWRVIK

SEQ ID NO: 953 - protein, 12 aa, Artificial Sequence
  synthetic peptide
RLIVRVIVRQLR

SEQ ID NO: 954 - protein, 12 aa, Artificial Sequence
  synthetic peptide
VVWIKIALGLLK

SEQ ID NO: 955 - protein, 12 aa, Artificial Sequence
  synthetic peptide
LWVGRVLGIILK

SEQ ID NO: 956 - protein, 12 aa, Artificial Sequence
  synthetic peptide
KIIIKLLGWGVK

SEQ ID NO: 957 - protein, 12 aa, Artificial Sequence
  synthetic peptide
KGWWKVIRRVLK

SEQ ID NO: 958 - protein, 12 aa, Artificial Sequence
  synthetic peptide
RKGVKIAVRAGR

SEQ ID NO: 959 - protein, 12 aa, Artificial Sequence
  synthetic peptide
RVGVKIIWQKAK

SEQ ID NO: 960 - protein, 12 aa, Artificial Sequence
  synthetic peptide
KVKVKAIWVGGR

SEQ ID NO: 961 - protein, 12 aa, Artificial Sequence
  synthetic peptide
KVIQKAAIQVWK

SEQ ID NO: 962 - protein, 12 aa, Artificial Sequence
  synthetic peptide
KVIGRVVLWGIK

SEQ ID NO: 963 - protein, 12 aa, Artificial Sequence
  synthetic peptide
RGLAKAVWRAWV

SEQ ID NO: 964 - protein, 12 aa, Artificial Sequence
  synthetic peptide
KGIVQVLWRAIR

SEQ ID NO: 965 - protein, 12 aa, Artificial Sequence
  synthetic peptide
KILWKIILAGLW

SEQ ID NO: 966 - protein, 12 aa, Artificial Sequence
  synthetic peptide
IIILKWIWRAIK

SEQ ID NO: 967 - protein, 12 aa, Artificial Sequence
  synthetic peptide
VIILKVLAQVVK

SEQ ID NO: 968 - protein, 12 aa, Artificial Sequence
  synthetic peptide
VWVWRVWVRIAR

SEQ ID NO: 969 - protein, 12 aa, Artificial Sequence
  synthetic peptide
KLWWKVLLKILK

SEQ ID NO: 970 - protein, 12 aa, Artificial Sequence
  synthetic peptide
RGAKRLVVKLIR

SEQ ID NO: 971 - protein, 12 aa, Artificial Sequence
  synthetic peptide
RGAVKKIWGIWK

SEQ ID NO: 972 - protein, 12 aa, Artificial Sequence
  synthetic peptide
KGLVKVVKVGVR

SEQ ID NO: 973 - protein, 12 aa, Artificial Sequence
  synthetic peptide
KVAAKAAIGVWK

SEQ ID NO: 974 - protein, 12 aa, Artificial Sequence
  synthetic peptide
KIWIKAVWQILK

SEQ ID NO: 975 - protein, 12 aa, Artificial Sequence
  synthetic peptide
WWKVLAGGLIRK

SEQ ID NO: 976 - protein, 12 aa, Artificial Sequence
  synthetic peptide
IIKAIIKWWWRK

SEQ ID NO: 977 - protein, 12 aa, Artificial Sequence
  synthetic peptide
ILKILWGVVWWK

SEQ ID NO: 978 - protein, 12 aa, Artificial Sequence
  synthetic peptide
WLKVGGVALLKA

SEQ ID NO: 979 - protein, 12 aa, Artificial Sequence
  synthetic peptide
LLKALIGLVARI

SEQ ID NO: 980 - protein, 12 aa, Artificial Sequence
  synthetic peptide
LAKIIWWQWIRR

SEQ ID NO: 981 - protein, 12 aa, Artificial Sequence
  synthetic peptide
IVRGKLVIVGKK

SEQ ID NO: 982 - protein, 12 aa, Artificial Sequence
  synthetic peptide
LIRIVKWVWARR

SEQ ID NO: 983 - protein, 12 aa, Artificial Sequence
  synthetic peptide
ALRAIGAWGALK

SEQ ID NO: 984 - protein, 12 aa, Artificial Sequence
  synthetic peptide
GAKVWGLAAWKV

SEQ ID NO: 985 - protein, 12 aa, Artificial Sequence
  synthetic peptide
LVRKGVIVAGKK

SEQ ID NO: 986 - protein, 12 aa, Artificial Sequence
  synthetic peptide
AWKALWKVIVKK

SEQ ID NO: 987 - protein, 12 aa, Artificial Sequence
  synthetic peptide
LKRGWGVVIVRK

SEQ ID NO: 988 - protein, 12 aa, Artificial Sequence
  synthetic peptide
LLKVIKIIWLRK

SEQ ID NO: 989 - protein, 12 aa, Artificial Sequence
  synthetic peptide
VVKLWVLGVLLK

SEQ ID NO: 990 - protein, 12 aa, Artificial Sequence
  synthetic peptide
ALRAVWKWGIKR

SEQ ID NO: 991 - protein, 12 aa, Artificial Sequence
  synthetic peptide
LLKVGLIGAARV

SEQ ID NO: 992 - protein, 12 aa, Artificial Sequence
  synthetic peptide
VGAVIGVVLVRR

SEQ ID NO: 993 - protein, 12 aa, Artificial Sequence
  synthetic peptide
IWKKLILGIWKK

SEQ ID NO: 994 - protein, 12 aa, Artificial Sequence
  synthetic peptide
AWKIWVGWVAQR

SEQ ID NO: 995 - protein, 12 aa, Artificial Sequence
  synthetic peptide
KLKAIGAVIWKK

SEQ ID NO: 996 - protein, 12 aa, Artificial Sequence
  synthetic peptide
KRLARAAGIIAR

SEQ ID NO: 997 - protein, 12 aa, Artificial Sequence
  synthetic peptide
RALAKALLARIK

SEQ ID NO: 998 - protein, 12 aa, Artificial Sequence
  synthetic peptide
KKAWKLVQIRIR

SEQ ID NO: 999 - protein, 12 aa, Artificial Sequence
  synthetic peptide
RRLGKILWWKVI

SEQ ID NO: 1000 - protein, 12 aa, Artificial Sequence
  synthetic peptide
IIVRVVKWIRWR

SEQ ID NO: 1001 - protein, 12 aa, Artificial Sequence
  synthetic peptide
ALIQVGRLIKLK

SEQ ID NO: 1002 - protein, 12 aa, Artificial Sequence
  synthetic peptide
IILKVIRILKWK

SEQ ID NO: 1003 - protein, 12 aa, Artificial Sequence
  synthetic peptide
VIVQALGRIRWK

SEQ ID NO: 1004 - protein, 12 aa, Artificial Sequence
  synthetic peptide
VVIQQLALWKAK

SEQ ID NO: 1005 - protein, 12 aa, Artificial Sequence
  synthetic peptide
IIGKIVWGVLIK

SEQ ID NO: 1006 - protein, 12 aa, Artificial Sequence
  synthetic peptide
ILIKALGIAKWL

SEQ ID NO: 1007 - protein, 12 aa, Artificial Sequence
  synthetic peptide
AVIQGWLIIRWK

SEQ ID NO: 1008 - protein, 12 aa, Artificial Sequence
  synthetic peptide
VGAKGWIIIRWK

SEQ ID NO: 1009 - protein, 12 aa, Artificial Sequence
  synthetic peptide
VLVRGIVAVKIK

SEQ ID NO: 1010 - protein, 12 aa, Artificial Sequence
  synthetic peptide
VLVKAWGIKKGR

SEQ ID NO: 1011 - protein, 12 aa, Artificial Sequence
  synthetic peptide
LWWRGVAVWKII

SEQ ID NO: 1012 - protein, 12 aa, Artificial Sequence
  synthetic peptide
KAAKAAGIVRGK

SEQ ID NO: 1013 - protein, 12 aa, Artificial Sequence
  synthetic peptide
IGIKVVIGWILK

SEQ ID NO: 1014 - protein, 12 aa, Artificial Sequence
  synthetic peptide
VWIRLGLWGRAK

SEQ ID NO: 1015 - protein, 12 aa, Artificial Sequence
  synthetic peptide
AIIRLVGLLAIR

SEQ ID NO: 1016 - protein, 12 aa, Artificial Sequence
  synthetic peptide
WVGKAVWGAKIL

SEQ ID NO: 1017 - protein, 12 aa, Artificial Sequence
  synthetic peptide
WWIKIALGIRGI

SEQ ID NO: 1018 - protein, 12 aa, Artificial Sequence
  synthetic peptide
WAIAGGVLIKAR

SEQ ID NO: 1019 - protein, 12 aa, Artificial Sequence
  synthetic peptide
WLLRGGALIKWI

SEQ ID NO: 1020 - protein, 12 aa, Artificial Sequence
  synthetic peptide
WAGKIVIIGKIA

SEQ ID NO: 1021 - protein, 12 aa, Artificial Sequence
  synthetic peptide
VAALGWALVKAR

SEQ ID NO: 1022 - protein, 12 aa, Artificial Sequence
  synthetic peptide
LGLKIIWVGKIL

SEQ ID NO: 1023 - protein, 12 aa, Artificial Sequence
  synthetic peptide
VVWRLVGIIRIA

SEQ ID NO: 1024 - protein, 12 aa, Artificial Sequence
  synthetic peptide
IWLKVGGIIIVK

SEQ ID NO: 1025 - protein, 12 aa, Artificial Sequence
  synthetic peptide
LLWRLLWGVKGL

SEQ ID NO: 1026 - protein, 12 aa, Artificial Sequence
  synthetic peptide
IIAWGVIVGKAR

SEQ ID NO: 1027 - protein, 12 aa, Artificial Sequence
  synthetic peptide
VWLKLVGWAKIV

SEQ ID NO: 1028 - protein, 12 aa, Artificial Sequence
  synthetic peptide
VWIKILGWLKIA

SEQ ID NO: 1029 - protein, 12 aa, Artificial Sequence
  synthetic peptide
ILIGILGLLKVR

SEQ ID NO: 1030 - protein, 12 aa, Artificial Sequence
  synthetic peptide
ALLKWIWVGWIR

SEQ ID NO: 1031 - protein, 12 aa, Artificial Sequence
  synthetic peptide
LILRAALRGRGR

SEQ ID NO: 1032 - protein, 12 aa, Artificial Sequence
  synthetic peptide
AGAKVWKIVKWK

SEQ ID NO: 1033 - protein, 12 aa, Artificial Sequence
  synthetic peptide
WGALWIGAVRIK

SEQ ID NO: 1034 - protein, 12 aa, Artificial Sequence
  synthetic peptide
GAVQAIWRLRAR

SEQ ID NO: 1035 - protein, 12 aa, Artificial Sequence
  synthetic peptide
LVIRQLWVWKVR

SEQ ID NO: 1036 - protein, 12 aa, Artificial Sequence
  synthetic peptide
GLARAWAWRKGK

SEQ ID NO: 1037 - protein, 12 aa, Artificial Sequence
  synthetic peptide
VWGKVVLWGKKR

SEQ ID NO: 1038 - protein, 12 aa, Artificial Sequence
  synthetic peptide
LLIKWWLAKRLR

SEQ ID NO: 1039 - protein, 12 aa, Artificial Sequence
  synthetic peptide
VIVRIVIGIIGK

SEQ ID NO: 1040 - protein, 12 aa, Artificial Sequence
  synthetic peptide
LAWQLIIGIKIR

SEQ ID NO: 1041 - protein, 12 aa, Artificial Sequence
  synthetic peptide
VILQLVWIKRLK

SEQ ID NO: 1042 - protein, 12 aa, Artificial Sequence
  synthetic peptide
VWVQIIRAAKIR

SEQ ID NO: 1043 - protein, 12 aa, Artificial Sequence
  synthetic peptide
IWAQKAVVVKIK

SEQ ID NO: 1044 - protein, 12 aa, Artificial Sequence
  synthetic peptide
IIWRVKWVWRIK

SEQ ID NO: 1045 - protein, 12 aa, Artificial Sequence
  synthetic peptide
VWWLGIIILKAK

SEQ ID NO: 1046 - protein, 12 aa, Artificial Sequence
  synthetic peptide
VAWQLIVVRKGK

SEQ ID NO: 1047 - protein, 12 aa, Artificial Sequence
  synthetic peptide
GIKKLVIGLKLK

SEQ ID NO: 1048 - protein, 12 aa, Artificial Sequence
  synthetic peptide
VWLQIIIRVRWK

SEQ ID NO: 1049 - protein, 12 aa, Artificial Sequence
  synthetic peptide
WLWKAVWIKKIK

SEQ ID NO: 1050 - protein, 12 aa, Artificial Sequence
  synthetic peptide
IVVKVILARRLR

SEQ ID NO: 1051 - protein, 12 aa, Artificial Sequence
  synthetic peptide
VIVQWKLWLRLR

SEQ ID NO: 1052 - protein, 12 aa, Artificial Sequence
  synthetic peptide
GIGKWLVLRRVK

SEQ ID NO: 1053 - protein, 12 aa, Artificial Sequence
  synthetic peptide
GWIKIRLGVKLK

SEQ ID NO: 1054 - protein, 12 aa, Artificial Sequence
  synthetic peptide
VVWVGVGIWRAK

SEQ ID NO: 1055 - protein, 12 aa, Artificial Sequence
  synthetic peptide
AGKRAGVVIKAR

SEQ ID NO: 1056 - protein, 12 aa, Artificial Sequence
  synthetic peptide
VWLRWLGLVVVK

SEQ ID NO: 1057 - protein, 12 aa, Artificial Sequence
  synthetic peptide
KVGRALGAAKAK

SEQ ID NO: 1058 - protein, 12 aa, Artificial Sequence
  synthetic peptide
GLAKAIALGRIV

SEQ ID NO: 1059 - protein, 12 aa, Artificial Sequence
  synthetic peptide
WLLKGGLVWRIV

SEQ ID NO: 1060 - protein, 12 aa, Artificial Sequence
  synthetic peptide
VIGKKIWAARLK

SEQ ID NO: 1061 - protein, 12 aa, Artificial Sequence
  synthetic peptide
WIIRKLAGVKVK

SEQ ID NO: 1062 - protein, 12 aa, Artificial Sequence
  synthetic peptide
AIVQVLGAIRWK

SEQ ID NO: 1063 - protein, 12 aa, Artificial Sequence
  synthetic peptide
GKKWRIIRWKWI

SEQ ID NO: 1064 - protein, 12 aa, Artificial Sequence
  synthetic peptide
WIRWLALRIRWL

SEQ ID NO: 1065 - protein, 12 aa, Artificial Sequence
  synthetic peptide
WRRLKIVKLKGG

SEQ ID NO: 1066 - protein, 12 aa, Artificial Sequence
  synthetic peptide
IKKLLLARLKLK

SEQ ID NO: 1067 - protein, 12 aa, Artificial Sequence
  synthetic peptide
GKRIWAIKKKIV

SEQ ID NO: 1068 - protein, 12 aa, Artificial Sequence
  synthetic peptide
WLKAWGGKIRVL

SEQ ID NO: 1069 - protein, 12 aa, Artificial Sequence
  synthetic peptide
LKWLIGIKLKGA

SEQ ID NO: 1070 - protein, 12 aa, Artificial Sequence
  synthetic peptide
VKKAAWGKWKLI

SEQ ID NO: 1071 - protein, 12 aa, Artificial Sequence
  synthetic peptide
IKKIWIVKWRKG

SEQ ID NO: 1072 - protein, 12 aa, Artificial Sequence
  synthetic peptide
IKRLVAWKKKVL

SEQ ID NO: 1073 - protein, 12 aa, Artificial Sequence
  synthetic peptide
VKRVVLIRIVAA

SEQ ID NO: 1074 - protein, 12 aa, Artificial Sequence
  synthetic peptide
KQRVVIVKVRIL

SEQ ID NO: 1075 - protein, 12 aa, Artificial Sequence
  synthetic peptide
ARKVIVIQVKAI

SEQ ID NO: 1076 - protein, 12 aa, Artificial Sequence
  synthetic peptide
IQKWVVIRWRLR

SEQ ID NO: 1077 - protein, 12 aa, Artificial Sequence
  synthetic peptide
GKRWLLVRVKKI

SEQ ID NO: 1078 - protein, 12 aa, Artificial Sequence
  synthetic peptide
VRKVWIGGVKVI

SEQ ID NO: 1079 - protein, 12 aa, Artificial Sequence
  synthetic peptide
IKAVVVGRAKIV

SEQ ID NO: 1080 - protein, 12 aa, Artificial Sequence
  synthetic peptide
VQAAWAGKWKVW

SEQ ID NO: 1081 - protein, 12 aa, Artificial Sequence
  synthetic peptide
LKKVWALRGIAV

SEQ ID NO: 1082 - protein, 12 aa, Artificial Sequence
  synthetic peptide
VRKIVWIRLKVG

SEQ ID NO: 1083 - protein, 12 aa, Artificial Sequence
  synthetic peptide
ARKIVWLKGRAV

SEQ ID NO: 1084 - protein, 12 aa, Artificial Sequence
  synthetic peptide
GVKGIALKIKVL

SEQ ID NO: 1085 - protein, 12 aa, Artificial Sequence
  synthetic peptide
IRKLLWIRALLG

SEQ ID NO: 1086 - protein, 12 aa, Artificial Sequence
  synthetic peptide
KRKQWKLWVRQI

SEQ ID NO: 1087 - protein, 12 aa, Artificial Sequence
  synthetic peptide
GAKIIRKVAQVA

SEQ ID NO: 1088 - protein, 12 aa, Artificial Sequence
  synthetic peptide
VKRVKQILWRLG

SEQ ID NO: 1089 - protein, 12 aa, Artificial Sequence
  synthetic peptide
IKAAKAGQWRRV

SEQ ID NO: 1090 - protein, 12 aa, Artificial Sequence
  synthetic peptide
RGRLKQKWWRRL

SEQ ID NO: 1091 - protein, 12 aa, Artificial Sequence
  synthetic peptide
LQRVIWQKWRKV

SEQ ID NO: 1092 - protein, 12 aa, Artificial Sequence
  synthetic peptide
RLAKRKGQAIWV

SEQ ID NO: 1093 - protein, 12 aa, Artificial Sequence
  synthetic peptide
QKIGRAVIWKVK

SEQ ID NO: 1094 - protein, 12 aa, Artificial Sequence
  synthetic peptide
QLRVAWKRAWWA

SEQ ID NO: 1095 - protein, 12 aa, Artificial Sequence
  synthetic peptide
KAVKKGRRAIVV

SEQ ID NO: 1096 - protein, 12 aa, Artificial Sequence
  synthetic peptide
VIRAKAVWGWVK

SEQ ID NO: 1097 - protein, 12 aa, Artificial Sequence
  synthetic peptide
VARAVQKRWRKK

SEQ ID NO: 1098 - protein, 12 aa, Artificial Sequence
  synthetic peptide
VKAKRWKWAQLA

SEQ ID NO: 1099 - protein, 12 aa, Artificial Sequence
  synthetic peptide
KRVQAKAWRLQR

SEQ ID NO: 1100 - protein, 12 aa, Artificial Sequence
  synthetic peptide
KKIRQWGKAAAW

SEQ ID NO: 1101 - protein, 12 aa, Artificial Sequence
  synthetic peptide
QQLRWKRVAKAI

SEQ ID NO: 1102 - protein, 12 aa, Artificial Sequence
  synthetic peptide
IQIQLVKRWAVI

SEQ ID NO: 1103 - protein, 12 aa, Artificial Sequence
  synthetic peptide
RLIQWGWKIWAV

SEQ ID NO: 1104 - protein, 12 aa, Artificial Sequence
  synthetic peptide
KLLGILKQAIVV

SEQ ID NO: 1105 - protein, 12 aa, Artificial Sequence
  synthetic peptide
VLLRVGARIVVG

SEQ ID NO: 1106 - protein, 12 aa, Artificial Sequence
  synthetic peptide
LLIAGKWWKLAI

SEQ ID NO: 1107 - protein, 12 aa, Artificial Sequence
  synthetic peptide
LKKIIVQAVGLI

SEQ ID NO: 1108 - protein, 12 aa, Artificial Sequence
  synthetic peptide
LKILIAQAKKGL

SEQ ID NO: 1109 - protein, 12 aa, Artificial Sequence
  synthetic peptide
IGQVVLVKIKIA

SEQ ID NO: 1110 - protein, 12 aa, Artificial Sequence
  synthetic peptide
VWLAQKIGKWIW

SEQ ID NO: 1111 - protein, 12 aa, Artificial Sequence
  synthetic peptide
KKAIKVVAIGRI

SEQ ID NO: 1112 - protein, 12 aa, Artificial Sequence
  synthetic peptide
WIIRWIKIWLKI

SEQ ID NO: 1113 - protein, 12 aa, Artificial Sequence
  synthetic peptide
VIAKIVLLRAGL

SEQ ID NO: 1114 - protein, 12 aa, Artificial Sequence
  synthetic peptide
RGARVIRWKLRR

SEQ ID NO: 1115 - protein, 12 aa, Artificial Sequence
  synthetic peptide
RAIIKQRWQRRW

SEQ ID NO: 1116 - protein, 12 aa, Artificial Sequence
  synthetic peptide
QRWKKWKVLKLR

SEQ ID NO: 1117 - protein, 12 aa, Artificial Sequence
  synthetic peptide
KIWLLKLRQRQK

SEQ ID NO: 1118 - protein, 12 aa, Artificial Sequence
  synthetic peptide
WRIKKQWIQIIV

SEQ ID NO: 1119 - protein, 12 aa, Artificial Sequence
  synthetic peptide
IILKRVQVQKIK

SEQ ID NO: 1120 - protein, 12 aa, Artificial Sequence
  synthetic peptide
KRIKKLLKVVLK

SEQ ID NO: 1121 - protein, 12 aa, Artificial Sequence
  synthetic peptide
QQKVIRLLWKAK

SEQ ID NO: 1122 - protein, 12 aa, Artificial Sequence
  synthetic peptide
RIWRRAWKARWK

SEQ ID NO: 1123 - protein, 12 aa, Artificial Sequence
  synthetic peptide
KIKLIQKQLRIK

SEQ ID NO: 1124 - protein, 12 aa, Artificial Sequence
  synthetic peptide
ALLAGRKRAVAV

SEQ ID NO: 1125 - protein, 12 aa, Artificial Sequence
  synthetic peptide
KAVAGARQRWAL

SEQ ID NO: 1126 - protein, 12 aa, Artificial Sequence
  synthetic peptide
AIGAARAWRQWA

SEQ ID NO: 1127 - protein, 12 aa, Artificial Sequence
  synthetic peptide
AVIVRAAKGGAR

SEQ ID NO: 1128 - protein, 12 aa, Artificial Sequence
  synthetic peptide
LLKLKQKGIVIA

SEQ ID NO: 1129 - protein, 12 aa, Artificial Sequence
  synthetic peptide
QWLVKWVIIKVV

SEQ ID NO: 1130 - protein, 12 aa, Artificial Sequence
  synthetic peptide
IQIWIIRVIWRW

SEQ ID NO: 1131 - protein, 12 aa, Artificial Sequence
  synthetic peptide
KVIQWIIVRRVL

SEQ ID NO: 1132 - protein, 12 aa, Artificial Sequence
  synthetic peptide
KVIKIVLVRVVK

SEQ ID NO: 1133 - protein, 12 aa, Artificial Sequence
  synthetic peptide
WLKRIVKVVVLK

SEQ ID NO: 1134 - protein, 12 aa, Artificial Sequence
  synthetic peptide
IKIVRRAKIIIW

SEQ ID NO: 1135 - protein, 12 aa, Artificial Sequence
  synthetic peptide
VKWKGKVIVVQL

SEQ ID NO: 1136 - protein, 12 aa, Artificial Sequence
  synthetic peptide
KIVQKKLRLVVI

SEQ ID NO: 1137 - protein, 12 aa, Artificial Sequence
  synthetic peptide
GKLKIKVKLGIA

SEQ ID NO: 1138 - protein, 12 aa, Artificial Sequence
  synthetic peptide
QVVVKKKAIQVV

SEQ ID NO: 1139 - protein, 12 aa, Artificial Sequence
  synthetic peptide
VAKVKKARWRLR

SEQ ID NO: 1140 - protein, 12 aa, Artificial Sequence
  synthetic peptide
IIKWIVVRQIRK

SEQ ID NO: 1141 - protein, 12 aa, Artificial Sequence
  synthetic peptide
KGKIRKIVLIRR

SEQ ID NO: 1142 - protein, 12 aa, Artificial Sequence
  synthetic peptide
  misc_feature (2)..(2): Xaa is L-2-amino-3-guanidinopropionic acid
VXLIVAVRIWRR

SEQ ID NO: 1143 - protein, 12 aa, Artificial Sequence
  synthetic peptide
  misc_feature (3)..(3): Xaa is L-2-amino-3-guanidinopropionic acid
VRXIVAVRIWRR

SEQ ID NO: 1144 - protein, 12 aa, Artificial Sequence
  synthetic peptide
  misc_feature (4)..(4): Xaa is L-2-amino-3-guanidinopropionic acid
VRLXVAVRIWRR

SEQ ID NO: 1145 - protein, 12 aa, Artificial Sequence
  synthetic peptide
  misc_feature (5)..(5): Xaa is L-2-amino-3-guanidinopropionic acid
VRLIXAVRIWRR

SEQ ID NO: 1146 - protein, 12 aa, Artificial Sequence
  synthetic peptide
  misc_feature (6)..(6): Xaa is L-2-amino-3-guanidinopropionic acid
VRLIVXVRIWRR

SEQ ID NO: 1147 - protein, 12 aa, Artificial Sequence
  synthetic peptide
  misc_feature (7)..(7): Xaa is L-2-amino-3-guanidinopropionic acid
VRLIVAXRIWRR

SEQ ID NO: 1148 - protein, 12 aa, Artificial Sequence
  synthetic peptide
  misc_feature (8)..(8): Xaa is L-2-amino-3-guanidinopropionic acid
VRLIVAVXIWRR

SEQ ID NO: 1149 - protein, 12 aa, Artificial Sequence
  synthetic peptide
  misc_feature (9)..(9): Xaa is L-2-amino-3-guanidinopropionic acid
VRLIVAVRXWRR

SEQ ID NO: 1150 - protein, 12 aa, Artificial Sequence
  synthetic peptide
  misc_feature (10)..(10): Xaa is L-2-amino-3-guanidinopropionic acid
VRLIVAVRIXRR

SEQ ID NO: 1151 - protein, 12 aa, Artificial Sequence
  synthetic peptide
  misc_feature (11)..(11): Xaa is L-2-amino-3-guanidinopropionic acid
VRLIVAVRIWXR

SEQ ID NO: 1152 - protein, 12 aa, Artificial Sequence
  synthetic peptide
  misc_feature (12)..(12): Xaa is L-2-amino-3-guanidinopropionic acid
VRLIVAVRIWRX

SEQ ID NO: 1153 - protein, 12 aa, Artificial Sequence
  synthetic peptide
  misc_feature (2)..(2): Xaa is L-2-Amino-4-guanidinobutyric acid
VXLIVAVRIWRR

SEQ ID NO: 1154 - protein, 12 aa, Artificial Sequence
  synthetic peptide
  misc_feature (3)..(3): Xaa is L-2-Amino-4-guanidinobutyric acid
VRXIVAVRIWRR

SEQ ID NO: 1155 - protein, 12 aa, Artificial Sequence
  synthetic peptide
  misc_feature (4)..(4): Xaa is L-2-Amino-4-guanidinobutyric acid
VRLXVAVRIWRR

SEQ ID NO: 1156 - protein, 12 aa, Artificial Sequence
  synthetic peptide
  misc_feature (5)..(5): Xaa is L-2-Amino-4-guanidinobutyric acid
VRLIXAVRIWRR

SEQ ID NO: 1157 - protein, 12 aa, Artificial Sequence
  synthetic peptide
  misc_feature (6)..(6): Xaa is L-2-Amino-4-guanidinobutyric acid
VRLIVXVRIWRR

SEQ ID NO: 1158 - protein, 12 aa, Artificial Sequence
  synthetic peptide
  misc_feature (7)..(7): Xaa is L-2-Amino-4-guanidinobutyric acid
VRLIVAXRIWRR

SEQ ID NO: 1159 - protein, 12 aa, Artificial Sequence
  synthetic peptide
  misc_feature (8)..(8): Xaa is L-2-Amino-4-guanidinobutyric acid
VRLIVAVXIWRR

SEQ ID NO: 1160 - protein, 12 aa, Artificial Sequence
  synthetic peptide
  misc_feature (9)..(9): Xaa is L-2-Amino-4-guanidinobutyric acid
VRLIVAVRXWRR

SEQ ID NO: 1161 - protein, 12 aa, Artificial Sequence
  synthetic peptide
  misc_feature (10)..(10): Xaa is L-2-Amino-4-guanidinobutyric acid
VRLIVAVRIXRR

SEQ ID NO: 1162 - protein, 12 aa, Artificial Sequence
  synthetic peptide
  misc_feature (11)..(11): Xaa is L-2-Amino-4-guanidinobutyric acid
VRLIVAVRIWXR

SEQ ID NO: 1163 - protein, 12 aa, Artificial Sequence
  synthetic peptide
  misc_feature (12)..(12): Xaa is L-2-Amino-4-guanidinobutyric acid
VRLIVAVRIWRX

SEQ ID NO: 1164 - protein, 12 aa, Artificial Sequence
  synthetic peptide
  misc_feature (2)..(2): Xaa is L-Homoarginine
VXLIVAVRIWRR

SEQ ID NO: 1165 - protein, 12 aa, Artificial Sequence
  synthetic peptide
  misc_feature (3)..(3): Xaa is L-Homoarginine
VRXIVAVRIWRR

SEQ ID NO: 1166 - protein, 12 aa, Artificial Sequence
  synthetic peptide
  misc_feature (4)..(4): Xaa is L-Homoarginine
VRLXVAVRIWRR

SEQ ID NO: 1167 - protein, 12 aa, Artificial Sequence
  synthetic peptide
  misc_feature (5)..(5): Xaa is L-Homoarginine
VRLIXAVRIWRR

SEQ ID NO: 1168 - protein, 12 aa, Artificial Sequence
  synthetic peptide
  misc_feature (7)..(7): Xaa is L-Homoarginine
VRLIVAXRIWRR

SEQ ID NO: 1169 - protein, 12 aa, Artificial Sequence
  synthetic peptide
  misc_feature (9)..(9): Xaa is L-Homoarginine
VRLIVAVRXWRR

SEQ ID NO: 1170 - protein, 12 aa, Artificial Sequence
  synthetic peptide
  misc_feature (10)..(10): Xaa is L-Homoarginine
VRLIVAVRIXRR

SEQ ID NO: 1171 - protein, 12 aa, Artificial Sequence
  synthetic peptide
  misc_feature (2)..(2): Xaa is L-2,3-diaminopropionic acid
VXLIVAVRIWRR

SEQ ID NO: 1172 - protein, 12 aa, Artificial Sequence
  synthetic peptide
  misc_feature (3)..(3): Xaa is L-2,3-diaminopropionic acid
VRXIVAVRIWRR

SEQ ID NO: 1173 - protein, 12 aa, Artificial Sequence
  synthetic peptide
  misc_feature (4)..(4): Xaa is L-2,3-diaminopropionic acid
VRLXVAVRIWRR

SEQ ID NO: 1174 - protein, 11 aa, Artificial Sequence
  synthetic peptide
  misc_feature (5)..(5): Xaa is L-2,3-diaminopropionic acid
VRLIXVRIWRR

SEQ ID NO: 1175 - protein, 12 aa, Artificial Sequence
  synthetic peptide
  misc_feature (6)..(6): Xaa is L-2,3-diaminopropionic acid
VRLIVXVRIWRR

SEQ ID NO: 1176 - protein, 12 aa, Artificial Sequence
  synthetic peptide
  misc_feature (7)..(7): Xaa is L-2,3-diaminopropionic acid
VRLIVAXRIWRR

SEQ ID NO: 1177 - protein, 12 aa, Artificial Sequence
  synthetic peptide
  misc_feature (8)..(8): Xaa is L-2,3-diaminopropionic acid
VRLIVAVXIWRR

SEQ ID NO: 1178 - protein, 12 aa, Artificial Sequence
  synthetic peptide
  misc_feature (9)..(9): Xaa is L-2,3-diaminopropionic acid
VRLIVAVRXWRR

SEQ ID NO: 1179 - protein, 12 aa, Artificial Sequence
  synthetic peptide
  misc_feature (2)..(2): Xaa is L-2,4-diaminobutyric acid
VXLIVAVRIWRR

SEQ ID NO: 1180 - protein, 12 aa, Artificial Sequence
  synthetic peptide
  misc_feature (3)..(3): Xaa is L-2,4-diaminobutyric acid
VRXIVAVRIWRR

SEQ ID NO: 1181 - protein, 12 aa, Artificial Sequence
  synthetic peptide
  misc_feature (4)..(4): Xaa is L-2,4-diaminobutyric acid
VRLXVAVRIWRR

SEQ ID NO: 1182 - protein, 12 aa, Artificial Sequence
  synthetic peptide
  misc_feature (5)..(5): Xaa is L-2,4-diaminobutyric acid
VRLIXAVRIWRR

SEQ ID NO: 1183 - protein, 12 aa, Artificial Sequence
  synthetic peptide
  misc_feature (6)..(6): Xaa is L-2,4-diaminobutyric acid
VRLIVXVRIWRR

SEQ ID NO: 1184 - protein, 12 aa, Artificial Sequence
  synthetic peptide
  misc_feature (7)..(7): Xaa is L-2,4-diaminobutyric acid
VRLIVAXRIWRR

SEQ ID NO: 1185 - protein, 12 aa, Artificial Sequence
  synthetic peptide
  misc_feature (8)..(8): Xaa is L-2,4-diaminobutyric acid
VRLIVAVXIWRR

SEQ ID NO: 1186 - protein, 12 aa, Artificial Sequence
  synthetic peptide
  misc_feature (9)..(9): Xaa is L-2,4-diaminobutyric acid
VRLIVAVRXWRR

SEQ ID NO: 1187 - protein, 12 aa, Artificial Sequence
  synthetic peptide
  misc_feature (10)..(10): Xaa is L-2,4-diaminobutyric acid
VRLIVAVRIXRR

SEQ ID NO: 1188 - protein, 12 aa, Artificial Sequence
  synthetic peptide
  misc_feature (11)..(11): Xaa is L-2,4-diaminobutyric acid
VRLIVAVRIWXR

SEQ ID NO: 1189 - protein, 12 aa, Artificial Sequence
  synthetic peptide
  misc_feature (12)..(12): Xaa is L-2,4-diaminobutyric acid
VRLIVAVRIWRX

SEQ ID NO: 1190 - protein, 12 aa, Artificial Sequence
  synthetic peptide
  misc_feature (3)..(3): xaa is a polar residue
VRXIVAVRIWRR

SEQ ID NO: 1191 - protein, 12 aa, Artificial Sequence
  synthetic peptide
  misc_feature (4)..(4): xaa is a polar residue
VRLXVAVRIWRR

SEQ ID NO: 1192 - protein, 12 aa, Artificial Sequence
  synthetic peptide
  misc_feature (5)..(5): xaa is a polar residue
VRLIXAVRIWRR

SEQ ID NO: 1193 - protein, 12 aa, Artificial Sequence
  synthetic peptide
  misc_feature (7)..(7): xaa is a polar residue
VRLIVAXRIWRR

SEQ ID NO: 1194 - protein, 12 aa, Artificial Sequence
  synthetic peptide
  misc_feature (9)..(9): xaa is a polar residue
VRLIVAVRXWRR

SEQ ID NO: 1195 - protein, 12 aa, Artificial Sequence
  synthetic peptide
  misc_feature (10)..(10): xaa is a polar residue
VRLIVAVRIXRR

(19) United States
(12) Patent Application Publication
(10) Pub. No.: US 2021/0138025 A1
(43) Pub. Date: May 13, 2021

(54) CATIONIC PEPTIDES WITH IMMUNOMODULATORY AND/OR ANTI-BIOFILM ACTIVITIES

(71) Applicant: THE UNIVERSITY OF BRITISH COLUMBIA, Vancouver, British Columbia (CA)

(72) Inventors: Robert HANCOCK, Vancouver (CA); Evan HANEY, Vancouver (CA); Ashley HILCHIE, East Lawrencetown (CA); Artem TCHERKASOV, Vancouver (CA); Yoan BRITO-SANCHEZ, Vancouver (CA)

(21) Appl. No.: 16/964,566

(22) PCT Filed: Jan. 25, 2019
(86) PCT No.: PCT/IB2019/050643

§ 371 Date: Jul. 23, 2020

Related U.S. Application Data
(60) Provisional application No. 62/622,687, filed on Jan. 26, 2018.

(51) Int. Cl. A61K 38/10; C07K 7/06; C07K 7/08; A61K 38/08; A61K 45/06; A61P 31/04

(52) U.S. Cl. CPC A61K 38/10; C07K 7/06; A61P 31/04; A61K 38/08; A61K 45/06; C07K 7/08

(57) ABSTRACT

The present disclosure relates generally to peptides and more specifically to anti-biofilm and/or immunomodulatory peptides.

### FIELD

The present invention relates generally to peptides, and more specifically to anti-biofilm and/or immunomodulatory peptides.

### BACKGROUND

The treatment of bacterial infections with antibiotics is one of the mainstays of human medicine. Unfortunately, the effectiveness of antibiotics has become limited due to an increase in bacterial antibiotic resistance in the face of a decreasing efforts and success in discovery of new classes of antibiotics. Today, infectious diseases are the second leading cause of death worldwide and the largest cause of premature deaths and loss of work productivity in industrialized countries. Nosocomial bacterial infections that are resistant to therapy result in annual costs of more than $2 billion and account for more than 100,000 direct and indirect deaths in North America alone, whereas a major complication of microbial diseases, namely sepsis, annually accounts for 750,000 cases and 210,000 deaths in North America and 5 million worldwide.

A major limitation in antibiotic development has been difficulties in finding new structures with equivalent properties to the conventional antibiotics, namely low toxicity for the host and a broad spectrum of action against bacterial pathogens. Recent novel antibiotic classes, including the oxazolidinones (linezolid), the streptogramins (synercid) and the glycolipopeptides (daptomycin) are all only active against Gram positive pathogens. One promising set of compounds is the cationic antimicrobial peptides that are mimics of peptides produced by virtually all complex organisms ranging from plants and insects to humans as a major component of their innate defenses against infection.

Cationic antimicrobial peptides, found in most species of life, represent a good template for a new generation of antimicrobials. They kill both Gram negative and Gram positive microorganisms rapidly and directly, do not easily select mutants, work against common clinically-resistant bacteria such as methicillin-resistant Staphylococcus aureus (MRSA) and vancomycin resistant Enterococcus (VRE), show a synergistic effect with conventional antibiotics, and can often activate host innate immunity without displaying immunogenicity (Hancock R E W. 2001; Fjell C D, et al. 2012.). Moreover, some peptides seem to counteract some of the more harmful aspects of inflammation (e.g. sepsis, endotoxaemia), which is extremely important since rapid killing of bacteria and subsequent liberation of bacterial components such as LPS or peptidoglycan can induce fatal immune dysregulation (Jarisch-Herxheimer reaction) (Gough M, et al. 1996) and also stimulate anti-infective immunity (Hilchie A L et al. 2013). Thus, they offer at least two separate approaches to treating infections with uses as broad spectrum anti-infectives and/or as adjuvants that selectively enhance aspects of innate immunity while suppressing potentially harmful inflammation. Although there is great hope for such peptides, there is clearly much room for improvement (Hancock, R. E. W., et al. 2012; Fjell C D, et all. 2012.).

Biofilm infections are especially recalcitrant to conventional antibiotic treatment (35,36), and are a major problem in trauma patients, including military personnel with major injuries (Høiby, N., et al. 2011; Antunes, L C M and R B R Ferreira. 2011). Microbial biofilms are surface-associated bacterial communities that grow in a protective polymeric matrix. The biofilm-mode of growth is a major lifestyle for bacteria in natural, industrial and clinical settings; indeed they are associated with 65% or more of all clinical infections. In the clinic, bacterial growth as biofilms, renders them difficult to treat with conventional antibiotics, and can result in as much as a 1000-fold decrease in susceptibility to antimicrobial agents, due to differentiation of bacteria within the biofilm, poor antibiotic penetration into the biofilm, and the stationary phase growth of bacteria underlying the surface layer. There are very few compounds developed that have activity against bacterial biofilms, unlike the peptides described here.

In 2008, it was shown that the 37 amino acid human host defense peptide LL-37 was able to both prevent the development of biofilms and promote dissociation of existing biofilms (Overhage, J., et al. 2008); a property that was apparently shared by a subset of the natural antimicrobial peptides (e.g., bovine indolicidin), but not by other cationic host defense peptides and antibiotics (e.g., polymyxin). Mechanistically, it was demonstrated that LL-37 likely entered bacteria at sub-inhibitory concentrations and altered the transcription of dozens of genes leading to decreased bacterial attachment, increased twitching motility, and decreases in the quorum sensing systems (Las and Rhl). Since this time anti-biofilm activity has been confirmed by several other investigators and extended to certain other peptides (e.g. Amer L. S., et al. 2010). LL-37 is able to protect against bacterial infections despite having no antimicrobial activity under physiological conditions (Bowdish, D. M. E., D. J. Davidson, Y. E. Lau, K. Lee, M. G. Scott, and R. E. W. Hancock. 2005. Impact of LL-37 on anti-infective immunity. J. Leukocyte Biol. 77:451-459).

It is well accepted that vaccine immunization is best achieved by co-administration of an adjuvant. The precise mechanism by which these adjuvants work has eluded immunologists but appears to work in part by upregulating elements of innate immunity that smooth the transition to adaptive (antigen-specific) immunity (Bendelac A and R. Medzhitov. 2002. Adjuvants of immunity: Harnessing innate immunity to promote adaptive immunity J. Exp. Med. 195:F19-F23). Within this concept there are several possible avenues by which adjuvants might work including the attraction of immune cells into the site at which a particular antigen is injected, through e.g. upregulation of chemokines, the appropriate activation of cells when they reach that site, which can be caused by local cell or tissue damage releasing endogenous adjuvants or through specific cell activation by the adjuvants, and the compartmentalization of immune responses to the site of immunization (the so-called “depot” effect). Due to their ability to selectively modulate cell responses, including induction of chemokine expression, cationic host defence peptides such as human LL-37 and defensins, have been examined for adjuvant activity and demonstrated to enhance adaptive immune responses to a variety of antigens [Nicholls, E. F., L. Madera and R. E. W. Hancock. 2010. Immunomodulators as adjuvants for vaccines and antimicrobial therapy. Ann. NY Acad. Sci. 1213:46-61].

Screening of a library of peptides indicated that peptides as small as 9 amino acids in length were active against P. aeruginosa (de la Fuente-Núñez, C., et al. 2012). These studies also indicated that antimicrobial and anti-biofilm properties were independently determined. For example, a 9-amino acid long peptide 1037 had very good anti-biofilm activity (IC₅₀=5 μg/ml), but essentially no antimicrobial activity against biofilm cells (MIC=304 μg/ml), whereas a related peptide HH10 had very good antimicrobial activity (MIC=0.8 μg/ml) but was devoid of anti-biofilm activity. These peptides also break down Campylobacter, Burkholderia and Listeria biofilms. Burkholderia is resistant to the antibiotic action of antimicrobial peptides against free swimming cells, confirming the independence of antimicrobial and anti-biofilm activity.

Further screening led to peptides that were very broad spectrum in being able to: (i) both prevent biofilm formation and kill multiple species of bacteria in biofilms and (MBEC <1 μg/ml), including P. aeruginosa and methicillin resistant Staphylococcus aureus and other major clinically relevant Gram negative and Gram positive bacteria, including the ESKAPE pathogens (Fuente-Núñez, C., et al. 2014; de la Fuente-Núñez, C., et al. 2015), (ii) work synergistically with several antibiotics in multiple species (de la Fuente-Núñez, C., et al. 2015; Reffuveille, F., et al. 2014), and (iii) are effective in animal models of biofilm infections (de la Fuente-Nú\ddot{n}ez, C., et al. 2015). The action of such peptides was found to be dependent on their ability to trigger the degradation of the nucleotide stress signal ppGpp. Structure activity relationships studies confirmed that there was no major overlap between anti-biofilm and antimicrobial (vs. planktonic bacteria) activities and indeed organisms completely resistant to antibiotic peptides were still able to be treated with anti-biofilm peptides. Thus the structure:activity relationships for the different types of activities of cationic peptides do not correspond such that it is possible to make an antimicrobial peptide with no anti-biofilm activity (de la Fuente-Núñez, C., et al. 2012) or an immune modulator peptide with no antimicriobial activity vs. planktonic bacteria (M. G., E. Dullaghan, et al. 2007), although it is possible to make peptides with both immunomodulatory and anti-biofilm activity (Haney, E. F., S et al. 2015; Mansour, S., et al. 2015.).

The innate immune system is a highly effective and evolved general defense system that involves a variety of effector functions including phagocytic cells, complement, etc., but is generally incompletely understood. Elements of innate immunity are always present at low levels and are activated very rapidly when stimulated by pathogens, acting to prevent these pathogens from causing disease. Generally speaking, many known innate immune responses are “triggered” by the binding of microbial signaling molecules, like lipopolysaccharide (LPS), to pattern recognition receptors such as Toll-like receptors (TLR) on the surface of host cells. Many of the effector functions of innate immunity are grouped together in the inflammatory response. However, too severe an inflammatory response can result in effects that are harmful to the body, and, in an extreme case, sepsis and potentially death can occur; indeed sepsis occurs in approximately 750,000 patients in North America annually with 210,000 deaths. Thus, a therapeutic intervention to boost innate immunity, which is based on stimulation of TLR signaling (for example using a TLR agonist), has the potential disadvantage that it could stimulate a potentially harmful inflammatory response and/or exacerbate the natural inflammatory response to infection.

Natural cationic host defense peptides (also known as antimicrobial peptides) are crucial molecules in host defenses against pathogenic microbe challenge. It has been hypothesized that since their direct antimicrobial activity is compromised by physiological salt concentrations (e.g. the 150 mM NaCl and 2 mM MgCl₂+CaCl₂) salt concentrations in blood), their most important activities are immunomodulatory (Bowdish D M E, et al. 2005).

A broad series of synthetic so-called innate defence regulator (IDR) peptides, as mimics of natural host defence peptides, which act to treat infections and inflammation in animal models, have been described. Although some IDR peptides are able to weakly kill planktonic bacteria, quantitative structure-activity relationship studies have suggested that antimicrobial and immunomodulatory activities are independently determined.

The host defence and IDR peptides have many anti-infective immunomodulatory activities, other than direct microbial killing, implying that such activities play a key role in innate immunity, including the suppression of acute inflammation and stimulation of protective immunity against a variety of pathogens (Hancock R E W, and Sahl H G. 2006). To demonstrate that synthetic variants of these peptides can protect without direct killing (i.e., by selectively modulating innate immunity), a bovine peptide homolog, innate defense regulator peptide (IDR)-1, which had no direct antibiotic activity, but was protective by both local and systemic administration in mouse models of infection with major Gram-positive and -negative pathogens, including MRSA, vancomycin-resistant Enterococcus (VRE), and Salmonella, was created (Scott et al. 2007). Protection by IDR-1 was prevented by in vivo depletion of monocytes and macrophages, but not neutrophils or lymphocytes indicating that the former were key effector cells. Gene and protein expression analysis in human and mouse monocytes and macrophages indicated that IDR-1 acted through mitogen-activated protein (MAP) kinase and other signaling pathways, to enhance the levels of monocyte chemokines while reducing pro-inflammatory cytokine responses. New IDR peptides implicated in protection in numerous animal models including E. coli, Salmonella, MRSA, VRE, multi-drug resistant tuberculosis, cystic fibrosis (CF), cerebral malaria, and perinatal brain injury from hypoxia-ischemia-LPS challenge (preterm birth model), and also have wound healing and vaccine adjuvant properties, have been described (Nijnik A., et al. 2010; Turner-Brannen, E., et al. 2011; Madera, L. and R. E. W. Hancock. 2012; Achtman, A. H., et al. 2012; Rivas-Santiago, B., J et al. 2013; Mayer, M. L., et al. 2013; Niyonsaba, F., L et al. 2013; Bolouri, H., et al. 2014; Kindrachuk, J., et al. 2009; Polewicz, M., et al. 2013; Steinstraesser, L., et al 2012).

Innate defence regulator peptide (IDR)-1 that had no direct antibiotic activity was nevertheless able, in mouse models, to protect against infections by major Gram-positive and -negative pathogens, including MRSA, VRE and Salmonella [Scott M G, E Dullaghan, N Mookherjee, N Glavas, M Waldbrook, A. Thompson, A Wang, K Lee, S Doria, P Hamill, J Yu, Y Li, O Donini, M M Guarna, B B Finlay, J R North, and R E W Hancock. 2007. An anti-infective peptide that selectively modulates the innate immune response. Nature Biotech. 25: 465-472]. IDR-1 peptide functioned by selectively modulating innate immunity, i.e. by suppressing potentially harmful inflammation while stimulating protective mechanisms such as recruitment of phagocytes and cell differentiation. This was also true of peptide 1018 which demonstrated superior protection in models of cerebral malaria and Staph aureus infection [Achtman, A H, S Pilat, C W Law, D J Lynn, L Janot, M Mayer, S Ma, J Kindrachuk, B B Finlay, F S L Brinkman, G K Smyth, R E W Hancock and L Schofield. 2012. Effective adjunctive therapy by an innate defense regulatory peptide in a pre-clinical model of severe malaria. Science Translational Medicine 4:135ra64] and (together with peptide HH2) against multi-drug resistant tuberculosis [Rivas-Santiago, B., J. E. Castañeda-Delgado, C. E. Rivas Santiago, M. Waldbrook, I. González-Curiel, J. C. León-Contreras, A. Enciso-Moreno, V. del Villar, J. Méndez-Ramos, R. E. W. Hancock, R. Hernandez-Pando. 2013. Ability of innate defence regulator peptides IDR-1002, IDR-HH2 and IDR-1018 to protect against Mycobacterium tuberculosis infections in animal models. PLoS One 8:e59119], as well as in increasing the rate of wound healing [Steinstraesser, L., T. Hirsch, M. Schulte, M. Kueckelhaus, F. Jacobsen, E. A. Mersch, I. Stricker, N. Afacan, H. Jenssen, R. E. W. Hancock and J. Kindrachuk. 2012. Innate defense regulator peptide 1018 in wound healing and wound infection. PLoS ONE 7:e39373]. LL-37 and 1018 appear to manifest this activity due to their ability to induce the production of certain chemokines which are able to recruit subsets of cells of innate immunity to infected tissues and to cause differentiation of recruited monocytes into particular subsets of macrophages with superior phagocytic activity [Pena O. M., N. Afacan, J. Pistolic, C. Chen, L. Madera, R. Falsafi, C. D. Fjell, and R. E. W. Hancock. 2013. Synthetic cationic peptide IDR-1018 modulates human macrophage differentiation. PLoS One 8:e52449]. A key chemokine for which its stimulated production in PBMC appears to correlate with protection in animal models in macrophage chemotactic protein 1 (MCP-1/CCL2).

The field of chemoinformatics involves computer-aided identification of new lead structures and their optimization into drug candidates (Engel T. 2006). One of the most broadly used chemoinformatics approaches is called Quantitative Structure-Activity Relationship (QSAR) modeling, which seeks to relate structural characteristics of a molecule (known as descriptors) to its measurable properties, such as biological activity. QSAR analysis has found a broad application in antimicrobial discovery. QSAR descriptors in combination with the approaches of the Artificial Intelligence have been used to successfully predict antimicrobial activity of cationic antimicrobial peptides (Cherkasov, A., et al. 2009.). The method has also been applied to anti-biofilm and immunomodulatory peptides (Haney et al., 2015).

A large number of publications have reported on sequence optimization strategies to enhance the potency of antimicrobial peptides (summarized in Fjell C D, et al. 2012). Most of these studies involve studying small peptide libraries with modifications made to residues deemed important based on properties known to contribute to antibacterial potency (i.e. acidic residues and hydrophobic residues, most notably Trp). Moreover, this large amount of data has also been exploited to generate quantitative structure activity relationship (QSAR) models which can accurately predict the antibacterial activity of peptides in silico and generate novel sequences with enhanced antibacterial potency (Cherkasov, A., et al. 2009; Fjell et al., 2012). By contrast, there relatively few peptide sequences that have been published that possess antibiofilm activity. International patent applications PCT/CA2007/001453, filed 21 Aug. 2007, published under No. WO 2008/022444 on 28 Feb. 2008 describe cationic antimicrobial peptides, and PCT/US2014/052993, filed 27 Aug. 2014, published under WO 2015/038339 on 19 Mar. 2015, describe cationic anti-biofilm and IDR peptides.

### SUMMARY

In one aspect, disclosed herein is an isolated antibiofilm or immunomodulatory peptide comprising 7 to 14 amino acids, wherein the antibiofilm or immunomodulatory peptide comprises an amino acid sequence as set forth in one or more of SEQ ID NOs: 6-1085 or a functional variant thereof. In an alternative aspect, the disclosure includes an isolated polynucleotide encoding the antibiofilm or immunomodulatory peptide as described herein.

In some embodiments of this aspect, the isolated antibiofilm or immunomodulatory peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In some embodiments, the isolated antibiofilm or immunomodulatory peptide may include a non-natural amino acid equivalent.

In some embodiments, the non-natural amino acid equivalent may be L-2-amino-3-guanidinopropionic acid, L-2-Amino-4-guanidinobutyric acid, L-Homoarginine, L-2,3-diaminopropionic acid or L-Ornithine.

In an alternative aspect, disclosed herein is an antibiofilm or immunomodulatory polypeptide X1-A-X2, where A includes an antibiofilm or immunomodulatory peptide as described herein; and where each X1 and X2 independently include an amino acid sequence of n amino acids, wherein n is 0 to 50.

In some embodiments, A may include a conservative amino acid substitution or peptide mimetic substitution having about 90% or greater amino acid sequence identity to an antibiofilm or immunomodulatory peptide as described herein.

In alternative embodiments of this aspect, the peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In an alternative aspect, disclosed herein is an antibiofilm or immunomodulatory peptide as set forth in Formula 1:

[chemical structure]

wherein:
Z₁, Z₄, Z₆ and Z₉ are each independently H, methyl-1H-indol-3-yl, isopropyl, methyl, 2-methylpropyl, or 1-methylpropyl;
B₃ is propyl-3-guanidine or α-aminobutyl;
J₅, and J₈ are each independently H, methyl-1H-indol-3-yl, isopropyl, methyl, 2-methylpropyl, 1-methylpropyl; propyl-3-guanidine, α-aminobutyl, propyl-3-guanidine, α-aminobutyl, or propyl-3-carboxamide;
U₂ is H, methyl-1H-indol-3-yl, isopropyl, methyl, 2-methylpropyl, 1-methylpropyl, or propyl-3-carboxamide;
Σ₁₀ is propyl-3-guanidine, α-aminobutyl, or propyl-3-carboxamide;
X₁ and X₂ are each independently 0 to 2 amino acids selected from the group consisting of 2-amino-3-(1h-indol-3-yl)propanoic acid, 2-amino-3-methylbutanoic acid, 2-aminopropanoic acid, 2-amino-4-methylpentanoic acid, 2-amino-3-methylpentanoic acid, aminoacetic acid, 2-amino-5-guanidinopentanoic acid, or 2,6-diaminohexanoic acid; wherein the peptide can also contain one substitution from the group Z₁=α-aminobutyl, B₃=2-methylpropyl, Z₆=propyl-3-guanidine, W₇ is H, methyl-1H-indol-3-yl, isopropyl, methyl, 2-methylpropyl, 1-methylpropyl, or propyl-3-carboxamide and Σ₁₀ is methyl.

In alternative embodiments of this aspect, the peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In an alternative aspect, disclosed herein is a method of inhibiting the growth of a bacterial biofilm or an abscess comprising contacting the bacterial biofilm or abscess with an inhibition effective amount of an antibiofilm or immunomodulatory peptide as described herein.

In some embodiments, the inhibiting effective amount of the antibiofilm or immunomodulatory peptide may be provided in combination with at least one antibiotic.

In some embodiments, the peptide may be bound to a solid support or surface.

In alternative embodiments of this aspect, the peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In an alternative aspect, disclosed herein is a method of enhancing innate immunity comprising contacting a cell with an effective amount of a peptide in accordance with the disclosure.

In alternative embodiments of this aspect, the peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In an alternative aspect, disclosed herein is a method of selectively suppressing a proinflammatory response comprising contacting a cell with an effective amount of a peptide in accordance with the disclosure.

In alternative embodiments of this aspect, the peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In some embodiments, the peptide can include a contiguous sequence of amino acids having the formula: AA1-AA2-AA3-AA4-AA5-AA6-AA7-AA8-AA9-AA10-AA11-AA12 and containing only the residues K, R, V, L, I, A, W and no more than two Q or G residues either on their own or in combination.

In an alternative aspect, disclosed herein is a polypeptide X1-A-X2 or a functional variant or mimetic thereof, wherein A represents at least one peptide having an amino acid sequence as set forth in SEQ ID NO: 6-1085, or in one or more of Tables 1, 2 or 8-15, or falls within a consensus sequence as described herein, or analogs, derivatives, enantiomers, amidated and unamidated variations and conservative variations thereof; and wherein each X1 and X2 independently of one another represents any amino acid sequence of n amino acids, n varying from 0 to 50, and n being identical or different in X1 and X2.

In some embodiments of this polypeptide, the functional variant or mimetic may be a conservative amino acid substitution or peptide mimetic substitution. In some embodiments of this polypeptide, the functional variant may have about 66% or greater amino acid identity. Truncation of amino acids from the N or C termini or from both can create these mimetics. In some embodiments of this polypeptide, the amino acids may be non-natural amino acid equivalents. In some embodiments of this polypeptide, n may be zero.

In alternative embodiments of this aspect, the peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In an alternative aspect, disclosed herein is a method of inhibiting the growth of bacterial biofilms comprising contacting a bacterial biofilm with an inhibiting effective amount of a peptide having an amino acid sequence set forth in SEQ ID NO: 6-1085, or in one or more of Tables 1, 2 or 8-15, or falls within a consensus sequence as described herein, or any combination thereof, or analogs, derivatives, enantiomers, amidated and unamidated variations and conservative variations thereof.

In some embodiments of this aspect, the bacterium may be Gram positive. In some embodiments of this aspect, the bacterium may be Staphylococcus aureus, Staphylococcus epidermidis, or Enterococcus faecalis. In some embodiments of this aspect, the bacterium may be Gram negative. In some embodiments of this aspect, the bacterium may be Pseudomonas aeruginosa, Escherichia coli, Salmonella enteritidis ssp Typhimurium, Acinetobacter baummanii, Klebsiella pneumoniae, Enterobacter sp., Campylobacter or Burkholderia cepacia complex.

In some embodiments of this aspect, the contacting includes a peptide in combination with at least one antibiotic. In some embodiments of this aspect, the antibiotic is selected from the group consisting of aminoglycosides, β-lactams, quinolones, and glycopeptides. In some embodiments of this aspect, the antibiotic may be selected from the group consisting of amikacin, gentamicin, kanamycin, netilmicin, tobramycin, streptomycin, azithromycin, clarithromycin, erythromycin, erythromycin estolate/ethyl-succinate/gluceptate/lactobionate/stearate, penicillin G, penicillin V, methicillin, nafcillin, oxacillin, cloxacillin, dicloxacillin, ampicillin, amoxicillin, ticarcillin, carbenicillin, mezlocillin, azlocillin, piperacillin, cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, cefsulodin, imipenem, aztreonam, fleroxacin, nalidixic acid, norfloxacin, ciprofloxacin, ofloxacin, enoxacin, lomefloxacin, cinoxacin, doxycycline, minocycline, tetracycline, vancomycin, chloramphenicol, clindamycin, trimethoprim, sulfamethoxazole, nitrofurantoin, rifampin and mupirocin and teicoplanin.

In some embodiments of this aspect, the peptide may be bound to a solid support. In some embodiments, the peptide may be bound covalently or noncovalently. In some embodiments of this aspect, the solid support may be a medical device.

In alternative embodiments of this aspect, the peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In some embodiments, the peptide may be capable of selectively enhancing innate immunity as determined by contacting a cell containing one or more genes that encode a polypeptide involved in innate immunity and protection against an infection, with the peptide of interest, wherein expression of the one or more genes or polypeptides in the presence of the peptide may be modulated as compared with expression of the one or more genes or polypeptides in the absence of the peptide, and wherein the modulated expression may result in enhancement of innate immunity. In further embodiments, the peptide does not stimulate a septic reaction. In further embodiments, the peptide may stimulate expression of the one or more genes or proteins, thereby selectively enhancing innate immunity. In further embodiments, the one or more genes or proteins may encode chemokines or interleukins that attract immune cells. In further embodiments, the one or more genes may be selected from the group consisting of MCP-1, MCP-3, and Gro-α.

In some embodiments, the peptide may selectively suppress proinflammatory responses, whereby the peptide may contact a cell treated with an inflammatory stimulus and containing a polynucleotide or polynucleotides that encode a polypeptide involved in inflammation and sepsis and which is normally upregulated in response to this inflammatory stimulus, and wherein the peptide may suppress the expression of this gene or polypeptide as compared with expression of the inflammatory gene in the absence of the peptide and wherein the modulated expression results in enhancement of innate immunity. In further embodiments, the peptide may inhibit the inflammatory or septic response. In further embodiments, the peptide may block the inflammatory or septic response. In further embodiments, the peptide may inhibit the expression of a pro-inflammatory gene or molecule. In further embodiments, the peptide may inhibit the expression of TNF-α. In further embodiments, the inflammation may be induced by a microbe or a microbial ligand acting on a Toll-like receptor. In further embodiments, the microbial ligand may be a bacterial endotoxin or lipopolysaccharide.

In an alternative aspect, disclosed herein is an isolated immunomodulatory polypeptide X1-A-X2, or a functional variant or mimetic thereof, wherein A represents at least one peptide having an amino acid sequence set forth in SEQ ID NO: 6-1085, or in one or more of Tables 1, 2 or 8-15, or falls within a consensus sequence as described herein, or analogs, derivatives, enantiomers, amidated and unamidated variations and conservative variations thereof each X1 and X2 independently of one another represents any amino acid sequence of n amino acids, n varying from 0 to 5, and n being identical or different in X1 and X2.

In some embodiments of this aspect, the functional variant or mimetic may be a conservative amino acid substitution or peptide mimetic substitution. In some embodiments of this aspect, the functional variant may have about 70% or greater amino acid sequence identity to X1-A-X2.

In alternative embodiments of this aspect, the peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In an alternative aspect, disclosed herein is a method of inhibiting the growth of bacterial biofilms comprising contacting the bacterial biofilm with an inhibiting effective amount of a peptide having an amino acid sequence of aspects one or four, or any combination thereof, or analogs, derivatives, enantiomers, amidated and unamidated variations and conservative variations thereof.

In some embodiments of this aspect, the bacterium may be Gram positive. In some embodiments of this aspect, the bacterium may be Staphylococcus aureus, Staphylococcus epidermidis, or Enterococcus faecaelis.

In some embodiments of this aspect, the bacterium may be Gram negative. In some embodiments of this aspect, the bacterium may be Pseudomonas aeruginosa, Escherichia coli, Salmonella enteritidis ssp Typhimurium, Acinetobacter baummanii, Klebsiella pneumoniae, Campylobacter, or Burkholderia cepacia complex.

In some embodiments of this aspect, the contacting may include a peptide in combination with at least one antibiotic. In some embodiments, the antibiotic may be selected from the group consisting of aminoglycosides, β-lactams, quinolones, and glycopeptides.

In some embodiments, the antibiotic may be selected from the group consisting of amikacin, gentamicin, kanamycin, netilmicin, tobramycin, streptomycin, azithromycin, clarithromycin, erythromycin, erythromycin estolate/ethyl-succinate/gluceptate/lactobionate/stearate, penicillin G, penicillin V, methicillin, nafcillin, oxacillin, cloxacillin, dicloxacillin, ampicillin, amoxicillin, ticarcillin, carbenicillin, mezlocillin, azlocillin, piperacillin, cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, cefsulodin, imipenem, aztreonam, fleroxacin, nalidixic acid, norfloxacin, ciprofloxacin, ofloxacin, enoxacin, lomefloxacin, cinoxacin, doxycycline, minocycline, tetracycline, vancomycin, chloramphenicol, clindamycin, trimethoprim, sulfamethoxazole, nitrofurantoin, rifampin and mupirocin and teicoplanin.

In some embodiments of this aspect, the peptide may be bound to a solid support. In some embodiments, the peptide is bound covalently or noncovalently. In some embodiments of this aspect, the solid support may be a medical device.

In alternative embodiments of this aspect, the peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In some embodiments, the peptide may be capable of selectively enhancing innate immunity as determined by contacting a cell containing one or more genes that encode a polypeptide involved in innate immunity and protection against an infection, with the peptide of interest, wherein expression of the one or more genes or polypeptides in the presence of the peptide may be modulated as compared with expression of the one or more genes or polypeptides in the absence of the peptide, and wherein the modulated expression may result in enhancement of innate immunity.

In some embodiments of this aspect, the peptide does not stimulate a septic reaction.

In some embodiments of this aspect, the peptide may stimulate expression of the one or more genes or proteins, thereby selectively enhancing innate immunity. In some embodiments, the one or more genes or proteins may encode chemokines or interleukins that attract immune cells. In some embodiments, the one or more genes may be selected from the group consisting of MCP-1, MCP-3, and Gro-α.

In some embodiments, the peptide may selectively suppress proinflammatory responses, whereby the peptide can contact a cell treated with an inflammatory stimulus and containing a polynucleotide or polynucleotides that encode a polypeptide involved in inflammation and sepsis and which is normally upregulated in response to this inflammatory stimulus, and wherein the peptides may suppress the expression of this gene or polypeptide as compared with expression of the inflammatory gene in the absence of the peptide and wherein the modulated expression may result in enhancement of innate immunity.

In some embodiments, the peptide may inhibit the inflammatory or septic response. In some embodiments, the peptide may inhibit the expression of a pro-inflammatory gene or molecule. In some embodiments, the peptide may inhibit the expression of TNF-α. In some embodiments, the inflammation may be induced by a microbe or amicrobial ligand acting on a Toll-like receptor. In some embodiments, the microbial ligand may be a bacterial endotoxin or lipopolysaccharide.

In alternative embodiments of this aspect, the peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In an alternative aspect, disclosed herein is an isolated molecule that may have anti-biofilm activity by virtue of inhibiting (p)ppGpp synthesis or causing (p)ppGpp degradation. In some embodiments, the molecule may be a peptide. In some embodiments, the peptide may have 7 to 12 amino acids, where the peptide has an amino acid sequence set forth in SEQ ID NO: 6-1085, or in one or more of Tables 1, 2 or 8-15, or falls within a consensus sequence as described herein, or analogs, derivatives, enantiomers, amidated and unamidated variations and conservative variations thereof.

In alternative embodiments of this aspect, the peptide may include an amino acid sequence as set forth in one or more of SEQ ID NOs: 25, 29, 30, 38, 39, 44, 45, 46, 57, 60, 65, 151 or 152 or a functional variant thereof. In alternative embodiments of this aspect, the peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

### BRIEF DESCRIPTION OF THE DRAWINGS

FIGS. 1A-B show the distribution of antibiofilm activities of SPOT-synthesized 1018 single amino acid substitution peptides comprising the Training Set. The antibiofilm activities of the peptides in the Training Set were used for the initial QSAR models. The antibiofilm activity was measured against S. aureus (MRSA SAP0017) at a concentration of ˜2.5 μM and the 1018 derivatives exhibited a large range of activities (A). The percentage of biofilm inhibited by SPOT-synthesized 1018 (31%) is indicated. When all the percentages of biofilm inhibition are plotted as an amino acid substitution matrix, those residues that contribute to the antibiofilm activity of 1018 become apparent (B). Each box represents an individual peptide shaded from the most active sequences (top 25^{th} percentile, in black) to moderately active (grey) and to least active (bottom 75^{th} percentile, in white).

FIG. 2 shows the distribution of antibiofilm activities of QSAR derived peptides comprising the Experimental Validation Set. The Experimental Validation Set contained 108 sequences of different predicted antibiofilm potency from throughout the 100,000 peptides in the Virtual Set. All peptides in the Experimental Validation Set were SPOT-synthesized and screened for their antibiofilm activity against S. aureus (MRSA SAP0017). A significant number of peptides were identified with significantly improved antibiofilm activity compared to the parent sequence, 1018.

FIGS. 3A-B show the antibiofilm activity of synthetic QSAR optimized antibiofilm peptides and identification of a peptide with enhanced antibiofilm activity. All peptides (3001-3007) were commercially synthesized to greater than 95% purity. The antibiofilm activity was initially evaluated in the static microtitre plate assay against S. aureus (MRSA SAP0017) and the residual biomass was stained with 0.1% crystal violet (A). Most of the QSAR derived peptides demonstrated antibiofilm activity similar to the parent peptide, 1018, while one peptide, 3002, exhibited enhanced antibiofilm activity and substantially inhibited biofilm growth at peptide concentrations at low as 1 μM MRSA biofilms were then grown in flow cells and treated with peptide 1018 and 3002 to evaluate the ability of each peptide to eradicate pre-formed biofilms. Peptide 3002 was found to substantially reduce preformed biofilms at 0.125 μM while 1018 was no longer effective at this same concentration (B).

FIG. 4 shows the aggregation properties of the QSAR optimized antibiofilm peptides as a function of phosphate ion concentration. Peptide samples were prepared to a final concentration of 1 mg/ml in the appropriate concentration of sodium phosphate buffer (pH 7.0) and aggregation was quantitated by measuring the increase in sample turbidity at 600 nm and compared to the same peptide sample in water. While many of the antibiofilm peptides aggregated under these conditions, the tested peptides exhibited lower turbidity (proportional to the level of aggregation) compared to the parent peptide, 1018.

FIGS. 5A-B show the protection by an anti-biofilm peptide in the mouse chronic abscess model vs Pseudomonas aeruginosa Mice were infected subcutaneously with P. aeruginosa Liverpool epidemic strain LESB58 and then treated 2 hours later with 10 mg/kg of 3002 (or controls 1018, DJK6) via intra-abscess injection. Representative images capturing dermonecrotic abscess lesions were taken 72 hours post-infection. Abscess sizes were measured three days post-infection using a caliper. After three days, bacteria were recovered from saline or peptide treated animals and enumerated. Peptide 3002 was the best of these peptides and superior to 1018 at the same concentration in reducing abscess size after 3 days (A) but there was no observable change in colony forming units (CFU) pre-abscess (B).

FIGS. 6A-D show the immunomodulatory activity of QSAR optimized antibiofilm peptides evaluated against PBMCs. The peptides (3001-3007) were commercially synthesized to greater than 95% purity. The cytotoxic and immunomodulatory activities of each peptide was evaluated at concentrations of 40 (black bars), 20 (dark gray bars) and 10 (light gray bars) μM. Hemolysis was evaluated against red blood cells (A) with vehicle treated cells (defined as 0%) and cells lysed with 2% Triton X-100 (defined as 100%, horizontal dashed line) serving as controls. Peptide cytotoxicity was measured against PBMCs using the LDH assay (B) and the same positive and negative controls. Chemokine production by peptide was evaluated by measuring peptide induced MCP1 production from PBMCs (C). Peptide suppression of pro-inflammatory cytokines was also evaluated by quantifying the LPS-induced IL-1β production in the presence of peptide and comparing to cells stimulated by LPS alone (D). The levels of chemokine and cytokine present in each sample were quantified by ELISA. All peptides were tested in triplicate and data are shown as the average±the standard error of the mean.

FIGS. 7A-D show the distribution of immunomodulatory activities of SPOT-synthesized 1018 single amino acid substitution peptides comprising the Training Set. The MCP1 inducing activities and IL-1B suppressing capacities against PBMCs were used to establish the initial QSAR models. The amount of MCP1 induced by the peptides exhibited a large range of activities (A) as did the level of IL-1B suppression from LPS-stimulated PBMCs (B). When levels of MCP1 induction (C) and IL-1B suppression (D) are plotted as an amino acid substitution matrix, those residues that contribute to the immunomodulatory activities of 1018 become apparent. Each box represents an individual peptide shaded from the most active sequences (top 25^{th} percentile, in black) to moderately active (grey) and to least active (bottom 75^{th} percentile, in whitle).

FIGS. 8A-E show the biological activity of QSAR optimized chemokine (MCP1) inducing peptides. All peptides (3008-3015) were commercially synthesized to greater than 95% purity. Antibiofilm activity (A) was evaluated against MRSA biofilms as described in FIG. 3 while cytotoxicity and immunomodulatory activity was measured in the same way as described in FIG. 6. Hemolysis was evaluated against red blood cells (B) with vehicle treated cells (defined as 0%) and cells lysed with 2% Triton X-100 (defined as 100%, horizontal dashed line) serving as controls. Peptide cytotoxicity was measured against PBMCs using the LDH assay (C) using the same positive and negative controls. Chemokine production by peptide was evaluated by measuring peptide induced MCP1 production from PBMCs (D). Peptide suppression of pro-inflammatory cytokines was also evaluated by quantifying the LPS-induced IL-1β production in the presence of peptide and comparing to cells stimulated by LPS alone (E). The levels of chemokine and cytokine present in each sample were quantified by ELISA. All peptides were tested in triplicate and data are shown as the average+/− the standard error of the mean.

FIGS. 9A-E show the biological activity of QSAR optimized pro-inflammatory cytokine (IL-1β) suppressing peptides. All peptides (3016-3024) were commercially synthesized to greater than 95% purity. Antibiofilm activity (A) was evaluated against MRSA biofilms as described in FIG. 3 while cytotoxicity and immunomodulatory activity was measured in the same way as described in FIG. 6. Hemolysis was evaluated against red blood cells (B) with vehicle treated cells (defined as 0%) and cells lysed with 2% Triton X-100 (defined as 100%, horizontal dashed line) serving as controls. Peptide cytotoxicity was measured against PBMCs using the LDH assay (C) using the same positive and negative controls. Chemokine production by peptide was evaluated by measuring peptide induced MCP1 production from PBMCs (D). Peptide suppression of pro-inflammatory cytokines was also evaluated by quantifying the LPS-induced IL-1β production in the presence of peptide and comparing to cells stimulated by LPS alone (E). The levels of chemokine and cytokine present in each sample were quantified by ELISA. All peptides were tested in triplicate and data are shown as the average+/− the standard error of the mean.

FIG. 10 shows the derivation of the consensus sequence of the most active QSAR derived peptides for sequences that displayed multiple biological activities. The amino acids are set out in accordance with the one letter amino acid code. Other single letter designations are: Z=hydrophobic residues (W, V, L, I, A or G); B=basic residues (R or K); J=Basic or hydrophobic residues (Z+B); U=Uncharged residues (Z+Q); O=polar residues (B+Q).

FIG. 11 shows exemplary chemical structures with anti-biofilm, immunodulatory (MCP-1 induction) and anti-inflammatory activity. The chemical structures of exemplary chemical structures are shown with the side chains represented as letters. The code for the substitution preferences at each position is indicated at the bottom of the figure. The peptides of Table 8 were at least 90% identical in the central 10 amino acid motif. Allowable variants in any of the active peptides are shown as single substitutions at the bottom of the figure.

FIGS. 12A-B show the antibiofilm activity distribution of SPOT-synthesized 1018 derivatives and percent biofilm inhibition of each derivative plotted as a substitution matrix. Shown is the percent MRSA biofilm growth of three biological replicates (+/−SD) in the presence of the highest concentration of SPOT-synthesized peptide evaluated (A) which represents a 10-fold dilution of the stock solution of SPOT-peptide. When plotted as a substitution matrix, this reveals residues important for antibiofilm activity and where non-natural cationic amino acids can be inserted into the sequence of 1018 (B). Each box corresponds to the percent biofilm growth observed for each peptide and the colour scale correspond to <25% biofilm growth in black, 50% biofilm growth in grey and >75% biofilm growth in white.

FIGS. 13A-D show the biological activity summary of the cationic amino acid substituted 1018 derivatives. The ability of 1018 and the cationic derivatives to inhibit biofilms formed by MRSA was evaluated by crystal violet staining using a microtitre plate assay (A). The antibiofilm activity data represents the average (+/−SEM) of three biological replicates. Peptide cytotoxicity was quantified by the LDH release assay at peptide concentrations of 10, 20 and 40 μM (B). In addition, the immumodulatory activity of the 1018 derivatives towards PBMCs was quantified by measuring the amount of MCP-1 chemokine induced by peptide alone (C) as well as the ability of the peptides to suppress the production of the pro-inflammatory cytokine, IL-1β, released from LPS-stimulated cells (D). The levels of the pro-inflammatory cytokine, IL-1β, have been normalized to the amount of cytokine induced by LPS stimulation alone (defined as 1.0). The cytotoxicity and immunomodulatory activity data represent the average (+/−SEM) of six biological replicates.

FIGS. 14A-C show the tryptophan emission fluorescence spectroscopy of 1018 and designed cationic amino acid derivatives. Representative Trp-emission spectra of 1018 recorded in Tris buffer or in the presence of SDS (25 mM) or DPC (10 mM) micelles (A). The maximum Trp-emission wavelength (λmax) of each peptide (B) as well as the relative emission intensity normalized to the λmax recorded in buffer (C) is shown to compare between the 1018 derivatives. Data shown are the average of three individual experiments (+/−SD).

FIGS. 15A-C show the effect of peptide treatment on abscess size and bacterial burden in an in vivo model of high density bacterial infection. CD-1 mice were injected with MRSA USA300 LAC at a density of 5×10⁷ CFU/50 μl to establish the abscess. After one hour, peptide (at 14 mg/kg) or vehicle (saline) control was injected intra-abscess and the abscess growth was monitored for 3 days. The representative photo of mice in the vehicle control group show prominent abscesses on the right flank while peptide treated abscesses were clearly smaller and less pronounced (A). Quantification of the abscess sizes revealed that both 1018 and 3002 treatments significantly reduced the abscess size in peptide treated mice based on a one-way ANOVA analysis (B). However, the bacterial burden within the peptide treated abscesses was unaffected by peptide treatment (C).

FIGS. 16A-L show the antibiofilm activity of selected synthetic peptides against pre-formed P. aeruginosa PAO1 biofilms. PAO1 biofilms were grown in 96-well microtitre plates for 24 hrs in BM2 minimal media (62 mM potassium phosphate, 7 mM ammonium sulphate, 0.4% glucose, 0.5 mM magnesium sulphate and 10 μM iron sulphate, pH 7.0). Planktonic cells were then rinsed three times with fresh BM2 media and then peptide treatments were added to each well and incubated for an additional 24 hrs. Biofilm growth was quantified at the end of the experiment by rinsing away planktonic cells and then staining with crystal violet (circles) to measure the amount of biofilm biomass present in each well or by measuring the conversion of a metabolic dye, triphenyl tetrazolium chloride (TTC, squares) to quantify the amount of biofilm cells that were metabolically active. (Note—for metabolic samples, TTC dye was added to a final concentration of 0.05% at the same time as the peptide treatments and incubated with peptide for 24 hrs). Shown are peptides that caused at least a 50% reduction in either biomass or metabolic activity within the peptide concentration range evaluated.

FIG. 17A-B show the antibiofilm activity distribution of SPOT-synthesized single amino acid substitution variants of peptide 3002 (A) and 3007 (B). Each box represents an individual peptide sequence with the amino acid appearing in the left-most column substituted at each position within the parent sequence, indicated along the top row. The values indicated for each sample represent the concentration of peptide required to inhibit 50% MRSA biofilm growth (IC₅₀) in a static microtitre plate assay. The colour scale represents the most active peptides (top 25%) in black, the mid peptides (50^{th} percentile) in grey and the bottom 25% (75^{th} percentile) in white.

FIG. 18A-B show the normalized antibiofilm activity distribution of SPOT-synthesized single amino acid substitution variants of peptide 3002 (A) and 3007 (B). Each box represents an individual peptide sequence with the amino acid appearing in the left-most column substituted at each position within the parent sequence, indicated along the top row. The values indicated for each peptide are normalized to the IC50 determined for the parent peptide (defined as 1). The colour scale indicates peptides that are more active than the parent peptide in black and less active than the parent peptide in grey.

FIGS. 19A-N show the anti-biofilm activity summary of various L-, D- and RI-peptides. D- and RI-forms of peptides 3001-3007 were SPOT-synthesized on peptide arrays and screened for their ability to inhibit MRSA (C623) and P. aeruginosa (PAO1) biofilms in a static microtitre plate assay. Purified (>95%) L-forms of each peptide were run for comparison as well as 1018 and RI-1018. Squares indicate L-peptides, circles indicate D-peptides and triangles indicate RI-peptides.

FIGS. 20A-G show the hemolytic activity summary of L-, D- and RI-peptides. D- and RI-forms of peptides 3001-3007 were SPOT-synthesized on peptide arrays and screened. Purified (>95%) L-forms of each peptide were run for comparison as well as 1018 and RI-1018. Squares indicate L-peptides, circles indicate D-peptides and triangles indicate RI-peptides.

FIGS. 21A-B show the tryptic stability of cationic substituted 1018 derivatives. Peptides were incubated in the absence or presence of bovine trypsin for 30 minutes. Peptide samples (10 μM) were incubated at 37° C. in the absence (black) or presence of trypsin (grey) and the samples were subjected to RP-HPLC analysis using a water-acetonitrile gradient (A). Absorbance values in the chromatogram have been normalized to the maximum absorbance (280 nm) observed in the peptide sample in the absence of trypsin. The amount of peptide in each sample was then quantified by comparing the area of the peak on the chromatogram for the undigested peptide to the corresponding peak in the digested sample (B). Data represent the average of three biological replicates (±SD) and statistical significance was calculated by one-way ANOVA comparing each peptide to the amount of 1018 digested under the same conditions (P-value: *=0.033, **=0.002, ***=<0.001).

### DETAILED DESCRIPTION

The present disclosure provides, in part, peptides that have broad spectrum activity against biofilms (and “anti-biofilm” peptide). In some embodiments, a peptide according to the present disclosure may have weaker activity against so-called planktonic, free-swimming cells. Exemplary peptides include those with their carboxyl terminus residue carboxy-amidated and having the amino acid sequences set forth in one or more of SEQ ID NOs: 6-1085, or a functional variant thereof. In some embodiments, the isolated antibiofilm or immunomodulatory peptide may include an amino acid sequence as set forth in one or more of SEQ ID NOs: 25, 29, 30, 38, 39, 44, 45, 46, 57, 60, 65, or a functional variant thereof. In alternative embodiments, the isolated antibiofilm or immunomodulatory peptide may include an amino acid sequence as set forth in one or more of SEQ ID NOs: 151 or 152 (peptides D-3006 or D-3007, respectively), or a functional variant thereof. In alternative embodiments, the isolated antibiofilm or immunomodulatory peptide may include an amino acid sequence as set forth in one or more of SEQ ID NOs: 24-47, 146-169, 196-219, 246-437, or a functional variant thereof.

In some embodiments, a peptide according to the present disclosure may exhibit enhanced activity when compared to a reference peptide, such as peptide 1018. By “enhance,” “enhanced” or “enhancing” means an increase in activity by any value between about 10% and about 90%, or of any value between about 30% and about 60%, or over about 100%, or an increase by about 1-fold, 2-fold, 5-fold, 8-fold, 10-fold or more, in comparison to a reference sample or molecule, such as a peptide, or a control. In some embodiments, the enhanced activity may be at least 5-fold. In some embodiments, the enhanced activity may be at least 8-fold.

In some embodiments, a peptide according to the present disclosure may exhibit anti-biofilm activity, for example, any one of the peptides including an amino acid sequence as set forth in one or more of SEQ ID NOs: 24-47, 146-169, 196-219, 246-437, or a functional variant thereof. In some embodiments, a peptide according to the present disclosure that exhibits broad spectrum anti-biofilm activities may include for example, any one of peptides 3013, 3015, 3016, D-3006 or D-3007, or a functional variant thereof. In some embodiments, a peptide according to the present disclosure that exhibits preferential activity against biofilms, compared to planktonic cells, may include for example, any one of peptides 3001-3008, 3011, 3016-3023, D-3006 or D-3007, or a functional variant thereof. In some embodiments, a peptide according to the present disclosure that exhibits enhanced anti-biofilm activities, when compared to a reference peptide, such as peptide 1018, may include for example, any one of peptides 3001-3007, D-3006 or D-3007, or a functional variant thereof. By “enhance,” “enhanced” or “enhancing” means an increase in anti-biofilm activity by any value between about 10% and about 90%, or of any value between about 30% and about 60%, or over about 100%, or an increase by about 1-fold, 2-fold, 5-fold, 8-fold, 10-fold or more, in comparison to a reference sample or molecule, such as a peptide, or a control. In some embodiments, the enhanced anti-biofilm activity may be at least 5-fold. In some embodiments, the enhanced anti-biofilm activity may be at least 8-fold.

In some embodiments, a peptide according to the present disclosure may exhibit lower aggregation when compared to a reference peptide, such as peptide 1018. In some embodiments, a peptide according to the present disclosure that exhibits lower aggregation, when compared to a reference peptide, such as peptide 1018, may include for example, any one of peptides 3001-3007, D-3006 or D-3007, or a functional variant thereof. In some embodiments, a peptide according to the present disclosure that exhibits lower aggregation, when compared to a reference peptide, such as peptide 1018, may include for example, any one of peptides 3002, 3003, 3004, D-3006 or D-3007 or a functional variant thereof. By “lower aggregation” means a decrease the tendency of a peptide to self-assemble, for example, through the interactions of their hydrophobic region(s) by any value between about 10% and about 90%, or of any value between about 30% and about 60%, or over about 100%, or an increase by about 1-fold, 2-fold, 5-fold, 8-fold, 10-fold or more, in comparison to a reference sample or molecule, such as a peptide, or a control.

In some embodiments, a peptide according to the present disclosure may reduce bacterial abscess formation when compared to a reference peptide, such as peptide 1018. In some embodiments, a peptide according to the present disclosure that reduces bacterial abscess formation, when compared to a reference peptide, such as peptide 1018, may include for example, any one of peptides 3002, D-3006 or D-3007, or a functional variant thereof. By “reduces bacterial abscess formation” or “reduction in bacterial abscess formation” is meant a decrease in abscess size by any value between about 10% and about 90%, or of any value between about 30% and about 60%, or over about 100%, or a decrease by about 1-fold, 2-fold, 5-fold, 8-fold, 10-fold or more, in comparison to a reference sample or molecule, such as a peptide, or a control.

In some embodiments, a peptide according to the present disclosure may additionally, or alternatively, have immunomodulatory activity. In some embodiments, a peptide according to the present disclosure that exhibits immunomodulatory activities, may include for example, any one of peptides including an amino acid sequence as set forth in one or more of SEQ ID NOs: 25, 29, 30, 38, 39, 44, 45, 46, 57, 60, 65, or a functional variant thereof. In alternative embodiments, the peptide that exhibits immunomodulatory activities may include an amino acid sequence as set forth in one or more of SEQ ID NOs: 151 or 152 (peptides D-3006 or D-3007, respectively), or a functional variant thereof. In alternative embodiments, the peptide that exhibits immunomodulatory activities may include an amino acid sequence as set forth in one or more of SEQ ID NOs: 24-47, 146-169, 196-219, 246-437, or a functional variant thereof.

In some embodiments, a peptide according to the present disclosure may additionally, or alternatively, have anti-inflammatory activity. In some embodiments, a peptide according to the present disclosure that exhibits anti-inflammatory activities, includes for example, any one of peptides including an amino acid sequence as set forth in one or more of SEQ ID NOs: 25, 29, 30, 38, 39, 44, 45, 46, 57, 60, 65, or a functional variant thereof. In alternative embodiments, the peptide that exhibits anti-inflammatory activities may include an amino acid sequence as set forth in one or more of SEQ ID NOs: 151 or 152 (peptides D-3006 or D-3007, respectively), or a functional variant thereof. In alternative embodiments, the peptide that exhibits anti-inflammatory activities may include an amino acid sequence as set forth in one or more of SEQ ID NOs: 24-47, 146-169, 196-219, 246-437, or a functional variant thereof.

In some embodiments, a peptide according to the present disclosure may stimulate chemokine expression, for example, MCP-1 or CCL5 expression. In some embodiments, a peptide according to the present disclosure that stimulates chemokine expression, such as MCP-1 expression, includes for example, any one of peptides 3008-3015, D-3006 or D-3007, or a functional variant thereof. In some embodiments, a peptide according to the present disclosure that stimulates chemokine expression, such as CCL5 expression, includes for example, any one of peptides 3009, 3010, 3016, 3017, D-3006 or D-3007, or a functional variant thereof. In some embodiments, a peptide according to the present disclosure may stimulate chemokine expression when compared to a reference peptide, such as peptide 1018. In some embodiments, a peptide according to the present disclosure that stimulates chemokine expression, when compared to a reference peptide, such as peptide 1018, includes for example, any one of peptides, 3008, 3010, 3012, 3013, 3015, D-3006 or D-3007, or a functional variant thereof. By “stimulate chemokine expression” or “stimulation of chemokine expression” is meant an increase in production of a chemokine by any value between about 10% and about 90%, or of any value between about 30% and about 60%, or over about 100%, or an increase by about 1-fold, 2-fold, 5-fold, 8-fold, 10-fold or more, in comparison to a reference sample or molecule, such as a peptide, or a control.

In some embodiments, a peptide according to the present disclosure may exhibit low toxicity. In some embodiments, a peptide according to the present disclosure that exhibits low toxicity includes for example, any one of peptides 3002, 3005, 3007-3011, 3015-3017, 3020-3024, D-3006 or D-3007, or a functional variant thereof. By “low toxicity” or “reduction in toxicity” is meant a decrease in peptide-induced cytotoxicity by any value between about 10% and about 90%, or of any value between about 30% and about 60%, or over about 100%, or a decrease by about 1-fold, 2-fold, 5-fold, 8-fold, 10-fold or more, in comparison to a reference sample or molecule, such as a peptide, or a control.

In some embodiments, a peptide according to the present disclosure may reduce proinflammatory cytokine expression, for example, IL1-β expression. In some embodiments, a peptide according to the present disclosure that reduces proinflammatory cytokine expression includes for example, any one of peptides 3016-3024, D-3006 or D-3007, or a functional variant thereof. In some embodiments, a peptide according to the present disclosure may reduce proinflammatory cytokine expression when compared to a reference peptide, such as peptide 1018. In some embodiments, a peptide according to the present disclosure that reduces proinflammatory cytokine expression, when compared to a reference peptide, such as peptide 1018, includes for example, any one of peptides 3016, 3018-3024, D-3006 or D-3007, or a functional variant thereof. By “reduce proinflammatory cytokine expression” or “reduction of proinflammatory cytokine expression” is meant a decrease in production of a proinflammatory chemokine by any value between about 10% and about 90%, or of any value between about 30% and about 60%, or over about 100%, or a decrease by about 1-fold, 2-fold, 5-fold, 8-fold, 10-fold or more, in comparison to a reference sample or molecule, such as a peptide, or a control.

In some embodiments, a peptide according to the present disclosure may exhibit both anti-biofilm and immunomodulatory activities. In some embodiments, a peptide according to the present disclosure that exhibits both anti-biofilm and immunomodulatory activities, includes for example, any one of the peptides including an amino acid sequence as set forth in one or more of SEQ ID NOs: 25, 29, 30, 38, 39, 44, 45, 46, 57, 60, 65, or a functional variant thereof. In alternative embodiments, the isolated antibiofilm and immunomodulatory peptide may include an amino acid sequence as set forth in one or more of SEQ ID NOs: 151 or 152 (peptides D-3006 or D-3007, respectively), or a functional variant thereof. In alternative embodiments, the isolated antibiofilm and immunomodulatory peptide may include an amino acid sequence as set forth in one or more of SEQ ID NOs: 24-47, 146-169, 196-219, 246-437, or a functional variant thereof.

“Anti-biofilm” relates to the ability to destroy, inhibit the growth of, or encourage the dispersal of, biofilms of living organisms, such as microorganisms. “Antimicrobial” as used herein means that a peptide of the present invention can inhibit, prevent, or destroy the growth or proliferation of planktonic (free swimming) microbes such as bacteria, fungi, viruses, parasites or the like.

“Immunomodulatory” or “Selective enhancement of innate immunity” as used herein means that the peptides of the invention are able to upregulate, in mammalian cells, genes and molecules that are natural components of the innate immune response and assist in the resolution of infections without excessive increases, or with actual decreases, of pro-inflammatory cytokines like TNFα that can cause potentially harmful inflammation and thus initiate a sepsis reaction in a subject. The peptides do not stimulate a septic reaction, but do stimulate expression of the one or more genes encoding chemokines or interleukins that attract immune cells including MCP-1, MCP-3, and CXCL-1. The peptides may also possess anti-sepsis activity including an ability to reduce the expression of TNFα in response to bacterial ligands like LPS.

In some aspects, the present disclosure provides a method of inhibiting the growth of or causing dispersal of a bacterium in a biofilm including contacting the biofilm with an inhibiting effective amount of at least one peptide of the disclosure alone, or in combination with at least one antibiotic. Classes of antibiotics that can be used in with the peptides of the disclosure include, but are not limited to, aminoglycosides, β-lactams, fluoroquinolones, vancomycin, and macrolides. In some embodiments of this aspect, the bacterium may be Gram positive. In some embodiments of this aspect, the bacterium may be Staphylococcus aureus, Staphylococcus epidermidis, or Enterococcus faecalis. In some embodiments of this aspect, the bacterium may be Gram negative. In some embodiments of this aspect, the bacterium may be Pseudomonas aeruginosa, Escherichia coli, Salmonella enteritidis ssp Typhimurium, Acinetobacter baummanii, Klebsiella pneumoniae, Enterobacter sp., Campylobacter or Burkholderia cepacia complex.

In some embodiments of this aspect, the contacting includes a peptide in combination with at least one antibiotic. In some embodiments of this aspect, the antibiotic is selected from the group consisting of aminoglycosides, β-lactams, quinolones, and glycopeptides. In some embodiments of this aspect, the antibiotic may be selected from the group consisting of amikacin, gentamicin, kanamycin, netilmicin, tobramycin, streptomycin, azithromycin, clarithromycin, erythromycin, erythromycin estolate/ethyl-succinate/gluceptate/lactobionate/stearate, penicillin G, penicillin V, methicillin, nafcillin, oxacillin, cloxacillin, dicloxacillin, ampicillin, amoxicillin, ticarcillin, carbenicillin, mezlocillin, azlocillin, piperacillin, cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, cefsulodin, imipenem, aztreonam, fleroxacin, nalidixic acid, norfloxacin, ciprofloxacin, ofloxacin, enoxacin, lomefloxacin, cinoxacin, doxycycline, minocycline, tetracycline, vancomycin, chloramphenicol, clindamycin, trimethoprim, sulfamethoxazole, nitrofurantoin, rifampin and mupirocin and teicoplanin. In some embodiments, the combination of a peptide according to the present disclosure with an antibiotic may provide synergistic therapy.

In some embodiments of this aspect, the peptide may be bound to a solid support. In some embodiments, the peptide may be bound covalently or noncovalently. In some embodiments of this aspect, the solid support may be a medical device.

In some embodiments, the present disclosure provides a method of modulating the innate immune response of human cells to enhance the production of a protective immune response while not inducing or inhibiting the potentially harmful proinflammatory response.

In some embodiments, the peptide may be capable of selectively enhancing innate immunity as determined by contacting a cell containing one or more genes that encode a polypeptide involved in innate immunity and protection against an infection, with the peptide of interest, wherein expression of the one or more genes or polypeptides in the presence of the peptide may be modulated as compared with expression of the one or more genes or polypeptides in the absence of the peptide, and wherein the modulated expression may result in enhancement of innate immunity. In further embodiments, the peptide does not stimulate a septic reaction. In further embodiments, the peptide may stimulate expression of the one or more genes or proteins, thereby selectively enhancing innate immunity. In further embodiments, the one or more genes or proteins may encode chemokines or interleukins that attract immune cells. In further embodiments, the one or more genes may be selected from the group consisting of MCP-1, MCP-3, and Gro-α.

In some embodiments, the peptide may selectively suppress proinflammatory responses, whereby the peptide may contact a cell treated with an inflammatory stimulus and containing a polynucleotide or polynucleotides that encode a polypeptide involved in inflammation and sepsis and which is normally upregulated in response to this inflammatory stimulus, and wherein the peptide may suppress the expression of this gene or polypeptide as compared with expression of the inflammatory gene in the absence of the peptide and wherein the modulated expression results in enhancement of innate immunity. In further embodiments, the peptide may inhibit the inflammatory or septic response. In further embodiments, the peptide may block the inflammatory or septic response. In further embodiments, the peptide may inhibit the expression of a pro-inflammatory gene or molecule. In further embodiments, the peptide may inhibit the expression of TNF-α. In further embodiments, the inflammation may be induced by a microbe or a microbial ligand acting on a Toll-like receptor. In further embodiments, the microbial ligand may be a bacterial endotoxin or lipopolysaccharide.

In some embodiments, the present disclosure provides a polynucleotide that encodes one or more of a peptide of the disclosure.

In some embodiments, the present disclosure provides a method of identifying an anti-biofilm peptide having 7 to 14 amino acids. The method may include contacting, under conditions sufficient for anti-biofilm activity, a test peptide with a microbe that will form or has formed one or more surface-associated biofilm colonies, and detecting a reduced amount of biofilm as compared to amount of biofilm in the absence of the test peptide. In one embodiment, the peptide may be synthesized on, or attached to, a solid support. In some embodiments, the peptides may retain anti-biofilm activity when cleaved from the solid support or may retain activity when still associated with the solid support. The microbe can be a Gram negative bacterium, such as Pseudomonas aeruginosa, Escherichia coli, Salmonella enteritidis ssp. Typhimurium, Acinetobacter baumanii, Burkholderia spp., Klebsiella pneumoniae, Enterobacter sp., or Campylobacter spp. In another embodiment, the microbe can be a Gram positive bacterium, such as Staphylococcus aureus, Staphylococcus epidermidis, or Enterococcus faecalis. The detection can include detecting residual bacteria by confocal microscopy of coverslips with adhered bacteria in flow cells, after specific staining, or by measuring residual bacteria adherent to the plastic surface of a microtiter plate by removing free swimming (planktonic) bacteria and staining residual bacteria with crystal violet.

In some embodiments, the present disclosure provides a method of selectively enhancing innate immunity by contacting a cell containing one or more genes that encodes a polypeptide involved in innate immunity and protection against an infection, with a peptide in accordance with the present disclosure, where expression of the one or more genes or polypeptides in the presence of the peptide is modulated as compared with expression of the one or more genes or polypeptides in the absence of the peptide, and where the modulated expression results in enhancement of innate immunity. In one aspect, the disclosure includes peptides identified by the methods. In another aspect, the peptide does not stimulate a septic reaction, but does stimulate the expression of one or more genes or polypeptides involved in protective immunity. Exemplary, but non-limiting, genes or polypeptides which are increased in expression include MCP1, MCP3 and Gro-α.

In some embodiments, the present disclosure provides a peptide that selectively suppress the proinflammatory response of a cell containing a polynucleotide or polynucleotides that encode a polypeptide involved in innate immunity. The method may include contacting the cell with a microbe, or a TLR ligand or agonists derived from those microbes, and further contacting the cells with a peptide, where the peptide decreases the expression of a proinflammatory gene encoding the polynucleotide or polypeptide as compared with expression of the proinflammatory gene or polypeptide in the absence of the peptide. In one aspect, the modulated expression results in suppression of proinflammatory and septic responses. In some embodiments, the peptide does not stimulate a sepsis reaction in a subject. Exemplary, but non-limiting, proinflammatory genes include TNFα.

In some embodiments, the peptide may selectively suppress proinflammatory responses, whereby the peptide can contact a cell treated with an inflammatory stimulus and containing a polynucleotide or polynucleotides that encode a polypeptide involved in inflammation and sepsis and which is normally upregulated in response to this inflammatory stimulus, and wherein the peptides may suppress the expression of this gene or polypeptide as compared with expression of the inflammatory gene in the absence of the peptide and wherein the modulated expression may result in enhancement of innate immunity.

In some embodiments, the peptide may inhibit the inflammatory or septic response. In some embodiments, the peptide may inhibit the expression of a pro-inflammatory gene or molecule. In some embodiments, the peptide may inhibit the expression of TNF-α. In some embodiments, the inflammation may be induced by a microbe or amicrobial ligand acting on a Toll-like receptor. In some embodiments, the microbial ligand may be a bacterial endotoxin or lipopolysaccharide.

In some embodiments, the peptide may may have anti-biofilm activity by virtue of inhibiting (p)ppGpp synthesis or causing (p)ppGpp degradation.

In some embodiments, the present disclosure provides a method of protecting a medical device from colonization with pathogenic biofilm-forming bacteria by coating at least one peptide onto the medical device.

### Peptides

The present disclosure provides an isolated peptide with anti-biofilm and/or immunomodulatory activity. Exemplary peptides may have an amino acid sequence set forth in any one of SEQ ID NO: 6-1085, or a functional variant thereof. In some embodiments, the isolated antibiofilm and/or immunomodulatory peptide may include an amino acid sequence as set forth in one or more of SEQ ID NOs: 25, 29, 30, 38, 39, 44, 45, 46, 57, 60, 65, or a functional variant thereof. In alternative embodiments, the isolated antibiofilm and/or immunomodulatory peptide may include an amino acid sequence as set forth in one or more of SEQ ID NOs: 151 or 152 (peptides D-3006 or D-3007, respectively), or a functional variant thereof. In alternative embodiments, the isolated antibiofilm and/or immunomodulatory peptide may include an amino acid sequence as set forth in one or more of SEQ ID NOs: 24-47, 146-169, 196-219, 246-437, or a functional variant thereof.

“Isolated” when used in reference to a peptide, refers to a peptide substantially free of proteins, lipids, nucleic acids, for example, with which it might be naturally associated. Those of skill in the art can make similar substitutions to achieve peptides with similar or greater anti-biofilm or immunomodulatory activity, given the sequence of a parent peptide. For example, the present disclosure includes a peptide with the amino acid sequence set forth in forth in any one of SEQ ID NO: 6-1085, or a functional variant thereof, as long as the bioactivity (e.g., anti-biofilm or immunomodulatory) of the peptide remains. Minor modifications of the primary amino acid sequence of the peptides of the disclosure may result in peptides that have substantially equivalent activity as compared to the specific peptides described herein. Such modifications may be deliberate, as by site-specific substitutions or may be spontaneous. Peptides produced by these modifications are included herein as long as the biological activity of the original peptide still exists.

A “functional variant” includes peptides containing D-amino acids, non-natural amino acids, amidated amino acids, unamidated amino acids, enantiomers, retro-inverso derivatives, analogs, conservative substitutions, etc.

Peptides can be synthesized in solid phase, or as an array of peptides made in parallel on cellulose sheets (Frank, R. 1992) or by solution phase chemistry. These methods have been used to create a large number of variants through sequence scrambling, truncations and systematic modifications of peptide sequence, and a luciferase-based screen to investigate their ability to kill Pseudomonas aeruginosa planktonic cells (Hilpert K, et al. 2005). In some embodiments, a peptide in accordance with the present disclosure may be 7 to 14 amino acids in length, or any value or range in between, such as 7, 8, 9, 10, 11, 12, 13 or 14 amino acids, or 7 to 12 amino acids, or 8 to 14 amino acids, etc.

The “amino acid” residues of the peptides identified herein may be in the natural L-configuration or isomeric D-configuration (“D-amino acids”). In keeping with standard polypeptide nomenclature (J. Biol. Chem., 243:3557-59, (1969), abbreviations and chemical names for side chains (affixed to the alpha carbon of the backbone) for natural amino acid residues are as shown in the following table.

| 1-Letter | 3-Letter | Amino Acid | Side chain chemical name |
|---|---|---|---|
| Y | Tyr | L-tyrosine | 1-methyl-4-hydroxybenzyl |
| G | Gly | L-glycine | hydrogen |
| F | Phe | L-phenylalanine | methylbenzyl |
| M | Met | L-methionine | ethylthiomethyl |
| A | Ala | L-alanine | methyl |
| S | Ser | L-serine | hydroxymethyl |
| I | Ile | L-isoleucine | 1-methylpropyl |
| L | Leu | L-leucine | 2-methylpropyl |
| T | Thr | L-threonine | 1-hydroxyethyl |
| V | Val | L-valine | isopropyl |
| P | Pro | L-proline | pyrrolidine |
| K | Lys | L-lysine | α-aminobutyl |
| H | His | L-histidine | methyl-1H-imidazol-4-yl |
| Q | Gln | L-glutamine | propyl-3-carboxamide |
| E | Glu | L-glutamic acid | propyl-3-carboxylate |
| W | Trp | L-tryptohan | methyl-1H-indol-3-yl |
| R | Arg | L-arginine | propyl-3-guanidine |
| D | Asp | L-aspartic acid | ethyl-2-carboxylate |
| N | Asn | L-asparagine | ethyl-2-carboxamide |
| C | Cys | L-cysteine | methylsulphydryl |

It should be noted that all amino acid residue sequences are represented herein by formulae whose left to right orientation is in the conventional direction of amino-terminus to carboxy-terminus. Peptides can be modified at the carboxy-terminus to remove the negative charge, often through amidation, esterification, acylation or the like.

In some embodiments, suitable amino acids for anti-biofilm and/or immunomodulatory activity include A, R, L, I, V, K, W, G, and Q.

Further, deletion of one or more amino acids can also result in a modification of the structure of the resultant molecule without significantly altering its biological activity. This can lead to the development of a smaller active molecule that would also have utility. For example, amino or carboxy terminal amino acids that may not be required for biological activity of the particular peptide can be removed. Peptides in accordance with the present disclosure may include any analog, homolog, mutant, isomer or derivative of the peptides disclosed herein, so long as bioactivity as described herein remains. In general, the peptides are synthesized using L or D form amino acids, however, mixed peptides containing both L- and D-form amino acids can be synthetically produced. In addition, C-terminal derivatives can be produced, such as C-terminal amidates, C-terminal acylates, and C-terminal methyl and acetyl esters, in order to increase the anti-biofilm or immunomodulatory activity of a peptide of the disclosure. The peptide can be synthesized such that the sequence is reversed whereby the last amino acid in the sequence becomes the first amino acid, and the penultimate amino acid becomes the second amino acid, and so on (a “retro-inverso” or “RI” derivative).

In certain embodiments, the peptides of the disclosure may include peptide analogs and peptide mimetics. Indeed, the peptides of the disclosure include peptides having any of a variety of different modifications, including those described herein.

Peptide analogs of the disclosure may be generally designed and produced by chemical modifications of a lead peptide, including, e.g., any of the particular peptides described herein, such as any of the following sequences disclosed in the tables. The present disclosure clearly establishes that these peptides in their entirety and derivatives created by modifying any side chains of the constituent amino acids have the ability to inhibit, prevent, or destroy the growth or proliferation of microbes such as bacteria, fungi, viruses, parasites or the like. The present disclosure further encompasses polypeptides up to about 50 amino acids in length that include the amino acid sequences and functional variants or peptide mimetics of the sequences described herein.

In another embodiment, a peptide of the present disclosure may be a pseudopeptide. Pseudopeptides or amide bond surrogates refers to peptides containing chemical modifications of some (or all) of the peptide bonds. The introduction of amide bond surrogates not only decreases peptide degradation but also may significantly modify some of the biochemical properties of the peptides, particularly the conformational flexibility and hydrophobicity.

To improve or alter the characteristics of the peptides of the present disclosure, protein engineering can be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased biological activity or increased/decreased stability. In addition, they can be purified in higher yields and show better solubility than the corresponding natural polypeptide, at least under certain purification and storage conditions. Further, the peptides of the present disclosure can be produced as multimers including dimers, trimers and tetramers. Multimerization can be facilitated by linkers, introduction of cysteines to permit creation of interchain disulphide bonds, or recombinantly though heterologous polypeptides such as Fc regions.

One or more amino acids can be deleted from the N-terminus or C-terminus without substantial loss of biological function (see, e.g., Ron, et al. 1993). Accordingly, polypeptides having one or more residues deleted from the amino terminus fall within the scope of the present disclosure. Similarly, many examples of biologically functional C-terminal deletion mutants are known (see, e.g., Dobeli, et al., 1988). Accordingly, the present disclosure provides polypeptides having one or more residues deleted from the carboxy terminus. The disclosure also provides polypeptides having one or more amino acids deleted from both the amino and the carboxyl termini as described herein.

Other mutants in addition to N- and C-terminal deletion forms of the protein discussed above are included in the present disclosure. Thus, the disclosure further includes variations of the polypeptides that show substantial anti-biofilm and/or immunomodulatory activity. Such mutants include deletions, insertions, inversions, repeats, and substitutions selected according to general rules known in the art so as to have little effect on activity.

There are two main approaches for studying the tolerance of an amino acid sequence to change, see, Bowie, et al., 1994. The first method relies on the process of evolution, in which mutations are either accepted or rejected by natural selection. The second approach uses genetic engineering to introduce amino acid changes at specific positions of a cloned gene and selections or screens to identify sequences that maintain functionality. The effects of such changes can easily be assessed by employing artificial neural networks and quantitative structure activity analyses (Cherkasov, A., et al. 2009).

Typically seen as “conservative substitutions” are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg, and replacements among the aromatic residues Phe, Tyr and Trp. Thus, the peptide of the present disclosure can be, for example: (i) one in which one or more of the amino acid residues are substituted with a conserved or non-conserved amino acid residue (preferably a conserved amino acid residue) and such substituted amino acid residue can or cannot be one encoded by the genetic code; or (ii) one in which one or more of the amino acid residues includes a substituent group; or (iii) one in which the polypeptide is fused with another compound, such as a compound to increase the half-life of the polypeptide (for example, polyethylene glycol); or (iv) one in which the additional amino acids are fused to the above form of the polypeptide, such as an IgG Fc fusion region peptide or leader or secretory sequence or a sequence which is employed for purification of the above form of the polypeptide or a pro-protein sequence.

Thus, the peptides of the present disclosure can include one or more amino acid substitutions, deletions, or additions, either from natural mutations or human manipulation. As indicated, changes are preferably of a minor nature, such as conservative amino acid substitutions that do not significantly affect the folding or activity of the peptide. The following groups of amino acids represent equivalent changes: (1) Gln, Asn; (2) Ser, Thr; (3) Val, Ile, Leu, Met, Ala, Phe; (4) Lys, Arg, His; (5) Phe, Tyr, Trp.

Arginine and/or lysine can be substituted with other basic non-natural amino acids including ornithine, citrulline, homoarginine, Nδ-[1-(4,4-dimethyl-2, 6-dioxocyclohexylidene)-ethyl-L-ornithine, Nε-methyltrityl-L-lysine, and diamino-butyrate although many other mimetic residues are available. Favourable substitutions utilized here include: L-2-amino-3-guanidinopropionic acid (GPro); L-2-Amino-4-guanidinobutyric acid (But), L-Homoarginine (Har),L-2,3-diaminopropionic acid (Dap), L-2,4-diaminobutyric acid (Dab), and L-Ornithine (Orn). Tryptophan residues can be substituted for homo-tryptophan, bromotryptophan and fluorotryptophan. The term “conservative variation” or “conservative substitution” also includes the use of a substituted amino acid in place of an unsubstituted parent amino acid provided that the substituted polypeptide at least retains most of the activity of the unsubstituted parent peptide. Such conservative substitutions are within the definition of the classes of the peptides of the disclosure.

The present disclosure further includes peptide fragments. More specifically, the present disclosure embodies purified, isolated, and recombinant peptides comprising at least any one integer between 6 and 504 (or the length of the peptides amino acid residues minus 1 if the length is less than 1000) of consecutive amino acid residues. The fragments may be at least 6, preferably at least 7 to 11, more preferably 12 to 14 consecutive amino acids.

In some embodiments, the peptide can include a contiguous sequence of amino acids having the formula: AA1-AA2-AA3-AA4-AA5-AA6-AA7-AA8-AA9-AA10-AA11-AA12 and containing only the residues K, R, V, L, I, A, W and no more than two Q or G residues either on their own or in combination.

In some embodiments, the disclosure provides a polypeptide X1-A-X2 or a functional variant or mimetic thereof, where A represents at least one peptide having an amino acid sequence as set forth in SEQ ID NO: 6-1085, or a functional variant thereof; and where each X1 and X2 independently of one another represents any amino acid sequence of n amino acids, n varying from 0 to 50, and n being identical or different in X1 and X2.

In some embodiments, the peptide may include an amino acid sequence as set forth in one or more of SEQ ID NOs: 25, 29, 30, 38, 39, 44, 45, 46, 57, 60, 65, or a functional variant thereof. In alternative embodiments, the peptide may include an amino acid sequence as set forth in one or more of SEQ ID NOs: 151 or 152 (peptides D-3006 or D-3007, respectively), or a functional variant thereof. In alternative embodiments, the peptide may include an amino acid sequence as set forth in one or more of SEQ ID NOs: 24-47, 146-169, 196-219, 246-437, or a functional variant thereof.

In some embodiments, the functional variant may be a conservative amino acid substitution or peptide mimetic substitution. In some embodiments, the functional variant may have about 66% or greater amino acid identity. In some embodiments of this aspect, the functional variant may have about 70% or greater amino acid sequence identity. Truncation of amino acids from the N or C termini or from both can create these mimetics. In some embodiments of this polypeptide, the amino acids may be non-natural amino acid equivalents. In some embodiments of this polypeptide, n may be zero. In some embodiments of this aspect, the functional variant or mimetic may be a conservative amino acid substitution or peptide mimetic substitution.

In some embodiments, the peptide according to the disclosure can be represented by a consensus sequence, as described herein, for example, Z₁U₂B₃Z₄J₅Z₆W₇J₈Z₉O₁₀ wherein Z=hydrophobic residues (W, V, L, I, A or G); B=basic residues (R or K); J=Basic or hydrophobic residues (Z+B); U=Uncharged residues (Z+Q); and O=pOlar residues (B+Q); HHHBHHBHBHJH, where “H” is a hydrophobic amino acid (W, L, I, V, A, or G); “B” is a basis amino acid (R or K); and “J” is a polar amino acid (Q, R, or K); HHBHBHBHHHHB, where “H” is a hydrophobic amino acid (W, L, I, V, A, or G); “B” is a basis amino acid (R or K); and “J” is a polar amino acid (Q, R, or K); BHHHBEHHHJHHB, where “H” is a hydrophobic amino acid (W, L, I, V, A, or G); “B” is a basis amino acid (R or K); and “J” is a polar amino acid (Q, R, or K); HHBHHHHHHHBB, where “H” is a hydrophobic amino acid (W, L, I, V, A, or G); “B” is a basis amino acid (R or K); and “J” is a polar amino acid (Q, R, or K); BBHHBHHHHBHB, where “H” is a hydrophobic amino acid (W, L, I, V, A, or G); “B” is a basis amino acid (R or K); and “J” is a polar amino acid (Q, R, or K); HHHJHHHHHBHB, where “H” is a hydrophobic amino acid (W, L, I, V, A, or G); “B” is a basis amino acid (R or K); and “J” is a polar amino acid (Q, R, or K); or HJBHHHHBHBHH, where “H” is a hydrophobic amino acid (W, L, I, V, A, or G); “B” is a basis amino acid (R or K); and “J” is a polar amino acid (Q, R, or K).

In some embodiments, the peptide according to the disclosure can be represented by a chemical structure as set forth in Formula 1:

[chemical structure]

wherein:
Z₁, Z₄, Z₆ and Z₉ are each independently H, methyl-1H-indol-3-yl, isopropyl, methyl, 2-methylpropyl, or 1-methylpropyl;
B₃ is propyl-3-guanidine or α-aminobutyl;
J₅, and J₈ are each independently H, methyl-1H-indol-3-yl, isopropyl, methyl, 2-methylpropyl, 1-methylpropyl; propyl-3-guanidine, α-aminobutyl, propyl-3-guanidine, α-aminobutyl, or propyl-3-carboxamide;
U₂ is H, methyl-1H-indol-3-yl, isopropyl, methyl, 2-methylpropyl, 1-methylpropyl, or propyl-3-carboxamide;
Σ₁₀ is propyl-3-guanidine, α-aminobutyl, or propyl-3-carboxamide;
X₁ and X₂ are each independently 0 to 2 amino acids selected from the group consisting of 2-amino-3-(1h-indol-3-yl)propanoic acid, 2-amino-3-methylbutanoic acid, 2-aminopropanoic acid, 2-amino-4-methylpentanoic acid, 2-amino-3-methylpentanoic acid, aminoacetic acid, 2-amino-5-guanidinopentanoic acid, or 2,6-diaminohexanoic acid; and where the peptide can also contain one substitution from the group Z₁=α-aminobutyl, B₃=2-methylpropyl, Z₆=propyl-3-guanidine, W₇ is H, methyl-1H-indol-3-yl, isopropyl, methyl, 2-methylpropyl, 1-methylpropyl, or propyl-3-carboxamide and Σ₁₀ is methyl.

In addition, it should be understood that in certain embodiments, the peptides of the present disclosure may include two or more modifications, including, but not limited to those described herein. By taking into the account the features of the peptide drugs on the market or under current development, it is clear that most of the peptides successfully stabilized against proteolysis consist of a mixture of several types of the above-described modifications. This conclusion is understood in the light of the knowledge that many different enzymes are implicated in peptide degradation.

In some embodiments, peptides of the disclosure can retain activities in the typical media used to test in vitro antibiofilm activity and/or tissue culture medium used to examine immunomodulatory activity, making them candidates for clinical therapeutic usage; in contrast most directly antimicrobial peptides are antagonized by physiological levels of salts.

### Peptides, Peptide Variants, and Peptide Mimetics

“Polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Amino acid mimetic refers to a chemical compound that has a structure that is different from the general chemical structure of a natural amino acid, but which functions in a manner similar to a naturally occurring amino acid. Non-natural residues are well described in the scientific and patent literature; a few exemplary non-natural compositions useful as mimetics of natural amino acid residues and guidelines are described below. Mimetics of aromatic amino acids can be generated by replacing with, e.g., D- or L-naphylalanine; D- or L-phenylglycine; D- or L-2 thieneylalanine; D- or L-1,-2,3-, or 4-pyreneylalanine; D- or L-3 thieneylalanine; D- or L-(2-pyridinyl)-alanine; D- or L-(3-pyridinyl)-alanine; D- or L-(2-pyrazinyl)-alanine; D- or L-(4-isopropyl)-phenylglycine; D-(trifluoromethyl)-phenylglycine; D-p-fluoro-phenylalanine; D-(trifluoromethyl)-phenylalanine; D- or L-p-biphenylphenylalanine; K- or L-p-methoxy-biphenylphenylalanine; D- or L-2-indole(alkyl)alanines; and, D- or L-alkylainines, where alkyl can be substituted or unsubstituted methyl, ethyl, propyl, hexyl, butyl, pentyl, isopropyl, iso-butyl, sec-isotyl, iso-pentyl, or a non-acidic amino acids. Aromatic rings of a non-natural amino acid include, e.g., thiazolyl, thiophenyl, pyrazolyl, benzimidazolyl, naphthyl, furanyl, pyrrolyl, and pyridyl aromatic rings.

“Peptide” as used herein includes peptides that are conservative variations of those peptides specifically exemplified herein. “Conservative variation” as used herein denotes the replacement of an amino acid residue by another, biologically similar residue, as discussed elsewhere herein. “Cationic” as is used to refer to any peptide that possesses sufficient positively charged amino acids to have a pI (isoelectric point) greater than about 9.0.

The biological activity of the anti-biofilm peptides can be determined by standard methods known to those of skill in the art, such as “minimal biofilm inhibitory concentration (MBIC)” or “minimal biofilm eradication concentration (MBEC)” assays described in the present examples, whereby the lowest concentration causing reduction or eradication of biofilms is observed for a given period of time and recorded as the MBIC or MBEC respectively.

The peptides and polypeptides of the disclosure, as defined above, include all “mimetic” and “peptidomimetic” forms. The terms “mimetic” and “peptidomimetic” refer to a synthetic chemical compound that has substantially the same structural and/or functional characteristics of the polypeptides of the peptides described herein. The mimetic can be either entirely composed of synthetic, non-natural analogues of amino acids, or, is a chimeric molecule of partly natural peptide amino acids and partly non-natural analogs of amino acids. The mimetic can also incorporate any number of natural amino-acid conservative substitutions as long as such substitutions do not substantially alter the mimetic's structure and/or activity. As with polypeptides of the disclosure that are conservative variants, routine experimentation will determine whether a mimetic is within the scope of the disclosure, i.e., that its structure and/or function is not substantially altered. Thus, a mimetic composition is within the scope of the disclosure if it has anti-biofilm or immunomodulatory activity.

Polypeptide mimetic compositions can also contain any combination of non-natural structural components, which are typically from three structural groups: a) residue linkage groups other than the natural amide bond (“peptide bond”) linkages; b) non-natural residues in place of naturally occurring amino acid residues; or c) residues that induce secondary structural mimicry, i.e., to induce or stabilize a secondary structure, e.g., a beta turn, gamma turn, beta sheet, alpha helix conformation, and the like. For example, a polypeptide can be characterized as a mimetic when all or some of its residues are joined by chemical means other than natural peptide bonds. Individual peptidomimetic residues can be joined by peptide bonds, other chemical bonds or coupling means, such as, e.g., glutaraldehyde, N-hydroxysuccinimide esters, bifunctional maleimides, N,N′-dicyclohexylcarbodiimide (DCC) or N,N′-diisopropylcarbodiimide (DIC). Linking groups that can be an alternative to the traditional amide bond (“peptide bond”) linkages include, e.g., ketomethylene (e.g., —C(═O)—CH₂— for —C(═O)—NH—), aminomethylene (CH₂—NH), ethylene, olefin (CH═CH), ether (CH₂—O), thioether (CH₂—S), tetrazole (CN₄—), thiazole, retroamide, thioamide, or ester (see, e.g., 40).

Mimetics of acidic amino acids can be generated by substitution by, e.g., non-carboxylate amino acids while maintaining a negative charge such as e.g. (phosphono)alanine; sulfated threonine. Carboxyl side groups (e.g., aspartyl or glutamyl) can also be selectively modified by reaction with carbodiimides (R′—N—C—N—R′) such as, e.g., 1-cyclohexyl-3(2-morpholin-yl-(4-ethyl) carbodiimide or 1-ethyl-3(4-azonia-4,4-dimetholpentyl) carbodiimide. Aspartyl or glutamyl can also be converted to asparaginyl and glutaminyl residues by reaction with ammonium ions.

Mimetics of basic amino acids can be generated by substitution with, e.g., (in addition to lysine and arginine) the amino acids ornithine (Orn), or citrulline or the side chain diaminobenzoate or diamino-3-guanidinopropionate (GPro) or diamino-4-guanidinobutyate (But), or L-Homoarginine (Har), or L-2,3-diaminopropionate (Dap), or L-2,4-diaminobutyrate (Dab). Asparaginyl and glutaminyl residues can be deaminated to the corresponding aspartyl or glutamyl residues.

Arginine residue mimetics can be generated by reacting arginyl with, e.g., one or more conventional reagents, including, e.g., phenylglyoxal, 2,3-butanedione, 1,2-cyclohexanedione, or ninhydrin, preferably under alkaline conditions. Tyrosine residue mimetics can be generated by reacting tyrosyl with, e.g., aromatic diazonium compounds or tetranitromethane. N-acetylimidizol and tetranitromethane can be used to form O-acetyl tyrosyl species and 3-nitro derivatives, respectively. Cysteine residue mimetics can be generated by reacting cysteinyl residues with, e.g., alpha-haloacetates such as 2-chloroacetic acid or chloroacetamide and corresponding amines; to give carboxymethyl or carboxyamidomethyl derivatives. Cysteine residue mimetics can also be generated by reacting cysteinyl residues with, e.g., bromo-trifluoroacetone, alpha-bromo-beta-(5-imidozoyl) propionic acid; chloroacetyl phosphate, N-alkylmaleimides, 3-nitro-2-pyridyl disulfide; methyl 2-pyridyl disulfide; p-chloromercuribenzoate; 2-chloromercuri-4 nitrophenol; or, chloro-7-nitrobenzo-oxa-1,3-diazole. Lysine mimetics can be generated (and amino terminal residues can be altered) by reacting lysinyl with, e.g., succinic or other carboxylic acid anhydrides. Lysine and other alpha-amino-containing residue mimetics can also be generated by reaction with imidoesters, such as methyl picolinimidate, pyridoxal phosphate, pyridoxal, chloroborohydride, trinitrobenzenesulfonic acid, O-methylisourea, 2,4, pentanedione, and transamidase-catalyzed reactions with glyoxylate. Mimetics of methionine can be generated by reaction with, e.g., methionine sulfoxide. Histidine residue mimetics can be generated by reacting histidyl with, e.g., diethylprocarbonate or para-bromophenacyl bromide. Other mimetics include, e.g., those generated by hydroxylation of lysine; phosphorylation of the hydroxyl groups of seryl or threonyl residues; methylation of the alpha-amino groups of lysine, arginine and histidine; acetylation of the N-terminal amine; methylation of main chain amide residues or substitution with N-methyl amino acids; or amidation of C-terminal carboxyl groups.

A component of a peptide of the disclosure can also be replaced by an amino acid (or peptidomimetic residue) of the opposite chirality. Thus, any amino acid naturally occurring in the L-configuration (which can also be referred to as the R or S, depending upon the structure of the chemical entity) can be replaced with the amino acid of the same chemical structural type or a peptidomimetic, but of the opposite chirality, referred to as the D-amino acid, but which can additionally be referred to as the R- or S-form, and vice versa.

The disclosure also provides peptides that are “substantially identical” to an exemplary peptide as described herein. A “substantially identical” amino acid sequence is a sequence that differs from a reference sequence by one or more conservative or non-conservative amino acid substitutions, deletions, or insertions, particularly when such a substitution occurs at a site that is not the active site of the molecule, and provided that the polypeptide essentially retains its functional properties. A conservative amino acid substitution, for example, substitutes one amino acid for another of the same class (e.g., substitution of one hydrophobic amino acid, such as isoleucine, valine, leucine, or methionine, for another, or substitution of one polar amino acid for another, such as substitution of arginine for lysine, glutamic acid for aspartic acid or glutamine for asparagine). One or more amino acids can be deleted, for example, from an anti-biofilm or immunomodulatory polypeptide having anti-biofilm or immunomodulatory activity, resulting in modification of the structure of the polypeptide, without significantly altering its biological activity. For example, amino- or carboxyl-terminal, or internal, amino acids that are not required for antimicrobial activity can be removed.

The skilled artisan will recognize that individual synthetic residues and peptides incorporating these mimetics can be synthesized using a variety of procedures and methodologies, which are well described in the scientific and patent literature, e.g., Organic Syntheses Collective Volumes, Gilman, et al. (Eds) John Wiley & Sons, Inc., NY. Peptides and peptide mimetics of the disclosure can also be synthesized using combinatorial methodologies. Various techniques for generation of peptide and peptidomimetic libraries are well known, and include, e.g., multipin, tea bag, and split-couple-mix techniques; see, e.g., al-Obeidi, Mol. Biotechnol. 1998; Hruby, 1997; Ostergaard, Mol. Divers. 3: 17-27, 1997; Ostresh, Methods Enzymol. 267: 220-234, 1996. Modified peptides can be further produced by chemical modification methods, see, e.g., Belousov, Nucleic Acids Res. 25: 3440-3444, 1997; Frenkel, Free Radic. Biol. Med. 19: 373-380, 1995; Blommers, Biochemistry 33: 7886-7896, 1994.

Peptides and polypeptides can be isolated from natural sources, be synthetic, or be recombinantly generated polypeptides. Peptides and proteins can be recombinantly expressed in vitro or in vivo. The peptides and polypeptides can be made and isolated using any method known in the art. Polypeptide and peptides can also be synthesized, whole or in part, using chemical methods well known in the art. See e.g., Caruthers, Nucleic Acids Res. Symp. Ser. 215-223, 1980; Horn, Nucleic Acids Res. Symp. Ser. 225-232, 1980; Banga, Therapeutic Peptides and Proteins, Formulation, Processing and Delivery Systems Technomic Publishing Co., Lancaster, Pa., 1995. For example, peptide synthesis can be performed using various solid-phase techniques (see e.g., Roberge, Science 269: 202, 1995; Merrifield, Methods Enzymol. 289: 3-13, 1997) and automated synthesis can be achieved, e.g., using the ABI 431A Peptide Synthesizer (Perkin Elmer) in accordance with the instructions provided by the manufacturer.

Peptides can be synthesized by such commonly used methods as t-BOC or FMOC protection of alpha-amino groups. Both methods involve stepwise syntheses whereby a single amino acid is added at each step starting from the C terminus of the peptide (See, Coligan, et al., Current Protocols in Immunology, Wiley Interscience, 1991, Unit 9). Peptides can also be synthesized by the well known solid phase peptide synthesis methods described in Merrifield, J. Am. Chem. Soc., 85:2149, (1962), and Stewart and Young, Solid Phase Peptides Synthesis, (Freeman, San Francisco, 1969, pp. 27-62, using a copoly(styrene-divinylbenzene) containing 0.1-1.0 mMol amines/g polymer. On completion of chemical synthesis, the peptides can be deprotected and cleaved from the polymer by treatment with liquid HF-10% anisole for about ¼-1 hours at 0° C. After evaporation of the reagents, the peptides are extracted from the polymer with 1% acetic acid solution which is then lyophilized to yield the crude material. This can normally be purified by such techniques as gel filtration on Sephadex G-15 using 5% acetic acid as a solvent. Lyophilization of appropriate fractions of the column will yield the homogeneous peptide or peptide derivatives, which can then be characterized by such standard techniques as amino acid analysis, thin layer chromatography, high performance liquid chromatography, ultraviolet absorption spectroscopy, molar rotation, solubility, and quantitated by the solid phase Edman degradation.

Analogs, polypeptide fragment of anti-biofilm or immunomodulatory protein having anti-biofilm or immunomodulatory activity, are generally designed and produced by chemical modifications of a lead peptide, including, e.g., any of the particular peptides described herein, such as any of the sequences set forth in SEQ ID NO: 6-1085.

As contemplated herein, “polypeptide” includes those having one or more chemical modification relative to another polypeptide, i.e., chemically modified polypeptides. The polypeptide from which a chemically modified polypeptide is derived may be a wildtype protein, a functional variant protein or a functional variant polypeptide, or polypeptide fragments thereof; an antibody or other polypeptide ligand according to the disclosure including without limitation single-chain antibodies, crystalline proteins and polypeptide derivatives thereof; or polypeptide ligands prepared according to the disclosure. Preferably, the chemical modification(s) confer(s) or improve(s) desirable attributes of the polypeptide but does not substantially alter or compromise the biological activity thereof. Desirable attributes include but are limited to increased shelf-life; enhanced serum or other in vivo stability; resistance to proteases; and the like. Such modifications include by way of non-limiting example N-terminal acetylation, glycosylation, and biotinylation.

An effective approach to confer resistance to peptidases acting on the N-terminal or C-terminal residues of a polypeptide is to add chemical groups at the polypeptide termini, such that the modified polypeptide is no longer a substrate for the peptidase. One such chemical modification is glycosylation of the polypeptides at either or both termini. Certain chemical modifications, in particular N-terminal glycosylation, have been shown to increase the stability of polypeptides in human serum (Powell et al., Pharma. Res. 10: 1268-1273, 1993). Other chemical modifications which enhance serum stability include, but are not limited to, the addition of an N-terminal alkyl group, consisting of a lower alkyl of from 1 to 20 carbons, such as an acetyl group, and/or the addition of a C-terminal amide or substituted amide group.

The presence of an N-terminal D-amino acid increases the serum stability of a polypeptide that otherwise contains L-amino acids, because exopeptidases acting on the N-terminal residue cannot utilize a D-amino acid as a substrate. Similarly, the presence of a C-terminal D-amino acid also stabilizes a polypeptide, because serum exopeptidases acting on the C-terminal residue cannot utilize a D-amino acid as a substrate. With the exception of these terminal modifications, the amino acid sequences of polypeptides with N-terminal and/or C-terminal D-amino acids are usually identical to the sequences of the parent L-amino acid polypeptide.

The terms “identical” or percent “identity”, in the context of two or peptide sequences, refers to two or more sequences or subsequences that are the same or have a specified percentage of amino acid residues or nucleotides that are the same (i.e., about 65% identity, preferably 75%, 85%, 90%, or higher identity over a specified region (e.g., nucleotide sequence encoding a peptide described herein or amino acid sequence), when compared and aligned for maximum correspondence over a comparison window or designated region) as measured using Muscle (http://www.bioinformatics.nl/tools/muscle.html) multiple alignment sequence comparison algorithm or by manual alignment and visual inspection. Such sequences are then said to be “substantially identical.” In some preferred embodiments, the identity is 87%. The term also includes sequences that have deletions and/or additions, as well as those that have substitutions as long as at least two thirds of the amino acids can be aligned. As described below, the preferred algorithms can account for gaps and the like. Preferably, for small peptides, identity exists over a region that is at least about 6 amino acids in length.

For peptide sequence comparison, typically one sequence acts as a reference sequence, to which test sequences are compared. When using a sequence comparison algorithm, test and reference sequences are entered into a computer in FASTA format and alignment is performed. Preferably, default program parameters can be used, or alternative parameters can be designated. The sequence comparison algorithm then aligns the sequences enabling a calculation of the percent sequence identities for the test sequences relative to the reference sequence, based on the program parameters.

### Polypeptide Mimetic

In general, a polypeptide mimetic (“peptidomimetic”) is a molecule that mimics the biological activity of a polypeptide but is no longer peptidic in chemical nature. By strict definition, a peptidomimetic is a molecule that contains no peptide bonds (that is, amide bonds between amino acids). However, the term peptidomimetic is sometimes used to describe molecules that are no longer completely peptidic in nature, such as pseudo-peptides, semi-peptides and peptoids. Examples of some peptidomimetics by the broader definition (where part of a polypeptide is replaced by a structure lacking peptide bonds) are described below. Whether completely or partially non-peptide, peptidomimetics provide a spatial arrangement of reactive chemical moieties that closely resembles the three-dimensional arrangement of active groups in the polypeptide on which the peptidomimetic is based. As a result of this similar active-site geometry, the peptidomimetic has effects on biological systems that are similar to the biological activity of the polypeptide.

There are several potential advantages for using a mimetic of a given polypeptide rather than the polypeptide itself. For example, polypeptides may exhibit two undesirable attributes, i.e., poor bioavailability and short duration of action. Peptidomimetics are often small enough to be both orally active and to have a long duration of action. There are also problems associated with stability, storage and immunoreactivity for polypeptides that are not experienced with peptidomimetics.

Candidate, lead and other polypeptides having a desired biological activity can be used in the development of peptidomimetics with similar biological activities. Techniques of developing peptidomimetics from polypeptides are known. Peptide bonds can be replaced by non-peptide bonds that allow the peptidomimetic to adopt a similar structure, and therefore biological activity, to the original polypeptide. Further modifications can also be made by replacing chemical groups of the amino acids with other chemical groups of similar structure. The development of peptidomimetics can be aided by determining the tertiary structure of the original polypeptide, either free or bound to a ligand, by NMR spectroscopy, crystallography and/or computer-aided molecular modeling. These techniques aid in the development of novel compositions of higher potency and/or greater bioavailability and/or greater stability than the original polypeptide (Dean, BioEssays, 16: 683-687, 1994; Cohen and Shatzmiller, J. Mol. Graph., 11: 166-173, 1993; Wiley and Rich, Med. Res. Rev., 13: 327-384, 1993; Moore, Trends Pharmacol. Sci., 15: 124-129, 1994; Hruby, Biopolymers, 33: 1073-1082, 1993; Bugg et al., Sci. Am., 269: 92-98, 1993).

Thus, through use of the methods described above, the present disclosure provides compounds exhibiting enhanced therapeutic activity in comparison to the polypeptides described above. The peptidomimetic compounds obtained by the above methods, having the biological activity of the above-named polypeptides and similar three-dimensional structure, are encompassed by this disclosure. It will be readily apparent to one skilled in the art that a peptidomimetic can be generated from any of the modified polypeptides described in the previous section or from a polypeptide bearing more than one of the modifications described from the previous section. It will furthermore be apparent that the peptidomimetics can be further used for the development of even more potent non-peptidic compounds, in addition to their utility as therapeutic compounds.

Specific examples of peptidomimetics derived from the polypeptides described in the previous section are presented below. These examples are illustrative and not limiting in terms of the other or additional modifications.

Proteases act on peptide bonds. It therefore follows that substitution of peptide bonds by pseudopeptide bonds confers resistance to proteolysis. A number of pseudopeptide bonds have been described that in general do not affect polypeptide structure and biological activity. The reduced isostere pseudopeptide bond is a suitable pseudopeptide bond that is known to enhance stability to enzymatic cleavage with no or little loss of biological activity (Couder, et al., Int. J. Polypeptide Protein Res. 41: 181-184, 1993). Thus, the amino acid sequences of these compounds may be identical to the sequences of their parent L-amino acid polypeptides, except that one or more of the peptide bonds are replaced by an isosteric pseudopeptide bond. Preferably the most N-terminal peptide bond is substituted, since such a substitution would confer resistance to proteolysis by exopeptidases acting on the N-terminus.

To confer resistance to proteolysis, peptide bonds may also be substituted by retro-inverso pseudopeptide bonds (Dalpozzo, et al., Int. J. Polypeptide Protein Res. 41: 561-566). According to this modification, the amino acid sequences of the compounds may be identical to the sequences of their L-amino acid parent polypeptides, except that one or more of the peptide bonds are replaced by a retro-inverso pseudopeptide bond. Preferably the most N-terminal peptide bond is substituted, since such a substitution will confer resistance to proteolysis by exopeptidases acting on the N-terminus.

Peptoid derivatives of polypeptides represent another form of modified polypeptides that retain the important structural determinants for biological activity, yet eliminate the peptide bonds, thereby conferring resistance to proteolysis (Simon, et al., Proc. Natl. Acad. Sci. USA, 89: 9367-9371, 1992). Peptoids are oligomers of N-substituted glycines. A number of N-alkyl groups have been described, each corresponding to the side chain of a natural amino acid.

### Polynucleotides

The disclosure includes polynucleotides encoding the peptides described herein. Exemplary polynucleotides encode peptides including those set forth in SEQ ID NO: 6-1085, or a functional variant thereof, where the peptides have antibiofilm or immunomodulatory activity. The peptides of the disclosure include those set forth in SEQ ID NO: 6-1085, or a functional variant thereof, as well as the broader groups of peptides having hydrophilic and hydrophobic substitutions, and conservative variations thereof.

“Isolated” when used in reference to a polynucleotide, refers to a polynucleotide substantially free of proteins, lipids, nucleic acids, for example, with which it is naturally associated. As used herein, “polynucleotide” refers to a polymer of deoxyribonucleotides or ribonucleotides, in the form of a separate fragment or as a component of a larger construct. DNA encoding a peptide of the disclosure can be assembled from cDNA fragments or from oligonucleotides which provide a synthetic gene which is capable of being expressed in a recombinant transcriptional unit. Polynucleotide sequences of the disclosure include DNA, RNA and cDNA sequences. A polynucleotide sequence can be deduced from the genetic code, however, the degeneracy of the code must be taken into account. Polynucleotides of the disclosure include sequences which are degenerate as a result of the genetic code. Such polynucleotides are useful for the recombinant production of large quantities of a peptide of interest, such as those set forth in SEQ ID NO: 6-1085, or a functional variant thereof.

In the present disclosure, the polynucleotides encoding the peptides of the disclosure may be inserted into a recombinant “expression vector”. The term “expression vector” refers to a plasmid, virus or other vehicle known in the art that has been manipulated by insertion or incorporation of genetic sequences. Such expression vectors are preferably plasmids that contain a promoter sequence that facilitates the efficient transcription of the inserted genetic sequence in the host. The expression vector typically contains an origin of replication, a promoter, as well as specific genes that allow phenotypic selection of the transformed cells. For example, the expression of the peptides can be placed under control of E. coli chromosomal DNA comprising a lactose or lac operon which mediates lactose utilization by elaborating the enzyme beta-galactosidase. The lac control system can be induced by IPTG. A plasmid can be constructed to contain the lacIq repressor gene, permitting repression of the lac promoter until IPTG is added. Other promoter systems known in the art include beta lactamase, lambda promoters, the protein A promoter, and the tryptophan promoter systems. While these are the most commonly used, other microbial promoters, both inducible and constitutive, can be utilized as well. The vector contains a replicon site and control sequences which are derived from species compatible with the host cell. In addition, the vector may carry specific gene(s) which are capable of providing phenotypic selection in transformed cells. For example, the beta-lactamase gene confers ampicillin resistance to those transformed cells containing the vector with the beta-lactamase gene. An exemplary expression system for production of the peptides is described in U.S. Pat. No. 5,707,855.

Transformation of a host cell with the polynucleotide may be carried out by conventional techniques known to those skilled in the art. For example, where the host is prokaryotic, such as E. coli, competent cells that are capable of DNA uptake can be prepared from cells harvested after exponential growth and subsequently treated by the CaCl₂ method using procedures known in the art. Alternatively, MgCl₂ or RbCl could be used.

In addition to conventional chemical methods of transformation, the plasmid vectors may be introduced into a host cell by physical means, such as by electroporation or microinjection. Electroporation allows transfer of the vector by high voltage electric impulse, which creates pores in the plasma membrane of the host and is performed according to methods known in the art. Additionally, cloned DNA can be introduced into host cells by protoplast fusion, using methods known in the art.

DNA sequences encoding the peptides can be expressed in vivo by DNA transfer into a suitable host cell. “Host cells” are those in which a vector can be propagated and its DNA expressed. The term also includes any progeny of the subject host cell. It is understood that not all progeny are identical to the parental cell, since there may be mutations that occur during replication. However, such progeny are included when the terms above are used. Exemplary host cells include E. coli, S. aureus and P. aeruginosa, although other Gram negative and Gram positive organisms known in the art can be utilized as long as the expression vectors contain an origin of replication to permit expression in the host.

The polynucleotide sequence encoding a peptide as described herein can be isolated from an organism or synthesized in the laboratory. Specific DNA sequences encoding the peptide of interest can be obtained by: 1) isolation of a double-stranded DNA sequence from the genomic DNA; 2) chemical manufacture of a DNA sequence to provide the necessary codons for the peptide of interest; and 3) in vitro synthesis of a double-stranded DNA sequence by reverse transcription of mRNA isolated from a donor cell. In the latter case, a double-stranded DNA complement of mRNA is eventually formed that is generally referred to as cDNA.

The synthesis of DNA sequences is frequently the method of choice when the entire sequence of amino acid residues of the desired peptide product is known. In the present disclosure, the synthesis of a DNA sequence has the advantage of allowing the incorporation of codons that are more likely to be recognized by a bacterial host, thereby permitting high level expression without difficulties in translation. In addition, virtually any peptide can be synthesized, including those encoding natural peptides, variants of the same, or synthetic peptides.

When the entire sequence of the desired peptide is not known, the direct synthesis of DNA sequences is not possible and the method of choice is the formation of cDNA sequences. Among the standard procedures for isolating cDNA sequences of interest is the formation of plasmid or phage containing cDNA libraries that are derived from reverse transcription of mRNA that is abundant in donor cells that have a high level of genetic expression. When used in combination with polymerase chain reaction technology, even rare expression products can be cloned. In those cases where significant portions of the amino acid sequence of the peptide are known, the production of labeled single or double-stranded DNA or RNA probe sequences duplicating a sequence putatively present in the target cDNA may be employed in DNA/DNA hybridization procedures which are carried out on cloned copies of the cDNA which have been denatured into a single stranded form (Jay, et al., Nuc. Acid Res., 11:2325, 1983).

### Methods of Use—Anti-Biofilm

The disclosure also provides a method of inhibiting the biofilm growth of bacteria including contacting the bacteria with an inhibiting effective amount of a peptide of the disclosure, including a peptide having an amino acid sequence set forth in SEQ ID NO: 6-1085, or in one or more of Tables 1, 2 or 8-15, or falling within a consensus sequence as described herein, and analogs, derivatives, enantiomers, retro-inverso derivatives, amidated and unamidated variations and conservative variations thereof, wherein the peptides have antibiofilm activity. In some embodiments, the peptide may include an amino acid sequence as set forth in one or more of SEQ ID NOs: 25, 29, 30, 38, 39, 44, 45, 46, 57, 60, 65, or a functional variant thereof. In alternative embodiments, the peptide may include an amino acid sequence as set forth in one or more of SEQ ID NOs: 151 or 152 (peptides D-3006 or D-3007, respectively), or a functional variant thereof. In alternative embodiments, the peptide may include an amino acid sequence as set forth in one or more of SEQ ID NOs: 24-47, 146-169, 196-219, 246-437, or a functional variant thereof.

The term “contacting” refers to exposing the bacteria to the peptide so that the peptide can effectively inhibit, kill, or cause dispersal of bacteria growing in the biofilm state. Contacting may be in vitro, for example by adding the peptide to a bacterial culture to test for susceptibility of the bacteria to the peptide or acting against biofilms that grow on abiotic surfaces. Contacting may be in vivo, for example administering the peptide to a subject with a bacterial disorder, such as septic shock or infection. Contacting may further involve coating an object (e.g., medical device) such as a catheter or prosthetic device to inhibit the production of biofilms by the bacteria with which it comes into contact, thus preventing it from becoming colonized with the bacteria. “Inhibiting” or “inhibiting effective amount” refers to the amount of peptide that is required to cause an anti-biofilm bacteriostatic or bactericidal effect. Examples of bacteria that may be inhibited include Escherichia coli, Pseudomonas aeruginosa, Klebsiella pneumoniae, Salmonella enteritidis subspecies Typhimurium, Campylobacter sp., Burkholderia complex bacteria, Acinetobacter baumanii, Staphylococcus aureus, Enterococcus facaelis, Listeria monocytogenes, and oral pathogens. Other potential targets are well known to the skilled microbiologist.

The method of inhibiting the growth of biofilm bacteria may further include the addition of antibiotics for combination or synergistic therapy. Antibiotics can work by either assisting the peptide in killing bacteria in biofilms or by inhibiting bacteria released from the biofilm due to accelerated dispersal by a peptide of the disclosure. Those antibiotics most suitable for combination therapy can be easily tested by utilizing modified checkerboard titration assays that use the determination of Fractional Inhibitory Concentrations to assess synergy as further described below. The appropriate antibiotic administered will typically depend on the susceptibility of the biofilms, including whether the bacteria is Gram negative or Gram positive, and will be discernible by one of skill in the art. Examples of particular classes of antibiotics useful for synergistic therapy with the peptides of the disclosure include aminoglycosides (e.g., tobramycin), penicillins (e.g., piperacillin), cephalosporins (e.g., ceftazidime), fluoroquinolones (e.g., ciprofloxacin), carbapenems (e.g., imipenem), tetracyclines, vancomycin, polymyxins and macrolides (e.g., erythromycin and clarithromycin). The method of inhibiting the growth of bacteria may further include the addition of antibiotics for combination or synergistic therapy. The appropriate antibiotic administered will typically depend on the susceptibility of the bacteria such as whether the bacteria is Gram negative or Gram positive, or whether synergy can be demonstrated in vitro, and will be easily discernable by one of skill in the art. Further to the antibiotics listed above, typical antibiotics include aminoglycosides (amikacin, gentamicin, kanamycin, netilmicin, tobramycin, streptomycin), macrolides (azithromycin, clarithromycin, erythromycin, erythromycin estolate/ethylsuccinate/gluceptate/lactobionate/stearate), beta-lactams such as penicillins (e.g., penicillin G, penicillin V, methicillin, nafcillin, oxacillin, cloxacillin, dicloxacillin, ampicillin, amoxicillin, ticarcillin, carbenicillin, mezlocillin, azlocillin and piperacillin), or cephalosporins (e.g., cephalothin, cefazolin, cefaclor, cefamandole, cefoxitin, cefuroxime, cefonicid, cefmetazole, cefotetan, cefprozil, loracarbef, cefetamet, cefoperazone, cefotaxime, ceftizoxime, ceftriaxone, ceftazidime, cefepime, cefixime, cefpodoxime, and cefsulodin) or carbapenems (e.g., imipenem, meropenem, panipenem), or monobactams (e.g., aztreonam). Other classes of antibiotics include quinolones (e.g., fleroxacin, nalidixic acid, norfloxacin, ciprofloxacin, ofloxacin, enoxacin, lomefloxacin and cinoxacin), tetracyclines (e.g., doxycycline, minocycline, tetracycline), and glycopeptides (e.g., vancomycin, teicoplanin), for example. Other antibiotics include chloramphenicol, clindamycin, trimethoprim, sulfamethoxazole, nitrofurantoin, rifampin, linezolid, synercid, polymyxin B, colistin, colimycin, methotrexate, daptomycin, phosphonomycin and mupirocin.

The peptides and/or analogs or derivatives thereof may be administered to any host, including a human or non-human animal, in an amount effective to inhibit not only the growth of a bacterium, but also a virus, parasite or fungus. These peptides are useful as antibiofilm agents, and immunomodulatory anti-infective agents, including anti-bacterial agents, antiviral agents, and antifungal agents.

The disclosure further provides a method of protecting objects from bacterial colonization. Bacteria grow on many surfaces as biofilms. The peptides of the disclosure are active in inhibiting bacteria on surfaces. Thus, the peptides may be used for protecting objects such as medical devices from biofilm colonization with pathogenic bacteria by, coating or chemically conjugating, or by any other means, at least one peptide of the disclosure to the surface of the medical device. Such medical devices include indwelling catheters, prosthetic devices, and the like. Removal of bacterial biofilms from medical equipment, plumbing in hospital wards and other areas where susceptible individuals congregate and the like is also a use for peptides of the disclosure.

### Methods of Use—Immunomodulatory

The present disclosure provides novel cationic peptides, characterized by a group of related sequences and generic formulas, that have ability to modulate (e.g., up- and/or down regulate) polypeptide expression, thereby regulating inflammatory responses, protective immunity and/or innate immunity. These peptides include those set forth in SEQ ID NO: 6-1085, or in one or more of Tables 1, 2 or 8-15, or within a consensus sequence as described herein, and analogs, derivatives, enantiomers, retro-inverso derivatives, amidated and unamidated variations and conservative variations thereof, wherein the peptides have immunomodulatory activity.

In some embodiments, the may include an amino acid sequence as set forth in one or more of SEQ ID NOs: 25, 29, 30, 38, 39, 44, 45, 46, 57, 60, 65, or a functional variant thereof. In alternative embodiments, the may include an amino acid sequence as set forth in one or more of SEQ ID NOs: 151 or 152 (peptides D-3006 or D-3007, respectively), or a functional variant thereof. In alternative embodiments, the peptide may include an amino acid sequence as set forth in one or more of SEQ ID NOs: 24-47, 146-169, 196-219, 246-437, or a functional variant thereof.

“Innate immunity” as used herein refers to the natural ability of an organism to defend itself against invasion by pathogens. Pathogens or microbes as used herein, may include, but are not limited to bacteria, fungi, parasites, and viruses. Innate immunity is contrasted with acquired/adaptive immunity in which the organism develops a defensive mechanism based substantially on antibodies and/or immune lymphocytes that is characterized by specificity, amplifiability and self vs. non-self discrimination. With innate immunity, rapid and broad, relatively nonspecific immunity is provided, molecules from other species can be functional (i.e. there is a substantial lack of self vs. non-self discrimination) and there is no immunologic memory of prior exposure. The hallmarks of innate immunity are effectiveness against a broad variety of potential pathogens, independence of prior exposure to a pathogen, and immediate effectiveness (in contrast to the specific immune response which takes days to weeks to be elicited). However agents that stimulate innate immunity can have an impact on adaptive immunity since innate immunity instructs adaptive immunity ensuring an enhanced adaptive immune response (the underlying principle that guides the selection of adjuvants that are used in vaccines to enhance vaccine responses by stimulating innate immunity). Also the effector molecules and cells of innate immunity overlap strongly with the effectors of adaptive immunity. A feature of many of the IDR peptides revealed here is their ability to selectively stimulate innate immunity, enhancing adaptive immunity to vaccine antigens.

In addition, innate immunity includes immune and inflammatory responses that affect other diseases, such as: vascular diseases: atherosclerosis, cerebral/myocardial infarction, chronic venous disease, pre-eclampsia/eclampsia, and vasculitis; neurological diseases: Alzheimer's disease, Parkinson's disease, epilepsy, and amyotrophic lateral sclerosis (ALS); respiratory diseases: asthma, pulmonary fibrosis, cystic fibrosis, chronic obstructive pulmonary disease, and acute respiratory distress syndrome; dermatologic diseases: psoriasis, acne/rosacea, chronic urticaria, and eczema; gastro-intestinal diseases: celiac disease, inflammatory bowel disease, pancreatitis, esophagitis, gastronintestinal ulceration, and fatty liver disease (alcoholic/obese); endocrine diseases: thyroiditis, paraneoplastic syndrome, type 2 diabetes, hypothyroidism and hyperthyroidism; systemic diseases: sepsis; genito/urinary diseases: chronic kidney disease, nephrotic/nephritic syndrome, benign prostatic hyperplasia, cystitis, pelvic inflammatory disease, urethritis and urethral stricture; and musculoskeletal diseases: osteoporosis, systemic lupus erythematosis; rheumatoid arthritis, inflammatory myopathy, muscular sclerosis, osteoarthritis, costal chondritis and ankylosing spondylitis.

The innate immune system prevents pathogens, in small to modest doses (i.e. introduced through dermal contact, ingestion or inhalation), from colonizing and growing to a point where they can cause life-threatening infections. The major problems with stimulating innate immunity in the past have been created by the excessive production of pro-inflammatory cytokines. Excessive inflammation is associated with detrimental pathology. Thus while the innate immune system is essential for human survival, the outcome of an overly robust and/or inappropriate immune response can paradoxically result in harmful sequelae like e.g. sepsis or chronic inflammation such as with cystic fibrosis. A feature of the IDR peptides revealed here is their ability to selectively stimulate innate immunity, enhancing protective immunity while suppressing the microbially-induced production of pro-inflammatory cytokines.

In innate immunity, the immune response is not dependent upon antigens. The innate immunity process may include the production of secretory molecules and cellular components and the recruitment and differentiation of immune cells. In innate immunity triggered by an infection, molecules on the surface of or within pathogens are recognized by receptors (for example, pattern recognition receptors such as Toll-like receptors) that have broad specificity, are capable of recognizing many pathogens, and are encoded in the germline. When cationic peptides are present in the immune response, they modify (modulate) the host response to pathogens. This change in the immune response induces the release of chemokines, which promote the recruitment of immune cells to the site of infection, enhances the differentiation of immune cells into ones that are more effective in fighting infectious organisms and repairing wounds, and at the same time suppress the potentially harmful production of pro-inflammatory cytokines.

Chemokines, or chemoattractant cytokines, are a subgroup of immune factors that mediate chemotactic and other pro-inflammatory phenomena (See, Schall, 1991, Cytokine 3:165-183). Chemokines are small molecules of approximately 70-80 residues in length and can generally be divided into two subgroups, a which have two N-terminal cysteines separated by a single amino acid (CxC) and β which have two adjacent cysteines at the N terminus (CC). RANTES, MIP-la and MIP-1α are members of the β subgroup (reviewed by Horuk, R., 1994, Trends Pharmacol. Sci, 15:159-165; Murphy, P. M., 1994, Annu. Rev. Immunol., 12:593-633). The amino terminus of the β chemokines RANTES, MCP-1, and MCP-3 have been implicated in the mediation of cell migration and inflammation induced by these chemokines. This involvement is suggested by the observation that the deletion of the amino terminal 8 residues of MCP-1, amino terminal 9 residues of MCP-3, and amino terminal 8 residues of RANTES and the addition of a methionine to the amino terminus of RANTES, antagonize the chemotaxis, calcium mobilization and/or enzyme release stimulated by their native counterparts (Gong et al., 1996 J. Biol. Chem. 271:10521-10527; Proudfoot et al., 1996 J. Biol. Chem. 271:2599-2603). Additionally, a chemokine-like chemotactic activity has been introduced into MCP-1 via a double mutation of Tyr 28 and Arg 30 to leucine and valine, respectively, indicating that internal regions of this protein also play a role in regulating chemotactic activity (Beall et al., 1992, J. Biol. Chem. 267:3455-3459).

The monomeric forms of all chemokines characterized thus far share significant structural homology, although the quaternary structures of α and β groups are distinct. While the monomeric structures of the β and α chemokines are very similar, the dimeric structures of the two groups are completely different. An additional chemokine, lymphotactin, which has only one N terminal cysteine has also been identified and may represent an additional subgroup (γ) of chemokines (Yoshida et al., 1995, FEBS Lett. 360:155-159; and Kelner et al., 1994, Science 266:1395-1399).

Receptors for chemokines belong to the large family of G-protein coupled, 7 transmembrane domain receptors (GCR's) (See, reviews by Horuk, R., 1994, Trends Pharmacol. Sci. 15:159-165; and Murphy, P. M., 1994, Annu. Rev. Immunol. 12:593-633). Competition binding and cross-desensitization studies have shown that chemokine receptors exhibit considerable promiscuity in ligand binding. Examples demonstrating the promiscuity among β chemokine receptors include: CC CKR-1, which binds RANTES and MIP-1α (Neote et al., 1993, Cell 72: 415-425), CC CKR-4, which binds RANTES, MIP-1α, and MCP-1 (Power et al., 1995, J. Biol. Chem. 270:19495-19500), and CC CKR-5, which binds RANTES, MIP-1α, and MIP-1β (Alkhatib et al., 1996, Science, in press and Dragic et al., 1996, Nature 381:667-674). Erythrocytes possess a receptor (known as the Duffy antigen) which binds both α and β chemokines (Horuk et al., 1994, J. Biol. Chem. 269:17730-17733; Neote et al., 1994, Blood 84:44-52; and Neote et al., 1993, J. Biol. Chem. 268:12247-12249). Thus the sequence and structural homologies evident among chemokines and their receptors allows some overlap in receptor-ligand interactions.

In one aspect, the present disclosure provides the use of compounds including peptides of the disclosure to suppress potentially harmful inflammatory responses by acting directly on host cells. In this aspect, a method of identification of a polynucleotide or polynucleotides that are regulated by one or more inflammation inducing agents is provided, where the regulation is altered by a cationic peptide. Such inflammation inducing agents include, but are not limited to endotoxic lipopolysaccharide (LPS), lipoteichoic acid (LTA), flagellin, polyinosinic:polycytidylic acid (PolyIC) and/or CpG DNA or intact bacteria or viruses or other bacterial or viral components. The identification is performed by contacting the host cell with the sepsis or inflammatory inducing agents and further contacting with a cationic peptide either before, simultaneously or immediately after. The expression of the polynucleotide or polypeptide in the presence and absence of the cationic peptide is observed and a change in expression is indicative of a polynucleotide or polypeptide or pattern of polynucleotides or polypeptides that is regulated by a sepsis or inflammatory inducing agent and inhibited by a cationic peptide. In another aspect, the disclosure provides a polynucleotide identified by the method.

Generally, in the methods of the disclosure, a cationic peptide is utilized to modulate the expression of a series of polynucleotides or polypeptides that are essential in the process of inflammation or protective immunity. The pattern of polynucleotide or polypeptide expression may be obtained by observing the expression in the presence and absence of the cationic peptide. The pattern obtained in the presence of the cationic peptide is then useful in identifying additional compounds that can inhibit expression of the polynucleotide and therefore block inflammation or stimulate protective immunity. It is well known to one of skill in the art that non-peptidic chemicals and peptidomimetics can mimic the ability of peptides to bind to receptors and enzyme binding sites and thus can be used to block or stimulate biological reactions. Where an additional compound of interest provides a pattern of polynucleotide or polypeptide expression similar to that of the expression in the presence of a cationic peptide, that compound is also useful in the modulation of an innate immune response to block inflammation or stimulate protective immunity. In this manner, the cationic peptides of the disclosure, which are known inhibitors of inflammation and enhancers of protective immunity are useful as tools in the identification of additional compounds that inhibit sepsis and inflammation and enhance innate immunity.

As can be seen in the Examples below, peptides of the disclosure have an ability to reduce the expression of polynucleotides or polypeptides regulated by LPS, particularly the quintessential pro-inflammatory cytokine TNFα. High levels of endotoxins in the blood are responsible for many of the symptoms seen during a serious infection or inflammation such as fever and an elevated white blood cell count, and many of these effects reflect or are caused by high levels of induced TNFα. Endotoxin (also called lipopolysaccharide) is a component of the cell envelope of Gram negative bacteria and is a potent trigger of the pathophysiology of sepsis. The basic mechanisms of inflammation and sepsis are interrelated.

In another aspect, the disclosure identifies agents that enhance innate immunity. Human cells that contain a polynucleotide or polynucleotides that encode a polypeptide or polypeptides involved in innate immunity are contacted with an agent of interest. Expression of the polynucleotide is determined, both in the presence and absence of the agent. The expression is compared and of the specific modulation of expression was indicative of an enhancement of innate immunity. In another aspect, the agent does not by itself stimulate an inflammatory response as revealed by the lack of upregulation of the pro-inflammatory cytokine TNF-α. In still another aspect the agent reduces or blocks the inflammatory or septic response. In yet another aspect the agent selectively stimulates innate immunity, thus promoting an adjuvant response and enhancing adaptive immunity to vaccine antigens.

In another aspect, the disclosure provides methods of direct polynucleotide or polypeptide regulation by cationic peptides and the use of compounds including cationic peptides to stimulate elements of innate immunity. In this aspect, the disclosure provides a method of identification of a pattern of polynucleotide or polypeptide expression for identification of a compound that enhances protective innate immunity. In the method of the disclosure, an initial detection of a pattern of polypeptide expression for cells contacted in the presence and absence of a cationic peptide is made. The pattern resulting from polypeptide expression in the presence of the peptide represents stimulation of protective innate immunity. A pattern of polypeptide expression is then detected in the presence of a test compound, where a resulting pattern with the test compound that is similar to the pattern observed in the presence of the cationic peptide is indicative of a compound that enhances protective innate immunity. In another aspect, the disclosure provides compounds that are identified in the above methods. In another aspect, the compound of the disclosure stimulates chemokine expression. Chemokines may include, but are not limited to Gro-α, MCP-1, and MCP-3. In still another aspect, the compound is a peptide, peptidomimetic, chemical compound, or a nucleic acid molecule.

It has been shown that cationic peptides can neutralize the host response to the signaling molecules of infectious agents as well as modify the transcriptional responses of host cells, mainly by down-regulating the pro-inflammatory response and/or up-regulating the anti-inflammatory response. Example 9 shows that the cationic peptides can selectively suppress the agonist stimulated induction of the inflammation inducing cytokine TNFα in host cells. Example 6 shows that the cationic peptides can aid in the host response to pathogens by inducing the release of chemokines, which promote the recruitment of immune cells to the site of infection.

It is seen from the examples below that cationic peptides have a substantial influence on the host response to pathogens in that they assist in regulation of the host immune response by inducing selective pro-inflammatory responses that for example promote the recruitment of immune cells to the site of infection but not inducing potentially harmful pro-inflammatory cytokines. The pathology associated with infections and sepsis appears to be caused in part by a potent pro-inflammatory response to infectious agents. Peptides can aid the host in a “balanced” response to pathogens by inducing an anti-inflammatory response and suppressing certain potentially harmful pro-inflammatory responses.

### Treatment Regimes

The disclosure provides pharmaceutical compositions comprising one or a combination of a peptide in accordance with the present disclosure, for example, formulated together with a pharmaceutically acceptable carrier. Some compositions include a combination of multiple (e.g., two or more) peptides of the disclosure.

As used herein “pharmaceutically acceptable carrier” includes any and all solvents, dispersion media, coatings, detergents, emulsions, lipids, liposomes and nanoparticles, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular or topical administration. In another embodiment, the carrier is suitable for oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is compatible with the active compound, use thereof in the pharmaceutical compositions is contemplated. Supplementary active compounds can also be incorporated into the compositions.

A “pharmaceutically acceptable salt” refers to a salt that retains the desired biological activity of the parent compound and does not impart any undesired toxicological effects (See, e.g., Berge, et al., J. Pharm. Sci., 66: 1-19, 1977). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

In prophylactic applications, pharmaceutical compositions or medicaments are administered to a patient susceptible to, or otherwise at risk of a disease or condition (i.e., as a result of bacteria, fungi, viruses, parasites or the like) in an amount sufficient to eliminate or reduce the risk, lessen the severity, or delay the outset of the disease, including biochemical, histologic and/or behavioral symptoms of the disease, its complications and intermediate pathological phenotypes presenting during development of the disease. In therapeutic applications, compositions or medicants are administered to a patient suspected of, or already suffering from such a disease or condition in an amount sufficient to cure, or at least partially arrest, the symptoms of the disease or condition (e.g., biochemical and/or histologic), including its complications and intermediate pathological phenotypes in development of the disease or condition. An amount adequate to accomplish therapeutic or prophylactic treatment is defined as a therapeutically- or prophylactically-effective dose. In both prophylactic and therapeutic regimes, agents are usually administered in several dosages until a sufficient response has been achieved. Typically, the response is monitored and repeated dosages are given if the response starts to wane.

The pharmaceutical composition of the present disclosure should be sterile and fluid to the extent that the composition is deliverable by syringe. In addition to water, the carrier can be an isotonic buffered saline solution, ethanol, polyol (for example, glycerol, propylene glycol, and liquid polyetheylene glycol, and the like), and suitable mixtures thereof. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

When the active compound is suitably protected, as described above, the compound can be orally administered, for example, with an inert diluent or an assimilable edible carrier.

Pharmaceutical compositions of the disclosure also can be administered in combination therapy, i.e., combined with other agents. For example, in treatment of bacteria, the combination therapy can include a composition of the present disclosure with at least one agent or other conventional therapy.

### Routes of Administration

A composition of the present disclosure can be administered by a variety of methods known in the art. The route and/or mode of administration vary depending upon the desired results. The phrases “parenteral administration” and “administered parenterally” mean modes of administration other than enteral and topical administration, usually by injection, and includes, without limitation, intravenous, intramuscular, intraabscess, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. The peptide of the disclosure can be administered parenterally by injection or by gradual infusion over time. The peptide can also be prepared with carriers that protect the compound against rapid release, such as a controlled release formulation, including implants, transdermal patches, and microencapsulated delivery systems Further methods for delivery of the peptide include orally, by encapsulation in microspheres or proteinoids, by aerosol delivery to the lungs, or transdermally by iontophoresis or transdermal electroporation., or directly injected into abscesses.

The peptides may also be delivered via transdermal or topical application. Transdermal and topical dosage forms of the disclosure include, but are not limited to, creams, lotions, ointments, gels, solutions, emulsions, suspensions, or other forms known to one of skill in the art. See, e.g., Remington's Pharmaceutical Sciences, 18th eds., Mack Publishing, Easton Pa. (1990); and Introduction to Pharmaceutical Dosage Forms, 4th ed., Lea & Febiger, Philadelphia (1985). Transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal and topical dosage forms encompassed by this disclosure are well known to those skilled in the pharmaceutical arts, and will depend on the particular tissue to which a given pharmaceutical composition or dosage form will be applied. For example, typical excipients include, but are not limited to, water, acetone, ethanol, ethylene glycol, propylene glycol, butane-1,3-diol, isopropyl myristate, isopropyl palmitate, lipids, nanoparticles, mineral oil, and mixtures thereof to form lotions, tinctures, creams, emulsions, gels or ointments, which are non-toxic and pharmaceutically acceptable. Moisturizers or humectants can also be added to pharmaceutical compositions and dosage forms if desired. Examples of such additional ingredients are well known in the art. See, e.g., See, e.g., Remington's Pharmaceutical Sciences, 18th eds., Mack Publishing, Easton Pa. (1990).

Depending on the specific tissue to be treated, additional components may be used prior to, in conjunction with, or subsequent to treatment with peptides as described herein. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water-soluble or insoluble sugar esters such as Tween 80 (polysorbate 80) and Span 60 (sorbitan monostearate).

To administer a peptide of the disclosure by certain routes of administration, it can be necessary to coat the compound with, or co-administer the compound with, a material to prevent its inactivation. The method of the disclosure also includes delivery systems such as microencapsulation of peptides into liposomes or a diluent. Microencapsulation also allows co-entrapment of antimicrobial molecules along with the antigens, so that these molecules, such as antibiotics, may be delivered to a site in need of such treatment in conjunction with the peptides of the disclosure. Liposomes in the blood stream are generally taken up by the liver and spleen. Pharmaceutically acceptable diluents include saline and aqueous buffer solutions. Liposomes include water-in-oil-in-water CGF emulsions as well as conventional liposomes (Strejan, et al., J. Neuroimmunol., 7: 27, 1984). Thus, the method of the disclosure is particularly useful for delivering antimicrobial peptides to such organs. Biodegradable, biocompatible polymers can be used, such as ethylene vinyl acetate, polyanhydrides, polyglycolic acid, collagen, polyorthoesters, and polylactic acid. Many methods for the preparation of such formulations are described by e.g., Sustained and Controlled Release Drug Delivery Systems, J. R. Robinson, Ed., 1978, Marcel Dekker, Inc., New York. Other methods of administration will be known to those skilled in the art.

Preparations for parenteral administration of a peptide of the disclosure include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Therapeutic compositions typically must be sterile, substantially isotonic, and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. Therapeutic compositions can also be administered with medical devices known in the art. For example, in a preferred embodiment, a therapeutic composition of the disclosure can be administered with a needleless hypodermic injection device, such as the devices disclosed in, e.g., U.S. Pat. Nos. 5,399,163, 5,383,851, 5,312,335, 5,064,413, 4,941,880, 4,790,824, or 4,596,556. Examples of implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

When the peptides of the present disclosure are administered as pharmaceuticals, to humans and animals, they can be given alone or as a pharmaceutical composition containing, for example, 0.01 to 99.5% (or 0.1 to 90%) of active ingredient in combination with a pharmaceutically acceptable carrier.

### Effective Dosages

“Therapeutically effective amount” as used herein for treatment of antimicrobial related diseases and conditions refers to the amount of peptide used that is of sufficient quantity to decrease the numbers of bacteria, viruses, fungi, and parasites in the body of a subject. The dosage ranges for the administration of peptides are those large enough to produce the desired effect. The amount of peptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's (Remington's Pharmaceutical Science, Mack Publishing Company, Easton, Pa.); Egleton, Peptides 18: 1431-1439, 1997; Langer Science 249: 1527-1533, 1990. The dosage regimen can be adjusted by the individual physician in the event of any contraindications.

Dosage regimens of the pharmaceutical compositions of the present disclosure are adjusted to provide the optimum desired response (e.g., a therapeutic response). For example, a single bolus can be administered, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the disclosure are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions of the present disclosure can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present disclosure employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of the compounds of the disclosure employed in the pharmaceutical composition at levels lower than that required to achieve the desired therapeutic effect and gradually increase the dosage until the desired effect is achieved. In general, a suitable daily dose of a compound of the disclosure is that amount of the compound which is the lowest dose effective to produce a therapeutic effect. Such an effective dose generally depends upon the factors described above. It is preferred that administration be intravenous, intramuscular, intraperitoneal, or subcutaneous, or administered proximal to the site of the target. If desired, the effective daily dose of a therapeutic composition can be administered as two, three, four, five, six or more sub-doses administered separately at appropriate intervals throughout the day, optionally, in unit dosage forms. While it is possible for a compound of the present disclosure to be administered alone, it is preferable to administer the compound as a pharmaceutical formulation (composition).

An effective dose of each of the peptides disclosed herein as potential therapeutics for use in treating microbial diseases and conditions is from about 1 μg/kg to 500 mg/kg body weight, per single administration, which can readily be determined by one skilled in the art. As discussed above, the dosage depends upon the age, sex, health, and weight of the recipient, kind of concurrent therapy, if any, and frequency of treatment. Other effective dosage range upper limits are 50 mg/kg body weight, 20 mg/kg body weight, 8 mg/kg body weight, and 2 mg/kg body weight.

The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

Some compounds of the disclosure can be formulated to ensure proper distribution in vivo. For example, the blood-brain barrier (BBB) excludes many highly hydrophilic compounds. To ensure that the therapeutic compounds of the disclosure cross the BBB (if desired), they can be formulated, for example, in liposomes. For methods of manufacturing liposomes, See, e.g., U.S. Pat. Nos. 4,522,811; 5,374,548; and 5,399,331. The liposomes can comprise one or more moieties which are selectively transported into specific cells or organs, thus enhance targeted drug delivery (See, e.g., Ranade, J. Clin. Pharmacol., 29: 685, 1989). Exemplary targeting moieties include folate or biotin (See, e.g., U.S. Pat. No. 5,416,016 to Low, et al.); mannosides (Umezawa, et al., Biochem. Biophys. Res. Commun., 153: 1038, 1988); antibodies (Bloeman, et al., FEBS Lett., 357: 140, 1995; Owais, et al., Antimicrob. Agents Chemother., 39: 180, 1995); surfactant protein A receptor (Briscoe, et al., Am. J. Physiol., 1233: 134, 1995), different species of which can comprise the formulations of the disclosure, as well as components of the invented molecules; p120 (Schreier, et al., J. Biol. Chem., 269: 9090, 1994); See also Keinanen, et al., FEBS Lett., 346: 123, 1994; Killion, et al., Immunomethods, 4: 273, 1994. In some methods, the therapeutic compounds of the disclosure are formulated in liposomes; in a more preferred embodiment, the liposomes include a targeting moiety. In some methods, the therapeutic compounds in the liposomes are delivered by bolus injection to a site proximal to the tumor or infection. The composition should be fluid to the extent that easy syringability exists. It should be stable under the conditions of manufacture and storage and should be preserved against the contaminating action of microorganisms such as bacteria and fungi.

“Anti-biofilm amount” as used herein refers to an amount sufficient to achieve a biofilm-inhibiting blood concentration in the subject receiving the treatment. The anti-bacterial amount of an antibiotic generally recognized as safe for administration to a human is well known in the art, and as is known in the art, varies with the specific antibiotic and the type of bacterial infection being treated.

Because of the broad spectrum anti-biofilm properties of the peptides, they may also be used as preservatives or to prevent formation of biofilms on materials susceptible to microbial biofilm contamination. The peptides of the disclosure can be utilized as broad spectrum anti-biofilm agents directed toward various specific applications. Such applications include use of the peptides as preservatives for processed foods (organisms including Salmonella, Yersinia, Shigella, Pseudomonas and Listeria), either alone or in combination with antibacterial food additives such as lysozymes; as a topical agent (Pseudomonas, Streptococcus, Staphylococcus) and to kill odor producing microbes (Micrococci). The relative effectiveness of the peptides of the disclosure for the applications described can be readily determined by one of skill in the art by determining the sensitivity of biofilms formed by any organism to one of the peptides.

### Formulation

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this disclosure can be administered in the form of a depot injection or implant preparation which can be formulated in such a manner as to permit a sustained or pulsatile release of the active ingredient.

Additional formulations suitable for other modes of administration include oral, intranasal, topical and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, detergents like Tween or Brij, PEGylated lipids, cellulose, magnesium carbonate, methyl cellulose 25 cP, carboxymethyl cellulose, hydroxypropyl methyl cellulose, hyluronic acid and hyperbranched polyglycerols. These compositions take the form of solutions, suspensions, tablets, pills, capsules, sustained release formulations or powders and contain 10%-95% of active ingredient, preferably 25%-70%.

Topical application can result in transdermal or intradermal delivery, or enable activity against local biofilm infections. Co-administration can be achieved by using the components as a mixture or as linked molecules obtained by chemical crosslinking or expression as a fusion protein.

Alternatively, transdermal delivery can be achieved using a skin patch or using transferosomes (Paul et al., Eur. J. Immunol. 25: 3521-24, 1995; Cevc et al., Biochem. Biophys. Acta 1368: 201-15, 1998).

The pharmaceutical compositions are generally formulated as sterile, substantially isotonic and in full compliance with all Good Manufacturing Practice (GMP) regulations of the U.S. Food and Drug Administration.

The disclosure provides a number of methods, reagents, and compounds that can be used for inhibiting microbial infections, and biofilm growth. It is to be understood that this disclosure is not limited to particular methods, reagents, compounds, compositions, or biological systems, which can, of course, vary. It is also to be understood that the terminology used herein is for the purpose of describing particular embodiments only and is not intended to be limiting. As used in this specification and the appended embodiments, the singular forms “a”, “an”, and “the” include plural referents unless the content clearly dictates otherwise. Thus, for example, reference to “a peptide” includes a combination of two or more peptides, and the like.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. In describing and claiming the present invention, the following terminology will be used.

From the foregoing description, various modifications and changes in the compositions and methods will occur to those skilled in the art. All such modifications coming within the scope of the appended embodiments are intended to be included therein. Each recited range includes all combinations and sub-combinations of ranges, as well as specific numerals contained therein.

### EXEMPLARY EMBODIMENTS

### Example 1: Materials, Methods and Peptides

Peptide Synthesis—All peptides used in this study as isolated peptides, for example as listed in Table 1 and Example 13, were synthesized by GenScript (Piscataway, N.J., USA), or other suitable companies, using solid phase Fmoc chemistry and purified to a purity >95% using reverse phase HPLC, or were synthesized on cellulose membranes by SPOT synthesis. Peptide mass was confirmed by mass spectrometry. SPOT peptide syntheses on cellulose were performed using a pipetting robot (Abimed, Langenfeld, Germany) and Whatman 50 cellulose membranes (Whatman, Maidstone, United Kingdom) as described previously (Kramer A, Schuster A, Reinecke U, Malin R, Volkmer-Engert R, Landgraf C, Schneider-Mergener J. 1994. Combinatorial cellulose-bound peptide libraries: screening tool for the identification of peptides that bind ligands with predefined specificity. Comp. Meth. Enzymol. 6, 388-395; Kramer A, Keitel T, Winkler K, Stocklein W, Hohne W, Schneider-Mergener J. 1997. Molecular basis for the binding promiscuity of an anti-p24 (HIV-1) monoclonal antibody. Cell 91, 799-809). Table 1 lists active synthetic peptides and their sequences.

TABLE 1
List of synthetic peptides and their sequences.
All peptides are amidated at the carboxy
terminus. Peptides in plain type comprise only
L-amino acids. Italics indicate D-amino acids.
The non-natural amino acid abbreviations are
as follows: Gpro, L-2-amino-3-guanidinopropionic
acid; Gbut, L-2-Amino-4-guanidinobutyric acid;
Har, L-Homoarginine; Dap, L-2,3-diaminopropionic
acid; Orn, L-Ornithine. Sequences 80-245 are D
amino acid containing peptides indicated in
italics. SEQ ID NOs: 80-145 combine two or more
favourable substitutions (highlighted in bold)
in the peptide sequences based on a substitution
screen of these parent peptides RI-1018, DJK5
or RI-1002. SEQ ID NOs: 146 to 245 are the D-
and Retro-Inverso forms of the QSAR optimized
peptides, SEQ ID NOs: 24-73. SEQ ID NOs: 1, 3-5
are known and are specifically excluded. SEQ ID
NOs: 246 to 437 are single amino acid
substitution variants of peptides 3002
(SEQ ID NO: 25) and 3007 (SEQ ID NO: 30).

| SEQ ID NO | Peptide name | Sequences |
|---|---|---|
| 1 | 1018 | VRLIVAVRIWRR |
| 3 | RI-1018 | RRWIRVAVILRV |
| 4 | DJK5 | VQWRAIRVRVIR |
| 5 | RI-1002 | KRIRWVILWRQV |
| 6 | 1018-k36 | VRLIVAVRIWROrn |
| 7 | 1018-k1 | DapRLIVAVRIWRR |
| 8 | 1018-r36 | VRLIVAVRIWRHar |
| 9 | 1018-k30 | VRLIVOrnVRIWRR |
| 10 | 1018-k12 | VRLIVAVRIWRDap |
| 11 | 1018-r35 | VRLIVAVRIWHarR |
| 12 | 1018-k35 | VRLIVAVRIWOrnR |
| 13 | 1018-k11 | VRLIVAVRIWDapR |
| 14 | 1018-k25 | OrnRLIVAVRIWRR |
| 15 | 1018-r32 | VRLIVAVHarIWRR |
| 16 | 1018-k32 | VRLIVAVOrnIWRR |
| 17 | 1018-r13 | GbutRLIVAVRIWRR |
| 18 | 1018-k10 | VRLIVAVRIDapRR |
| 19 | 1018-r1 | GproRLIVAVRIWRR |
| 20 | 1018-r30 | VRLIVHarVRIWRR |
| 21 | 1018-k13 | DabRLIVAVRIWRR |
| 22 | 1018-k26 | VOrnLIVAVRIWRR |
| 23 | 1018-r25 | HarRLIVAVRIWRR |
| 24 | 3001 | VIKWLLKILRAI |
| 25 | 3002 | ILVRWIRWRIQW |
| 26 | 3003 | WKKVQWLKRLLL |
| 27 | 3004 | IQRWWKVWLKVI |
| 28 | 3005 | RRQWRGWVRIWL |
| 29 | 3006 | IWLRLKVVLKRK |
| 30 | 3007 | VLKIKVKIWVVK |
| 31 | 3008 | KKWQLLIKWKLR |
| 32 | 3009 | AVAKWALKLWKQ |
| 33 | 3010 | QLARLARVVWGL |
| 34 | 3011 | VLQIKKVLRLLL |
| 35 | 3012 | RVKAIKWRKIVV |
| 36 | 3013 | LWQLWLKLKLKG |
| 37 | 3014 | KIQRRAWKQWRK |
| 38 | 3015 | KIVIRIILQVIK |
| 39 | 3016 | AVKWLGWILAKK |
| 40 | 3017 | LAGLIVKWAGVR |
| 41 | 3018 | WVGVIIKWGLKL |
| 42 | 3019 | WQGWAKIWVVRI |
| 43 | 3020 | LIVIQLLKKWWK |
| 44 | 3021 | RRIIKILLWKLR |
| 45 | 3022 | IAWQLLWGWRVR |
| 46 | 3023 | VQRIIWLRVKIV |
| 47 | 3024 | IKIIWKALGQVI |
| 48 | MILBFmax5 | IQLKLIWVKRKW |
| 49 | BFmax-9 | VIKVLIKRWLKL |
| 50 | BFmax-6 | VQWIQIVVWRKR |
| 51 | IBFmax-15 | GLIIKIIKKRLW |
| 52 | Imax-5 | VKGAIKRGIWVK |
| 53 | BFmax-7 | KVQIIKQLIAKK |
| 54 | BFmax-16 | KRLQWVKVKKIR |
| 55 | Imax-10 | IVKWIAQWKLVG |
| 56 | Mmax-16 | KKQKKIWRRILV |
| 57 | Mmax-3 | GRVLKIVWRKGR |
| 58 | Mmax-18 | RQVRVKRWRARW |
| 59 | BFmax-2 | KVVWWKVIIKVL |
| 60 | Imax-7 | ALAIKVWIKILQ |
| 61 | MILBFmax-9 | IRILVLRKAIIV |
| 62 | MILBFmax-13 | IVKKVKLIWGVK |
| 63 | Imin-3 | VVGLRVRWVRLW |
| 64 | Imax-20 | WAVRALKVKWAL |
| 65 | IBFmax-13 | WWIKIVVIRVRR |
| 66 | MILBFmax-8 | QIIKVVWRAVII |
| 67 | IBFmax-11 | QQVKWWLIRWLA |
| 68 | IBFmax-17 | IKWVLRKIVQII |
| 69 | BFmax-5 | VARWKIIIAKLW |
| 70 | Imax-6 | KIWGLLKLGIAL |
| 71 | Imin-4 | RARQIRWLRKRV |
| 72 | IBFmax-8 | RVLIKWKKVIVV |
| 73 | MILBFmax-1 | LKLKAILKIIRV |
| 74 | 1018-K | VKLIVAVKIWKK |
| 75 | 1018-Dap | VDapLIVAVDapIWDapDap |
| 76 | 1018-Orn | VOrnLIVAVOrnIWOrnOrn |
| 77 | 1018-5K | VKLIVKVKIWKK |
| 78 | 1018-5Dap | VDapLIVDapVDapIWDapDap |
| 79 | 1018-5Orn | VOrnLIVOrnVOrnIWOrnOrn |
| 80 | RI-1018-R4L3 | RR [symbol] RVAVILRV |
| 81 | RI-1018-R4W6G7 | RRW [symbol] R [symbol] VILRV |
| 82 | RI-1018-R4W6 | RRW [symbol] R [symbol] AVILRV |
| 83 | RI-1018-A4L3 | RR [symbol] RVAVILRV |
| 84 | RI-1018-A4G7 | RRW [symbol] RV [symbol] VILRV |
| 85 | RI-1018-A4W6 | RRW [symbol] R [symbol] AVILRV |
| 86 | RI-1018-A4W6R7 | RRW [symbol] R [symbol] VILRV |
| 87 | RI-1018-R7R4 | RRW [symbol] RV [symbol] VILRV |
| 88 | RI-1018-R7A4 | RRW [symbol] RV [symbol] VILRV |
| 89 | RI-1018-R7L3 | RR [symbol] IRV [symbol] VILRV |
| 90 | RI-1018-R7W6 | RRWIR [symbol] VILRV |
| 91 | RI-1018-K7R4 | RRW [symbol] RV [symbol] VILRV |
| 92 | RI-1018-K7A4 | RRW [symbol] RV [symbol] VILRV |
| 93 | RI-1018-K7L3 | RR [symbol] IRV [symbol] VILRV |
| 94 | RI-1018-K7W6 | RRWIR [symbol] VILRV |
| 95 | RI-1018-G7R4 | RRW [symbol] RV [symbol] VILRV |
| 96 | RI-1018-R7R4L3 | RR [symbol] RV [symbol] VILRV |
| 97 | RI-1018-K7R4L3 | RR [symbol] RV [symbol] VILRV |
| 98 | RI-1018-R7R4W6 | RRW [symbol] R [symbol] VILRV |
| 99 | RI-1018-K7R4W6 | RRW [symbol] R [symbol] VILRV |
| 100 | DJK5-R517 | VQWR [symbol] I [symbol] VRVIR |
| 101 | DJK5-R5L7 | VQWR [symbol] I [symbol] VRVIR |
| 102 | DJK5-R519 | VQWR [symbol] IRV [symbol] VIR |
| 103 | DJK5-R51719 | VQWR [symbol] I [symbol] V [symbol] VIR |
| 104 | DJK5-R5L7I9 | VQWR [symbol] I [symbol] V [symbol] VIR |
| 105 | DJK5-I7V5 | VQWR [symbol] I [symbol] VRVIR |
| 106 | DJK5-I7L5 | VQWR [symbol] I [symbol] VRVIR |
| 107 | DJK5-I9V5 | VQWR [symbol] IRV [symbol] VIR |
| 108 | DJK5-I9L5 | VQWR [symbol] IRV [symbol] VIR |
| 109 | RI-1002-R3R5 | KR [symbol] R [symbol] VILWRQV |
| 110 | RI-1002-R3V9 | KR [symbol] RWVIL [symbol] RQV |
| 111 | RI-1002-R3I9 | KR [symbol] RWVIL [symbol] RQV |
| 112 | RI-1002-R3L9 | KR [symbol] RWVIL [symbol] RQV |
| 113 | RI-1002-Q3R5K9 | KR [symbol] RRVIL [symbol] RQV |
| 114 | RI-1002-R5K11 | KRIR [symbol] VILWR [symbol] V |
| 115 | RI-1002-R3K11 | KR [symbol] RWVILWR [symbol] V |
| 116 | RI-1002-G3K11 | KR [symbol] RWVILWR [symbol] V |
| 117 | RI-1002-Q3K11 | KR [symbol] RWVILWR [symbol] V |
| 118 | RI-1002-A3K11 | KR [symbol] RWVILWR [symbol] V |
| 119 | RI-1002-V9K11 | KRIRWVIL [symbol] R [symbol] V |
| 120 | RI-1002-I9K11 | KRIRWVIL [symbol] R [symbol] V |
| 121 | RI-1002-L9K11 | KRIRWVIL [symbol] R [symbol] V |
| 122 | RI-1002-Q3R5 | KR [symbol] R [symbol] VILWRQV |
| 123 | RI-1002-G3R5 | KR [symbol] R [symbol] VILWRQV |
| 124 | RI-1002-A3R5 | KR [symbol] R [symbol] VILWRQV |
| 125 | RI-1002-V9R5 | KRIR [symbol] VIL [symbol] RQV |
| 126 | RI-1002-I9R5 | KRIR [symbol] VIL [symbol] RQV |
| 127 | RI-1002-L9R5 | KRIR [symbol] VIL [symbol] RQV |
| 128 | RI-1002-Q3V9 | KR [symbol] RWVIL [symbol] RQV |
| 129 | RI-1002-G3V9 | KR [symbol] RWVIL [symbol] RQV |
| 130 | RI-1002-A3V9 | KR [symbol] RWVIL [symbol] RQV |
| 131 | RI-1002-Q3I9 | KR [symbol] RWVIL [symbol] RQV |
| 132 | RI-1002-G3I9 | KR [symbol] RWVIL [symbol] RQV |
| 133 | RI-1002-A3I9 | KR [symbol] RWVIL [symbol] RQV |
| 134 | RI-1002-Q3L9 | KR [symbol] RWVIL [symbol] RQV |
| 135 | R1-1002-G3L9 | KR [symbol] RWVIL [symbol] RQV |
| 136 | R1-1002-A3L9 | KR [symbol] RWVIL [symbol] RQV |
| 137 | R1-1002-Q3R5V9 | KR [symbol] R [symbol] VIL [symbol] RQV |
| 138 | R1-1002-G3R5V9 | KR [symbol] R [symbol] VIL [symbol] RQV |
| 139 | R1-1002-A3R5V9 | KR [symbol] R [symbol] VIL [symbol] RQV |
| 140 | R1-1002-Q3R519 | KR [symbol] R [symbol] VIL [symbol] RQV |
| 141 | R1-1002-G3R519 | KR [symbol] R [symbol] VIL [symbol] RQV |
| 142 | R1-1002-A3R519 | KR [symbol] R [symbol] VIL [symbol] RQV |
| 143 | R1-1002-Q3R5L9 | KR [symbol] R [symbol] VIL [symbol] RQV |
| 144 | R1-1002-G3R5L9 | KR [symbol] R [symbol] VIL [symbol] RQV |
| 145 | R1-1002-A3R5L9 | KR [symbol] R [symbol] VIL [symbol] RQV |
| 146 | D-3001 | VIKWLLKILRAI |
| 147 | D-3002 | ILVRWIRWRIQW |
| 148 | D-3003 | WKKVQWLKRLLL |
| 149 | D-3004 | IQRWWKVWLKVI |
| 150 | D-3005 | RRQWRGWVRIWL |
| 151 | D-3006 | IWLRLKVVLKRK |
| 152 | D-3007 | VLKIKVKIWVVK |
| 153 | D-3008 | KKWQLLIKWKLR |
| 154 | D-3009 | AVAKWALKLWKQ |
| 155 | D-3010 | QLARLARVVWGL |
| 156 | D-3011 | VLQIKKVLRLLL |
| 157 | D-3012 | RVKAIKWRKIVV |
| 158 | D-3013 | LWQLWLKLKLKG |
| 159 | D-3014 | KIQRRAWKQWRK |
| 160 | D-3015 | KIVIRIILQVIK |
| 161 | D-3016 | AVKWLGWILAKK |
| 162 | D-3017 | LAGLIVKWAGVR |
| 163 | D-3018 | WVGVIIKWGLKL |
| 164 | D-3019 | WQGWAKIWVVRI |
| 165 | D-3020 | LIVIQLLKKWWK |
| 166 | D-3021 | RRIIKILLWKLR |
| 167 | D-3022 | IAWQLLWGWRVR |
| 168 | D-3023 | VQRIIWLRVKIV |
| 169 | D-3024 | IKIIWKALGQVI |
| 170 | D-MILBFmax5 | IQLKLIWVKRKW |
| 171 | D-BFmax-9 | VIKVLIKRWLKL |
| 172 | D-BFmax-6 | VQWIQIVVWRKR |
| 173 | D-IBFmax-15 | GLIIKIIKKRLW |
| 174 | D-Imax-5 | VKGAIKRGIWVK |
| 175 | D-BFmax-7 | KVQIIKQLIAKK |
| 176 | D-BFmax-16 | KRLQWVKVKKIR |
| 177 | D-Imax-10 | IVKWIAQWKLVG |
| 178 | D-Mmax-16 | KKQKKIWRRILV |
| 179 | D-Mmax-3 | GRVLKIVWRKGR |
| 180 | D-Mmax-18 | KQVRVKRWRARW |
| 181 | D-BFmax-2 | KVVWWKVIIKVL |
| 182 | D-Imax-7 | ALAIKVWIKILQ |
| 183 | D-MILBFmax-9 | IRILVLRKAIIV |
| 184 | D-MILBFmax-13 | IVKKVKLIWGVK |
| 185 | D-Imin-3 | VVGLRVRWVRLW |
| 186 | D-Imax-20 | WAVRALKVKWAL |
| 187 | D-IBFmax-13 | WWIKIVVIRVRR |
| 188 | D-MILBFmax-8 | QIIKVVWRAVII |
| 189 | D-IBFmax-11 | QQVKWWLIRWLA |
| 190 | D-IBFmax-17 | IKWVLRKIVQII |
| 191 | D-BFmax-5 | VARWKIIIAKLW |
| 192 | D-Imax-6 | KIWGLLKLGIAL |
| 193 | D-Imin-4 | RARQIRWLRKRV |
| 194 | D-IBFmax-8 | RVLIKWKKVIVV |
| 195 | D-MILBFmax-1 | KLKLAILKIIRV |
| 196 | RI-3001 | IARLIKLLWKIV |
| 197 | RI-3002 | WQIRWRIWRVLI |
| 198 | RI-3003 | LLLRKLWQVKKW |
| 199 | RI-3004 | IVKLWVKWWRQI |
| 200 | RI-3005 | LWIRVWGRWQRR |
| 201 | RI-3006 | KRKLVVKLRLWI |
| 202 | RI-3007 | KVVWIKVKIKLV |
| 203 | RI-3008 | RLKWKILLQWKK |
| 204 | RI-3009 | QKWLKLAWKAVA |
| 205 | RI-3010 | LGWVVRALRALQ |
| 206 | RI-3011 | LLLRLVKKIQLV |
| 207 | RI-3012 | VVIKRWKIAKVR |
| 208 | RI-3013 | GKLKLKLWLQWL |
| 209 | RI-3014 | KRWQKWARRQIK |
| 210 | RI-3015 | KIVQLIIRIVIK |
| 211 | RI-3016 | KKALIWGLWKVA |
| 212 | RI-3017 | RVGAWKVILGAL |
| 213 | RI-3018 | LKLGWKIIVGVW |
| 214 | RI-3019 | IRVVWIKAWGQW |
| 215 | RI-3020 | KWWKKLLQIVIL |
| 216 | RI-3021 | RLKWLLIKIIRR |
| 217 | RI-3022 | RVRWGWLLQWAI |
| 218 | RI-3023 | VIKVRLWIIRQV |
| 219 | RI-3024 | IVQGLAKWHICI |
| 220 | RI-MILBFmax5 | WKRKVWILKLQI |
| 221 | RI-BFmax-9 | LKLWRKILVKIV |
| 222 | RI-BFmax-6 | RKRWVVIQIWQV |
| 223 | RI-IBFmax-15 | WLRKKIIKIILG |
| 224 | RI-Imax-5 | KVWIGRKIAGKV |
| 225 | RI-BFmax-7 | KKAILQKIIQVK |
| 226 | RI-BFmax-16 | RIKKVKVWQLRK |
| 227 | RI-Imax-10 | GVLKWQAIWKVI |
| 228 | RI-Mmax-16 | VLIRRWIKKQKK |
| 229 | RI-Mmax-3 | RGKRWVIKLVRG |
| 230 | RI-Mmax-18 | WRARWRKVRVQR |
| 231 | RI-BFmax-2 | LVKIIVKWWVVK |
| 232 | RI-Imax-7 | QLIKIWVKIALA |
| 233 | RI-MILBFmax-9 | VIIAKRLVLIRI |
| 234 | RI-MILBFmax-13 | KVGWILKVKKVI |
| 235 | RI-Imin-3 | WLRVWRVRLGVV |
| 236 | RI-Imax-20 | LAWKVKLARVAW |
| 237 | RI-IBFmax-13 | RRVRIVVIKIWW |
| 238 | RI-MILBFmax-8 | IIVARWVVKIIQ |
| 239 | RI-IBFmax-11 | ALWRILWWKVQQ |
| 240 | RI-IBFmax-1 7 | IIQVIKRLVWKI |
| 241 | RI-BFmax-5 | WLKAIIIKWRAV |
| 242 | RI-Imax-6 | LAIGLKLLGWIK |
| 243 | RI-Imin-4 | VRKRLWRIQRAR |
| 244 | RI-IBFmax-8 | VVIVKKWKILVR |
| 245 | RI-MILBFmax-1 | VRIIKLIAKLKL |
| 246 | 3002-G1 | GLVRWIRWRIQW |
| 247 | 3002-G2 | IGVRWIRWRIQW |
| 248 | 3002-G3 | ILGRWIRWRIQW |
| 249 | 3002-G4 | ILVGWIRWRIQW |
| 250 | 3002-G5 | ILVRGIRWRIQW |
| 251 | 3002-G6 | ILVRWGRWRIQW |
| 252 | 3002-G7 | ILVRWIGWRIQW |
| 253 | 3002-G8 | ILVRWIRGRIQW |
| 254 | 3002-G9 | ILVRWIRWGIQW |
| 255 | 3002-G10 | ILVRWIRWRGQW |
| 256 | 3002-G11 | ILVRWIRWRIGW |
| 257 | 3002-G12 | ILVRWIRWRIQG |
| 258 | 3002-A1 | ALVRWIRWRIQW |
| 259 | 3002-A2 | IAVRWIRWRIQW |
| 260 | 3002-A3 | ILARWIRWRIQW |
| 261 | 3002-A4 | ILVAWIRWRIQW |
| 262 | 3002-A5 | ILVRAIRWRIQW |
| 263 | 3002-A6 | ILVRWARWRIQW |
| 264 | 3002-A7 | ILVRWIAWRIQW |
| 265 | 3002-A8 | ILVRWIRARIQW |
| 266 | 3002-A9 | ILVRWIRWAIQW |
| 267 | 3002-A10 | ILVRWIRWRAQW |
| 268 | 3002-A11 | ILVRWIRWRIAW |
| 269 | 3002-A12 | ILVRWIRWRIQA |
| 270 | 3002-R1 | RLVRWIRWRIQW |
| 271 | 3002-R2 | IRVRWIRWRIQW |
| 272 | 3002-R3 | ILRRWIRWRIQW |
| 273 | 3002-R5 | ILVRRIRWRIQW |
| 274 | 3002-R6 | ILVRWRRWRIQW |
| 275 | 3002-R8 | ILVRWIRRRIQW |
| 276 | 3002-R10 | ILVRWIRWRRQW |
| 277 | 3002-R11 | ILVRWIRWRIRW |
| 278 | 3002-R12 | ILVRWIRWRIQR |
| 279 | 3002-K1 | KLVRWIRWRIQW |
| 280 | 3002-K2 | IKVRWIRWRIQW |
| 281 | 3002-K3 | ILKRWIRWRIQW |
| 282 | 3002-K4 | ILVKWIRWRIQW |
| 283 | 3002-K5 | ILVRKIRWRIQW |
| 284 | 3002-K6 | ILVRWKRWRIQW |
| 285 | 3002-K7 | ILVRWIKWRIQW |
| 286 | 3002-K8 | ILVRWIRKRIQW |
| 287 | 3002-K9 | ILVRWIRWKIQW |
| 288 | 3002-K10 | ILVRWIRWRKQW |
| 289 | 3002-K11 | ILVRWIRWRIKW |
| 290 | 3002-K12 | ILVRWIRWRIQK |
| 291 | 3002-V1 | VLVRWIRWRIQW |
| 292 | 3002-V2 | IVVRWIRWRIQW |
| 293 | 3002-V4 | ILVVWIRWRIQW |
| 294 | 3002-V5 | ILVRVIRWRIQW |
| 295 | 3002-V6 | ILVRWVRWRIQW |
| 296 | 3002-V7 | ILVRWIVWRIQW |
| 297 | 3002-V8 | ILVRWIRVRIQW |
| 298 | 3002-V9 | ILVRWIRWVIQW |
| 299 | 3002-V10 | ILVRWIRWRVQW |
| 300 | 3002-V11 | ILVRWIRWRIVW |
| 301 | 3002-V12 | ILVRWIRWRIQV |
| 302 | 3002-I2 | IIVRWIRWRIQW |
| 303 | 3002-I3 | ILIRWIRWRIQW |
| 304 | 3002-I4 | ILVIWIRWRIQW |
| 305 | 3002-I5 | ILVRIIRWRIQW |
| 306 | 3002-I7 | ILVRWIIWRIQW |
| 307 | 3002-I8 | ILVRWIRIRIQW |
| 308 | 3002-I9 | ILVRWIRWIIQW |
| 309 | 3002-I11 | ILVRWIRWRIIW |
| 310 | 3002-I12 | ILVRWIRWRIQI |
| 311 | 3002-L1 | LLVRWIRWRIQW |
| 312 | 3002-L3 | ILLRWIRWRIQW |
| 313 | 3002-L4 | ILVLWIRWRIQW |
| 314 | 3002-L5 | ILVRLIRWRIQW |
| 315 | 3002-L6 | ILVRWLRWRIQW |
| 316 | 3002-L7 | ILVRWILWRIQW |
| 317 | 3002-L8 | ILVRWIRLRIQW |
| 318 | 3002-L9 | ILVRWIRWLIQW |
| 319 | 3002-L10 | ILVRWIRWRLQW |
| 320 | 3002-L11 | ILVRWIRWRILW |
| 321 | 3002-L12 | ILVRWIRWRIQL |
| 322 | 3002-W1 | WLVRWIRWRIQW |
| 323 | 3002-W2 | IWVRWIRWRIQW |
| 324 | 3002-W3 | ILWRWIRWRIQW |
| 325 | 3002-W4 | ILVWWIRWRIQW |
| 326 | 3002-W6 | ILVRWWRWRIQW |
| 327 | 3002-W7 | ILVRWIWWRIQW |
| 328 | 3002-W9 | ILVRWIRWWIQW |
| 329 | 3002-W10 | ILVRWIRWRWQW |
| 330 | 3002-W11 | ILVRWIRWRIWW |
| 331 | 3002-Q1 | QLVRWIRWRIQW |
| 332 | 3002-Q2 | IQVRWIRWRIQW |
| 333 | 3002-Q3 | ILQRWIRWRIQW |
| 334 | 3002-Q4 | ILVQWIRWRIQW |
| 335 | 3002-Q5 | ILVRQIRWRIQW |
| 336 | 3002-Q6 | ILVRWQRWRIQW |
| 337 | 3002-Q7 | ILVRWIQWRIQW |
| 338 | 3002-Q8 | ILVRWIRQRIQW |
| 339 | 3002-Q9 | ILVRWIRWQIQW |
| 340 | 3002-Q10 | ILVRWIRWRQQW |
| 341 | 3002-Q12 | ILVRWIRWRIQQ |
| 342 | 3007-G1 | GLKIKVKIWVVK |
| 343 | 3007-G2 | VGKIKVKIWVVK |
| 344 | 3007-G3 | VLGIKVKIWVVK |
| 345 | 3007-G4 | VLKGKVKIWVVK |
| 346 | 3007-G5 | VLKIGVKIWVVK |
| 347 | 3007-G6 | VLKIKGKIWVVK |
| 348 | 3007-G7 | VLKIKVGIWVVK |
| 349 | 3007-G8 | VLKIKVKGWVVK |
| 350 | 3007-G9 | VLKIKVKIGVVK |
| 351 | 3007-G10 | VLKIKVKIWGVK |
| 352 | 3007-G11 | VLKIKVKIWVGK |
| 353 | 3007-G12 | VLKIKVKIWVVG |
| 354 | 3007-A1 | ALKIKVKIWVVK |
| 355 | 3007-A2 | VAKIKVKIWVVK |
| 356 | 3007-A3 | VLAIKVKIWVVK |
| 357 | 3007-A4 | VLKAKVKIWVVK |
| 358 | 3007-A5 | VLKIAVKIWVVK |
| 359 | 3007-A6 | VLKIKAKIWVVK |
| 360 | 3007-A7 | VLKIKVAIWVVK |
| 361 | 3007-A8 | VLKIKVKAWVVK |
| 362 | 3007-A9 | VLKIKVKIAVVK |
| 363 | 3007-A10 | VLKIKVKIWAVK |
| 364 | 3007-A11 | VLKIKVKIWVAK |
| 365 | 3007-A12 | VLKIKVKIWVVA |
| 366 | 3007-R1 | RLKIKVKIWVVK |
| 367 | 3007-R2 | VRKIKVKIWVVK |
| 368 | 3007-R3 | VLRIKVKIWVVK |
| 369 | 3007-R4 | VLKRKVKIWVVK |
| 370 | 3007-R5 | VLKIRVKIWVVK |
| 371 | 3007-R6 | VLKIKRKIWVVK |
| 372 | 3007-R7 | VLKIKVRIWVVK |
| 373 | 3007-R8 | VLKIKVKRWVVK |
| 374 | 3007-R9 | VLKIKVKIRVVK |
| 375 | 3007-R10 | VLKIKVKIWRVK |
| 376 | 3007-R11 | VLKIKVKIWVRK |
| 377 | 3007-R12 | VLKIKVKIWVVR |
| 378 | 3007-K1 | KLKIKVKIWVVK |
| 379 | 3007-K2 | VKKIKVKIWVVK |
| 380 | 3007-K4 | VLKKKVKIWVVK |
| 381 | 3007-K6 | VLKIKKKIWVVK |
| 382 | 3007-K8 | VLKIKVKKWVVK |
| 383 | 3007-K9 | VLKIKVKIKVVK |
| 384 | 3007-K10 | VLKIKVKIWKVK |
| 385 | 3007-K11 | VLKIKVKIWVKK |
| 386 | 3007-V2 | VVKIKVKIWVVK |
| 387 | 3007-V3 | VLVIKVKIWVVK |
| 388 | 3007-V4 | VLKVKVKIWVVK |
| 389 | 3007-V5 | VLKIVVKIWVVK |
| 390 | 3007-V7 | VLKIKVVIWVVK |
| 391 | 3007-V8 | VLKIKVKVWVVK |
| 392 | 3007-V9 | VLKIKVKIVVVK |
| 393 | 3007-V12 | VLKIKVKIWVVV |
| 394 | 3007-I1 | ILKIKVKIWVVK |
| 395 | 3007-I2 | VIKIKVKIWVVK |
| 396 | 3007-I3 | VLIIKVKIWVVK |
| 397 | 3007-I5 | VLKIIVKIWVVK |
| 398 | 3007-I6 | VLKIKIKIWVVK |
| 399 | 3007-I7 | VLKIKVIIWVVK |
| 400 | 3007-I9 | VLKIKVKIIVVK |
| 401 | 3007-I10 | VLKIKVKIWIVK |
| 402 | 3007-I11 | VLKIKVKIWVIK |
| 403 | 3007-I12 | VLKIKVKIWVVI |
| 404 | 3007-L1 | LLKIKVKIWVVK |
| 405 | 3007-L3 | VLLIKVKIWVVK |
| 406 | 3007-L4 | VLKLKVKIWVVK |
| 407 | 3007-L5 | VLKILVKIWVVK |
| 408 | 3007-L6 | VLKIKLKIWVVK |
| 409 | 3007-L7 | VLKIKVLIWVVK |
| 410 | 3007-L8 | VLKIKVKLWVVK |
| 411 | 3007-L9 | VLKIKVKILVVK |
| 412 | 3007-L10 | VLKIKVKIWLVK |
| 413 | 3007-L11 | VLKIKVKIWVLK |
| 414 | 3007-L12 | VLKIKVKIWVVL |
| 415 | 3007-W1 | WLKIKVKIWVVK |
| 416 | 3007-W2 | VWKIKVKIWVVK |
| 417 | 3007-W3 | VLWIKVKIWVVK |
| 418 | 3007-W4 | VLKWKVKIWVVK |
| 419 | 3007-W5 | VLKIWVKIWVVK |
| 420 | 3007-W6 | VLKIKWKIWVVK |
| 421 | 3007-W7 | VLKIKVWIWVVK |
| 422 | 3007-W8 | VLKTKVKWWVVK |
| 423 | 3007-W10 | VLKIKVKIWWVK |
| 424 | 3007-W11 | VLKIKVKIWVWK |
| 425 | 3007-W12 | VLKIKVKIWVVW |
| 426 | 3007-Q1 | QLKIKVKIWVVK |
| 427 | 3007-Q2 | VQKIKVKIWVVK |
| 428 | 3007-Q3 | VLQIKVKIWVVK |
| 429 | 3007-Q4 | VLKQKVKIWVVK |
| 430 | 3007-Q5 | VLKIQVKIWVVK |
| 431 | 3007-Q6 | VLKIKQKIWVVK |
| 432 | 3007-Q7 | VLKIKVQIWVVK |
| 433 | 3007-Q8 | VLKIKVKQWVVK |
| 434 | 3007-Q9 | VLKIKVKIQVVK |
| 435 | 3007-Q10 | VLKIKVKIWQVK |
| 436 | 3007-Q11 | VLKIKVKIWVQK |
| 437 | 3007-Q12 | VLKIKVKIWVVQ |

### Example 2: Computational Assessment of Active Peptides

Using a sequence optimization strategy that uses SPOT-synthesized peptide arrays to systematically and quantitatively measure the antibiofilm and immunomodulatory activities of synthetic peptides, we have generated 96 single amino acid variants of 1018, a synthetic peptide with potent antibiofilm activity, and measured the antibiofilm activity of all of these derivatives using a high-throughput crystal violet staining assay. Molecular descriptors (MDs) of all the 1018 derivatives were calculated and subsequently used to model the measured antibiofilm activity. The best QSAR models were then used to predict the antibiofilm activity of 100,000 virtual peptides in silico. A subset of the predicted sequences were then synthesized and tested for their antibiofilm activity to confirm the accuracy of the QSAR models.

Experimental Data Processing and Peptide Set Definitions. The activity data from the set of 96 single amino acid substituted peptides derived from peptide 1018, as well as 1018 itself (SEQ ID NO: 1, Table 2), were prepared for modeling purposes (described herein) and used as a Training Set for the initial quantitative structure activity relationship (QSAR) modelling. The experimental values were defined as the percent of MRSA biofilm inhibition which was determined as described in Example 5 and revealed in FIGS. 1A-B.

TABLE 2
Sequences of single amino acid substitution
variants of 1018 comprising the peptides that
were SPOT-synthesized and evaluated for their
antibiofilm activity against S. aureus.

| SEQ ID NO | Peptide name | Sequences |
|---|---|---|
| 438 | 1018 | VRLIVAVRIWRR |
| 439 | 1018-G1 | GRLIVAVRIWRR |
| 440 | 1018-G2 | VGLIVAVRIWRR |
| 441 | 1018-G3 | VRGIVAVRIWRR |
| 442 | 1018-G4 | VRLGVAVRIWRR |
| 443 | 1018-G5 | VRLIGAVRIWRR |
| 444 | 1018-G6 | VRLIVGVRIWRR |
| 445 | 1018-G7 | VRLIVAGRIWRR |
| 446 | 1018-G8 | VRLIVAVGIWRR |
| 447 | 1018-G9 | VRLIVAVRGWRR |
| 448 | 1018-G10 | VRLIVAVRIGRR |
| 449 | 1018-G11 | VRLIVAVRIWGR |
| 450 | 1018-G12 | VRLIVAVRIWRG |
| 451 | 1018-A1 | ARLIVAVRIWRR |
| 452 | 1018-A2 | VALIVAVRIWRR |
| 453 | 1018-A3 | VRAIVAVRIWRR |
| 454 | 1018-A4 | VRLAVAVRIWRR |
| 455 | 1018-A5 | VRLIAAVRIWRR |
| 456 | 1018-A7 | VRLIVAARIWRR |
| 457 | 1018-A8 | VRLIVAVAIWRR |
| 458 | 1018-A9 | VRLIVAVRAWRR |
| 459 | 1018-A10 | VRLIVAVRIARR |
| 460 | 1018-A11 | VRLIVAVRIWAR |
| 461 | 1018-A12 | VRLIVAVRIWRA |
| 462 | 1018-R1 | RRLIVAVRIWRR |
| 463 | 1018-R3 | VRRIVAVRIWRR |
| 464 | 1018-R4 | VRLRVAVRIWRR |
| 465 | 1018-R5 | VRLIRAVRIWRR |
| 466 | 1018-R6 | VRLIVRVRIWRR |
| 467 | 1018-R7 | VRLIVARRIWRR |
| 468 | 1018-R9 | VRLIVAVRRWRR |
| 469 | 1018-R10 | VRLIVAVRIRRR |
| 470 | 1018-K1 | KRLIVAVRIWRR |
| 471 | 1018-K2 | VKLIVAVRIWRR |
| 472 | 1018-K3 | VRKIVAVRIWRR |
| 473 | 1018-K4 | VRLKVAVRIWRR |
| 474 | 1018-K5 | VRLIKAVRIWRR |
| 475 | 1018-K6 | VRLIVKVRIWRR |
| 476 | 1018-K7 | VRLIVAKRIWRR |
| 477 | 1018-K8 | VRLIVAVKIWRR |
| 478 | 1018-K9 | VRLIVAVRKWRR |
| 479 | 1018-K10 | VRLIVAVRIKRR |
| 480 | 1018-K11 | VRLIVAVRIWKR |
| 481 | 1018-K12 | VRLIVAVRIWRK |
| 482 | 1018-L1 | LRLIVAVRIWRR |
| 483 | 1018-L2 | VLLIVAVRIWRR |
| 484 | 1018-L4 | VRLLVAVRIWRR |
| 485 | 1018-L5 | VRLILAVRIWRR |
| 486 | 1018-L6 | VRLIVLVRIWRR |
| 487 | 1018-L7 | VRLIVALRIWRR |
| 488 | 1018-L8 | VRLIVAVLIWRR |
| 489 | 1018-L9 | VRLIVAVRLWRR |
| 490 | 1018-L10 | VRLIVAVRILRR |
| 491 | 1018-L11 | VRLIVAVRIWLR |
| 492 | 1018-L12 | VRLIVAVRIWRL |
| 493 | 1018-I1 | IRLIVAVRIWRR |
| 494 | 1018-I2 | VILIVAVRIWRR |
| 495 | 1018-I3 | VRIIVAVRIWRR |
| 496 | 1018-I5 | VRLIIAVRIWRR |
| 497 | 1018-I6 | VRLIVIVRIWRR |
| 498 | 1018-I7 | VRLIVAIRIWRR |
| 499 | 1018-I8 | VRLIVAVIIWRR |
| 500 | 1018-I10 | VRLIVAVRIIRR |
| 501 | 1018-I11 | VRLIVAVRIWIR |
| 502 | 1018-I12 | VRLIVAVRIWRI |
| 503 | 1018-V2 | VVLIVAVRIWRR |
| 504 | 1018-V3 | VRVIVAVRIWRR |
| 505 | 1018-V4 | VRLVVAVRIWRR |
| 506 | 1018-V6 | VRLIVVVRIWRR |
| 507 | 1018-V8 | VRLIVAVVIWRR |
| 508 | 1018-V9 | VRLIVAVRVWRR |
| 509 | 1018-V10 | VRLIVAVRIVRR |
| 510 | 1018-V11 | VRLIVAVRIWVR |
| 511 | 1018-V12 | VRLIVAVRIWRV |
| 512 | 1018-W1 | WRLIVAVRIWRR |
| 513 | 1018-W2 | VWLIVAVRIWRR |
| 514 | 1018-W3 | VRWIVAVRIWRR |
| 515 | 1018-W4 | VRLWVAVRIWRR |
| 516 | 1018-W5 | VRLIWAVRIWRR |
| 517 | 1018-W6 | VRLIVWVRIWRR |
| 518 | 1018-W7 | VRLIVAWRIWRR |
| 519 | 1018-W8 | VRLIVAVWIWRR |
| 520 | 1018-W9 | VRLIVAVRWWRR |
| 521 | 1018-W11 | VRLIVAVRIWWR |
| 522 | 1018-W12 | VRLIVAVRIWRW |
| 523 | 1018-Q1 | QRLIVAVRIWRR |
| 524 | 1018-Q2 | VQLIVAVRIWRR |
| 525 | 1018-Q3 | VRQIVAVRIWRR |
| 526 | 1018-Q4 | VRLQVAVRIWRR |
| 527 | 1018-Q5 | VRLIQAVRIWRR |
| 528 | 1018-Q6 | VRLIVQVRIWRR |
| 529 | 1018-Q7 | VRLIVAQRIWRR |
| 530 | 1018-Q8 | VRLIVAVQIWRR |
| 531 | 1018-Q9 | VRLIVAVRQWRR |
| 532 | 1018-Q10 | VRLIVAVRIQRR |
| 533 | 1018-Q11 | VRLIVAVRIWQR |
| 534 | 1018-Q12 | VRLIVAVRIWRQ |

Additionally, a new set of 100,000 virtual peptides (referred to as the Virtual Set) were generated using a defined set of sequence constraints that would ensure that the Virtual Set sequences would have similar physicochemical characteristics to the parent peptide, 1018 (Table 3). All of the Virtual Set sequences were generated using custom a custom script within the Python environment and afterwards optimized using SVL scripts. Peptides conforming to this set were used as the test set to evaluate the in silico system's ability to predict new sequences.

TABLE 3
Peptide sequence constraints used to generate the 100,000 peptide
sequences comprising the Virtual Set of peptide sequences.

| Characteristic | Constraints |
|---|---|
| Peptide Length | 12 Residues |
| Amino acid composition | G, A, R, K, L, I, V, W, Q |
| Percent hydrophobic | 4 < or + (A + L + I + V + W) < or = |
| residues | 9 (33-75%) |
| Percent cationic residues | 2 < or = (R + K) < or = 6 (17-50%) |
| Hydrophobic Regions | No more than 4 hydrophobic amino acids |
| | together |
| Tryptophan | W < or = 3 |
| Glycine and Glutamine | G < or = 2, Q < or = 2, G + Q < or = 2 |
| Amino acid diversity | At least 5 different types of amino acids |

Molecular Descriptors Computation. Initially, the peptides sequences in the Training Set were saved as sdf files using the MOE software package (Molecular Operating Environment 2013.08. Chemical Computing Group Inc. Montreal, Canada). To accomplish the corresponding modeling steps, the peptide structures were optimized using a custom SVL script (supplementary materials). MDs for the peptides in the Training Set were calculated using MOE 2013.08 and Dragon 6.0 software (TALETE srl. 2011. Milano, Italy). Additionally, inductive QSAR MDs were computed in this study (Cherkasov, A R, VI Galkin, and RA Cherkasov. 1998. A New approach to the theoretical estimation of inductive constants. J. Phys. Org. Chem. 11:437-47.; Cherkasov, A. 2003. Inductive electronegativity scale. Iterative calculation of inductive partial charges. J. Chem. Inf. Comput. Sci. 43:2039-47.; Cherkasov, A. 2005. Inductive descriptors: 10 successful years in QSAR. Curr. Comput. Aided-Drug Des. 1:21-42). All these MDs have been successfully applied in chemoinformatics studies related to antimicrobial peptides (Cherkasov et al. 2009) and other therapeutic areas (Baldi, P et al. 2000. Assessing the accuracy of prediction algorithms for classification: an overview. Bioinformatics 16:412-424). A list of the Molecular Descriptors used in the QSAR models to define the antibiofilm activity of synthetic peptides is found in Table 4.

TABLE 4
Molecular Descriptors used in the QSAR models
to define the activity of synthetic peptides.

| Names | Family | Description |
|---|---|---|
| TDB02s | 3D autocorrelations | 3D Topological distance based |
| | | descriptors - lag 2 weighted |
| | | by I-state |
| RDF040v | RDF descriptors | Radial Distribution Function - |
| | | 040/weighted by van der Waals |
| | | volume |
| RDF130s | RDF descriptors | Radial Distribution Function - |
| | | 130/weighted by I-state |
| Mor09v | 3D-MoRSE descriptors | signal 09/weighted by |
| | | van der Waals volume |
| Mor02s | 3D-MoRSE descriptors | signal 02/weighted by I-state |
| Mor06s | 3D-MoRSE descriptors | signal 06/weighted by I-state |
| HATS0s | GETAWAY descriptors | leverage-weighted autocorrelation |
| | | of lag 0/weighted by I-state |

In total, more than 2,500 MDs were calculated for all the peptides in the Training Set. The calculated MDs were then filtered to exclude those with zero variance and low occurrence (MDs represented by less than 24% of compounds). Also, MDs with correlation coefficient of 1.0 between each other were eliminated. The remaining MDs were tested on their ability to classify the peptides into active or inactive based on a threshold value (see below). The seven MDs identified in the final classifier were calculated for the peptides in the Virtual Set in the same manner as those described for the Training Set.

Statistical Analysis and Data Modelling. To obtain binary predictions, the experimental values for the Training Set were used and different threshold values of antibiofilm potency were explored ranging from the top 5 to top 20% of the ranked 1018-derived peptides. The dependent variable was then assigned a value of 1 or −1 when the peptide had greater or lower experimental value than the threshold, respectively. Statistical parameters like the ‘hit rate’ and fprate were checked for each classification model. Statistical analysis was carried out with STATISTICA version 10.0 (StatSoft Inc. Tulsa, Okla. USA) and Linear Discriminant Analysis (LDA) was used to find the classifier functions. The forward stepwise and best subset methods were employed for the attribute selection. The tolerance parameter was set to 0.01. By using the models, one compound could be classified as either active if ΔP %>0 (being ΔP %=[P (Active)−P (Inactive)]×100), otherwise the compound was deemed inactive. P (active) and P (inactive) are the probabilities with which the equations classify a compound as active and inactive, respectively. The quality of the models was determined according to Wilks' λ, the square of the Mahalanobis distance D2, Fisher ratio (F), significance level (p) and the percentage of good classification (accuracy, Q). Therefore, parameters like sensitivity ‘hit rate’ (SE), specificity (SP), false positive rate (fprate) and Matthews' correlation coefficient (MCC) were taken into account23. Those models with high statistical significance but having as few MDs as possible were preferred. Additionally, 10-fold cross-validation was performed on the final set using the top 5% as the optimum threshold value. Briefly, to perform the cross-validation procedure, 10% of the peptides in the Training Set were randomly selected as validation data set while the rest of the peptides were used as a corresponding Training Set. This was repeated a total of 10 times resulting in 10 validation sets and 10 Training Sets created.

Example 11 shows the computationally calculated activity rankings of a subset of the the QSAR peptides.

### Example 3: Computational Testing and In Vitro Screening of Novel Effective Optimized Peptides

In Silico Testing and in vitro Screening of Optimized Peptides. In order to test the predictive accuracy of the proposed models, all the peptides in the Virtual Set were tested in silico and the combined predictions were ranked together into a single list according to their probability of being active or inactive. A set of 108 peptides (SEQ ID No 24-73 and C1-C57 listed in Tables 1 and 7) from the 100,000 peptide Virtual Set were chosen to evaluate the system's capability to distinguish active from inactive optimized sequences. This Experimental Validation Set included 55 peptides in the top 10% of predicted antibiofilm sequences, 20 sequences from the bottom 20% of predicted sequences and the remaining 33 peptides distributed in the remaining middle 70%. The 108 peptides comprising the ES were SPOT-synthesized and their antibiofilm activity was evaluated against MRSA using the crystal violet assay described in Example 5 and illustrated in FIG. 2 while their immunomodulatory activity is described in Examples 8 and 9. The testing demonstrated that the in silico rankings mirrored the measured activities.

### Example 4: Anti-Biofilm Activity

Following computational ranking of the top antibiofilm peptides in the Virtual Set, a sampling of 108 peptides (the Experimental Validation Set) with varying predicted potency against biofilms were SPOT-synthesized and their antibiofilm activity was experimentally determined (see herein).

Methods of assessment of anti-biofilm activity: MRSA S. aureus strain SAP0017 biofilm formation was initially analyzed using a static abiotic solid surface assay as described elsewhere (de la Fuente-Nunez et al., 2012) and shown graphically in FIG. 2. Dilutions (1/100) of overnight cultures were incubated in BM2 biofilm-adjusted medium [62 mM potassium phosphate buffer (pH 7), 7 mM (NH4)2SO4, 2 mM MgSO4, 10 μM FeSO4, 0.4% (wt/vol) glucose, 0.5% (wt/vol) Casamino Acids], or a nutrient rich medium such as Tryptic soy broth supplemented with 1% glucose, in polypropylene microtiter plates (Falcon, United States) in the absence (control) or presence of peptide. Peptide was added at time zero (prior to adding the diluted, overnight cultures) in varying concentrations, and the amount biofilm formation was recorded after 22-46 h incubation for most bacteria. To quantify biofilm growth, planktonic cells were removed and biofilm cells adhering to the side of the wells were stained with crystal violet, and absorbance at 595 nm was measured using a microtiter plate reader (Bio-Tek Instruments Inc., United States).

Antibiofilm activity: As can be seen in FIGS. 2 and 3A-B and Tables 5 and 7, screening of a series of peptides derived by computational predictions from peptide IDR-1018 indicated clearly that peptides differed widely in their activity as also revealed through computational analysis in Example 11. Peptides ranged from very active to inactive and the most active peptides were clearly superior to previously investigated peptides such as 1037 (de la Fuente-Nunez et al, 2011) and 1018. Many single amino acid substitution peptides showed similar or improved activities, compared to their parent sequences (FIGS. 1A-B). This data set was used to generate QSAR models as described above that predicted many highly active anti-biofilm peptides e.g. in Table 5 and Example 11. Additional characterization of 1018 derivatives is shown in FIGS. 12A-B, 13A-D, and 14A-C.

To validate the antibiofilm activity of the most active peptides in the Experimental Validation Set, the seven most active peptides (Peptides 3001-3007, SEQ ID NOs. 24-30) from this peptide set were chemically synthesized to >95% purity and the antibiofilm activity of these pure peptide samples was assessed. The sequences and antibiofilm activity of these seven QSAR optimized antibiofilm peptides are shown in Table 5 and data concerning these peptides are found in FIGS. 3A-B, 4, 5A-B, and 6A-D. When evaluated in the static microtitre plate assay, most of the new antibiofilm peptides exhibited antibiofilm activity similar to or better than 1018 (FIG. 3A). Peptide 3002 exhibited an enhanced ability to inhibit MRSA biofilm formation compared to 1018. 3002 strongly inhibited biofilm growth at a concentration of 1 μM, which represents an 8-fold improvement on the antibiofilm potency compared to 1018 (FIG. 3A). Antibiofilm activity of select synthetic peptides against pre-formed P. aeruginosa PAO1 biofilms is shown in FIGS. 16A-L.

TABLE 5
Screening of QSAR derived optimized peptides and
cationic amino acid substituted 1018 derivatives
for enhanced antibiofilm activity. All peptides
were SPOT synthesized on cellulose membranes and
resuspended in water. The antibiofilm activity
was evaluated against a clinical MRSA strain
using the crystal violet assay at a peptide
concentration of ~12.5 μM. Any peptide that
reduced biofilm growth by 60% or more compared
to control is highlighted in bold. Other peptide
activites are described in Examples 8 and 9.

| | | % Residual MRSA |
|---|---|---|
| | | biofilm growth |
| | | compared to |
| Peptide name | Sequence | untreated control |
| 1018 | VRLIVAVRIWRR | 37 |
| 1018-k36 | VRLIVAVRIWROrn | 20 |
| 1018-k1 | DapRLIVAVRIWRR | 23 |
| 1018-r36 | VRLIVAVRIWRHar | 27 |
| 1018-k30 | VRLIVOrnVRIWRR | 27 |
| 1018-k12 | VRLIVAVRIWRDap | 30 |
| 1018-r35 | VRLIVAVRIWHarR | 33 |
| 1018-k35 | VRLIVAVRIWOrnR | 34 |
| 1018-k11 | VRLIVAVRIWDapR | 44 |
| 1018-k25 | OrnRLIVAVRIWRR | 44 |
| 1018-r32 | VRLIVAVHarIWRR | 45 |
| 1018-k32 | VRLIVAVOrnIWRR | 46 |
| 1018-r13 | GbutRLIVAVRIWRR | 47 |
| 1018-k10 | VRLIVAVRIDapRR | 49 |
| 1018-r1 | GproRLIVAVRIWRR | 50 |
| 1018-r30 | VRLIVHarVRIWRR | 57 |
| 1018-k13 | DabRLIVAVRIWRR | 59 |
| 1018-k26 | VOrnLIVAVRIWRR | 63 |
| 1018-r25 | HarRLIVAVRIWRR | 64 |
| 3001 | VIKWLLKILRAI | 2 |
| 3002 | ILVRWIRWRIQW | 7 |
| 3003 | WKKVQWLKRLLL | 19 |
| 3004 | IQRWWKVWLKVI | 29 |
| 3005 | RRQWRGWVRIWL | 33 |
| 3006 | IWLRLKVVLKRK | 35 |
| 3007 | VLKIKVKIWVVK | 42 |
| 3008 | KKWQLLIKWKLR | 49 |
| 3009 | AVAKWALKLWKQ | 102 |
| 3010 | QLARLARVVWGL | 102 |
| 3011 | VLQIKKVLRLLL | 45 |
| 3012 | RVKAIKWRKIVV | 77 |
| 3013 | LWQLWLKLKLKG | 76 |
| 3014 | KIQRRAWKQWRK | 98 |
| 3015 | KIVIRIILQVIK | 102 |
| 3016 | AVKWLGWILAKK | 102 |
| 3017 | LAGLIVKWAGVR | 100 |
| 3018 | WVGVIIKWGLKL | 100 |
| 3019 | WQGWAKIWVVRI | 100 |
| 3020 | LIVIQLLKKWWK | 60 |
| 3021 | RRIIKILLWKLR | 70 |
| 3022 | IAWQLLWGWRVR | 96 |
| 3023 | VQRIIWLRVKIV | 98 |
| 3024 | IKIIWKALGQVI | 100 |
| MILBFmax5 | IQLKLIWVKRKW | 43 |
| BFmax-9 | VIKVLIKRWLKL | 58 |
| BFmax-6 | VQWIQIVVWRKR | 57 |
| IBFmax-15 | GLIIKIIKKRLW | 59 |
| Imax-5 | VKGAIKRGIWVK | 99 |
| BFmax-7 | KVQIIKQLIAKK | 100 |
| BFmax-16 | KRLQWVKVKKIR | 99 |
| Imax-10 | IVKWIAQWKLVG | 100 |
| Mmax-16 | KKQKKIWRRILV | 105 |
| Mmax-3 | GRVLKIVWRKGR | 101 |
| Mmax-18 | RQVRVKRWRARW | 100 |
| BFmax-2 | KVVWWKVIIKVL | 93 |
| Imax-7 | ALAIKVWIKILQ | 100 |
| MILBFmax-9 | IRILVLRKAIIV | 101 |
| MILBFmax-13 | IVKKVKLIWGVK | 98 |
| Imin-3 | VVGLRVRWVRLW | 96 |
| Imax-20 | WAVRALKVKWAL | 98 |
| IBFmax-13 | WWIKIVVIRVRR | 81 |
| MILBFmax-8 | QIIKVVWRAVII | 101 |
| IBFmax-11 | QQVKWWLIRWLA | 93 |
| IBFmax-17 | IKWVLRKIVQII | 74 |
| BFmax-5 | VARWKIIIAKLW | 98 |
| Imax-6 | KIWGLLKLGIAL | 99 |
| Imin-4 | RARQIRWLRKRV | 102 |
| IBFmax-8 | RVLIKWKKVIVV | 101 |
| MILBFmax-1 | LKLKAILKIIRV | 95 |

Biofilms were cultivated for 72 h in the presence of 2-20 μg/mL of peptides at 37° C. in flow chambers with channel dimensions of 1×4×40 mm, as previously described but with minor modifications. Silicone tubing (VWR, 0.062 ID×0.125 OD×0.032 wall) was autoclaved and the system was assembled and sterilized by pumping a 0.5% hypochlorite solution through the system at 6 rpm for 1 hour using a Watson Marlow 205S peristaltic pump. The system was then rinsed at 6 rpm with sterile water and medium for 30 min each. Flow chambers were inoculated by injecting 400 μl of mid-log culture diluted to an OD₆₀₀ of 0.02 with a syringe. After inoculation, chambers were left without flow for 2 h after which medium was pumped though the system at a constant rate of 0.75 rpm (3.6 ml/h). Microscopy was done with a Leica DMI 4000 B widefield fluorescence microscope equipped with filter sets for monitoring of blue [Excitation (Ex) 390/40, Emission (Em) 455/50], green (Ex 490/20, Em 525/36), red (Ex 555/25, Em 605/52) and far red (Ex 645/30, Em 705/72) fluorescence, using the Quorum Angstrom Optigrid (MetaMorph) acquisition software. Images were obtained with a 63×1.4 objective. Deconvolution was done with Huygens Essential (Scientific Volume Imaging B.V.) and 3D reconstructions were generated using the Imaris software package (Bitplane AG).

To confirm the results from the crystal violet staining assay, MRSA biofilms were grown for two days in flow cells and then treated with 3002 or 1018. Biofilms grown in flow cells are generally considered to be a better model of biofilm growth since the bacteria were allowed to adhere to the surface of the flow cell chamber and mature into biofilms as fresh growth media is passed through the flow cell chamber. In agreement with the static microtitre plate assays, 3002 exhibited potent antibiofilm activity against 2-day old MRSA biofilms, effectively eradicating the biofilms at a peptide concentration of 0.125 μM (FIG. 3B). In comparison, biofilms treated with 0.125 μM 1018 were virtually identical to untreated controls (FIG. 3B). This dramatic improvement in antibiofilm potency of 3002 compared to 1018 demonstrates that not only can QSAR modeling of peptides be used to accurately identify novel antibiofilm sequences but it could potentially be used to significantly improve the potency of next generation antibiofilm peptides with enhanced therapeutic potential.

We have also observed activity for 1018 (peptide SEQ ID NO: 1) against multiple multidrug resistant isolates of many Gram negative and Gram positive including MDR strains of Pseudomonas aeruginosa and Acinetobacter baumannii, carbapenemase expressing Klebsiella pneumoniae, Enterobacter cloacae with de-repressed chromosomal β-lactamase, and vancomycin resistant Enterococcus, in addition to activity vs. oral biofilms formed on hydroxyapatite disks. This teaches that these peptides will show similar broad spectrum activity.

Similarly non-natural amino acid substitution peptides of 1018, as described in SEQ ID NO 6-23 and 74-79, maintained anti-biofilm activity while having improved protease resistance.

We also designed D amino acid equivalents that were predicted to have equivalent or improved anti-biofilm activity (SEQ ID NO: 146-245).

Peptides array methods were also utilized to design double substituted derivatives of the previously demonstrated protease-resistant active peptides RI-1018, DJK-5 and DJK-6 (de la Fuente-Nunez et al, 2015. Chemistry and Biology 22:196-205), to design D-amino acid containing peptides with two favourable amino acid substitutions (SEQ ID NO: 80-145) that are likely to have immunomodulatory activity.

Investigation of the anti-biofilm activity of SPOT-synthesized single amino acid substitution variants of peptide 3002 and 3007 (FIGS. 17A and B, respectively) indicated that, for peptide 3002 (FIG. 17A), amino acid substitutions are widely tolerated at positions 1, 2, and 11. Cationic residues (R and K) are preferred at positions 7 and 9 as well as marginally preferred at position 4 (although G and W are acceptable at this position as well). Hydrophobic residues are preferred at positions 3, 6, 8, 10 and 11. The W at position 8 can only be substituted for L or A to retain appreciable activity. For peptide 3007 (FIG. 17B), amino acid substitutions are widely tolerated at positions 1, 11 and 12. Cationic residues (R and K) are preferred at positions 3, 5 and 7 with Q, G and A residues tolerated at positions 5 and 7 as well. Hydrophobic residues are preferred at positions 2, 4, 8, and 10. The V at position 6 can only be substituted for an A residues while the W at position 9 can be substituted for Q, A or L to retain appreciable activity similar to the parent sequence. Substitution resulting in a greater than 15% improvement in antibiofilm activity (0.85 or lower) include K2, W6, L7, L10, I12, L12 and V12 substitutions for peptide 3002 (FIG. 18A). For peptide 3007, improved variants include K1, G1, W1, W2, V4, A6, A9 and R11 substitutions (FIG. 18B).

In addition, D- and RI-forms of peptides 3001-3007 were SPOT-synthesized on peptide arrays and screened for their ability to inhibit MRSA (C623) and P. aeruginosa (PAO1) biofilms in a static microtitre plate assay. Purified (>95%) L-forms of each peptide were run for comparison as well as 1018 and RI-1018. The D- and RI-forms of 3006 and 3007 exhibited the best antibiofilm activity against MRSA and PAO1 under these conditions. The hemolytic activity of the SPOT-peptides as well as purified L-forms was assessed in vitro against red blood cells isolated from healthy volunteers. All MRSA experiments were carried out in 10% tryptic soy broth supplemented with 0.1% glucose while PAO1 biofilms were grown in BM2 minimal media. the D and RI forms of 3006 and 3007 exhibited good antibiofilm activity towards both S. aureus and P. aeruginosa (FIGS. 19A-N) and were not hemolytic (FIGS. 20A-G). Consistent with the screening results, D- and RI-forms of 3006 and 3007 exhibited broad spectrum biofilm inhibition activity in vitro better than the corresponding L-forms with D-3006 and D-3007 being the most active under the conditions evaluated (Table 17). S. aureus biofilms were grown in 10% tryptic soy broth supplemented with 0.1% glucose while all other bacteria were grown in BM2 minimal media (62 mM potassium phosphate, 7 mM ammonium sulphate, 0.5 mM magnesium sulphate, 0.4% glucose, pH 7.0).

TABLE 17
Biofilm inhibition activity of L-, D- and RI- forms of peptide
1018, 3006 and 3007 against S. aureus (methicillin resistant
clinical isolate C623) E. coli O157:H7, P. aeruginosa (PAO1)
and Salmonella typhimurium (ATCC 14028). Values shown are
the peptide concentration (μM) that inhibited more than
90% of adhered biofilm biomass, quantified by crystal violet
staining, in a static microtitre plate assay

| | S. aureus | E. coli | P. aeruginosa | S. typhimurium |
|---|---|---|---|---|
| Peptide | (C623) | (O157:H7) | (PAO1) | (ATCC 14028) |
| RI-3007 | 16 | 16 | 32 | 16 |
| D-3007 | 8 | 4 | 8 | 4 |
| L-3007 | 64 | 8 | >32 | 16 |
| RI-3006 | 8 | 4 | 4 | 4 |
| D-3006 | 4 | 2 | 2 | 2 |
| L-3006 | 8 | 16 | 4 | 4 |
| RI-1018 | 8 | 1 | 4 | 2 |
| D-1018 | 64 | 16 | 32 | 16 |
| L-1018 | 32 | 1 | 4 | 1 |
| HE1 | NA | 1 | 4 | 2 |

The following strains were assessed vs. Staphylococcus epidermidis (Se), Pseudomonas aeruginosa (Pa), Streptococcus dysgalactiae (Sd), Pasteurella multocida (Pm), Streptococcus agalactiae (Sa), Streptococcus uberis (Su), Streptococcus suis (Ss), Mannheimia haemolytica (Mh), Bordatella bronchiseptica (Bb), Histophilus somnus (Hs), and Staphylococcus pseudintermedius (Sp) (Table 18) for Minimal Inhibitory Concentrations (MIC). Italics=excellent activity (less than or equal to 4 μg/ml). Peptide 3013 exhibited the most antibacterial effects against 6 of the 13 bacterial strains evaluated. The rest of the peptides were largely inactive.

TABLE 18
Minimal inhibitory concentrations of synthetic peptides
against various pathogenic strains of bacteria.

| | MIC (μg/ml) | | | | | | | | | | | | |
|---|---|---|---|---|---|---|---|---|---|---|---|---|---|
| Peptide | Se | Pa | Sd | Pm | Sa | Su | Ss | Mh | Bb | Hs | Sp | Ec | Sau |
| 3009 | >64 | >64 | 32 | >64 | >64 | 32 | >64 | >64 | >64 | 64 | 4 | >64 | >64 |
| 3010 | >64 | >64 | 32 | >64 | >64 | 64 | >64 | >64 | >64 | 64 | 64 | >64 | >64 |
| 3013 | 2 | >64 | 2 | 32 | 2 | 4 | 64 | >64 | 16 | 2 | <1 | >64 | >64 |
| 3015 | >64 | 64 | 32 | 64 | >64 | 64 | 64 | >64 | 64 | >64 | 16 | >64 | >64 |
| 3016 | >64 | >64 | >64 | >64 | 8 | 16 | 32 | >64 | 64 | 16 | 8 | 64 | >64 |
| 3017 | >64 | >64 | >64 | >64 | 32 | 64 | >64 | >64 | >64 | >64 | >64 | >64 | >64 |
| DJK5 | 2 | 4 | <1 | 8 | 2 | <1 | <1 | 8 | 4 | 32 | <1 | <1 | 2 |

The same strains as in Table 18 were assessed for Minimal Biofilm Inhibitory Concentrations (MBIC) (Table 19). Italics=excellent activity (less than or equal to 4 μg/ml). ND=Not determined. Peptides 3013, 3015 and 3016 inhibited biofilm growth in the largest number of bacterial strains (5, 5 and 6 out of 13 strains, respectively).

TABLE 19
Minimum biofilm inhibitory concentration (MBIC) of synthetic
peptides against various bacterial strains.

| | MBIC (μg/ml) | | | | | | | | | | | | |
|---|---|---|---|---|---|---|---|---|---|---|---|---|---|
| Peptide | Se | Pa | Sd | Pm | Sa | Su | Ss | Mh | Bb | Hs | Sp | Ec | Sau |
| 3009 | >64 | >64 | 16 | 16 | >64 | 16 | 16 | ND | 64 | 16 | 8 | 32 | 64 |
| 3010 | >64 | >64 | 16 | 4 | >64 | 32 | >64 | >64 | >64 | 64 | 64 | 32 | 64 |
| 3013 | >64 | >64 | 2 | 2 | 32 | 8 | 16 | >64 | >64 | 4 | 1 | >64 | 32 |
| 3015 | 2 | 32 | 4 | 1 | >64 | 4 | 64 | 16 | >64 | 16 | 8 | 64 | 64 |
| 3016 | 4 | 32 | >64 | 8 | 64 | 8 | 32 | >64 | 64 | 4 | 8 | 32 | 4 |
| 3017 | >64 | >64 | >64 | >64 | >64 | 16 | >64 | >64 | >64 | 32 | 64 | >64 | 4 |
| DJK5 | 2 | 8 | 2 | 2 | 2 | 2 | 4 | 4 | 2 | <1 | <1 | <1 | <1 |

In addition, several peptides expressed preferential activity vs. biofilms (MBIC) cf. planktonic cells (MIC) (Table 20).

TABLE 20
Synthetic peptides screened for activity vs. E. coli and
S. aureus planktonic cells (MIC) and biofilms (MBIC).
Italics = excellent activity (less than or equal to 4 μg/ml).

| | MIC | MIC | MBIC | MBIC |
|---|---|---|---|---|
| Peptides | E. coli | S. aureus | E. coli | S. aureus |
| DJK5 | <1 | 2 | <1 | <1 |
| 3001 | 4 | 2 | 2 | <1 |
| 3002 | 8 | 8 | 4 | 4 |
| 3003 | 4 | 16 | 2 | 1 |
| 3004 | 4 | 8 | 2 | 1 |
| 3005 | 4 | 8 | 2 | 1 |
| 3006 | 2 | 8 | 1 | 1 |
| 3007 | 8 | 16 | 4 | 2 |
| 3008 | 8 | 8 | 4 | 2 |
| 3009 | >64 | >64 | 32 | 64 |
| 3010 | >64 | >64 | 32 | 64 |
| 3011 | 8 | 64 | 4 | 2 |
| 3012 | 64 | >64 | 32 | 8 |
| 3013 | >64 | >64 | >64 | 32 |
| 3014 | 64 | >64 | 64 | 64 |
| 3015 | >64 | >64 | 64 | 64 |
| 3016 | 64 | >64 | 32 | 4 |
| 3017 | >64 | >64 | >64 | 4 |
| 3018 | 4 | 2 | 2 | 1 |
| 3019 | 4 | 4 | 2 | 2 |
| 3020 | 2 | 16 | 1 | <1 |
| 3021 | <1 | 8 | 2 | 1 |
| 3022 | 8 | 8 | 4 | 4 |
| 3023 | 2 | 4 | 1 | 2 |
| 3024 | 4 | 16 | 2 | 8 |

### Example 5: Controlling Aggregation

Previous studies have demonstrated that peptides tend to self-assemble through the interactions of their hydrophobic region(s) (Payne R W, and MC Manning. 2009. Peptide formulation: challenges and strategies. Innovations in Pharmacological Technology 28:64-68.). This property has also been observed by us for IDR-1018 and we have observed that the degree of aggregation is solvent and concentration dependent. Although IDR-1018 aggregation is commonly observed under cell culture conditions, the basis for peptide self-assembly is not well understood but appears to be related to the amphipathic nature of IDR-1018 and perhaps the stretch of 5 consecutive hydrophobic amino acids in the sequence. One method that can overcome aggregation is to utilize pharmaceutically-valuable excipients that can successfully prevent IDR-1018 aggregation and enhance the activities of IDR-1018 while exhibiting low cytotoxicity. Exemplary formulations are discussed herein.

A second method is to change the sequence of 1018 such that it loses or diminishes the property of aggregation but retains activity. We tested a subset of the peptides for aggregation in the presence of phosphate buffer which causes progressive aggregation of peptide 1018. Peptides 3001-3007 caused considerably lower aggregation than 1018 (FIG. 4), while retaining anti-biofilm activity (FIGS. 3A-B). In particular, peptides 3002, 3003, and 3004 showed almost no aggregation when added to phosphate buffer and showed superior and/or equivalent anti-biofilm activity compared to peptide 1018.

### Example 6: Animal Models

To confirm the potential utility of these peptides in treating infections we have utilized a new model to determine the efficacy of peptides. Recent studies have shown that certain synthetic peptides target the stringent response as the basis for their broad-spectrum anti-biofilm activity (de la Fuente-Nunez 2014, 2015). The stringent response is a conserved stress response employed by various bacteria to respond and cope with conditions of amino-acid starvation, carbon-source, fatty acid, oxygen or iron limitation, iron limitation, heat shock, fatty acid limitation, antimicrobial challenge, and other environmental stressors (Potrykus K and M Cashel. 2008. (p)ppGpp: Still Magical? Annual Review of Microbiology 62: 35-51). In many bacteria, the stringent response is signaled by secondary-messenger molecules guanosine tetratetraphosphate (ppGpp; its precursor is guanosine pentaphosphate) which serves as a pleiotropic transcriptional regulator by binding to RNA polymerase. This leads to the repression of resource-consuming processes (translation, lipid, and cell wall biosynthesis, and to some extent replication, and transcription and translation) and diverts resources towards biosynthesis (amino acid biosynthesis and transport, glycolysis and diverse stress genes) to ensure survival. Importantly, the stringent response and biofilm formation are tightly interconnected processes. As and (p)ppGpp is required for biofilm initiation and maintenance, since bacterial mutants defective in the stringent response, are also incapable of forming biofilms (de la Fuente-Nunez et al. 2014).

We found that the stringent response was crucial for Staphylococcus aureus skin cutaneous abscess formation in mice and because of this certain peptides used as controls here were able to reduce abscess lesion formation but had only modest effects on bacterial counts [Mansour, S. C., D. Pletzer, C. de la Fuente-Núñez, P. Kim, G. Y. C. Cheung, H.-S. Joo, M. Otto and R. E. W. Hancock. 2016. Bacterial abscess formation is controlled by the stringent stress response and can be targeted therapeutically. eBiomedicine 12:219-226].

We assessed the activity of peptide 3002 against abscess infections by the Gram negative bacterium Pseudomonas aeruginosa (FIGS. 5A-B) or MRSA (FIGS. 15A-C). This peptide was able to visibly reduce tissue injury and dermonecrosis by reducing the size of abscesses for this bacterium, compared to controls, and like other active peptides had little effect on viable bacterial counts viable bacterial counts. The peptide worked via intraperitoneal or intra-abscess (subcutaneous) injection. Taken together, these results show that the anti-biofilm peptides described herein may be effective in animal models and treatment and exhibit broad-spectrum activity vs bacterial abscesses and biofilm infections.

### Example 7: Enhancement of Innate Immunity

We tested if the novel peptides described herein had the ability to induce MCP-1 chemokine production in human peripheral blood mononuclear cells.

Venous blood (20 ml) from healthy volunteers was collected in Vacutainer® collection tubes containing sodium heparin as an anticoagulant (Becton Dickinson, Mississauga, ON) in accordance with UBC ethical approval and guidelines. Blood was diluted 1:1 with complete RPMI 1640 medium and separated by centrifugation over a Ficoll-Paque® Plus (Amersham Biosciences, Piscataway, N.J., USA) density gradient. White blood cells were isolated from the buffy coat, washed twice in PBS and then resuspended in RPMI 1640 complete medium (containing 10% fetal bovine serum), and the number of peripheral blood mononuclear cells (PBMC) was determined by Trypan blue exclusion. PBMCs (5×10⁵) were seeded into 12-well tissue culture dishes (Falcon; Becton Dickinson) at 0.75-1×10⁶ cells/ml at 37° C. in 5% CO₂. The above conditions were chosen to mimic conditions for circulating blood monocytes entering tissues at the site of infection via extravasation.

Following incubation of the cells under various treatment regimens, the tissue culture supernatants were centrifuged at 1000×g for 5 min, then at 10,000×g for 2 min to obtain cell-free samples. Supernatants were aliquoted and then stored at −20° C. prior to assay for various chemokines by capture ELISA (eBioscience and BioSource International Inc., CA, USA respectively)

The top antibiofilm peptides identified by the QSAR models (Peptides 3001-3007) were evaluated for their cytotoxic effects on PBMCs and red blood cells as well as their abilities to induce MCP1 from PBMCs and suppress LPS-induced IL-1B production from PBMCs (FIG. 6).

The 1018 single amino acid substitution derivatives (Table 2) that were SPOT-synthesized on cellulose membranes were tested for their ability to induce MCP1 production from PBMCs as well as suppress LPS-induced IL-1β pro-inflammatory cytokine production (FIGS. 7A-B). This data set was used to establish QSAR models for both activity types (chemokine induction and anti-inflammatory activity) in a similar fashion as the antibiofilm models described in Example 4.

The same Experimental Validation Set containing peptides that were predicted to be most active based on the QSAR models (SEQ ID NO: 24-73 cf predicted less active peptides C1-C57) were SPOT-synthesized and their biological activities were evaluated in vitro as described herein.

As shown in Table 6, most of the QSAR derived peptides in the Experimental Validation Det stimulated the expression of the macrophage chemokine MCP-1 at a concentration of ˜25 μM (˜40 μg/ml) and 27 of these peptides were superior to 1018 by up to 10-fold (Table 6). This was a dramatic improvement in activity compared to QSAR predicted inactive or weakly active peptides (Table 7).

TABLE 6
Screening of QSAR derived optimized peptides for enhanced
immunomodulatory activity. All peptides were SPOT synthesized on cellulose
membranes and resuspended in water. The peptides were also screened against
PBMCs from 3 separate human donors for immunomodulatory activity and
toxicity at a concentration of ~25 μM. MCP1 chemokine induction by peptide
alone was measured and any sequence that led to substantial increase in
MCP1 induction (>2000 pg/ml) are highlighted in bold. The ability of peptides
to suppress the production of the pro-inflammatory cytokine IL1β from LPS
stimulated cells was also quantified and any sequence that strongly
suppressed cytokine production (Fold change >0.75) is highlighted in bold.
Finally, peptide induced cytotoxicity was measured by the lactate
dehydrogenase (LDH) assay and any peptide with strong toxicity
(>20% LDH release) is highlighted in bold.

| | | MCP1 | Fold Change in | % |
|---|---|---|---|---|
| | | production in | IL1-β relative to | Toxicity |
| | | PBMC | untreated LPS- | (LDH |
| Peptide Name | Sequence | (pg/ml) | stimulated cells | Release) |
| 1018 | VRLIVAVRIWRR | 1974 | 0.92 | 21.8 |
| 3001 | VIKWLLKILRAI | 5935 | 2.27 | 64.4 |
| 3002 | ILVRWIRWRIQW | 1152 | 0.64 | −3.7 |
| 3003 | WKKVQWLKRLLL | 12298 | 0.96 | 22.5 |
| 3004 | IQRWWKVWLKVI | 1287 | 0.64 | 5.1 |
| 3005 | RRQWRGWVRIWL | 4294 | 1.08 | −4.2 |
| 3006 | IWLRLKVVLKRK | 3687 | 1.23 | 2.4 |
| 3007 | VLKIKVKIWVVK | 3276 | 1.13 | 2 |
| 3008 | KKWQLLIKWKLR | 24444 | 2.23 | 18.8 |
| 3009 | AVAKWALKLWKQ | 8635 | 0.75 | −3.2 |
| 3010 | QLARLARVVWGL | 7767 | 0.85 | −2.4 |
| 3011 | VLQIKKVLRLLL | 7696 | 1.33 | 15.7 |
| 3012 | RVKAIKWRKIVV | 6639 | 0.85 | 2.8 |
| 3013 | LWQLWLKLKLKG | 6387 | 0.61 | 14.1 |
| 3014 | KIQRRAWKQWRK | 5695 | 1.09 | −3.7 |
| 3015 | KIVIRIILQVIK | 5543 | 0.83 | −3.8 |
| 3016 | AVKWLGWILAKK | 852 | 0.34 | 0.8 |
| 3017 | LAGLIVKWAGVR | 784 | 0.36 | 5.9 |
| 3018 | WVGVIIKWGLKL | 726 | 0.5 | −0.3 |
| 3019 | WQGWAKIWVVRI | 280 | 0.5 | −3.1 |
| 3020 | LIVIQLLKKWWK | 1464 | 0.55 | 10.2 |
| 3021 | RRIIKILLWKLR | 1581 | 0.57 | 13.1 |
| 3022 | IAWQLLWGWRVR | 600 | 0.6 | −4.8 |
| 3023 | VQRIIWLRVKIV | 127 | 0.61 | −3 |
| 3024 | IKIIWKALGQVI | 804 | 0.63 | −1.7 |
| MILBFmax5 | IQLKLIWVKRKW | 715 | 0.85 | −1.3 |
| BFmax-9 | VIKVLIKRWLKL | 1127 | 0.64 | 7.4 |
| BFmax-6 | VQWIQIVVWRKR | 3778 | 1.18 | −4.4 |
| IBFmax-15 | GLIIKIIKKRLW | 458 | 0.67 | 25.1 |
| Imax- 5 | VKGAIKRGIWVK | 5194 | 1.32 | 3 |
| BFmax-7 | KVQIIKQLIAKK | 4713 | 1.04 | −3.9 |
| BFmax-16 | KRLQWVKVKKIR | 4703 | 1.24 | 0.6 |
| Imax- 10 | IVKWIAQWKLVG | 4338 | 1.06 | −1 |
| Mmax- 16 | KKQKKIWRRILV | 4056 | 1.02 | 1.8 |
| Mmax-3 | GRVLKIVwRKGR | 3882 | 0.64 | 4.2 |
| Mmax- 18 | RQVRVKRWRARW | 3735 | 1.39 | −4.9 |
| BFmax-2 | KVVWWKVIIKVL | 3362 | 0.81 | −3.5 |
| Imax-7 | ALAIKVWIKILQ | 2638 | 1.01 | −1.1 |
| MILBFmax-9 | IRILVLRKAIIV | 2444 | 1.47 | 23.9 |
| MILBFmax-13 | IVKKVKLIWGVK | 2220 | 0.76 | −3.2 |
| Imin-3 | VVGLRVRWVRLW | 2069 | 1.25 | −4.3 |
| Imax-20 | WAVRALKVKWAL | 2005 | 1.07 | −2.7 |
| IBFmax-13 | WWIKIVVIRVRR | 575 | 0.63 | −2 |
| MILBFmax-8 | QIIKVVWRAVII | 56 | 0.65 | −2.3 |
| IBFmax-11 | QQVKWWLIRWLA | 328 | 0.66 | −0.1 |
| IBFmax-17 | IKWVLRKIVQII | 181 | 0.66 | 3.6 |
| BFmax-5 | VARWKIIIAKLW | 566 | 0.7 | 2 |
| Imax-6 | KIWGLLKLGIAL | 368 | 0.71 | 1.1 |
| Imin-4 | RARQIRWLRKRV | 140 | 0.74 | −4.9 |
| IBFmax-8 | RVLIKWKKVIVV | 1235 | 0.75 | −1.8 |
| MILBFmax-1 | LKLKAILKIIRV | 763 | 0.75 | 1.5 |

TABLE 7
Synthetic control peptides with low immunomodulatory or antibiofilm
activity. All peptides were SPOT synthesized on cellulose sheet and
their biological activities were determined in the same way as
activities described in Tables 5 and 6.

| | | | | | Fold Change in | |
|---|---|---|---|---|---|---|
| | | | | MCPI | IL1-β relative | |
| | | | | production in | to untreated | % |
| SEQ | Peptide | Alternate | | PBMC | LPS-stimulated | Biofilm |
| ID NO | Name | Name | Sequence | (pg/ml) | cells | Inhibition |
| 2 | C1 | Mmax-1 | RARIGIWKKWWA | 1477 | 0.81 | 101 |
| 1086 | C2 | Mmax-2 | KRKQWKLWVRQI | 171 | 1.5 | 100 |
| 1087 | C3 | Mmax-4 | GAKIIRKVAQVA | 577 | 1.08 | 103 |
| 1088 | C4 | Mmax-5 | VKRVKQILWRLG | 1386 | 0.91 | 101 |
| 1089 | C5 | Mmax-7 | IKAAKAGQWRRV | 683 | 1.04 | 101 |
| 1090 | C6 | Mmax-8 | RGRLKQKWWRRL | 569 | 1.25 | 102 |
| 1091 | C7 | Mmax-9 | LQRVIWQKWRKV | 370 | 0.94 | 105 |
| 1092 | C8 | Mmax-10 | RLAKRKGQAIWV | 233 | 1.42 | 105 |
| 1093 | C9 | Mmax-11 | QKIGRAVIWKVK | 758 | 1.46 | 103 |
| 1094 | C10 | Mmax-12 | QLRVAWKRAWWA | 1313 | 1.34 | 87 |
| 1095 | C11 | Mmax-13 | KAVKKGRRAIVV | 197 | 1.12 | 106 |
| 1096 | C12 | Mmax-14 | VIRAKAVWGWVK | 498 | 1.01 | 104 |
| 1097 | C13 | Mmax-15 | VARAVQKRWRKK | 221 | 1.55 | 105 |
| 1098 | C14 | Mmax-17 | VKAKRWKWAQLA | 209 | 1.2 | 104 |
| 1099 | C15 | Mmax-20 | KRVQAKAWRLQR | 297 | 0.94 | 100 |
| 1100 | C16 | Mmin-1 | KKIRQWGKAAAW | 571 | 0.99 | 100 |
| 1101 | C17 | Mmin-3 | QQLRWKRVAKAI | 1677 | 1.05 | 100 |
| 1102 | C18 | Mmin-5 | IQIQLVKRWAVI | 1768 | 0.92 | 100 |
| 1103 | C19 | Imax-2 | RLIQWGWKIWAV | 738 | 0.94 | 99 |
| 1104 | C20 | Imax-3 | KLLGILKQAIVV | 106 | 0.78 | 100 |
| 1105 | C21 | Imax-4 | VLLRVGARIVVG | 345 | 0.83 | 99 |
| 1106 | C22 | Imax-8 | LLIAGKWWKLAI | 153 | 0.83 | 103 |
| 1107 | C23 | Imax-11 | LKKIIVQAVGLI | 653 | 0.95 | 97 |
| 1108 | C24 | Imax-12 | LKILIAQAKKGL | 1280 | 1.4 | 101 |
| 1109 | C25 | Imax-14 | IGQVVLVKIKIA | 1762 | 1.21 | 98 |
| 1110 | C26 | Imax-15 | VWLAQKIGKWIW | 1153 | 1.41 | 100 |
| 1111 | C27 | Imax-16 | KKAIKVVAIGRI | 50 | 1.06 | 102 |
| 1112 | C28 | Imax-18 | WIIRWIKIWLKI | 444 | 0.88 | 72 |
| 1113 | C29 | Imax-19 | VIAKIVLLRAGL | 1809 | 0.99 | 100 |
| 1114 | C30 | Imin-1 | RGARVIRWKLRR | 1153 | 1.23 | 100 |
| 1115 | C31 | Imin-5 | RAIIKQRWQRRW | 1649 | 1.09 | 103 |
| 1116 | C32 | BFmax-1 | QRWKKWKVLKLR | 1614 | 1.11 | 102 |
| 1117 | C33 | BFmax-3 | KIWLLKLRQRQK | 1685 | 1.23 | 102 |
| 1118 | C34 | BFmax-4 | WRIKKQWIQIIV | 217 | 1.13 | 99 |
| 1119 | C35 | BFmax-13 | IILKRVQVQKIK | 481 | 1.1 | 101 |
| 1120 | C36 | BFmax-14 | KRIKKLLKVVLK | 719 | 0.92 | 100 |
| 1121 | C37 | BFmax-15 | QQKVIRLLWKAK | 216 | 0.83 | 102 |
| 1122 | C38 | BFmax-18 | RIWRRAWKARWK | 251 | 1.06 | 102 |
| 1123 | C39 | BFmax-20 | KIKLIQKQLRIK | 286 | 0.84 | 98 |
| 1124 | C40 | BFmin-1 | ALLAGRKRAVAV | 39 | 0.89 | 102 |
| 1125 | C41 | BFmin-2 | KAVAGARQRWAL | 353 | 0.82 | 101 |
| 1126 | C42 | BFmin-3 | AIGAARAWRQWA | 114 | 0.85 | 102 |
| 1127 | C43 | BFmin-5 | AVIVRAAKGGAR | 100 | 0.99 | 103 |
| 1128 | C44 | IBFmax-2 | LLKLKQKGIVIA | 365 | 0.94 | 102 |
| 1129 | C45 | IBFmax-3 | QWLVKWVIIKVV | 499 | 1 | 102 |
| 1130 | C46 | IBF max-4 | IQIWIIRVIWRW | 697 | 0.91 | 102 |
| 1131 | C47 | IBFmax-5 | KVIQWIIVRRVL | 1193 | 0.76 | 102 |
| 1132 | C48 | IBF max-7 | KVIKIVLVRVVK | 1752 | 1.07 | 99 |
| 1133 | C49 | IBF max-10 | WLKRIVKVVVLK | 1557 | 0.8 | 103 |
| 1134 | C50 | IBF max-18 | IKIVRRAKIIIW | 222 | 0.91 | 94 |
| 1135 | C51 | IBF max-20 | VKWKGKVIVVQL | 862 | 0.81 | 79 |
| 1136 | C52 | MILBFmax-3 | KIVQKKLRLVVI | 291 | 0.76 | 99 |
| 1137 | C53 | MILBFmax-4 | GKLKIKVKLGIA | 113 | 0.94 | 98 |
| 1138 | C54 | MILBFmax-7 | QVVVKKKAIQVV | 651 | 0.95 | 101 |
| 1139 | C55 | MILBFmax-10 | VAKVKKARWRLR | 208 | 0.92 | 102 |
| 1140 | C56 | MILBFmax-11 | IIKWIVVRQIRK | 57 | 0.85 | 102 |
| 1141 | C57 | MILBFmax-12 | KGKIRKIVLIRR | 157 | 0.9 | 102 |
| 1142 | C58 | 1018-r2 | VGproLIVAVRIWRR | — | — | 96 |
| 1143 | C59 | 1018-r3 | VRGproIVAVRIWRR | — | — | 104 |
| 1144 | C60 | 1018-r4 | VRLGproVAVRIWRR | — | — | 104 |
| 1145 | C61 | 1018-r5 | VRLIGproAVRIWRR | — | — | 101 |
| 1146 | C62 | 1018-r6 | VRLIVGproVRIWRR | — | — | 106 |
| 1147 | C63 | 1018-r7 | VRLIVAGproRIWRR | — | — | 105 |
| 1148 | C64 | 1018-r8 | VRLIVAVGproIWRR | — | — | 107 |
| 1149 | C65 | 1018-r9 | VRLIVAVRGproWRR | — | — | 107 |
| 1150 | C66 | 1018-r10 | VRLIVAVRIGproRR | — | — | 107 |
| 1151 | C67 | 1018-r11 | VRLIVAVRIWGproR | — | — | 105 |
| 1152 | C68 | 1018-r12 | VRLIVAVRIWRGpro | — | — | 106 |
| 1153 | C69 | 1018-r14 | VGbutLIVAVRIWRR | — | — | 81 |
| 1154 | C70 | 1018-r15 | VRGbutIVAVRIWRR | — | — | 98 |
| 1155 | C71 | 1018-r16 | VRLGbutVAVRIWRR | — | — | 103 |
| 1156 | C72 | 1018-r17 | VRLIGbutAVRIWRR | — | — | 105 |
| 1154 | C73 | 1018-r18 | VRLIVGbutVRIWRR | — | — | 104 |
| 1158 | C74 | 1018-r19 | VRLIVAGbutRIWRR | — | — | 106 |
| 1159 | C75 | 1018-r20 | VRLIVAVGbutIWRR | — | — | 103 |
| 1160 | C76 | 1018-r21 | VRLIVAVRGbutWRR | — | — | 104 |
| 1161 | C77 | 1018-r22 | VRLIVAVRIGbutRR | — | — | 101 |
| 1162 | C78 | 1018-r23 | VRLIVAVRIWGbutR | — | — | 101 |
| 1163 | C79 | 1018-r24 | VRLIVAVRIWRGbut | — | — | 72 |
| 1164 | C80 | 1018-r26 | VHarLIVAVRIWRR | — | — | 99 |
| 1165 | C81 | 1018-r27 | VRHarIVAVRIWRR | — | — | 107 |
| 1166 | C82 | 1018-r28 | VRLHarVAVRIWRR | — | — | 106 |
| 1167 | C83 | 1018-r29 | VRLIHarAVRIWRR | — | — | 106 |
| 1168 | C84 | 1018-r31 | VRLIVAHarRIWRR | — | — | 105 |
| 1169 | C85 | 1018-r33 | VRLIVAVRHarWRR | — | — | 99 |
| 1170 | C86 | 1018-r34 | VRLIVAVRIHarRR | — | — | 75 |
| 1171 | C87 | 1018-k2 | VDapLIVAVRIWRR | — | — | 73 |
| 1172 | C88 | 1018-k3 | VRDapIVAVRIWRR | — | — | 104 |
| 1173 | C89 | 1018-k4 | VRLDapVAVRIWRR | — | — | 99 |
| 1174 | C90 | 1018-k5 | VRLIDapAVRIWRR | — | — | 97 |
| 1175 | C91 | 1018-k6 | VRLIVDapVRIWRR | — | — | 76 |
| 1176 | C92 | 1018-k7 | VRLIVADapRIWRR | — | — | 103 |
| 1177 | C93 | 1018-k8 | VRLIVAVDapIWRR | — | — | 71 |
| 1178 | C94 | 1018-k9 | VRLIVAVRDapWRR | — | — | 92 |
| 1179 | C95 | 1018-k14 | VDabLIVAVRIWRR | — | — | 71 |
| 1180 | C96 | 1018-k15 | VRDabIVAVRIWRR | — | — | 89 |
| 1181 | C97 | 1018-k16 | VRLDabVAVRIWRR | — | — | 102 |
| 1182 | C98 | 1018-k17 | VRLIDabAVRIWRR | — | — | 103 |
| 1183 | C99 | 1018-k18 | VRLIVDabVRIWRR | — | — | 100 |
| 1184 | C100 | 1018-k19 | VRLIVADabRIWRR | — | — | 104 |
| 1185 | C101 | 1018-k20 | VRLIVAVDabIWRR | — | — | 103 |
| 1186 | C102 | 1018-k21 | VRLIVAVRDabWRR | — | — | 104 |
| 1187 | C103 | 1018-k22 | VRLIVAVRIDabRR | — | — | 102 |
| 1188 | C104 | 1018-k23 | VRLIVAVRIWDabR | — | — | 93 |
| 1189 | C105 | 1018-k24 | VRLIVAVRIWRDab | — | — | 96 |
| 1190 | C106 | 1018-k27 | VROIVAVRIWRR | — | — | 104 |
| 1191 | C107 | 1018-k28 | VRLOVAVRIWRR | — | — | 71 |
| 1192 | C108 | 1018-k29 | VRLIOAVRIWRR | — | — | 104 |
| 1193 | C109 | 1018-k31 | VRLIVAORIWRR | — | — | 104 |
| 1194 | C110 | 1018-k33 | VRLIVAVROWRR | — | — | 100 |
| 1195 | C111 | 1018-k34 | VRLIVAVRIORR | — | — | 92 |

To confirm the most active chemokine inducing peptides from this screen, the best chemokine inducers (Peptides 3008-3015, SEQ ID NO: 31-38) were synthesized in larger amounts and to high purity (>95%). All of these QSAR-optimized MCP1 inducing peptides were tested for their ability to induce chemokine production from PBMCs (FIG. 8), revealing that peptides 3008, 3010, 3012, 3013 and 3015 displayed stronger MCP1 inducing abilities than 1018. In addition, the anti-biofilm activity against MRSA biofilms as well as the cytotoxicity and anti-inflammatory properties were evaluated for all these peptides (FIG. 8).

It would be predicted that D-amino acid peptides SEQ ID NO: 80-245, and non-natural amino acid substitution peptides SEQ ID NO: 6-23 and 74-79, would have immunomodulatory activity. Both classes of peptides would be likely to be more stable in the face of host proteases.

### Example 8: Anti-Inflammatory Impact on Innate Immunity

It is well known that cationic antimicrobial peptides have the ability to boost immunity while suppressing inflammatory responses to bacterial signaling molecules like lipopolysaccharide and lipoteichoic acids as well as reducing inflammation and endotoxaemia (Hancock, R. E. W., A. Nijnik and D. J. Philpott. 2012. Modulating immunity as a therapy for bacterial infections. Nature Rev. Microbiol. 10:243-254). This suppression of inflammatory responses has stand-alone potential as it can result in protection in the neuro-inflammatory cerebral malaria model [Achtman et al., 2012] and with hyperinflammatory responses induced by flagellin in cystic fibrosis epithelial cells [Mayer, M. L., C. J. Blohmke, R. Falsafi, C. D. Fjell, L. Madera, S. E. Turvey, and R. E. W. Hancock. 2013. Rescue of dysfunctional autophagy by IDR-1018 attenuates hyperinflammatory responses from cystic fibrosis cells. J. Immunol. 190:1227-1238].

LPS from P. aeruginosa strain H103 was highly purified free of proteins and lipids using the Darveau-Hancock method. Briefly, P. aeruginosa was grown overnight in LB broth at 37° C. Cells were collected and washed and the isolated LPS pellets were extracted with a 2:1 chloroform:methanol solution to remove contaminating lipids. Purified LPS samples were quantitated using an assay for the specific sugar 2-keto-3-deoxyoctosonic acid (KDO assay) and then resuspended in endotoxin-free water (Sigma-Aldrich).

Human PBMC were obtained as described above and treated with P. aeruginosa LPS (10 or 100 ng/ml) with or without peptides for 24 hr after which supernatants were collected and IL-1β levels were assessed by ELISA.

The data in Table 6 demonstrated that while LPS as expected induced large levels of the proinflammatory cytokine Interleukin 1β (IL1-β) none of the peptides significantly increased this pro-inflammatory response. Importantly, 23 peptides from the QSAR Experimental Validation Set showed superior activity to 1018 in reducing proinflammatory cytokine IL1-β production from LPS-stimulated PBMCs.

The activity of a subset of the most active anti-inflammatory peptides from Table 6 (Peptides 3016-3024, SEQ ID No. 39-47) was confirmed by synthesizing these peptides in larger amounts and to high purity (>95%). These peptides were tested for their anti-inflammatory properties, revealing a concentration-dependent decrease in LPS-stimulated IL1-β production from human PBMCs (FIG. 9). This revealed that nearly all of these QSAR optimized anti-inflammatory peptides were either equivalent to or better than 1018 at suppressing IL1-β productions from LPS-stimulated PBMCs. Only peptide 3017 showed reduced anti-inflammatory activity relative to 1018 at all the concentrations tested. The anti-biofilm activity as well as cytotoxicity and chemokine inducing abilities of these peptides were also assessed, revealing numerous peptides with multiple biological activities (FIG. 9).

A subset of peptides was tested for stimulation of the chemokine CCL5 (Table 21). All of the tested peptides, on their own, induced chemokine CCL5 (indicative of immune cell recruiting pro-protective activities) from Bovine and Canine cells, except for peptides 3013 and 3015 treated bovine cells. Additionally, most of the tested peptides exhibited anti-inflammatory effects in stimulated cells. The exceptions were again peptides 3013 and 3015 towards LPS stimulated bovine cells. Furthermore peptide 3016 did not suppress CCL5 production from ConA stimulated monocytes while peptides 3009 and 3017 were not as effective in ConA stimulated T-cells.

TABLE 21
Stimulation of CCL5 production alone by synthetic peptide and peptide mediated
modulation of CCL5 production in the presence of LPS (bovine cells) or Con A (canine
cells). Results in the absence of LPS are italicized if the amount of CCL5 produced
was 150% or more of that of the no peptide control. Results are italicized if
the peptide increased or substantially (>50%) maintained the production of
CCL5 stimulated by LPS (bovine cells) or ConA (dog cells) compared to the no peptide
control. All peptides were evaluated at a concentration of 32 μg/ml.

| | Concentration of CCL5 in the absence | | | | % CCL5 production relative to that | | | |
|---|---|---|---|---|---|---|---|---|
| | of stimulation by LPS or ConA | | | | of LPS/ConA | | | |
| | Bovine | | Canine | | Bovine/LPS | | Canine/ConA | |
| Peptides | Monocyte | T-cells | Monocyte | T-cells | Monocyte | T-cells | Monocyte | T-cells |
| 3009 | 1212 | 208 | 152 | 174 | 79 | 77 | 117 | 14 |
| 3010 | 647 | 150 | 142 | 354 | 96 | 92 | 138 | 81 |
| 3013 | 215 | 50 | 234 | 140 | 37 | 35 | 123 | 95 |
| 3015 | 468 | 86 | 342 | 123 | 28 | 42 | 96 | 78 |
| 3016 | 933 | 200 | 112 | 186 | 115 | 97 | 43 | 110 |
| 3017 | 1224 | 158 | 411 | 175 | 104 | 122 | 98 | 31 |
| DJK5 | 825 | 195 | 442 | 290 | 105 | 79 | 81 | 26 |
| NONE | 781 | 266 | 111 | 126 | 10054 | 8532 | 1241 | 3995 |

A subset of peptides was tested for stimulation of cytokine production in monocytes (Table 22). For the tested cytokines, the peptides tended to show anti-inflammatory activity rather than protective activity.

TABLE 22
Ability of peptides to stimulate cytokine production in monocytes. Shown is
the concentration of cytokines in pg/ml produced by monocytes in the absence
of any additional stimulant (Background is subtracted with the actual values
obtained included in the row NONE). Results are only italicized when cytokine
induction lead to a 50% increase over cell background (no peptide) levels.

| | Concentration of Monocyte cytokine (pg/ml) in absence of additional stimulant | | | | | | | | | | | |
|---|---|---|---|---|---|---|---|---|---|---|---|---|
| | Bovine | | | | | | Canine | | | | | |
| Peptides | IL-6 | TNFα | IFNγ | IL-2 | IL-4 | IL-13 | IL-6 | TNFα | IFNγ | IL-2 | IL-4 | IL-13 |
| 3009 | −43 | −262 | 480 | −109 | −58 | 52 | −65 | −141 | −308 | 47 | −32 | 155 |
| 3010 | 58 | −199 | −43 | 87 | −152 | 19 | −125 | −114 | −263 | −90 | −1 | 166 |
| 3013 | 50 | −31 | −35 | −13 | 2 | 83 | −112 | −96 | −274 | −4 | 59 | 142 |
| 3015 | 98 | −80 | 71 | −99 | −158 | 90 | −80 | −167 | −288 | 344 | −260 | 421 |
| 3016 | 47 | −306 | 433 | −122 | −9 | 11 | −123 | −70 | −96 | 109 | −201 | 13 |
| 3017 | 40 | −150 | 188 | −100 | −155 | 3 | −22 | −66 | −231 | −30 | −97 | 322 |
| DJK5 | 75 | −218 | −3 | −103 | −196 | 294 | −113 | −73 | −310 | 86 | −241 | −25 |
| NONE | 303 | 442 | 168 | 234 | 322 | 112 | 173 | 216 | 453 | 175 | 453 | 158 |

Many of the tested synthetic peptides exhibited good anti-inflammatory effects towards stimulated monocytes from cows and dogs (Table 23).

TABLE 23
Anti-inflammatory activity of 32 μg/ml of synthetic peptides in LPS (bovine)
or ConA (Canine) stimulated monocytes. Shown is the % monocyte cytokine decrease
in presence of LPS or ConA with italicized values representing good anti-inflammatory
activity. A negative % value indicates an increase in cytokine production.

| | % Monocyte cytokine decrease in presence of LPS/ConA (32 μg peptide) | | | | | | | | | | | |
|---|---|---|---|---|---|---|---|---|---|---|---|---|
| | Bovine | | | | | | Canine | | | | | |
| Peptides | IL-6 | TNFα | IFNγ | IL-2 | IL-4 | IL-13 | IL-6 | TNFα | IFNγ | IL-2 | IL-4 | IL-13 |
| 3009 | 35.3 | 37.4 | 83.1 | 13.5 | 47.6 | 82.3 | 26.0 | 52.7 | 12.9 | 31.6 | 61.5 | 12.8 |
| 3010 | 23.6 | 60.1 | 88.6 | 87.4 | −12.2 | 84.8 | 30.9 | 25.6 | 18.4 | 40.0 | 47.5 | 32.6 |
| 3013 | 6.4 | 50.9 | 93.6 | 21.8 | 46.8 | 85.6 | −5.9 | 40.1 | −1.9 | 32.3 | 13.5 | 42.1 |
| 3015 | 7.3 | 26.2 | 92.0 | 87.7 | 73.1 | 78.3 | −16.5 | 19.0 | 38.0 | 20.2 | 34.9 | 27.4 |
| 3016 | 16.5 | 11.8 | 89.6 | 77.3 | 73.7 | 74.6 | 12.4 | 17.0 | 57.4 | 53.2 | 43.5 | 15.6 |
| 3017 | 28.3 | 17.8 | 80.0 | 88.4 | 58.9 | 79.8 | −2.2 | 7.1 | 65.7 | 34.2 | 58.5 | 4.0 |
| DJK5 | 21.0 | 68.1 | 31.6 | 34.0 | 3.9 | 65.5 | 39.9 | 44.3 | 13.7 | 38.9 | 32.6 | 36.5 |
| NONE_{(pg/ml)} | 6046 | 4990 | 3897 | 987 | 874 | 880 | 2624 | 6724 | 2310 | 2160 | 3773 | 1537 |

Many of the tested synthetic peptides had a modest ability to stimulate T cell cytokines in dogs or cows (Table 24).

TABLE 24
Ability of synthetic peptides to stimulate cytokine production in T-lymphocytes. Shown
is the concentration of cytokines in pg/ml produced by monocytes in the absence of any
additional stimulant (Background is subtracted with the actual values obtained included
in the row NONE). Results in the absence of LPS are italicized if the amount of cytokine
produced was increased by 50% or more compared to the no peptide control.

| | Concentration of T-cell cytokines (pg/ml) in the absence of additional stimulant | | | | | | | | | | | |
|---|---|---|---|---|---|---|---|---|---|---|---|---|
| | Bovine | | | | | | Canine | | | | | |
| Peptides | IL-6 | TNFα | IFNγ | IL-2 | IL-4 | IL-13 | IL-6 | TNFα | IFNγ | IL-2 | IL-4 | IL-13 |
| 3009 | −139 | −18 | −100 | −32 | −21 | 43 | 99 | −149 | −59 | 23 | −58 | −108 |
| 3010 | −7 | 203 | −113 | 28 | −32 | 12 | 317 | 606 | 111 | 1 | 13 | −147 |
| 3013 | −33 | 19 | −80 | −2 | 177 | 49 | 103 | 194 | −111 | 30 | −48 | 61 |
| 3015 | −54 | −4 | −54 | −9 | 253 | 68 | 1 | 367 | −123 | 173 | 0 | 0 |
| 3016 | −48 | 280 | 90 | −35 | 18 | 57 | −44 | 35 | −89 | 3 | −30 | −118 |
| 3017 | −36 | 112 | −33 | −13 | −35 | 18 | −18 | −178 | −77 | 123 | −90 | −116 |
| DJK5 | −92 | 113 | −12 | −66 | −73 | 178 | 149 | −46 | 11 | 13 | 71 | −100 |
| NONE | 469 | 123 | 178 | 112 | 146 | 99 | 174 | 483 | 201 | 109 | 341 | 253 |

Many of the tested synthetic peptides exhibited good anti-inflammatory In activities in T-cells (Table 25).

TABLE 25
Anti-inflammatory activity of 32 μg/ml of the peptides towards T-lymphocytes. Shown is the % T-cell
cytokine decrease in the presence of LPS or ConA. Shown is the % monocyte cytokine decrease in presence
of LPS or ConA with italicized values indicating good anti-inflammatory activity. As above, all of
the synthetic peptides exhibited good anti-inflammatory activity under the conditions tested.

| | % Cytokine decrease in the presence of LPS/ConA (32 μg of peptide) | | | | | | | | | | | |
|---|---|---|---|---|---|---|---|---|---|---|---|---|
| | Bovine T-cells | | | | | | Canine T-cells | | | | | |
| Peptides | IL-6 | TNFα | IFNγ | IL-2 | IL-4 | IL-13 | IL-6 | TNFα | IFNγ | IL-2 | IL-4 | IL-13 |
| 3009 | −1.6 | 41.1 | 55.6 | 89.6 | 67.6 | 92.7 | 31.9 | 54.2 | 56.2 | 92.7 | 62.5 | 25.1 |
| 3010 | 3.8 | 54.6 | 67.6 | 59.5 | 75.9 | 93.1 | 44.1 | 32.2 | 43.2 | 91.6 | 65.8 | 36.0 |
| 3013 | 4.9 | 17.5 | 52.3 | 37.5 | 69.2 | 89.3 | 38.8 | 43.4 | 58.9 | 90.5 | 50.9 | 43.8 |
| 3015 | −0.9 | −41.0 | 83.9 | 85.9 | 70.3 | 89.4 | 45.5 | 24.7 | 67.0 | 93.3 | 10.8 | 64.3 |
| 3016 | 0.6 | 46.8 | 96.7 | 73.8 | 59.4 | 91.6 | 26.4 | 0.4 | 66.8 | 56.1 | 47.6 | 28.0 |
| 3017 | 11.1 | −10.9 | 86.8 | 83.7 | 23.3 | 72.8 | 42.4 | 9.9 | 73.9 | 49.9 | 55.4 | 25.5 |
| DJK5 | −16.4 | 38.2 | 34.9 | 80.7 | 37.7 | 85.3 | 24.8 | 7.1 | 60.2 | 62.3 | 65.5 | 48.2 |
| NONE_{(pg/ml)} | 6012 | 2696 | 3470 | 1100 | 1201 | 1524 | 3139 | 5915 | 3775 | 2417 | 3343 | 2389 |

New peptides were iteratively designed from our best immunomodulatory IDR peptides by QSAR methods. This enabled the assessment of peptides with excellent computationally determined biological activity (Table 8).

It was also predicted that D-amino acid peptides (SEQ ID NO: 80-245) and non-natural amino acid substitution peptides (SEQ ID NO: 6-23 and 74-79) would have anti-inflammatory activity. Both classes of peptides would be likely to be more stable in the face of host proteases.

### Example 9: Reduced Cytotoxicity

Cytotoxicity was assessed using the Lactate dehydrogenase assay. This was done using the same cell-free supernatants as for cytokine detection except that the supernatants were tested the same day as they were obtained to avoid freeze-thawing. Lactate dehydrogenase (LDH) assay (Roche cat #11644793001) is a colorimetric method of measuring cytotoxicity/cytolysis based on measurement of LHD activity released from cytosol of damaged cells into the supernatant. LDH released from permeable cells into the tissue culture supernatant will act to reduce the soluble pale yellow tetrazolium salt in the LDH assay reagent mixture into the soluble red coloured formazan salt product. Amount of colour formed is detected as increased absorbance measured at ˜500 nm. The calculations were done using the following formula Cytotoxicity %=(exp value−CTR)/(Triton−CTR)*100%. Anything under 10% is considered acceptable.

Cytotoxicity towards red blood cells (RBCs) was also assessed for the most active QSAR derived peptides (3001-3024, SEQ ID NO: 24-47) by measuring peptide induced hemolysis. The calculations were done using the following formula: Hemolysis %=(exp value−CTR)/(Triton−CTR)*100%. Anything under 10% is considered acceptable.

Of the new QSAR derived peptides (3001-3024, SEQ ID NO: 24-47), many of the new sequences exhibited low levels of cytoxicity towards PBMCs and/or hemolysis towards RBCs (FIGS. 6, 8 and 9). In particular, peptides 3002, 3005, 3007-3011, 3015-3017, 3020-3024 exhibited low toxicity, similar to the levels seen for 1018.

### Example 10: Activities of Qsar-Derived Peptides

Among the most active sequences with low toxicity and good overall activity profiles (ie. Combined anti-biofilm and immunomodulatory activities) peptides 3002, 3007, 3015, 3016 3012, 3022 and 3023 were found to align to a consensus sequence of 10 amino acids (FIG. 10) wherein a stretch of 10 residues in each peptide shares at least 90% sequence identity with the sequence Z₁U₂B₃Z₄J₅Z₆W₇J₈Z₉O₁₀ wherein Z=hydrophobic residues (W, V, L, I, A or G); B=basic residues (R or K); J=Basic or hydrophobic residues (Z+B); U=Uncharged residues (Z+Q); and O=pOlar residues (B+Q).

Exemplary chemical structures are presented in FIG. 11.

This consensus sequence was tested by examining the most active sequences determined computationally by the QSAR models and identified peptides Mmax-3, Imax-7, IBFmax-13 and BFmax-5 (SEQ ID NO: 57, 60, 65 and 67) from the QSAR Experimental Validation Set as well as an additional 368 sequences (SEQ ID NO: 535-903, Table 8) which include a stretch of 10 amino acids that share 90% sequence identity with the consensus sequence (Z₁U₂B₃Z₄J₅Z₆W₇J₈Z₉O₁₀). It is therefore established that these sequences are likely to have excellent anti-biofilm and immunomodulatory properties while also displaying low toxicity.

TABLE 8
Peptides with activity as immunomodulatory
and/or antibiofilm peptides as assessed
computationally by QSAR models. The QSAR
models were used to define the activities
of 100,000 virtual peptides and those peptide
sequences that were predicted as being most
active for each activity type were filtered
against the consensus sequence described in
Example 9. The predicted activity rankings
(by percentile) are shown for anti-biofilm
activity, IL1B suppression and MCP1 induction.
All these most active peptides were within the
90th percentile or greater for at least one of
the activities modeled using QSAR methods.
Peptides 3002, 3007, 3015 3016, 3021, 3022 and
3023 (SEQ ID NO: 25, 30, 38, 39, 44, 45 and
46) were peptides that were computationally
determined to be highly active and proved to be
so when synthesized and tested using microbio-
logical and immunological assays as described
herein. SEQ ID NO: 57, 60, 65 and 69 were part
of the QSAR validation set of peptides and
agree with the consensus sequence. Of the
5560 peptides with the highest scoring
computationally-assessed activities, SEQ ID NO:
535-903 additionally matched the consensus
sequence identified for peptides possessing
multiple activities.

| | | | Activity | | |
|---|---|---|---|---|---|
| | | | Rankings | | |
| | | | (% of | | |
| SEQ | | | optimal) | | |
| ID | Peptide | | Anti- | | |
| No | Name | Sequences | biofilm | IL1B | MCP1 |
| 25 | 3002 | ILVRWIRWRIQW | 99 | 10 | 18 |
| 30 | 3007 | VLKIKVKIWVVK | 99 | 97 | 16 |
| 38 | 3015 | KIVIRIILQVIK | 99 | 76 | 7 |
| 39 | 3016 | AVKWLGWILAKK | 25 | 99 | 61 |
| 44 | 3021 | RRIIKILLWKLR | 99 | 14 | 29 |
| 45 | 3022 | IAWQLLWGWRVR | 68 | 99 | 10 |
| 46 | 3023 | VQRIIWLRVKIV | 99 | 98 | 35 |
| 57 | Mmax-3 | GRVLKIVWRKGR | 43 | 28 | 99 |
| 60 | Imax-7 | ALAIKVWIKILQ | 54 | 99 | 38 |
| 65 | IBFmax-13 | WWIKIVVIRVRR | 99 | 96 | 56 |
| 69 | BFmax-5 | VARWKIIIAKLW | 99 | 89 | 33 |
| 535 | 55154 | LKIWKIVRWRLR | 99 | 53 | 22 |
| 536 | 86486 | LLLLRIRLLKWR | 99 | 11 | 6 |
| 537 | 2332 | VILLRAKLIKVR | 99 | 72 | 40 |
| 538 | 58972 | IWWKIGKWLVRK | 99 | 37 | 28 |
| 539 | 36115 | VIAKVIVRKVKK | 99 | 83 | 91 |
| 540 | 30043 | VRWQKLRRWVIR | 99 | 6 | 75 |
| 541 | 79907 | QIWIKIKILKLK | 99 | 70 | 17 |
| 542 | 10204 | VLKVIIRRWRLI | 99 | 64 | 16 |
| 543 | 3402 | RRWLKLRIVLLK | 99 | 10 | 31 |
| 544 | 9751 | IVKVVVQKIKGK | 99 | 85 | 92 |
| 545 | 63254 | KQLLRLLIKIVK | 99 | 64 | 32 |
| 546 | 21325 | VIIRWLGIRLKW | 99 | 60 | 42 |
| 547 | 22264 | IVQKVWIWRVRK | 99 | 9 | 76 |
| 548 | 24314 | AWRIRLKWLKLQ | 99 | 32 | 21 |
| 549 | 66066 | RQIWKWILQKIK | 99 | 50 | 3 |
| 550 | 60412 | LWRWVVRKIRRW | 99 | 18 | 68 |
| 551 | 86703 | KILKALIQKVQR | 99 | 27 | 63 |
| 552 | 6114 | IIWRILVQVLQK | 99 | 17 | 2 |
| 553 | 81515 | IWIIKVLWLKWK | 99 | 88 | 11 |
| 554 | 96579 | WWKLILWQVKQA | 99 | 79 | 2 |
| 555 | 60015 | RWKVIIWRIQIW | 99 | 15 | 99 |
| 556 | 91674 | KWWVKLLVKRIQ | 99 | 65 | 16 |
| 557 | 31035 | RIVRVRGVIIKW | 99 | 13 | 93 |
| 558 | 3642 | WIRWLALRIRWL | 99 | 58 | 22 |
| 559 | 54343 | LVAKIVVVKWKQ | 99 | 79 | 67 |
| 560 | 46779 | LQRVKLKWWKWG | 99 | 4 | 3 |
| 561 | 10463 | VVKIWIQRIKLV | 99 | 33 | 97 |
| 562 | 51509 | AVQWRIRWWKLK | 99 | 1 | 15 |
| 563 | 56231 | QILKLKIKRLRI | 99 | 27 | 53 |
| 564 | 70167 | KQVVKWRLKKVK | 99 | 55 | 96 |
| 565 | 52008 | AWRAILWKLKWR | 99 | 32 | 7 |
| 566 | 94851 | WIKLLVKIWKVK | 99 | 77 | 90 |
| 567 | 93408 | ILRVKLRLIQRK | 99 | 22 | 38 |
| 568 | 46991 | VQVIKAWAKILK | 99 | 95 | 40 |
| 569 | 28539 | LIAIKVKIRWIK | 99 | 70 | 27 |
| 570 | 46544 | IIKLKVVLIKIQ | 99 | 96 | 10 |
| 571 | 76813 | IIIKVIGRRVQW | 99 | 40 | 10 |
| 572 | 72287 | AVVWKLWWIKKK | 99 | 38 | 64 |
| 573 | 5711 | QWLIKRVLWKVR | 99 | 93 | 36 |
| 574 | 64777 | GWIWRLLWRVIK | 99 | 17 | 53 |
| 575 | 57303 | AWKIIGLIIRQV | 99 | 68 | 43 |
| 576 | 47480 | KVQRWIVRLIKV | 99 | 44 | 47 |
| 577 | 91455 | VVIQQLALWKAK | 99 | 93 | 75 |
| 578 | 99994 | RIWRLKWRRWQK | 99 | <1 | 43 |
| 579 | 29406 | IWKLRVVIVQKI | 99 | 43 | 5 |
| 580 | 77320 | KIRIKLWRVRWK | 99 | 22 | 74 |
| 581 | 53323 | ALQKVRVLVAKI | 99 | 92 | 18 |
| 582 | 84323 | KKGVKIKLLLVK | 99 | 96 | 89 |
| 583 | 43685 | IKWVRWWLKRLQ | 99 | 36 | 15 |
| 584 | 79318 | WWQRVRILWLRK | 99 | 62 | 11 |
| 585 | 91319 | VKGWRWRKWRIQ | 99 | 49 | 83 |
| 586 | 71536 | KWWRIRGKWARL | 99 | 13 | 35 |
| 587 | 54676 | VVRIAWKRVQIK | 99 | 11 | 54 |
| 588 | 55147 | KILRWKRWRWRI | 99 | <1 | 50 |
| 589 | 54601 | AIIGLKVWLIRI | 58 | 99 | 23 |
| 590 | 73185 | LIWRVIIGIWQK | 32 | 99 | 37 |
| 591 | 83954 | WALKVKVWLIGW | 47 | 99 | 18 |
| 592 | 30296 | IIRIGKWGAKVI | 28 | 99 | 89 |
| 593 | 10024 | LILKILLKVWKG | 83 | 99 | 42 |
| 594 | 49701 | VALVGWWRLQLK | 49 | 99 | 28 |
| 595 | 31446 | LLAVKLKVGVAR | 43 | 99 | 42 |
| 596 | 59611 | LVKWKLAWAKGL | 33 | 99 | 23 |
| 597 | 87287 | AIWKVKAWVVGW | 19 | 99 | 68 |
| 598 | 33945 | VAKIAVKWLQLI | 41 | 99 | 48 |
| 599 | 13165 | VWGVWLWRIKKL | 93 | 99 | 2 |
| 600 | 40115 | GRVIKVKGWLVV | 28 | 99 | 85 |
| 601 | 95633 | ILRGIVGLAKIK | 76 | 99 | 23 |
| 602 | 84839 | WLGKAALKILQL | 23 | 99 | 27 |
| 603 | 90088 | VQAWKAWIARVK | 32 | 99 | 98 |
| 604 | 44456 | KAAKVIWWGIAA | 28 | 99 | 85 |
| 605 | 27094 | IAKVVWWKVWGL | 17 | 99 | <1 |
| 606 | 12596 | IVAIKAKVQALR | 70 | 99 | 78 |
| 607 | 1606 | GVLKAVVVKVKL | 45 | 99 | 73 |
| 608 | 18472 | WWKIGAVLIKRA | 41 | 99 | 20 |
| 609 | 18546 | WLKAWGGKIRVL | 16 | 99 | 17 |
| 610 | 89289 | ALRARVAVAKIK | 60 | 99 | 64 |
| 611 | 49298 | VKIGRWVQWAWK | 18 | 99 | 21 |
| 612 | 89635 | VVWIKIALGLLK | 72 | 99 | 25 |
| 613 | 10356 | LIILKIWWWGAK | 44 | 99 | 3 |
| 614 | 64704 | AWVLKVWIWKGQ | 57 | 99 | 11 |
| 615 | 70183 | KWLIIGIWWVKG | 34 | 99 | 9 |
| 616 | 47172 | IAKIWILGLKVK | 57 | 99 | 57 |
| 617 | 16251 | IARWGLLAAKGI | <1 | 99 | 87 |
| 618 | 81687 | LVRVGGIVVKKW | 13 | 99 | 14 |
| 619 | 53678 | LWGIKGWKLKLW | 54 | 99 | 76 |
| 620 | 75637 | VVIKILIGVLRA | 76 | 99 | 52 |
| 621 | 39480 | LKIWKIAAKVGQ | 55 | 99 | 24 |
| 622 | 25833 | KIAKIAALKIRA | 22 | 99 | 81 |
| 623 | 57100 | LALKGVLKWLKG | 27 | 99 | 12 |
| 624 | 23528 | AVLLKVGVWLVR | 12 | 99 | 77 |
| 625 | 12611 | VKLWKILGVAVK | 69 | 99 | 70 |
| 626 | 38334 | AWGWKVKVIGAK | 40 | 99 | 4 |
| 627 | 66084 | WAWKVVGLILKI | 4 | 99 | 6 |
| 628 | 79156 | GIRKLIWWGRAV | 11 | 99 | 76 |
| 629 | 55479 | LWVGRVLGIILK | 53 | 99 | 52 |
| 630 | 25356 | GVWVRLALWKLV | 27 | 99 | 41 |
| 631 | 50189 | IQVARAGAAIIR | 2 | 99 | 56 |
| 632 | 93656 | IKVQLLGVWVIR | 87 | 99 | 16 |
| 633 | 40845 | GIIKWVAKWVRI | 29 | 99 | 97 |
| 634 | 45300 | ILVIKAGGLLIK | 62 | 99 | 36 |
| 635 | 55028 | LIVKAIAVRGKI | 35 | 99 | 62 |
| 636 | 38097 | VAGIKWAVWKLR | 23 | 99 | 67 |
| 637 | 40090 | WGVVAKWWKIQI | 42 | 99 | 1 |
| 638 | 28751 | KIWKLAIVGWKI | 61 | 99 | 33 |
| 639 | 88406 | KIIIKLLGWGVK | 66 | 99 | 83 |
| 640 | 40718 | WWIKGIIIKLKK | 89 | 99 | 20 |
| 641 | 24795 | LLRAWIVRIQGL | 10 | 99 | 15 |
| 642 | 25852 | IIKIQIWWIRII | 90 | 99 | 88 |
| 643 | 47775 | WLVKIVVQWAQK | 94 | 99 | 1 |
| 644 | 62594 | GAIIKWKLIAVR | 75 | 99 | 70 |
| 645 | 90672 | AWKVGVWLVRAG | 3 | 99 | 54 |
| 646 | 97873 | WWVVKAKTALAR | 56 | 99 | 64 |
| 647 | 53009 | RWGLKWVAWKAI | 15 | 99 | 1 |
| 648 | 69899 | LIAKIRVIVGRA | 44 | 99 | 74 |
| 649 | 76724 | VIQVIRIWGARL | 39 | 99 | 40 |
| 650 | 73159 | GILKLKVVWGKI | 59 | 99 | 98 |
| 651 | 21168 | WIRLGILAVKAW | 32 | 99 | 6 |
| 652 | 33227 | AIVKVKIAWGRI | 20 | 99 | 91 |
| 653 | 72410 | AALAKLKIWGLG | 2 | 99 | 57 |
| 654 | 42190 | ILKAIIKIIQWG | 95 | 99 | 27 |
| 655 | 79785 | ALKLAVLGIRLL | 48 | 99 | 83 |
| 656 | 39889 | WGWRAIARIWQI | 8 | 99 | 43 |
| 657 | 48143 | IVIKGGIWKIAR | 39 | 99 | 42 |
| 658 | 16191 | KILKAVLGGIRW | 14 | 99 | 23 |
| 659 | 77466 | AWAVKWRVARVK | 60 | 99 | 40 |
| 660 | 59667 | IWVKAKLARIKA | 92 | 99 | 30 |
| 661 | 60218 | IQVVKLWKWQLA | 59 | 99 | <1 |
| 662 | 14956 | LLIKGVVIKIQV | 69 | 99 | 89 |
| 663 | 88723 | AVKWVVAGARLV | 8 | 99 | 51 |
| 664 | 94283 | AIIKILWRLWKI | 85 | 99 | 23 |
| 665 | 53359 | AGWKWQVWRAKK | 62 | 99 | 54 |
| 666 | 48027 | LIKWAIWKVGKI | 19 | 99 | 95 |
| 667 | 98696 | GLQKWVWRIWKI | 35 | 86 | 99 |
| 668 | 95545 | AVRIKVWKWGRR | 50 | 30 | 99 |
| 669 | 15603 | VQAARGLAKKAR | 1 | 66 | 99 |
| 670 | 81581 | VLRGKLLWVQVI | 79 | 59 | 99 |
| 671 | 67290 | AWAKAKGKGVRV | 29 | 45 | 99 |
| 672 | 85299 | AVKWKIWAGQIL | 54 | 67 | 99 |
| 673 | 33574 | ILRILIKVVRKK | 97 | 63 | 99 |
| 674 | 64014 | KALKVIWRGVKG | 75 | 76 | 99 |
| 675 | 97330 | KIIIIKVWLGRA | 85 | 54 | 99 |
| 676 | 84468 | GVKRVAVWRAKK | 12 | 65 | 99 |
| 677 | 54185 | ILVKIWIRWGRA | 48 | 60 | 99 |
| 678 | 61242 | LIRWRAVVLKVA | 47 | 35 | 99 |
| 679 | 74707 | AAVKRIRIWLGK | 21 | 82 | 99 |
| 680 | 54732 | RKGVKIAVRAGR | 10 | 74 | 99 |
| 681 | 50152 | RVGVKHWQKAK | 12 | 49 | 99 |
| 682 | 76537 | AGLKKKIWVGRK | 8 | 62 | 99 |
| 683 | 76515 | KKVRIGGWIARI | 14 | 89 | 99 |
| 684 | 34617 | GKLGKLVWWKRR | 96 | 18 | 99 |
| 685 | 57648 | WRLIKIWLWRKQ | 88 | 19 | 99 |
| 686 | 61521 | KIALARLWAWRR | 17 | 19 | 99 |
| 687 | 85007 | VVIKLKIRRGRR | 67 | 29 | 99 |
| 688 | 96840 | IRIARWAIKIWQ | 77 | 33 | 99 |
| 689 | 81232 | ALQAIKVWAWKL | 8 | 62 | 99 |
| 690 | 29057 | VLQLGIWAAKRK | 20 | 86 | 99 |
| 691 | 50265 | VIAKIGIWIGRV | 48 | 21 | 99 |
| 692 | 47294 | QIWKKKAWAGRI | 24 | 70 | 99 |
| 693 | 26986 | ALVGKIGIWRIL | 26 | 87 | 99 |
| 694 | 4907 | LRAIKIKWWRWV | 68 | 79 | 99 |
| 695 | 37706 | GAVLKLVWRLVR | 52 | 65 | 99 |
| 696 | 45876 | AKIQKIIWGRIR | 41 | 89 | 99 |
| 697 | 86206 | ALQKLWAKRARW | 15 | 19 | 99 |
| 698 | 98484 | RRGRLKLWIVRI | 35 | 64 | 99 |
| 699 | 38908 | RGARWGIKKAKV | 10 | 45 | 99 |
| 700 | 9907 | KKLWKAKAKVVR | 90 | 40 | 99 |
| 701 | 32001 | LWKKVVWIGKKK | 43 | 39 | 99 |
| 702 | 1510 | QLARRKAWKVKG | 51 | 5 | 99 |
| 703 | 99155 | KVIQKAAIQVWK | 50 | 89 | 99 |
| 704 | 69909 | RGKIALWGWKRI | 63 | 1 | 99 |
| 705 | 19069 | AAILKIGIWLGK | 9 | 60 | 99 |
| 706 | 18656 | LLWIKIAWIRGK | 93 | 78 | 99 |
| 707 | 51078 | KVIGRVVLWGIK | 67 | 38 | 99 |
| 708 | 7515 | IAKLKIVGGKKK | 65 | 78 | 99 |
| 709 | 82422 | KVVRWAKWVAKK | 66 | 5 | 99 |
| 710 | 97070 | WIAKIKVWKILK | 58 | 71 | 99 |
| 711 | 80011 | AIRGVVVRWKQA | 74 | 13 | 99 |
| 712 | 82490 | GALAKARVKGAK | <1 | 36 | 99 |
| 713 | 51671 | AQIWRLKIWWVR | 37 | 2 | 99 |
| 714 | 94279 | LQRALVGWVKWK | 85 | 80 | 99 |
| 715 | 43479 | ALAKIAGGKVRI | 17 | 62 | 99 |
| 716 | 97318 | KQRAKIWRLRKV | 69 | 15 | 99 |
| 717 | 92749 | VQRVAGKKARRI | 41 | 17 | 99 |
| 718 | 52288 | GIRVIWQVIKKR | 74 | 70 | 99 |
| 719 | 9760 | LARLAWWRQKAR | 25 | 21 | 99 |
| 720 | 45789 | RIVRAAWKGARI | 11 | 57 | 99 |
| 721 | 67541 | VGRARIAIIKWL | 17 | 66 | 99 |
| 722 | 39705 | VWGKVVLWGKKR | 49 | 69 | 99 |
| 723 | 3739 | IAKVVWVRAQRG | 4 | 46 | 99 |
| 724 | 18140 | KRLARAAGIIAR | 13 | 25 | 99 |
| 725 | 74243 | KAVKIAIKVWKR | 49 | 34 | 99 |
| 726 | 15767 | KWIGAKIWIAKG | 25 | 86 | 99 |
| 727 | 8197 | LALGKIWKGRAI | <1 | 54 | 99 |
| 728 | 48034 | VARQRIIWWKWR | 19 | 8 | 99 |
| 729 | 31851 | LWKWKLWRAVRL | 61 | 5 | 99 |
| 730 | 12988 | IAKAWWKRLQAI | 80 | 52 | 99 |
| 731 | 17636 | AQKIKWRVWKGL | 28 | 30 | 99 |
| 732 | 42372 | LKVVKVAAKLGR | 18 | 57 | 99 |
| 733 | 17278 | AARIRAWAGWGK | 0 | 25 | 99 |
| 734 | 33460 | LVARVKIKGIRI | 41 | 18 | 99 |
| 735 | 86853 | LIQKAKVKWVRQ | 69 | 28 | 99 |
| 736 | 58522 | ARQVKIGIWILR | 16 | 64 | 99 |
| 737 | 78847 | IWVWKIIQWRLR | 98 | 98 | 10 |
| 738 | 20988 | WILQKWLWIRLQ | 97 | 97 | <1 |
| 739 | 7655 | LQKLLKWLVQKW | 98 | 93 | 2 |
| 740 | 23581 | LVKVIVIKIQKW | 98 | 94 | 61 |
| 741 | 55675 | VIIKWRVIIAKR | 97 | 93 | 64 |
| 742 | 42645 | ILRLLWWKVVIR | 96 | 95 | 12 |
| 743 | 26629 | ILLQRLKLWIQR | 98 | 91 | 13 |
| 744 | 66801 | ILIKIWAGVVQK | 95 | 98 | 57 |
| 745 | 98462 | IWAQKAVVVKIK | 95 | 97 | 34 |
| 746 | 64076 | WIRIIIRVIKIA | 96 | 93 | 14 |
| 747 | 3832 | WQRLLKWLGKRK | 97 | 89 | 46 |
| 748 | 93477 | VKKGLGWLVKIK | 95 | 93 | 51 |
| 749 | 10110 | VVLKWIIRKIKI | 95 | 98 | 72 |
| 750 | 83102 | LLKLLGQLAKVV | 95 | 97 | 95 |
| 751 | 89876 | IIIKIVGVVWKW | 98 | 88 | 22 |
| 752 | 4480 | RALAKALLARIK | 94 | 98 | 20 |
| 753 | 59197 | KILKILARVLRW | 95 | 92 | 27 |
| 754 | 14280 | VAKLRLQLIKLV | 95 | 93 | 14 |
| 755 | 12275 | VVKAVIGKIKIQ | 95 | 94 | 39 |
| 756 | 21518 | LKWLKVGIRKVR | 94 | 95 | 69 |
| 757 | 68206 | WLIKARVQLLRK | 95 | 91 | 11 |
| 758 | 20217 | ILLLKVLLVKWR | 93 | 98 | 34 |
| 759 | 66980 | QWVKVLIIKWKK | 96 | 89 | 35 |
| 760 | 71029 | QGWRIRLKWIKI | 94 | 94 | 0 |
| 761 | 99420 | IIKLKLRVAQKL | 96 | 87 | 91 |
| 762 | 80262 | VLGWKIKGAKVK | 93 | 98 | 59 |
| 763 | 26018 | IWLKWKIQIAQA | 94 | 91 | 43 |
| 764 | 18965 | LWIRILVRVVRL | 94 | 91 | 2 |
| 765 | 46558 | LIIGVKGWKLKV | 98 | 85 | 66 |
| 766 | 81125 | WIVQKIKRWLVK | 95 | 88 | 7 |
| 767 | 73599 | LLWLRARWKIVK | 93 | 96 | 14 |
| 768 | 14729 | LWKIVLIGGKWW | 95 | 89 | <1 |
| 769 | 157 | RWALRIVWRRWQ | 93 | 92 | 3 |
| 770 | 43768 | RVIRIIIAKLKL | 97 | 84 | 89 |
| 771 | 98995 | LRIVWVKLWILK | 95 | 87 | 42 |
| 772 | 75082 | ALKIVIWQIKQL | 93 | 94 | 45 |
| 773 | 39201 | VKVQRWRVLVIK | 93 | 93 | 47 |
| 774 | 35140 | HILKWIWRAIK | 98 | 83 | 88 |
| 775 | 79114 | ALVVKVVIGKIR | 98 | 83 | 59 |
| 776 | 76662 | KIVKWVLVKVKI | 92 | 97 | 80 |
| 777 | 361 | IRLIRLKIIIAK | 98 | 83 | 76 |
| 778 | 15648 | IKIVKVLGLALK | 93 | 90 | 94 |
| 779 | 81938 | WLQKVKLWKVIK | 93 | 91 | 54 |
| 780 | 84389 | ALRLVVKVWKKR | 93 | 91 | 27 |
| 781 | 48724 | LKVLKVKILGAK | 97 | 82 | 90 |
| 782 | 38516 | LLKLQWWAVKKA | 93 | 89 | 56 |
| 783 | 47225 | IVRIKIWVKKVL | 92 | 94 | 82 |
| 784 | 15 | VHLKVLAQVVK | 91 | 98 | 34 |
| 785 | 17240 | KQKIRVWIAKRI | 92 | 91 | 81 |
| 786 | 8388 | IAKIKVILVQLQ | 93 | 89 | 64 |
| 787 | 14569 | VLKIKWGRVRWW | 93 | 87 | 1 |
| 788 | 45863 | IGKLKLQLIKLR | 96 | 72 | 98 |
| 789 | 76457 | VIRKIKIWKVQK | 94 | 67 | 96 |
| 790 | 78045 | VLVKVKLRAVRI | 93 | 44 | 97 |
| 791 | 72242 | GARIWLQKIKLA | 90 | 71 | 97 |
| 792 | 99634 | VWKVIWRAGQLK | 94 | 75 | 96 |
| 793 | 62004 | RIIQRWVKWILQ | 90 | 39 | 97 |
| 794 | 48944 | IVIAKVWIVRKA | 93 | 84 | 96 |
| 795 | 85707 | LQKWKIKKVRIR | 97 | 14 | 95 |
| 796 | 76134 | RLVKIRLWRLWK | 89 | 18 | 97 |
| 797 | 83278 | ALWIKILKWVWK | 89 | 73 | 97 |
| 798 | 29814 | VWVWRVWVRIAR | 89 | 83 | 96 |
| 799 | 89122 | KIIKGVARRIRR | 98 | 35 | 95 |
| 800 | 31151 | LIIRAVWKIVRK | 97 | 25 | 95 |
| 801 | 91025 | GVRLKWLLWRRR | 92 | 5 | 95 |
| 802 | 99565 | RLIIKAKIRKVK | 97 | 49 | 94 |
| 803 | 22903 | LLWGKGKWRAWK | 91 | 35 | 95 |
| 804 | 88700 | RQIRIWVWRIQK | 87 | 51 | 97 |
| 805 | 75072 | VIKVKWWGVRVL | 86 | 49 | 97 |
| 806 | 5707 | VIKWRIWRRKIR | 93 | 5 | 94 |
| 807 | 42195 | LLLKLIIRLARR | 90 | 48 | 95 |
| 808 | 1503 | VAKLQIWLVKQK | 84 | 64 | 98 |
| 809 | 43576 | RGVKWKWKIVKK | 85 | 64 | 97 |
| 810 | 43439 | IQKWVVIRWRLR | 96 | 6 | 94 |
| 811 | 53700 | LAKWLRWIWRRQ | 89 | 11 | 95 |
| 812 | 38400 | VQIWKLKLLKAK | 87 | 32 | 96 |
| 813 | 73250 | IGRWKVQKAKWK | 93 | 5 | 94 |
| 814 | 22823 | VIIGKVKWRLIK | 98 | 35 | 93 |
| 815 | 89901 | VWWRLWVQRAQI | 85 | 60 | 96 |
| 816 | 35001 | AQVLKVVKWKIR | 96 | 15 | 93 |
| 817 | 15921 | AAVRLKGIIIKL | 83 | 93 | 98 |
| 818 | 40013 | KGAKWIVKKVKR | 85 | 21 | 96 |
| 819 | 16818 | AWIRLKAWRVRR | 89 | 1 | 94 |
| 820 | 99038 | KLWWKVLLKILK | 92 | 46 | 94 |
| 821 | 85232 | KWVKVRVVWLKW | 80 | 95 | 97 |
| 822 | 57661 | GIKLAWKKGRKL | 90 | 67 | 93 |
| 823 | 84530 | VVWVGVGIWRAK | 27 | 98 | 98 |
| 824 | 66168 | LGVVWKLWIIRV | 65 | 96 | 98 |
| 825 | 61522 | VGKAVARIGRRV | 21 | 91 | 98 |
| 826 | 91182 | KAIRLGAVKGKK | 27 | 90 | 98 |
| 827 | 31756 | WVKAVWRRAQWL | 19 | 85 | 98 |
| 828 | 45729 | RVWVIGIWGVRK | 28 | 86 | 98 |
| 829 | 22486 | LVRLAAKRARGI | 4 | 90 | 97 |
| 830 | 99458 | AALKVAGLAIKQ | 16 | 82 | 98 |
| 831 | 28730 | VVRGLGLIAKLV | 9 | 93 | 97 |
| 832 | 97204 | VVALKLWKVRRG | 59 | 80 | 98 |
| 833 | 52024 | KAAQLGLWVWKK | 24 | 84 | 97 |
| 834 | 66620 | RVLKLVGIAAKR | 60 | 86 | 97 |
| 835 | 58368 | IGKAVIWRGKRL | 37 | 82 | 98 |
| 836 | 54436 | AAKGAWKRIKGA | 4 | 82 | 97 |
| 837 | 60867 | IKALKVAVRAVQ | 20 | 88 | 96 |
| 838 | 33625 | LLQKAAIKWAKK | 61 | 81 | 97 |
| 839 | 45758 | RVIKALIGKGRK | 18 | 92 | 96 |
| 840 | 87920 | LLRAAWGVWRKV | 60 | 98 | 96 |
| 841 | 20448 | KLVRVWARLGQK | 62 | 92 | 96 |
| 842 | 4649 | RKIAKAGIWVGI | <1 | 86 | 96 |
| 843 | 27162 | GAIIKVWAGRKL | 12 | 78 | 98 |
| 844 | 76644 | ILWKAAWKAGRV | 3 | 76 | 98 |
| 845 | 39807 | WLAKLAAVRIQR | 46 | 89 | 96 |
| 846 | 64230 | GIQRIRVIWAKA | 20 | 78 | 97 |
| 847 | 95809 | VKVQKWVAKVAR | 44 | 74 | 98 |
| 848 | 9638 | KVGVRGAIRKIR | 9 | 95 | 95 |
| 849 | 72043 | IARAKIKWAKIL | 56 | 78 | 97 |
| 850 | 55864 | VLGKAVVKGVKV | 6 | 78 | 97 |
| 851 | 80806 | WKKAKIWIVQVK | 69 | 78 | 97 |
| 852 | 37731 | AAVRAKLKKVRI | 27 | 73 | 98 |
| 853 | 43014 | ILWLKVGWWVQK | 48 | 92 | 95 |
| 854 | 83382 | RWRRIGWWLIKQ | 51 | 86 | 95 |
| 855 | 52816 | AAKAKARAVKVL | 7 | 73 | 97 |
| 856 | 25609 | KVAAKAAIGVWK | 1 | 90 | 95 |
| 857 | 8861 | ALAGALKIWLGR | <1 | 76 | 97 |
| 859 | 53622 | GLQRIIVWRLVV | 53 | 81 | 96 |
| 860 | 35038 | KIGKAAKWILKI | 31 | 81 | 96 |
| 861 | 87856 | WWRIAAVKGRRV | 9 | 82 | 96 |
| 862 | 8846 | AVKALIKILKAQ | 33 | 87 | 95 |
| 863 | 94379 | IGLGRVAWAKAR | 1 | 70 | 98 |
| 864 | 70595 | LVRWAWLGARQV | 2 | 72 | 97 |
| 865 | 69714 | GKAIKLALKLLQ | 75 | 95 | 95 |
| 866 | 94713 | AAAKVKALGLKA | 35 | 72 | 97 |
| 867 | 12879 | VALAKVWIGKVG | 29 | 92 | 95 |
| 868 | 94639 | AARAAGIWIRVW | 10 | 73 | 97 |
| 869 | 8545 | AVIGKWGVWRAR | 27 | 74 | 96 |
| 870 | 25807 | QKAKAVVWRGKK | <1 | 71 | 97 |
| 871 | 98229 | VRAQRVKILIVQ | 40 | 70 | 98 |
| 872 | 40774 | VVRAGGRLIRAV | 5 | 74 | 96 |
| 873 | 48247 | IALIKKGGWLLK | 62 | 74 | 96 |
| 874 | 98537 | LKGVLARVWVIQ | 25 | 95 | 94 |
| 875 | 9964 | KVIKGIIVGLRL | 89 | 87 | 92 |
| 876 | 96382 | VAILKGWVLKIL | 81 | 75 | 94 |
| 877 | 13455 | VIRVVWLKVQLG | 79 | 73 | 96 |
| 878 | 5301 | IVRLIIQIWRLV | 81 | 84 | 92 |
| 879 | 17755 | KIQLVKIWLVKI | 94 | 80 | 89 |
| 880 | 57360 | AIIRALWKVVKR | 86 | 65 | 93 |
| 881 | 11592 | QIVKRKIWLAKK | 81 | 90 | 92 |
| 882 | 68158 | IQVLKIWGIKAK | 76 | 75 | 96 |
| 883 | 42393 | LRWWKWLAKIVR | 96 | 74 | 89 |
| 884 | 7223 | LVKWKIRVGQVI | 90 | 86 | 89 |
| 885 | 10587 | IVKVIWLKVKKQ | 85 | 78 | 91 |
| 886 | 17414 | IGIWKVWIQIWK | 80 | 88 | 91 |
| 887 | 35676 | RRLGKILWWKVI | 89 | 90 | 88 |
| 888 | 25030 | KIWIKAVWQILK | 92 | 74 | 89 |
| 889 | 73002 | IQVWRIKWQKLR | 95 | 62 | 90 |
| 890 | 70711 | AVRLLIKIWRVK | 82 | 97 | 90 |
| 891 | 40679 | GVKGIALKIKVL | 79 | 63 | 94 |
| 892 | 81050 | WLIVKWKVWKAR | 77 | 61 | 96 |
| 893 | 37139 | KWLAWKVWAIKQ | 90 | 74 | 88 |
| 894 | 10304 | VIRIKLKIWQWQ | 84 | 61 | 91 |
| 895 | 37399 | VILKVVWAGAKI | 76 | 95 | 91 |
| 896 | 89944 | LKVWRVILWGRR | 82 | 54 | 94 |
| 897 | 16173 | RRVRAKVWVLRW | 94 | 64 | 88 |
| 898 | 88116 | KWQKIWIGRVKV | 77 | 97 | 90 |
| 899 | 15948 | LKLQKIAALKIR | 74 | 73 | 93 |
| 900 | 60235 | WWIQVIIWRIRL | 79 | 89 | 89 |
| 901 | 27190 | VWKIWVKRVRVI | 98 | 77 | 86 |
| 902 | 63434 | ILVQRVKILIWK | 96 | 55 | 90 |
| 903 | 335 | ILKVKWIVGRKQ | 98 | 58 | 88 |

In some embodiments, peptides according to the present disclosure have the consensus sequence: HHHBHHBHBHJH, where “H” is a hydrophobic amino acid (W, L, I, V, A, or G); “B” is a basis amino acid (R or K); and “J” is a polar amino acid (Q, R, or K).

Exemplary peptides having the consensus sequence HHHBHHBHBHJH are listed in Table 9.

TABLE 9

| | SEQ | | | % Match |
|---|---|---|---|---|
| Number | ID | Name | Sequence | Consensus |
| 1 | 25 | 3002 | ILVRWIRWRIQW | 100.00 |
| 2 | 904 | 22086 | VIAKLWRIKWKW | 100.00 |
| 3 | 905 | 21325 | VIIRWLGIRLKW | 91.67 |
| 4 | 906 | 60259 | VIVRAVKQRLKL | 91.67 |
| 5 | 907 | 68843 | IIWKVGRIGAKL | 91.67 |
| 6 | 908 | 36245 | AIVWGARLRLKW | 91.67 |
| 7 | 909 | 41156 | IAWKWARVWIKA | 91.67 |
| 8 | 910 | 1606 | GVLKAVVVKVKL | 91.67 |
| 9 | 911 | 14135 | LLIRAIKVGLQV | 91.67 |
| 10 | 912 | 55028 | LIVKAIAVRGKI | 91.67 |
| 11 | 913 | 50627 | GIVRLAKLKGLV | 91.67 |
| 12 | 914 | 14956 | LLIKGVVIKIQV | 91.67 |
| 13 | 915 | 88125 | ILVRAVRGKALV | 91.67 |
| 14 | 916 | 43479 | ALAKIAGGKVRI | 91.67 |
| 15 | 917 | 85804 | IAARWIKAKWRG | 100.00 |
| 16 | 918 | 83557 | GGVKVLRVKVRV | 100.00 |
| 17 | 919 | 8394 | GILRLAKVKIKG | 100.00 |
| 18 | 920 | 89902 | IQVRIVRVKWKI | 91.67 |
| 19 | 921 | 47190 | GALRKIRVKWRV | 91.67 |
| 20 | 922 | 95608 | AVIKLGKIAWRG | 91.67 |
| 21 | 923 | 11316 | LVLLGIRAKVRA | 91.67 |
| 22 | 924 | 87625 | VWAKIWRLKAAI | 91.67 |
| 23 | 925 | 78370 | IAGKLVRVIWQI | 91.67 |
| 24 | 926 | 22485 | KAGKLLKLKVQV | 91.67 |

In some embodiments, peptides according to the present disclosure have the consensus sequence: HHBHBHBHHHHB, where “H” is a hydrophobic amino acid (W, L, I, V, A, or G); “B” is a basis amino acid (R or K); and “J” is a polar amino acid (Q, R, or K).

Exemplary peptides having the consensus sequence HHBHBHBHHHHB are listed in Table 10.

TABLE 10

| | SEQ | | | % Match |
|---|---|---|---|---|
| Number | ID | Name | Sequence | Consensus |
| 1 | 30 | 3007 | VLKIKVKIWVVK | 100.00 |
| 2 | 927 | 54905 | QIKIKVKILLIK | 91.67 |
| 3 | 928 | 25556 | IIKIKQKVLLVK | 91.67 |
| 4 | 929 | 3419 | LWKIRLKLVVKK | 91.67 |
| 5 | 930 | 69604 | IIKIWWKIGWLK | 91.67 |
| 6 | 931 | 2822 | WGKIKWRLLVGW | 91.67 |
| 7 | 932 | 31446 | LLAVKLKVGVAR | 91.67 |
| 8 | 933 | 41265 | LVRIKAGGLLVK | 91.67 |
| 9 | 934 | 38334 | AWGWKVKVIGAK | 91.67 |
| 10 | 935 | 45744 | VWKAKGLAAIGR | 91.67 |
| 11 | 936 | 57036 | VIKAALKALWVK | 91.67 |
| 12 | 937 | 62594 | GAIIKWKLIAVR | 91.67 |
| 13 | 938 | 97873 | WWVVKAKIALAR | 91.67 |
| 14 | 939 | 51123 | AAKAKIKGLGIW | 91.67 |
| 15 | 940 | 17278 | AARIRAWAGWGK | 91.67 |
| 16 | 941 | 55923 | ALKGLLKIVVIR | 91.67 |
| 17 | 942 | 21068 | LLKIILKIVLLR | 91.67 |
| 18 | 943 | 63416 | IIKGKLKVGLLL | 91.67 |
| 19 | 944 | 83539 | LLRIRVKAVIVK | 100.00 |
| 20 | 945 | 42434 | AIKVRIKIQLLK | 91.67 |
| 21 | 946 | 97273 | GIKWKWGVLVIR | 91.67 |
| 22 | 947 | 69998 | VVKWKLKKIIAR | 91.67 |
| 23 | 948 | 93176 | IVKQKVKVAGVK | 91.67 |
| 24 | 949 | 57227 | VIRGKQKVIVLR | 91.67 |

In some embodiments, peptides according to the present disclosure have the consensus sequence: BHHHBHHHJHHB, where “H” is a hydrophobic amino acid (W, L, I, V, A, or G); “B” is a basis amino acid (R or K); and “J” is a polar amino acid (Q, R, or K).

Exemplary peptides having the consensus sequence BHHHBHHHJHHB are listed in Table 11.

TABLE 11

| | SEQ | | | % Match |
|---|---|---|---|---|
| Number | ID | Name | Sequence | Consensus |
| 1 | 38 | 3015 | KIVIRIILQVIK | 100.00 |
| 2 | 950 | 27065 | RGIVKRWIRLLK | 91.67 |
| 3 | 951 | 35364 | RIIVKIIKKGIK | 91.67 |
| 4 | 952 | 64777 | GWIWRLLWRVIK | 91.67 |
| 5 | 953 | 64580 | RLIVRVIVRQLR | 91.67 |
| 6 | 954 | 89635 | VVWIKIALGLLK | 91.67 |
| 7 | 955 | 55479 | LWVGRVLGIILK | 91.67 |
| 8 | 956 | 88406 | KIIIKLLGWGVK | 91.67 |
| 9 | 957 | 43843 | KGWWKVIRRVLK | 91.67 |
| 10 | 958 | 54732 | RKGVKIAVRAGR | 91.67 |
| 11 | 959 | 50152 | RVGVKIIWQKAK | 91.67 |
| 12 | 960 | 25730 | KVKVKAIWVGGR | 91.67 |
| 13 | 961 | 99155 | KVIQKAAIQVWK | 91.67 |
| 14 | 962 | 51078 | KVIGRVVLWGIK | 91.67 |
| 15 | 963 | 25899 | RGLAKAVWRAWV | 91.67 |
| 16 | 964 | 86964 | KGIVQVLWRAIR | 91.67 |
| 17 | 965 | 76372 | KILWKIILAGLW | 91.67 |
| 18 | 966 | 35140 | IIILKWIWRAIK | 91.67 |
| 19 | 967 | 15 | VIILKVLAQVVK | 91.67 |
| 20 | 968 | 29814 | VWVWRVWVRIAR | 91.67 |
| 21 | 969 | 99038 | KLWWKVLLKILK | 91.67 |
| 22 | 970 | 21073 | RGAKRLVVKLIR | 91.67 |
| 23 | 971 | 86762 | RGAVKKIWGIWK | 91.67 |
| 24 | 972 | 45416 | KGLVKVVKVGVR | 91.67 |
| 25 | 973 | 25609 | KVAAKAAIGVWK | 91.67 |
| 26 | 974 | 25030 | KIWIKAVWQILK | 91.67 |

In some embodiments, peptides according to the present disclosure have the consensus sequence: HHBHHHHHHHBB, where “H” is a hydrophobic amino acid (W, L, I, V, A, or G); “B” is a basis amino acid (R or K); and “J” is a polar amino acid (Q, R, or K).

Exemplary peptides having the consensus sequence HHBHHHHHHHBB are listed in Table 12.

TABLE 12

| | SEQ | | | % Match |
|---|---|---|---|---|
| Number | ID | Name | Sequence | Consensus |
| 1 | 39 | 3016 | AVKWLGWILAKK | 100.00 |
| 2 | 975 | 2948 | WWKVLAGGLIRK | 100.00 |
| 3 | 976 | 30374 | IIKAIIKWWWRK | 91.67 |
| 4 | 977 | 46475 | ILKILWGVVWWK | 91.67 |
| 5 | 978 | 42377 | WLKVGGVALLKA | 91.67 |
| 6 | 979 | 75392 | LLKALIGLVARI | 91.67 |
| 7 | 980 | 2464 | LAKIIWWQWIRR | 91.67 |
| 8 | 981 | 26526 | IVRGKLVIVGKK | 91.67 |
| 9 | 982 | 86460 | LIRIVKWVWARR | 91.67 |
| 10 | 983 | 87309 | ALRAIGAWGALK | 91.67 |
| 11 | 984 | 50927 | GAKVWGLAAWKV | 91.67 |
| 12 | 985 | 11366 | LVRKGVIVAGKK | 91.67 |
| 13 | 986 | 71828 | AWKALWKVIVKK | 91.67 |
| 14 | 987 | 68085 | LKRGWGVVIVRK | 91.67 |
| 15 | 988 | 44133 | LLKVIKIIWLRK | 91.67 |
| 16 | 989 | 44769 | VVKLWVLGVLLK | 91.67 |
| 17 | 990 | 44016 | ALRAVWKWGIKR | 91.67 |
| 18 | 991 | 2739 | LLKVGLIGAARV | 91.67 |
| 19 | 992 | 97007 | VGAVIGVVLVRR | 91.67 |
| 20 | 993 | 52573 | IWKKLILGIWKK | 91.67 |
| 21 | 994 | 91922 | AWKIWVGWVAQR | 91.67 |
| 22 | 995 | 99382 | KLKAIGAVIWKK | 91.67 |

In some embodiments, peptides according to the present disclosure have the consensus sequence: BBHHBHHHHBHB, where “H” is a hydrophobic amino acid (W, L, I, V, A, or G); “B” is a basis amino acid (R or K); and “J” is a polar amino acid (Q, R, or K).

Exemplary peptides having the consensus sequence BBHHBHHHHBHB are listed in Table 13.

TABLE 13

| | SEQ | | | % Match |
|---|---|---|---|---|
| Number | ID | Name | Sequence | Consensus |
| 1 | 44 | 3021 | RRIIKILLWKLR | 100.00 |
| 2 | 996 | 18140 | KRLARAAGIIAR | 91.67 |
| 3 | 997 | 4480 | RALAKALLARIK | 91.67 |
| 4 | 998 | 94275 | KKAWKLVQIRIR | 91.67 |
| 5 | 999 | 35676 | RRLGKILWWKVI | 91.67 |

In some embodiments, peptides according to the present disclosure have the consensus sequence: HHHJHHHHHBHB, where “H” is a hydrophobic amino acid (W, L, I, V, A, or G); “B” is a basis amino acid (R or K); and “J” is a polar amino acid (Q, R, or K).

Exemplary peptides having the consensus sequence HHHJHHHHHBHB are listed in Table 14.

TABLE 14

| | SEQ | | | % Match |
|---|---|---|---|---|
| Number | ID | Name | Sequence | Consensus |
| 1 | 45 | 3022 | IAWQLLWGWRVR | 100.00 |
| 2 | 1000 | 24222 | IIVRVVKWIRWR | 91.67 |
| 3 | 1001 | 86958 | ALIQVGRLIKLK | 91.67 |
| 4 | 1002 | 90793 | IILKVIRILKWK | 91.67 |
| 5 | 1003 | 90286 | VIVQALGRIRWK | 91.67 |
| 6 | 1004 | 91455 | VVIQQLALWKAK | 91.67 |
| 7 | 1005 | 78929 | IIGKIVWGVLIK | 91.67 |
| 8 | 1006 | 63612 | ILIKALGIAKWL | 91.67 |
| 9 | 1007 | 23946 | AVIQGWLIIRWK | 100.00 |
| 10 | 1008 | 94416 | VGAKGWIIIRWK | 100.00 |
| 11 | 1009 | 5315 | VLVRGIVAVKIK | 100.00 |
| 12 | 1010 | 27638 | VLVKAWGIKKGR | 91.67 |
| 13 | 1011 | 447 | LWWRGVAVWKII | 91.67 |
| 14 | 1012 | 34174 | KAAKAAGIVRGK | 91.67 |
| 15 | 1013 | 58043 | IGIKVVIGWILK | 91.67 |
| 16 | 1014 | 71201 | VWIRLGLWGRAK | 100.00 |
| 17 | 1015 | 78477 | AIIRLVGLLAIR | 91.67 |
| 18 | 1016 | 356 | WVGKAVWGAKIL | 91.67 |
| 19 | 1017 | 93459 | WWIKIALGIRGI | 91.67 |
| 20 | 1018 | 6979 | WAIAGGVLIKAR | 91.67 |
| 21 | 1019 | 95751 | WLLRGGALIKWI | 91.67 |
| 22 | 1020 | 34608 | WAGKIVIIGKIA | 91.67 |
| 23 | 1021 | 41894 | VAALGWALVKAR | 91.67 |
| 24 | 1022 | 3447 | LGLKIIWVGKIL | 91.67 |
| 25 | 1023 | 42619 | VVWRLVGIIRIA | 91.67 |
| 26 | 1024 | 9922 | IWLKVGGIIIVK | 91.67 |
| 27 | 1025 | 12001 | LLWRLLWGVKGL | 91.67 |
| 28 | 1026 | 8571 | IIAWGVIVGKAR | 91.67 |
| 29 | 1027 | 93480 | VWLKLVGWAKIV | 91.67 |
| 30 | 1028 | 50599 | VWIKILGWLKIA | 91.67 |
| 31 | 1029 | 54824 | ILIGILGLLKVR | 91.67 |
| 32 | 1030 | 422 | ALLKWIWVGWIR | 91.67 |
| 33 | 1031 | 78177 | LILRAALRGRGR | 91.67 |
| 34 | 1032 | 31014 | AGAKVWKIVKWK | 91.67 |
| 35 | 1033 | 31408 | WGALWIGAVRIK | 91.67 |
| 36 | 1034 | 4369 | GAVQAIWRLRAR | 91.67 |
| 37 | 1035 | 91000 | LVIRQLWVWKVR | 91.67 |
| 38 | 1036 | 35128 | GLARAWAWRKGK | 91.67 |
| 39 | 1037 | 39705 | VWGKVVLWGKKR | 91.67 |
| 40 | 1038 | 26156 | LLIKWWLAKRLR | 91.67 |
| 41 | 1039 | 45991 | VIVRIVIGIIGK | 91.67 |
| 42 | 1040 | 41880 | LAWQLIIGIKIR | 100.00 |
| 43 | 1041 | 30037 | VILQLVWIKRLK | 91.67 |
| 44 | 1042 | 10483 | VWVQIIRAAKIR | 91.67 |
| 45 | 1043 | 98462 | IWAQKAVVVKIK | 91.67 |
| 46 | 1044 | 84502 | IIWRVKWVWRIK | 91.67 |
| 47 | 1045 | 5965 | VWWLGIIILKAK | 91.67 |
| 48 | 1046 | 69112 | VAWQLIVVRKGK | 91.67 |
| 49 | 1047 | 18653 | GIKKLVIGLKLK | 91.67 |
| 50 | 1048 | 29330 | VWLQIIIRVRWK | 91.67 |
| 51 | 1049 | 61562 | WLWKAVWIKKIK | 91.67 |
| 52 | 1050 | 48966 | IVVKVILARRLR | 91.67 |
| 53 | 1051 | 19308 | VIVQWKLWLRLR | 91.67 |
| 54 | 1052 | 53710 | GIGKWLVLRRVK | 91.67 |
| 55 | 1053 | 75851 | GWIKIRLGVKLK | 91.67 |
| 56 | 1054 | 84530 | VVWVGVGIWRAK | 91.67 |
| 57 | 1055 | 97984 | AGKRAGVVIKAR | 91.67 |
| 58 | 1056 | 17073 | VWLRWLGLVVVK | 91.67 |
| 59 | 1057 | 96429 | KVGRALGAAKAK | 91.67 |
| 60 | 1058 | 9512 | GLAKAIALGRIV | 91.67 |
| 61 | 1059 | 39758 | WLLKGGLVWRIV | 91.67 |
| 62 | 1060 | 27391 | VIGKKIWAARLK | 91.67 |
| 63 | 1061 | 76566 | WIIRKLAGVKVK | 91.67 |
| 64 | 1062 | 58584 | AIVQVLGAIRWK | 100.00 |

In some embodiments, peptides according to the present disclosure have the consensus sequence: HJBHHHHBHBHH, where “H” is a hydrophobic amino acid (W, L, I, V, A, or G); “B” is a basis amino acid (R or K); and “J” is a polar amino acid (Q, R, or K).

Exemplary peptides having the consensus sequence HJBHHHHBHBHH are listed in Table 15.

TABLE 15

| | SEQ | | | % Match |
|---|---|---|---|---|
| Number | ID | Name | Sequence | Consensus |
| 1 | 46 | 3023 | VQRIIWLRVKIV | 100.00 |
| 2 | 1063 | 36770 | GKKWRIIRWKWI | 91.67 |
| 3 | 1064 | 3642 | WIRWLALRIRWL | 91.67 |
| 4 | 1065 | 19259 | WRRLKIVKLKGG | 91.67 |
| 5 | 1066 | 52943 | IKKLLLARLKLK | 91.67 |
| 6 | 1067 | 38568 | GKRIWAIKKKIV | 91.67 |
| 7 | 1068 | 18546 | WLKAWGGKIRVL | 91.67 |
| 8 | 1069 | 46719 | LKWLIGIKLKGA | 91.67 |
| 9 | 1070 | 34795 | VKKAAWGKWKLI | 100.00 |
| 10 | 1071 | 50242 | IKKIWIVKWRKG | 91.67 |
| 11 | 1072 | 88384 | IKRLVAWKKKVL | 91.67 |
| 12 | 1073 | 78335 | VKRVVLIRIVAA | 91.67 |
| 13 | 1074 | 21710 | KQRVVIVKVRIL | 91.67 |
| 14 | 1075 | 6715 | ARKVIVIQVKAI | 91.67 |
| 15 | 1076 | 43439 | IQKWVVIRWRLR | 91.67 |
| 16 | 1077 | 15410 | GKRWLLVRVKKI | 91.67 |
| 17 | 1078 | 78709 | VRKVWIGGVKVI | 91.67 |
| 18 | 1079 | 44555 | IKAVVVGRAKIV | 91.67 |
| 19 | 1080 | 48723 | VQAAWAGKWKVW | 91.67 |
| 20 | 1081 | 65318 | LKKVWALRGIAV | 91.67 |
| 21 | 1082 | 9747 | VRKIVWIRLKVG | 100.00 |
| 22 | 1083 | 94865 | ARKIVWLKGRAV | 100.00 |
| 23 | 1084 | 40679 | GVKGIALKIKVL | 91.67 |
| 24 | 1085 | 72658 | IRKLLWIRALLG | 91.67 |

### Example 11: Adjuvanticity as a Result of Enhancement of Innate Immunity

Peptides, as described herein, were shown to upregulate chemokines in human PBMC (Table 6), consistent with an ability to act as adjuvants.

TABLE 16
MICs of QSAR optimized antibiofilm and immunomodulatory
peptides towards planktonic methicillin resistant S. aureus.
MICs were determined using the broth microdilution
method in both Mueller Hinton Broth (MHB) and in Tryptic
Soy Broth (TSB) supplemented with 1% glucose. Reported
MIC values (μM) are the mean value obtained from
three individual biological replicates.

MIC (μM)

| Peptide | MHB | TSB with 1% Glucose |
|---|---|---|
| 1018 | 4 | >64 |
| 3001 | 1 | 2 |
| 3002 | 2 | 4 |
| 3003 | 4 | 16 |
| 3004 | 4 | 16 |
| 3005 | 4 | 64 |
| 3006 | 4 | 32 |
| 3007 | 16 | >64 |
| 3008 | 2 | 4 |
| 3009 | >64 | >64 |
| 3010 | >64 | >64 |
| 3011 | 8 | 16 |
| 3012 | >64 | >64 |
| 3013 | 32 | >64 |
| 3014 | >64 | >64 |
| 3015 | 64 | >64 |
| 3016 | >64 | >64 |
| 3017 | >64 | >64 |
| 3018 | 2 | 4 |
| 3019 | 8 | 32 |
| 3020 | 8 | >64 |
| 3021 | 4 | 32 |
| 3022 | 4 | 8 |
| 3023 | 16 | >64 |
| 3024 | 16 | 32 |
| Vancomycin | 0.34 | 0.68 |

### Example 12: Tryptic Stability of Cationic Substituted 1018. Derivatives

Peptides were incubated in the absence or presence of bovine trypsin for 30 minutes. Peptide samples (10 μM) were incubated at 37° C. in the absence (black) or presence of trypsin (grey) and the samples were subjected to RP-HPLC analysis using a water-acetonitrile gradient (FIG. 21A). Absorbance values in the chromatogram have been normalized to the maximum absorbance (280 nm) observed in the peptide sample in the absence of trypsin. The amount of peptide in each sample was then quantified by comparing the area of the peak on the chromatogram for the undigested peptide to the corresponding peak in the digested sample (FIG. 21B). Data represent the average of three biological replicates (±SD) and statistical significance was calculated by one-way ANOVA comparing each peptide to the amount of 1018 digested under the same conditions (P-value: *=0.033, **=0.002, ***=<0.001).

Substitution of non-natural amino acids as well as specific incorporation of Lys at certain positions improved the proteolytic stability towards trypsin degradation. Peptide 1018-Lys4, 1018-Lys5 and 1018-Dpr5 (SEQ ID NO: 74, 77 and 78) were the most stable under the experimental conditions evaluated.

### REFERENCES

Hancock R E W. 2001. Cationic peptides: effectors in innate immunity and novel antimicrobials. Lancet Infectious Diseases 1, 156-164;
Fjell C D, Hiss J A, Hancock R E W and Schneider G. 2012. Designing antimicrobial peptides: Form follows function. Nature Rev. Drug Discov. 11:37-51
Gough M, Hancock R E W, Kelly N M. 1996. Anti-endotoxic potential of cationic peptide antimicrobials. Infect. Immun. 64, 4922-4927
Hilchie A L, K Wuerth, and R E W Hancock. 2013. Immune modulation by multifaceted cationic host defence (antimicrobial) peptides. Nature Chem. Biol. 9:761-8
Hancock, R. E. W., A. Nijnik and D. J. Philpott. 2012. Modulating immunity as a therapy for bacterial infections. Nature Rev. Microbiol. 10:243-254; Fjell C D, et al. 2012. Nat. Rev. Drug Discov. 11:37-51
Høiby, N., et al. 2011. The clinical impact of bacterial biofilms. International J Oral Science 3:55-65.;
Antunes, L C M and R B R Ferreira. 2011. Biofilms and bacterial virulence. Reviews Med Microbiol 22:12-16
Overhage, J., A. Campisano, M. Bains, E. C. W. Torfs, B. H. A. Rehm, and R. E. W. Hancock. 2008. The human host defence peptide LL-37 prevents bacterial biofilm formation. Infect. Immun. 76:4176-4182
Amer L. S., B. M. Bishop, and M. L. van Hoek. 2010. Antimicrobial and antibiofilm activity of cathelicidins and short, synthetic peptides against Francisella. Biochem Biophys Res Commun 396:246-51
de la Fuente-Núñez, C., V. Korolik, M. Bains, U. Nguyen, E. B. M. Breidenstein, S. Horsman, S. Lewenza, L. Burrows and R. E. W. Hancock. 2012. Inhibition of bacterial biofilm formation and swarming motility by a small synthetic cationic peptide. Antimicrob. Agents Chemother. 56:2696-2704
Fuente-Núñez, C., F. Reffuveille, E. F. Haney, S. K. Straus and R. E. W. Hancock. 2014. Broad-spectrum anti-biofilm peptide that targets a cellular stress response. PLoS Pathogens 10(5):e1004152.;
de la Fuente-Núñez, C., F. Reffuveille, S. C. Mansour, S. L. Reckseidler-Zenteno, D. Hernández, G. Brackman, T. Coenye and R. E. W. Hancock. 2015. D-enantiomeric peptides that eradicate wild-type and multi-drug resistant bacterial biofilms and protect against lethal Pseudomonas aeruginosa infections. Chem Biol. 22:196-205.
Reffuveille, F., C. de la Fuente-Núñez, S. Mansour and R. E. W. Hancock. 2014. A broad-spectrum anti-biofilm peptide enhances antibiotic action against bacterial biofilms. Antimicrob. Agents Chemother. 58:5363-5371
M. G., E. Dullaghan, N. Mookherjee, N. Glavas, M. Waldbrook, A. Thompson, A. Wang, K. Lee, S. Doria, P. Hamill, J. Yu, Y. Li, O. Donini, M. M. Guarna, B. B. Finlay, J. R. North, and R. E. W. Hancock. 2007. An anti-infective peptide that selectively modulates the innate immune response. Nature Biotech. 25: 465-472
Haney, E. F., S. Mansour, A. L. Hilchie, C. de la Fuente-Núñez and R. E. W. Hancock. 2015. High throughput screening methods for assessing antibiofilm and immunomodulatory activities of synthetic peptides. Peptides 71:276-285.;
Mansour, S., C. de la Fuente-Núñez and R. E. W. Hancock. 2015. Peptide IDR-1018: modulating the immune system and targeting bacterial biofilms to treat antibiotic-resistant bacterial infections. J. Pept. Res. 21:323-329
Bowdish D M E, Davidson D J, and Hancock R E W. 2005. A re-evaluation of the role of host defence peptides in mammalian immunity. Current Protein Pept. Sci. 6:35-51
Hancock R E W, and Sahl H G. 2006. Antimicrobial and host-defence peptides as novel anti-infective therapeutic strategies. Nature Biotech. 24:1551-1557.
Scott, et al. 2007. Nature Biotech. 25: 465-472
Nijnik A., L. Madera, S. Ma, M. Waldbrook, M. Elliott, S. C. Mullaly, J. Kindrachuk, H. Jenssen, R. E. W. Hancock. 2010. Synthetic cationic peptide IDR-1002 provides protection against bacterial infections through chemokine induction and enhanced leukocyte recruitment. J. Immunol. 184:2539-2550.;
Turner-Brannen, E., K.-Y. Choi, D. N. D. Lippert, J. P. Cortens, R. E. W. Hancock, H. El-Gabalawy and N. Mookherjee. 2011. Modulation of IL-1β-induced inflammatory responses by a synthetic cationic innate defence regulator peptide, IDR-1002, in synovial fibroblasts. Arthritis Res. Ther. 13:R129.;
Madera, L. and R. E. W. Hancock. 2012. Synthetic immunomodulatory peptide IDR-1002 enhances monocyte migration and adhesion on fibronectin. J. Innate Immun. 4:553-568.;
Achtman, A. H., S. Pilat, C. W. Law, D. J. Lynn, L. Janot, M. Mayer, S. Ma, J. Kindrachuk, B. B. Finlay, F. S. L. Brinkman, G. K. Smyth, R. E. W. Hancock and L. Schofield. 2012. Effective adjunctive therapy by an innate defense regulatory peptide in a pre-clinical model of severe malaria. Science Transl. Med. 4:135ra64.;
Rivas-Santiago, B., J. E. Castañeda-Delgado, C. E. Rivas Santiago, M. Waldbrook, I. González-Curiel, J. C. León-Contreras, A. Enciso-Moreno, V. del Villar, J. Méndez-Ramos, R. E. W. Hancock, R. Hernandez-Pando. 2013. Ability of innate defence regulator peptides IDR-1002, IDR-HH2 and IDR-1018 to protect against Mycobacterium tuberculosis infections in animal models. PLoS One 8:e59119.;
Mayer, M. L., C. J. Blohmke, R. Falsafi, C. D. Fjell, L. Madera, S. E. Turvey, and R. E. W. Hancock. 2013. Rescue of dysfunctional autophagy by IDR-1018 attenuates hyperinflammatory responses from cystic fibrosis cells. J. Immunol. 190:1227-1238.;
Niyonsaba, F., L. Madera, K. Okumura, H. Ogawa, and R. E. W. Hancock. 2013. The innate defense regulator peptides IDR-HH2, IDR-1002 and IDR-1018 modulate human neutrophil functions. J. Leukocyte Biol. in press PMID: 23616580.;
Bolouri, H., K. Sävman, W. Wang, A. Thomas, N. Maurer, E. Dullaghan, C. D. Fjell, H. Hagberg, R. E. W. Hancock, K. L. Brown, and C. Mallard. 2014. Innate defence regulator peptide 1018 protects against perinatal brain injury. Ann. Neurol. 75:395-410;
Kindrachuk, J., H. Jenssen, M. Elliott, R. Townsend, A. Nijnik, S. F. Lee, V. Gerdts, L. A. Babiuk, S. A. Halperin and R. E. W. Hancock. 2009. A novel vaccine adjuvant comprised of a synthetic innate defence regulator peptide and CpG oligonucleotide links innate and adaptive immunity. Vaccine 27:4662-4671.;
Polewicz, M., A. Gracia, S. Garlapati, J. van Kessel, S. Strom, S. A. Halperin, R. E. W. Hancock, A. A. Potter, L. A. Babiuk, and V. Gerdts. 2013. Novel vaccine formulations against pertussis offer earlier onset of immunity and provide protection in the presence of maternal antibodies. Vaccine. 2013 PMID: 23684829.;
Steinstraesser, L., T. Hirsch, M. Schulte, M. Kueckelhaus, F. Jacobsen, E. A. Mersch, I. Stricker, N. Afacan, H. Jenssen, R. E. W. Hancock and J. Kindrachuk. 2012. Innate defense regulator peptide 1018 in wound healing and wound infection. PLoS ONE 7:e39373
Engel T. Basic Overview of Chemoinformatics. Journal of Chemical Information and Modelling, 46:2267-2277, 2006
Cherkasov, A., K. Hilpert, H. Jenssen, C. D. Fjell, M. Waldbrook, S. C. Mullaly, R. Volkmer and R. E. W. Hancock. 2009. Use of artificial intelligence in the design of small peptide antibiotics effective against a broad spectrum of highly antibiotic resistant Superbugs. ACS Chemical Biol. 4:65-74
Frank, R. 1992. Spot synthesis: an easy technique for the positionally addressable, parallel chemical synthesis on a membrane support. Tetrahedron. 48:9217-9232;
Hilpert K, R Volkmer-Engert, T Walter, and R E W Hancock. 2005. High-throughput generation of small antibacterial peptides with improved activity. Nature Biotech 23:1008-1012;
Stewart P S, and J W Costerton. 2001. Antibiotic resistance of bacteria in biofilms. Lancet 358:135-138.;
Høiby N, T Bjarnsholt, M Givskov, S Molin, and O Ciofu. 2010. Antibiotic resistance of bacterial biofilms. International Journal of Antimicrobial 35:322-32.
J. Biol. Chem., 243:3557-59, (1969);
Ron, et al., Biol Chem., 268: 2984-2988, 1993
Bowie, et al., Science, 247: 1306-1310, 1994;
Spatola (1983) in Chemistry and Biochemistry of Amino Acids, Peptides and Proteins, Vol. 7, pp 267-357, “Peptide Backbone Modifications,” Marcell Dekker, NY;
Organic Syntheses Collective Volumes, Gilman, et al. (Eds) John Wiley & Sons, Inc., NY
al-Obeidi, Mol. Biotechnol. 9: 205-223, 1998; Hruby, Curr. Opin. Chem. Biol. 1: 114-119, 1997;
Ostergaard, Mol. Divers. 3: 17-27, 1997; Ostresh, Methods Enzymol. 267: 220-234, 1996;
Belousov, Nucleic Acids Res. 25: 3440-3444, 1997;
Frenkel, Free Radic. Biol. Med. 19: 373-380, 1995; Blommers, Biochemistry 33: 7886-7896, 1994;
Caruthers, Nucleic Acids Res. Symp. Ser. 215-223, 1980; Horn, Nucleic Acids Res. Symp. Ser. 225-232, 1980;
Banga, Therapeutic Peptides and Proteins, Formulation, Processing and Delivery Systems Technomic Publishing Co., Lancaster, Pa., 1995;
Roberge, Science 269: 202, 1995;
Merrifield, Methods Enzymol. 289: 3-13, 1997;
Coligan, et al., Current Protocols in Immunology, Wiley Interscience, 1991, Unit 9;
Merrifield, J. Am. Chem. Soc., 85:2149, (1962);
Stewart and Young, Solid Phase Peptides Synthesis, (Freeman, San Francisco, 1969, pp. 27-62;
Powell et al., Pharma. Res. 10: 1268-1273, 1993;
Dean, BioEssays, 16: 683-687, 1994;
Cohen and Shatzmiller, J. Mol. Graph., 11: 166-173, 1993;
Wiley and Rich, Med. Res. Rev., 13: 327-384, 1993;
Moore, Trends Pharmacol. Sci., 15: 124-129, 1994;
Hruby, Biopolymers, 33: 1073-1082, 1993;
Bugg et al., Sci. Am., 269: 92-98, 1993;
Couder, et al., Int. J. Polypeptide Protein Res. 41: 181-184, 1993;
Dalpozzo, et al., Int. J. Polypeptide Protein Res. 41: 561-566;
Simon, et al., Proc. Natl. Acad. Sci. USA, 89: 9367-9371, 1992;
Jay, et al., Nuc. Acid Res., 11:2325, 1983;
Schall, 1991, Cytokine 3:165-183;
Horuk, R., 1994, Trends Pharmacol. Sci, 15:159-165;
Murphy, P. M., 1994, Annu. Rev. Immunol., 12:593-633;
Gong et al., 1996 J. Biol. Chem. 271:10521-10527;
Proudfoot et al., 1996 J. Biol. Chem. 271:2599-2603;
Beall et al., 1992, J. Biol. Chem. 267:3455-3459;
Yoshida et al., 1995, FEBS Lett. 360:155-159;
Kelner et al., 1994, Science 266:1395-1399;
Neote et al., 1993, Cell 72: 415-425;
Power et al., 1995, J. Biol. Chem. 270:19495-19500
Alkhatib et al., 1996, Science,
Dragic et al., 1996, Nature 381:667-674
Horuk et al., 1994, J. Biol. Chem. 269:17730-17733;
Neote et al., 1994, Blood 84:44-52;
Neote et al., 1993, J. Biol. Chem. 268:12247-12249
Berge, et al., J. Pharm. Sci., 66: 1-19, 1977
Remington's Pharmaceutical Sciences, 18th eds., Mack Publishing, Easton Pa. (1990);
Introduction to Pharmaceutical Dosage Forms, 4th ed., Lea & Febiger, Philadelphia (1985).
Strejan, et al., J. Neuroimmunol., 7: 27, 1984
Sustained and Controlled Release Drug Delivery Systems, J. R. Robinson, Ed., 1978, Marcel Dekker, Inc., New York
Egleton, Peptides 18: 1431-1439, 1997;
Langer Science 249: 1527-1533, 1990
Ranade, J. Clin. Pharmacol., 29: 685, 1989
Umezawa, et al., Biochem. Biophys. Res. Commun., 153: 1038, 1988
Bloeman, et al., FEBS Lett., 357: 140, 1995;
Owais, et al., Antimicrob. Agents Chemother., 39: 180, 1995
Briscoe, et al., Am. J. Physiol., 1233: 134, 1995
Schreier, et al., J. Biol. Chem., 269: 9090, 1994
Keinanen, et al., FEBS Lett., 346: 123, 1994;
Killion, et al., Immunomethods, 4: 273, 1994
Hanes, Advanced Drug Delivery Reviews 28: 97-119, 1997
Paul et al., Eur. J. Immunol. 25: 3521-24, 1995;
Cevc et al., Biochem. Biophys. Acta 1368: 201-15, 1998
Kramer A, Schuster A, Reinecke U, Malin R, Volkmer-Engert R, Landgraf C, Schneider-Mergener J. 1994. Combinatorial cellulose-bound peptide libraries: screening tool for the identification of peptides that bind ligands with predefined specificity. Comp. Meth. Enzymol. 6, 388-395;
Kramer A, Keitel T, Winkler K, Stocklein W, Hohne W, Schneider-Mergener J. 1997. Molecular basis for the binding promiscuity of an anti-p24 (HIV-1) monoclonal antibody. Cell 91, 799-809)

All publications and patent documents cited herein are hereby incorporated by reference in their entirety for all purposes to the same extent as if each were so individually denoted.

Although the foregoing invention has been described in detail by way of example for purposes of clarity of understanding, it will be apparent to the artisan that certain changes and modifications are comprehended by the disclosure and can be practiced without undue experimentation within the scope of the embodiments, which are presented by way of illustration not limitation.

1. An isolated antibiofilm or immunomodulatory peptide comprising 7 to 14 amino acids, wherein the antibiofilm or immunomodulatory peptide comprises an amino acid sequence as set forth in one or more of SEQ ID NOs: 151, 6-150, 152-1085 or a functional variant thereof.

2. An isolated polynucleotide encoding the peptide of claim 1.

3. The isolated antibiofilm or immunomodulatory peptide of claim 1, comprising an amino acid sequence as set forth in one or more of SEQ ID NOs: 25, 29, 30, 38, 39, 44, 45, 46, 57, 60, 65, 151 or 152 or a functional variant thereof.

4. The isolated antibiofilm or immunomodulatory peptide of claim 3, comprising a non-natural amino acid equivalent.

5. The isolated antibiofilm or immunomodulatory peptide of claim 4, wherein the non-natural amino acid equivalent is L-2-amino-3-guanidinopropionic acid, L-2-Amino-4-guanidinobutyric acid, L-Homoarginine, L-2,3-diaminopropionic acid or L-Ornithine.

6. A polypeptide represented by X1-A-X2, wherein A comprises the antibiofilm or immunomodulatory peptide of claim 1; and wherein each of X1 and X2 independently comprises an amino acid sequence of n amino acids, wherein n is 0 to 50.

7. The polypeptide of claim 6 wherein the functional variant comprises a conservative amino acid substitution or peptide mimetic substitution having about 90% or greater amino acid sequence identity to the amino acid sequence as set forth in one or more of SEQ ID NOs: 6-1085.

8. An antibiofilm or immunomodulatory peptide as set forth in Formula 1: [chemical structure]
wherein:
Z₁, Z₄, Z₆ and Z₉ are each independently H, methyl-1H-indol-3-yl, isopropyl, methyl, 2-methylpropyl, or 1-methylpropyl;
B₃ is propyl-3-guanidine or α-aminobutyl;
J₅, and J₈ are each independently H, methyl-1H-indol-3-yl, isopropyl, methyl, 2-methylpropyl, 1-methylpropyl; propyl-3-guanidine, α-aminobutyl, propyl-3-guanidine, α-aminobutyl, or propyl-3-carboxamide;
U₂ is H, methyl-1H-indol-3-yl, isopropyl, methyl, 2-methylpropyl, 1-methylpropyl, or propyl-3-carboxamide;
Σ₁₀ is propyl-3-guanidine, α-aminobutyl, or propyl-3-carboxamide;
X₁ and X₂ are each independently 0 to 2 amino acids selected from the group consisting of 2-amino-3-(1h-indol-3-yl)propanoic acid, 2-amino-3-methylbutanoic acid, 2-aminopropanoic acid, 2-amino-4-methylpentanoic acid, 2-amino-3-methylpentanoic acid, aminoacetic acid, 2-amino-5-guanidinopentanoic acid, or 2,6-diaminohexanoic acid; and
wherein the peptide can also contain one substitution from the group Z₁=α-aminobutyl, B₃=2-methylpropyl, Z₆=propyl-3-guanidine, W₇ is H, methyl-1H-indol-3-yl, isopropyl, methyl, 2-methylpropyl, 1-methylpropyl, or propyl-3-carboxamide and Σ₁₀ is methyl.

9. A method of inhibiting the growth of a bacterial biofilm or an abscess, or of enhancing innate immunity, or of selectively suppressing a proinflammatory response, comprising contacting the bacterial biofilm or abscess or a cell with an inhibition effective amount of a peptide of comprising the antibiofilm or immunomodulatory peptide of claim 1.

10. The method of claim 9, further comprising contacting the bacterial biofilm or abscess with the inhibiting effective amount of the peptide in combination with at least one antibiotic.

11. The method of claim 9, wherein the peptide is bound to a solid support or surface.

12. The method of claim 9, wherein the antibiofilm or immunomodulatory peptide comprises an amino acid sequence as set forth in one or more of SEQ ID NOs: 25, 29, 30, 38, 39, 44, 45, 46, 57, 60, 65, 151 or 152 or a functional variant thereof.

13. (canceled)

14. (canceled)

15. (canceled)

16. (canceled)

17. (canceled)

18. (canceled)

19. The method of claim 9, wherein the antibiofilm or immunomodulatory peptide comprises a non-natural amino acid equivalent.

20. The method of claim 19, wherein the non-natural amino acid equivalent is L-2-amino-3-guanidinopropionic acid, L-2-Amino-4-guanidinobutyric acid, L-Homoarginine, L-2,3-diaminopropionic acid or L-Ornithine.

21. The method of claim 9, wherein the functional variant comprises a conservative amino acid substitution or peptide mimetic substitution having about 90% or greater amino acid sequence identity to the amino acid sequence as set forth in one or more of SEQ ID NOs: 6-1085.

22. The method of claim 10, wherein the peptide is bound to a solid support or surface.

23. The method of claim 10, wherein the antibiofilm or immunomodulatory peptide comprises an amino acid sequence as set forth in one or more of SEQ ID NOs: 25, 29, 30, 38, 39, 44, 45, 46, 57, 60, 65, 151 or 152 or a functional variant thereof.

24. The method of claim 10, wherein the antibiofilm or immunomodulatory peptide comprises a non-natural amino acid equivalent.
